(19) United States
(12) Patent Application Publication
(10) Pub. No.: US 2026/0226076 A1
(43) Pub. Date: Aug. 6, 2026

(54) FUSED MULTICYCLIC COMPOUNDS AND THEIR USE AS PARP1 INHIBITORS

(71) Applicant: LAEKNA THERAPEUTICS SHANGHAI CO., LTD., Shanghai (CN)

(72) Inventors: Ming LI, Shanghai (CN); Yan CHEN, Shanghai (CN); Chun-Yen CHEN, Shanghai (CN); Xiang-Ju Justin GU, Shanghai (CN)

(21) Appl. No.: 19/128,527

(22) PCT Filed: Nov. 8, 2023
(86) PCT No.: PCT/CN2023/130493

§ 371 Date: May 8, 2025

(30) Foreign Application Priority Data
Nov. 9, 2022 (WO) PCT/CN2022/130788
Feb. 15, 2023 (WO) PCT/CN2023/076158

(51) Int. Cl. C07D 519/00; A61K 31/444; A61K 31/473; A61K 31/4741; A61K 31/4745; A61K 31/498; A61K 31/4985; A61K 31/519; A61K 31/538; A61K 31/5383; A61P 35/00; C07D 487/04

(52) U.S. Cl. CPC C07D 519/00; A61K 31/444; A61K 31/473; A61K 31/4741; A61K 31/4745; A61K 31/498; A61K 31/4985; A61K 31/519; A61K 31/538; A61K 31/5383; A61P 35/00; C07D 487/04

(57) ABSTRACT

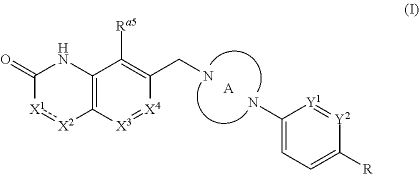

Provided herein are certain fused multicyclic compounds with a multicyclic linker, such as a compound of Formula (I), as PARP1 inhibitors, pharmaceutical compositions comprising the compounds, and method of use of the compounds or pharmaceutical compositions in the treatment of diseases or disorders.

### CROSS-REFERENCE TO RELATED APPLICATIONS

This application claims priority to International Patent Application No. PCT/CN2022/130788 filed on Nov. 9, 2022 and International Patent Application No. PCT/CN2023/076158 filed on Feb. 15, 2023, the entirely of each of which is incorporated herein by reference.

### FIELD

Provided herein are certain fused multicyclic compounds with a multicyclic linker as PARP1 inhibitors, pharmaceutical compositions comprising the compounds, and method of use of the compounds or pharmaceutical compositions in the treatment of diseases or disorders.

### BACKGROUND

Poly (ADP-ribose) polymerase enzymes (PARPs) catalyze the poly ADP-ribosylation (PARylation), with nicotinamide adenine dinucleotide (NAD) serving as a substrate. The DNA damage-induced parylation is largely mediated by PARP1 (also known as ARTD1), which is mainly activated by DNA single and double strand breaks and accounts for >90% of DNA damage-induced PARylation (Nucleic acids research 44, 10386-10405).

Auto-PARylation by the catalytic activity of PARP releases PARP from the DNA, allowing access to additional repair proteins and resolution of DNA breaks and stalled replication forks (Sci. Transl. Med. 8 (362)(2016)362ps17.). Inhibition of PARP family enzymes has been explored as a strategy for selective killing of cancer cells by inactivation of complementary DNA repair pathways. The PARP inhibitors (PARPi) that is now used in clinic inhibit PARylation through competitive binding to NAD+ binding site of PARP1 and PARP2, which has improved clinical benefits for BRCA mutated tumors. In addition to catalytic inhibition, “PARP trapping” is another important mechanism involving PARPi. It has been reported that PARPi are more cytotoxic than PARP depletion because of their ability to trap PARP enzymes on damaged DNA that interferes with DNA replication (Cancer Res. 2012; 72:5588-5599).

BRCA1 and BRCA2 play essential roles in DNA replication and double-strand breaks (DSB) repair. Both factors promote homologous recombination repair (HR), a DNA repair pathway active during S/G2 phases of the cell cycle, which also provides a pathway to restart the stalled replication forks. Several studies have shown that BRCA-deficient cells, and more broadly, cells with HR deficiency (HRD), appear to be highly sensitive to PARP inhibition (Nat Rev Cancer 2004; 4:814-9).

Hematological toxicities are common adverse events involving PARPi therapy. PARP2 has been shown to be required for hematopoietic stem/progenitor cells (HSPC) survival under steady-state conditions and in response to stress (Blood. 2013 Jul. 4; 122 (1): 44-54.). It is believed that PARP inhibitors having improved selectivity for PARP1 may possess improved efficacy and reduced toxicity compared to other clinical PARP1/2 inhibitors. It is believed that selective potent inhibition of PARP1 would lead to trapping of PARP1 on DNA, resulting in DNA double-strand breaks (DSBs) through collapse of replication forks in S-phase. It is also believed that PARP1-DNA trapping is an effective mechanism for selectivity killing of tumor cells having HRD.

Therefore, there exists an unmet medical need for effective and safe PARP inhibitors, especially PARP inhibitors having selectivity for PARP1.

### SUMMARY

In one embodiment, provided herein are fused multicyclic compounds with a multicyclic linker as PARP1 inhibitors.

In one embodiment, provided herein are compounds of Formula (I):

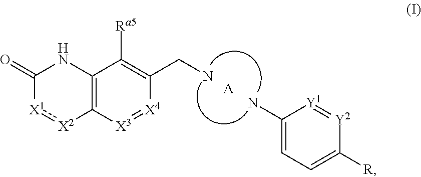

or a stereoisomer, or a mixture of stereoisomers thereof, or a pharmaceutically acceptable salt thereof, wherein [symbol] , X¹, X², X³, X⁴, Ras, Ring A, Y¹, Y², and R are as defined herein or elsewhere.

Also provided herein are pharmaceutical compositions comprising a compound provided herein and a pharmaceutically acceptable excipient.

Also provided herein are methods of inhibiting a PARPI protein, comprising contacting the PARP1 protein with a compound provided herein or a pharmaceutical composition provided herein.

Also provided herein are methods of treating PARP1 mediated disease or cancer, comprising administering to a subject having the disease or cancer a therapeutically effective amount of a compound provided herein or a pharmaceutical composition provided herein.

### DETAILED DESCRIPTION

### Definitions

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

As used herein, and in the specification and the accompanying claims, the indefinite articles “a” and “an” and the definite article “the” include plural as well as single referents, unless the context clearly indicates otherwise.

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments.

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

As used herein, the phrase “and/or” as used in a phrase such as “A and/or B” herein is intended to include both A and B; A or B; A (alone); and B (alone). Likewise, the phrase “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

It should be noted that if there is a discrepancy between a depicted structure and a name for that structure, the depicted structure is to be accorded more weight.

As used herein, and unless otherwise specified, the term “alkyl” refers to a straight or branched hydrocarbon chain radical consisting solely of carbon and hydrogen atoms, which is saturated. In one embodiment, the alkyl group has, for example, from one to twenty-four carbon atoms (C₁-C₂₄ alkyl), four to twenty carbon atoms (C₄-C₂₀ alkyl), six to sixteen carbon atoms (C₆-C₁₆ alkyl), six to nine carbon atoms (C₆-C₉ alkyl), one to fifteen carbon atoms (C₁-C₁₅ alkyl), one to twelve carbon atoms (C₁-C₁₂ alkyl), one to eight carbon atoms (C₁-C₈alkyl) or one to six carbon atoms (C₁-C₆ alkyl) and which is attached to the rest of the molecule by a single bond. Examples of alkyl groups include, but are not limited to, methyl, ethyl, n-propyl, 1-methylethyl (isopropyl), n-butyl, n-pentyl, 1,1-dimethylethyl (t-butyl), 3-methylhexyl, 2-methylhexyl, and the like. Unless otherwise specified, an alkyl group is optionally substituted.

As used herein, and unless otherwise specified, the term “alkenyl” refers to a straight or branched hydrocarbon chain radical consisting solely of carbon and hydrogen atoms, which contains one or more carbon-carbon double bonds. The term “alkenyl” also embraces radicals having “cis” and “trans” configurations, or alternatively, “E” and “Z” configurations, as appreciated by those of ordinary skill in the art. In one embodiment, the alkenyl group has, for example, from two to twenty-four carbon atoms (C₂-C₂₄ alkenyl), four to twenty carbon atoms (C₄-C₂₀ alkenyl), six to sixteen carbon atoms (C₆-C₁₆ alkenyl), six to nine carbon atoms (C₆-C₉ alkenyl), two to fifteen carbon atoms (C₂-C₁₅ alkenyl), two to twelve carbon atoms (C₂-C₁₂ alkenyl), two to eight carbon atoms (C₂-C₈ alkenyl) or two to six carbon atoms (C₂-C₆ alkenyl) and which is attached to the rest of the molecule by a single bond. Examples of alkenyl groups include, but are not limited to, ethenyl, prop-1-enyl, but-1-enyl, pent-1-enyl, penta-1,4-dienyl, and the like. Unless otherwise specified, an alkenyl group is optionally substituted.

As used herein, and unless otherwise specified, the term “alkynyl” refers to a straight or branched hydrocarbon chain radical consisting solely of carbon and hydrogen atoms, which contains one or more carbon-carbon triple bonds. In one embodiment, the alkynyl group has, for example, from two to twenty-four carbon atoms (C₂-C₂₄ alkynyl), four to twenty carbon atoms (C₄-C₂₀ alkynyl), six to sixteen carbon atoms (C₆-C₁₆ alkynyl), six to nine carbon atoms (C₆-C₉ alkynyl), two to fifteen carbon atoms (C₂-C₁₅ alkynyl), two to twelve carbon atoms (C₂-C₁₂ alkynyl), two to eight carbon atoms (C₂-C₈ alkynyl) or two to six carbon atoms (C₂-C₆ alkynyl) and which is attached to the rest of the molecule by a single bond. Examples of alkynyl groups include, but are not limited to, ethynyl, propynyl, butynyl, pentynyl, and the like. Unless otherwise specified, an alkynyl group is optionally substituted.

As used herein, and unless otherwise specified, the term “cycloalkyl” refers to a non-aromatic monocyclic or polycyclic hydrocarbon radical consisting solely of carbon and hydrogen atoms, and which is saturated. Cycloalkyl group may include fused, bridged, or spiro ring systems. In one embodiment, the cycloalkyl has, for example, from 3 to 15 ring carbon atoms (C₃-C₁₅ cycloalkyl), from 3 to 10 ring carbon atoms (C₃-C₁₀ cycloalkyl), or from 3 to 8 ring carbon atoms (C₃-C₈ cycloalkyl). The cycloalkyl is attached to the rest of the molecule by a single bond. Examples of monocyclic cycloalkyl radicals include, but are not limited to, cyclopropyl, cyclobutyl, cyclopentyl, cyclohexyl, cycloheptyl, and cyclooctyl. Examples of polycyclic cycloalkyl radicals include, but are not limited to, adamantyl, norbornyl, decalinyl, 7,7-dimethyl-bicyclo[2.2.1]heptanyl, and the like. Unless otherwise specified, a cycloalkyl group is optionally substituted.

As used herein, and unless otherwise specified, the term “cycloalkenyl” refers to a non-aromatic monocyclic or polycyclic hydrocarbon radical consisting solely of carbon and hydrogen atoms, and which includes one or more carbon-carbon double bonds. Cycloalkenyl may include fused, bridged, or spiro ring systems. In one embodiment, the cycloalkenyl has, for example, from 3 to 15 ring carbon atoms (C₃-C₁₅ cycloalkenyl), from 3 to 10 ring carbon atoms (C₃-C₁₀ cycloalkenyl), or from 3 to 8 ring carbon atoms (C₃-C₈ cycloalkenyl). The cycloalkenyl is attached to the rest of the molecule by a single bond. Examples of monocyclic cycloalkenyl radicals include, but are not limited to, cyclopropenyl, cyclobutenyl, cyclopentenyl, cyclohexenyl, cycloheptenyl, cyclooctenyl, and the like. Unless otherwise specified, a cycloalkenyl group is optionally substituted. Similarly, as used herein, and unless otherwise specified, the term “cycloalkynyl” refers to a non-aromatic monocyclic or polycyclic hydrocarbon radical consisting solely of carbon and hydrogen atoms, and which includes one or more carbon-carbon triple bonds.

As used herein, and unless otherwise specified, the term “heteroalkyl” refers to an alkyl radical that has one or more skeletal chain atoms selected from an atom other than carbon, e.g., oxygen, nitrogen, sulfur, and phosphorus, or combinations thereof. A numerical range can be given to refer to the chain length in total. For example, a —CH₂OCH₂CH₃ radical is referred to as a “C4” heteroalkyl. Connection to the parent molecular structure can be through either a heteroatom or a carbon in the heteroalkyl chain. One or more heteroatom(s) in the heteroalkyl radical can be optionally oxidized. One or more nitrogen atoms, if present, can also be optionally quaternized. Unless otherwise specified, a heteroalkyl group is optionally substituted.

As used herein, and unless otherwise specified, the term “aryl” refers to a monocyclic aromatic group and/or multicyclic aromatic group that contain at least one aromatic hydrocarbon ring. In certain embodiments, the aryl has from 6 to 18 ring carbon atoms (C₆-C₁₈ aryl), from 6 to 14 ring carbon atoms (C₆-C₁₄ aryl), or from 6 to 10 ring carbon atoms (C₆-C₁₀ aryl). Examples of aryl groups include, but are not limited to, phenyl, naphthyl, fluorenyl, azulenyl, anthryl, phenanthryl, pyrenyl, biphenyl, and terphenyl. The term “aryl” also refers to bicyclic, tricyclic, or other multicyclic hydrocarbon rings, where at least one of the rings is aromatic and the others of which may be saturated, partially unsaturated, or aromatic, for example, dihydronaphthyl, indenyl, indanyl, or tetrahydronaphthyl (tetralinyl). Unless otherwise specified, an aryl group is optionally substituted.

As used herein, and unless otherwise specified, the term “heteroaryl” refers to a monocyclic aromatic group and/or multicyclic aromatic group that contains at least one aromatic ring, wherein at least one aromatic ring contains one or more (e.g., one, one or two, one to three, or one to four) heteroatoms independently selected from O, S, and N. The heteroaryl may be attached to the main structure at any heteroatom or carbon atom. In certain embodiments, the heteroaryl has from 5 to 20, from 5 to 15, or from 5 to 10 ring atoms. The term “heteroaryl” also refers to bicyclic, tricyclic, or other multicyclic rings, where at least one of the rings is aromatic and the others of which may be saturated, partially unsaturated, or aromatic, wherein at least one aromatic ring contains one or more heteroatoms independently selected from O, S, and N. Examples of monocyclic heteroaryl groups include, but are not limited to, pyrrolyl, pyrazolyl, pyrazolinyl, imidazolyl, oxazolyl, isoxazolyl, thiazolyl, thiadiazolyl, isothiazolyl, furanyl, thienyl, oxadiazolyl, pyridyl, pyrazinyl, pyrimidinyl, pyridazinyl, and triazinyl. Examples of bicyclic heteroaryl groups include, but are not limited to, indolyl, benzothiazolyl, benzoxazolyl, benzothienyl, quinolinyl, tetrahydroisoquinolinyl, isoquinolinyl, benzimidazolyl, benzopyranyl, indolizinyl, benzofuranyl, isobenzofuranyl, chromonyl, coumarinyl, cinnolinyl, quinoxalinyl, indazolyl, purinyl, pyrrolopyridinyl, furopyridinyl, thienopyridinyl, dihydroisoindolyl, and tetrahydroquinolinyl. Examples of tricyclic heteroaryl groups include, but are not limited to, carbazolyl, benzindolyl, phenanthrollinyl, acridinyl, phenanthridinyl, and xanthenyl. Unless otherwise specified, a heteroaryl group is optionally substituted.

As used herein, and unless otherwise specified, the term “heterocyclyl” refers to a monocyclic and/or multicyclic non-aromatic group that contains one or more (e.g., one, one or two, one to three, or one to four) heteroatoms independently selected from nitrogen, oxygen, phosphorous, and sulfur. The heterocyclyl may be attached to the main structure at any heteroatom or carbon atom. A heterocyclyl group can be a monocyclic, bicyclic, tricyclic, tetracyclic, or other multicyclic ring system, wherein the multicyclic ring systems can be a fused, bridged or spiro ring system. Heterocyclyl multicyclic ring systems can include one or more heteroatoms in one or more rings. A heterocyclyl group can be saturated or partially unsaturated. Saturated heterocyclyl groups can be termed “heterocycloalkyl”. Partially unsaturated heterocyclyl groups can be termed “heterocycloalkenyl” if the heterocyclyl contains at least one double bond, or “heterocycloalkynyl” if the heterocyclyl contains at least one triple bond. In one embodiment, the heterocyclyl has, for example, 3 to 18 ring atoms (3- to 18-membered heterocyclyl), 4 to 18 ring atoms (4- to 18-membered heterocyclyl), 5 to 18 ring atoms (5- to 18-membered heterocyclyl), 4 to 8 ring atoms (4- to 8-membered heterocyclyl), or 5 to 8 ring atoms (5- to 8-membered heterocyclyl). Examples of heterocyclyl groups include, but are not limited to, imidazolidinyl, oxazolidinyl, thiazolidinyl, pyrazolidinyl, isoxazolidinyl, isothiazolidinyl, morpholinyl, pyrrolidinyl, tetrahydrofuryl, and piperidinyl. Unless otherwise specified, a heterocyclyl group is optionally substituted.

Whenever it appears herein, a numerical range such as “3 to 18” refers to each integer in the given range; e.g., a heterocyclyl with “3 to 18 ring atoms” means that the heterocyclyl group can consist of 3 ring atoms, 4 ring atoms, 5 ring atoms, 6 ring atoms, 7 ring atoms, 8 ring atoms, 9 ring atoms, 10 ring atoms, etc., up to and including 18 ring atoms. Similarly, a C₁-C₆ alkyl means that the alkyl group can consist of 1 carbon atom, 2 carbon atoms, 3 carbon atoms, 4 carbon atoms, 5 carbon atoms, and 6 carbon atoms.

As used herein and unless otherwise specified, a “cycloalkylalkyl” group is a radical of the formula: -alkyl-cycloalkyl, wherein alkyl and cycloalkyl are defined above. Substituted cycloalkylalkyl groups may be substituted at the alkyl, the cycloalkyl, or both the alkyl and the cycloalkyl portions of the group. Representative cycloalkylalkyl groups include but are not limited to cyclopropylmethyl, cyclobutylmethyl, cyclopentylmethyl, cyclohexylmethyl, cyclopropylethyl, cyclobutylethyl, cyclopentylethyl, cyclohexylethyl, cyclopentylpropyl, cyclohexylpropyl and the like.

As used herein and unless otherwise specified, an “aralkyl” group is a radical of the formula: -alkyl-aryl, wherein alkyl and aryl are defined above. Substituted aralkyl groups may be substituted at the alkyl, the aryl, or both the alkyl and the aryl portions of the group. Representative aralkyl groups include but are not limited to benzyl and phenethyl groups and aralkyl groups wherein the aryl group is fused to a cycloalkyl group such as indan-4-yl ethyl.

As used herein and unless otherwise specified, other similar composite terms mirror the above description for “cycloalkylalkyl” and “aralkyl”. For example, a “heterocyclylalkyl” group is a radical of the formula: -alkyl-heterocyclyl, wherein alkyl and heterocyclyl are defined above. A “heteroarylalkyl” group is a radical of the formula: -alkyl-heteroaryl, wherein alkyl and heteroaryl are defined above. A “heterocycloalkylalkyl” group is a radical of the formula: -alkyl-heterocycloalkyl, wherein alkyl and heterocycloalkyl are defined above.

As used herein, and unless otherwise specified, the term “halogen”, “halide” or “halo” refers to fluorine, chlorine, bromine, and/or iodine. As used herein, and unless otherwise specified, the terms “haloalkyl,” “haloalkenyl,” “haloalkynyl,” and “haloalkoxy” refer to alkyl, alkenyl, alkynyl, and alkoxy structures that are substituted with one or more halo groups or with combinations thereof.

As used herein, and unless otherwise specified, the term “alkoxy” refers to —O-(alkyl), wherein alkyl is defined above. As used herein, and unless otherwise specified, the term “aryloxy” refers to —O-(aryl), wherein aryl is defined above.

As used herein, and unless otherwise specified, the term “alkyl sulfonyl” refers to —SO₂-alkyl, wherein alkyl is defined above.

As used herein, and unless otherwise specified, the term “carboxyl” and “carboxy” refers to —COOH.

As used herein, and unless otherwise specified, the term “alkoxycarbonyl” refers to —C(═O)O-(alkyl), wherein alkyl is defined above. As used herein, and unless otherwise specified, the term “arylalkyloxy” refers to —O-(alkyl)-(aryl), wherein alkyl and aryl are defined above. As used herein, and unless otherwise specified, the term “cycloalkyloxy” refers to —O-(cycloalkyl), wherein cycloalkyl is defined above. As used herein, and unless otherwise specified, the term “cycloalkylalkyloxy” refers to —O-(alkyl)-(cycloalkyl), wherein cycloalkyl and alkyl are defined above.

As used herein, and unless otherwise specified, the term “acyl” refers to —C(O)—R^{a}, wherein R^{a} can be, but is not limited to, hydrogen, alkyl, heteroalkyl, alkenyl, alkynyl, aryl, cycloalkyl, heteroaryl, heterocyclyl, each of which is defined above. In certain embodiments, R^{a} may be unsubstituted or substituted with one or more substituents.

As used herein, and unless otherwise specified, the term “acyloxy” refers to —O—C(O)—R^{a}, wherein R^{a} can be, but is not limited to, hydrogen, alkyl, heteroalkyl, alkenyl, alkynyl, aryl, cycloalkyl, heteroaryl, heterocyclyl, each of which is defined above. In certain embodiments, R^{a} may be unsubstituted or substituted with one or more substituents.

As used herein, and unless otherwise specified, the term “amino” refers to —N(R^{#})(R^{#}), wherein each R^{#} independently can be, but is not limited to, hydrogen, alkyl, heteroalkyl, alkenyl, alkynyl, aryl, cycloalkyl, heteroaryl, heterocyclyl, each of which is defined above. When a —N(R^{#})(R^{#}) group has two R^{#} other than hydrogen, they can be combined with the nitrogen atom to form a ring. In one embodiment, the ring is a 3-, 4-, 5-, 6-, 7-, or 8-membered ring. In one embodiment, one or more ring atoms are heteroatoms independently selected from O, S, or N. The term “amino” also includes N-oxide (—N⁺(R^{#})(R^{#})O⁻). In certain embodiments, each R^{#} or the ring formed by —N(R^{#})(R^{#}) independently may be unsubstituted or substituted with one or more substituents.

As used herein, and unless otherwise specified, the term “amide” or “amido” refers to —C(O)N(R^{#})₂ or —NR^{#}C(O)R^{#}, wherein each R^{#} independently can be, but is not limited to, hydrogen, alkyl, heteroalkyl, alkenyl, alkynyl, aryl, cycloalkyl, heteroaryl, heterocyclyl, each of which is defined above. When a —C(O)N(R^{#})₂ group has two R^{#} other than hydrogen, they can be combined with the nitrogen atom to form a ring. In one embodiment, the ring is a 3-, 4-, 5-, 6-, 7-, or 8-membered ring. In one embodiment, one or more ring atoms are heteroatoms independently selected from O, S, or N. In certain embodiments, each R^{#} or the ring formed by —N(R^{#})(R^{#}) independently may be unsubstituted or substituted with one or more substituents.

As used herein, and unless otherwise specified, the term “aminoalkyl” refers to -(alkyl)-(amino), wherein alkyl and amino are defined above. As used herein, and unless otherwise specified, the term “aminoalkoxy” refers to —O-(alkyl)-(amino), wherein alkyl and amino are defined above.

As used herein, and unless otherwise specified, the term “alkylamino” refers to —NH(alkyl) or —N(alkyl)(alkyl), wherein alkyl is defined above. Examples of such alkylamino groups include, but are not limited to, —NHCH₃, —NHCH₂CH₃, —NH(CH₂)₂CH₃, —NH(CH₂)₃CH₃, —NH(CH₂)₄CH₃, —NH(CH₂)₅CH₃, —N(CH₃)₂, —N(CH₂CH₃)₂, —N((CH₂)₂CH₃)₂, —N(CH₃)(CH₂CH₃), and the like.

As used herein, and unless otherwise specified, the term “arylamino” refers to —NH(aryl) or —N(aryl)(aryl), wherein aryl is defined above. As used herein, and unless otherwise specified, similar composite terms such as “arylalkylamino” and “cycloalkylamino” mirrors the descriptions above for “alkylamino” and “arylamino”.

As used herein, and unless otherwise specified, the term “sulfanyl”, “sulfide”, or “thio” refers to —S—R^{a}, wherein R^{a} can be, but is not limited to, alkyl, heteroalkyl, alkenyl, alkynyl, aryl, cycloalkyl, heteroaryl, heterocyclyl, each of which is defined above. In certain embodiments, R^{a} may be unsubstituted or substituted with one or more substituents.

As used herein, and unless otherwise specified, the term “sulfoxide” refers to —S(O)—R^{a}, wherein R^{a} can be, but is not limited to, alkyl, heteroalkyl, alkenyl, alkynyl, aryl, cycloalkyl, heteroaryl, heterocyclyl, each of which is defined above. In certain embodiments, R^{a} may be unsubstituted or substituted with one or more substituents.

As used herein, and unless otherwise specified, the term “sulfonyl” or “sulfone” refers to —S(O)₂—R^{a}, wherein R^{a} can be, but is not limited to, alkyl, heteroalkyl, alkenyl, alkynyl, aryl, cycloalkyl, heteroaryl, heterocyclyl, each of which is defined above. In certain embodiments, R^{a} may be unsubstituted or substituted with one or more substituents.

As used herein, and unless otherwise specified, the term “sulfonamido” or “sulfonamide” refers to —S(═O)₂—N(R″)₂ or —N(R^{#})—S(═O)₂—R^{#}, wherein each R^{#} independently can be, but is not limited to, hydrogen, alkyl, heteroalkyl, alkenyl, alkynyl, aryl, cycloalkyl, heteroaryl, heterocyclyl, each of which is defined above. When a —S(═O)₂—N(R^{#})₂ group has two R^{#} other than hydrogen, they can be combined with the nitrogen atom to form a ring. In one embodiment, the ring is a 3-, 4-, 5-, 6-, 7-, or 8-membered ring. In one embodiment, one or more ring atoms are heteroatoms independently selected from O, S, or N. In certain embodiments, each R^{#} or the ring formed by —N(R^{#})(R^{#}) independently may be unsubstituted or substituted with one or more substituents.

“Azide” refers to a —N₃ radical.

“Cyano” refers to a —CN radical.

“Nitro” refers to the —NO₂ radical.

“Oxa” refers to the —O— radical.

“Oxo” refers to the ═O radical.

As used herein, and unless otherwise specified, the term “optional” or “optionally” (e.g., optionally substituted) means that the subsequently described event of circumstances may or may not occur, and that the description includes instances where said event or circumstance occurs and instances in which it does not. For example, “optionally substituted alkyl” means that the alkyl radical may or may not be substituted and that the description includes both substituted alkyl radicals and alkyl radicals having no substitution.

When the groups described herein are said to be “substituted,” they may be substituted with any appropriate substituent or substituents. Illustrative examples of substituents include, but are not limited to, those found in the exemplary compounds and embodiments provided herein, as well as halogen (chloro, iodo, bromo, or fluoro); alkyl; alkenyl; alkynyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aryloxyamine, aralkoxyamine; N-oxide; hydrazine; hydrazide; hydrazone; azide; isocyanate; isothiocyanate; cyanate; thiocyanate; oxo (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrimidinyl, benzimidazolyl, benzothiophenyl, or benzofuranyl) aryloxy; aralkyloxy; heterocyclyloxy; and heterocyclyl alkoxy.

As used herein, and unless otherwise specified, the term “isomer” refers to different compounds that have the same molecular formula. “Stereoisomers” are isomers that differ only in the way the atoms are arranged in space. “Atropisomers” are stereoisomers from hindered rotation about single bonds. “Enantiomers” are a pair of stereoisomers that are non-superimposable mirror images of each other. A mixture of a pair of enantiomers in any proportion can be known as a “racemic” mixture. “Diastereoisomers” are stereoisomers that have at least two asymmetric atoms, but which are not mirror-images of each other. The absolute stereochemistry can be specified according to the Cahn-Ingold-Prelog R—S system. When a compound is an enantiomer, the stereochemistry at each chiral carbon can be specified by either R or S. Resolved compounds whose absolute configuration is unknown can be designated (+) or (−) depending on the direction (dextro- or levorotatory) which they rotate plane polarized light at the wavelength of the sodium D line. However, the sign of optical rotation, (+) and (−), is not related to the absolute configuration of the molecule, R and S. Certain compounds described herein contain one or more asymmetric centers and can thus give rise to enantiomers, diastereomers, and other stereoisomeric forms that can be defined, in terms of absolute stereochemistry at each asymmetric atom, as (R)- or (S)-. The present chemical entities, pharmaceutical compositions and methods are meant to include all such possible isomers, including racemic mixtures, optically substantially pure forms and intermediate mixtures. Optically active (R)- and(S)-isomers can be prepared, for example, using chiral synthons or chiral reagents, or resolved using conventional techniques.

“Stereoisomers” can also include E and Z isomers, or a mixture thereof, and cis and trans isomers or a mixture thereof. In certain embodiments, a compound described herein is isolated as either the E or Z isomer. In other embodiments, a compound described herein is a mixture of the E and Z isomers.

Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

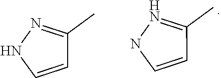

As used herein, and unless otherwise specified, the term “pharmaceutically acceptable salt” includes both acid and base addition salts.

Examples of pharmaceutically acceptable acid addition salts include, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

Examples of pharmaceutically acceptable base addition salt include, but are not limited to, salts prepared from addition of an inorganic base or an organic base to a free acid compound. Salts derived from inorganic bases include, but are not limited to, the sodium, potassium, lithium, ammonium, calcium, magnesium, iron, zinc, copper, manganese, aluminum salts and the like. In one embodiment, the inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. In one embodiment, the organic bases are isopropylamine, diethylamine, ethanolamine, trimethylamine, dicyclohexylamine, choline and caffeine.

As used herein, and unless otherwise specified, the term “subject” refers to an animal, including, but not limited to, a primate (e.g., human), cow, sheep, goat, horse, dog, cat, rabbit, rat, or mouse. The terms “subject” and “patient” are used interchangeably herein in reference, for example, to a mammalian subject, such as a human subject. In one embodiment, the subject is a mammal. In one embodiment, the subject is a human.

As used herein, and unless otherwise specified, the terms “treat,” “treating,” and “treatment” refer to the eradication or amelioration of a disease or disorder, or of one or more symptoms associated with the disease or disorder. In general, treatment occurs after the onset of the disease or disorder. In certain embodiments, the terms refer to minimizing the spread or worsening of the disease or disorder resulting from the administration of one or more prophylactic or therapeutic agents to a subject with such a disease or disorder.

As used herein, and unless otherwise specified, the terms “prevent,” “preventing,” and “prevention” refer to the prevention of the onset, recurrence or spread of a disease or disorder, or of one or more symptoms thereof. In general, prevention occurs prior to the onset of the disease or disorder.

As used herein, and unless otherwise specified, the terms “manage,” “managing,” and “management” refer to preventing or slowing the progression, spread or worsening of a disease or disorder, or of one or more symptoms thereof. Sometimes, the beneficial effects that a subject derives from a prophylactic or therapeutic agent do not result in a cure of the disease or disorder.

As used herein, and unless otherwise specified, the term “therapeutically effective amount” are meant to include the amount of a compound that, when administered, is sufficient to prevent development of, or alleviate to some extent, one or more of the symptoms of the disorder, disease, or condition being treated. The term “therapeutically effective amount” also refers to the amount of a compound that is sufficient to elicit the biological or medical response of a cell, tissue, system, animal, or human, which is being sought by a researcher, veterinarian, medical doctor, or clinician.

As used herein, and unless otherwise specified, the term “IC₅₀” refers an amount, concentration, or dosage of a compound that is required for 50% inhibition of a maximal response in an assay that measures such response.

As used herein, and unless otherwise specified, the term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition, Lippincott Williams & Wilkins: Philadelphia, PA, 2005; Handbook of Pharmaceutical Excipients, 5th Edition, Rowe et al., Eds., The Pharmaceutical Press and the American Pharmaceutical Association: 2005; and Handbook of Pharmaceutical Additives, 3rd Edition, Ash and Ash Eds., Gower Publishing Company: 2007; Pharmaceutical Preformulation and Formulation, Gibson Ed., CRC Press LLC: Boca Raton, FL, 2004.

Unless otherwise stated, structures depicted herein are also meant to include compounds which differ only in the presence of one or more isotopically enriched atoms. Examples of isotopes that can be incorporated into compounds provided herein include isotopes of hydrogen, carbon, nitrogen, oxygen, phosphorus, sulfur, fluorine, and chlorine, such as, e.g., ²H, ³H, ¹³C, ¹⁴C, ¹⁵N, ¹⁸O, ¹⁷O, ³¹P, ³²P, ³⁵S, ¹⁸F, and ³⁶Cl, respectively. For example, provided herein are compounds having the present structures except for the replacement or enrichment of a hydrogen by deuterium or tritium at one or more atoms in the molecule, or the replacement or enrichment of a carbon by ¹³C or ¹⁴C at one or more atoms in the molecule. In one embodiment, provided herein are isotopically labeled compounds having one or more hydrogen atoms replaced by or enriched by deuterium. In one embodiment, provided herein are isotopically labeled compounds having one or more hydrogen atoms replaced by or enriched by tritium. In one embodiment, provided herein are isotopically labeled compounds having one or more carbon atoms replaced or enriched by ¹³C. In one embodiment, provided herein are isotopically labeled compounds having one or more carbon atoms replaced or enriched by ¹⁴C.

As used herein, and unless otherwise specified, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

### Compounds

In one embodiment, provided herein are fused multicyclic compounds with a multicyclic linker as PARP1 inhibitors. In one embodiment, provided herein are bicyclic or tricyclic compounds with an octahydropyrrolo[3,4-c]pyrrole (in one particular embodiment, a specific stereoisomer of octahydropyrrolo[3,4-c]pyrrole) linker as PARP1 inhibitors.

In one embodiment, provided herein is a compound of Formula (I):

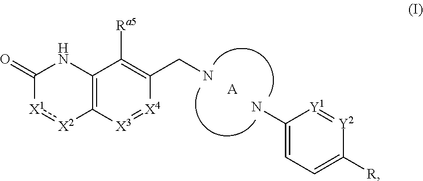

or a stereoisomer, or a mixture of stereoisomers thereof, or a pharmaceutically acceptable salt thereof, wherein:
[symbol] is a single bond or a double bond;
X¹ is CR^{a1}, C(R^{a1})₂, or NR^{a1};
X² is CR^{a2}, C(R^{a2})₂, N, NR^{a2}, or O;
each R^{a1} is independently hydrogen, C₁-C₆ alkyl, C₃-C₈ cycloalkyl, or C₁-C₆ alkoxy;
each R^{a2} is independently hydrogen, C₁-C₆ alkyl, C₃-C₈ cycloalkyl, or C₁-C₆ alkoxy;
or R^{a1} and R^{a2} together with the atoms to which they are attached to form a 3- to 6-membered Ring B;
X³ is CR^{a3} or N; R^{a3} is hydrogen, C₁-C₆ alkyl, or halogen;
X⁴ is CR^{a4} or N; R^{a4} is hydrogen, C₁-C₆ alkyl, or halogen;
R^{a5} is hydrogen, C₁-C₆ alkyl, or halogen;
Ring A is a fused, bridged or spiro heterocyclyl;
Y¹ is CR² or N;
Y² is CR² or N;
each R² is independently hydrogen, halogen, C₁-C₆ alkyl, C₁-C₆ haloalkyl, C₁-C₆ alkoxy, or C₃-C₈ cycloalkyl;
R is a halogen, —CN, C₁-C₆ alkyl, C₁-C₆ alkoxy, —C(═O)—R³, —C(═O)—OR³, —C(═O)—NHR³, —S(O)₂—R³, —NH—C(═O)—R³, or nitro;

R³ is hydrogen, C₁-C₆ alkyl, (C₁-C₆ alkoxy)-(C₁-C₆ alkyl), C₃-C₈ cycloalkyl, or 4- to 10-membered heterocyclyl; and
wherein the alkyl, cycloalkyl, alkoxy, heterocyclyl, Ring A, and Ring B are optionally substituted;
provided that, when

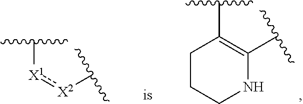

then (i) Ring A is a fused or spiro heterocyclyl; or (ii) R is —C(═O)—NHR³.

In one embodiment, provided herein is a compound of Formula (I):

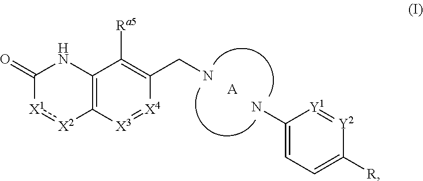

or a stereoisomer, or a mixture of stereoisomers thereof, or a pharmaceutically acceptable salt thereof, wherein:
[symbol] is a single bond or a double bond;
X¹ is CR^{a1}, C(R^{a1})₂, or NR^{a1};
X² is CR^{a2}, C(R^{a2})₂, N, NR^{a2}, or O;
each R^{a1} is independently hydrogen, C₁-C₆ alkyl, C₃-C₈ cycloalkyl, or C₁-C₆ alkoxy;
each R^{a2} is independently hydrogen, C₁-C₆ alkyl, C₃-C₈ cycloalkyl, or C₁-C₆ alkoxy;
or R^{a1} and R^{a2} together with the atoms to which they are attached to form a 3- to 6-membered Ring B;
X³ is CR^{a3} or N; R^{a3} is hydrogen, C₁-C₆ alkyl, or halogen;
X⁴ is CR^{a4} or N; R^{a4} is hydrogen, C₁-C₆ alkyl, or halogen;
R^{a5} is hydrogen, C₁-C₆ alkyl, or halogen;
Ring A is a fused, bridged or spiro heterocyclyl;
Y¹ is CR² or N;
Y² is CR² or N;
each R² is independently hydrogen, halogen, C₁-C₆ alkyl, C₁-C₆ haloalkyl, C₁-C₆ alkoxy, or C₃-C₈ cycloalkyl;
R is a halogen, —CN, C₁-C₆ alkyl, C₁-C₆ alkoxy, —C(═O)—R³, —C(═O)—OR³, —C(═O)—NHR³, —S(O)₂—R³, —NH—C(═O)—R³, or nitro;
R³ is hydrogen, C₁-C₆ alkyl, (C₁-C₆ alkoxy)-(C₁-C₆ alkyl), or C₃-C₈ cycloalkyl; and
wherein the alkyl, cycloalkyl, alkoxy, Ring A, and Ring B are optionally substituted;
provided that, when

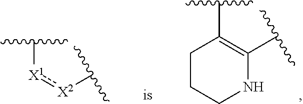

then (i) Ring A is a fused or spiro heterocyclyl; or (ii) R is —C(═O)—NHR³.

In one embodiment, X¹ is CR^{a1}. In one embodiment, X¹ is C—(C₁-C₆ alkyl). In one embodiment, X¹ is C—(C₃-C₈ cycloalkyl). In one embodiment, X¹ is C—(C₁-C₆ alkoxy). In one embodiment, X¹ is C-(n-propyl) or C-(iso-propyl). In one embodiment, X¹ is C-(n-butyl), C-(iso-butyl), or C-(tert-butyl). In one embodiment, X¹ is C—(C₅ alkyl). In one embodiment, X¹ is C—(C₆ alkyl). In one embodiment, X¹ is C-(cyclopropyl). In one embodiment, X¹ is C-(cyclobutyl). In one embodiment, X¹ is C-(cyclopentyl). In one embodiment, X¹ is C-(cyclohexyl). In one embodiment, X¹ is C-(cycloheptyl). In one embodiment, X¹ is C-(cyclooctyl). In one embodiment, X¹ is C-methoxyl. In one embodiment, X¹ is C-ethoxyl. In one embodiment, X¹ is C—(C₃ alkoxy). In one embodiment, X¹ is C—(C₄ alkoxy). In one embodiment, X¹ is C—(C₅ alkoxy). In one embodiment, X¹ is C—(C₆ alkoxy).

In one embodiment, X¹ is C(R^{a1})₂. In one embodiment, X¹ is C—(C₁-C₆ alkyl)₂. In one embodiment, X¹ is C—(C₃-C₈ cycloalkyl)₂. In one embodiment, X¹ is C—(C₁-C₆ alkoxy)₂.

In one embodiment, X¹ is NR^{a1}. In one embodiment, X¹ is N—(C₁-C₆ alkyl). In one embodiment, X¹ is N—(C₃-C₈ cycloalkyl). In one embodiment, X¹ is N—(C₁-C₆ alkoxy).

In one embodiment, X¹ is CH. In one embodiment, X¹ is C—CH₃. In one embodiment, X¹ is C—C₂H₅. In one embodiment, X¹ is C—OCH₃. In one embodiment, X¹ is NH. In one embodiment, X¹ is CH—CH₃. In one embodiment, X¹ is CH—C₂H₅.

In one embodiment, X² is CR^{a2}. In one embodiment, X² is C—(C₁-C₆ alkyl). In one embodiment, X² is C—(C₃-C₈ cycloalkyl). In one embodiment, X² is C—(C₁-C₆ alkoxy).

In one embodiment, X² is C-(n-propyl) or C-(iso-propyl). In one embodiment, X² is C-(n-butyl), C-(iso-butyl), or C-(tert-butyl). In one embodiment, X² is C—(C₅ alkyl). In one embodiment, X² is C—(C₆ alkyl). In one embodiment, X² is C-(cyclopropyl). In one embodiment, X² is C-(cyclobutyl). In one embodiment, X² is C-(cyclopentyl). In one embodiment, X² is C-(cyclohexyl). In one embodiment, X² is C-(cycloheptyl). In one embodiment, X² is C-(cyclooctyl). In one embodiment, X² is C-methoxyl. In one embodiment, X² is C-ethoxyl. In one embodiment, X² is C—(C₃ alkoxy). In one embodiment, X² is C—(C₄ alkoxy). In one embodiment, X² is C—(C₅ alkoxy). In one embodiment, X² is C—(C₆ alkoxy).

In one embodiment, X² is C(R^{a2})₂. In one embodiment, X² is C—(C₁-C₆ alkyl)₂. In one embodiment, X² is C—(C₃-C₈ cycloalkyl)₂. In one embodiment, X² is C—(C₁-C₆ alkoxy)₂.

In one embodiment, X² is NR^{a2}. In one embodiment, X² is N—(C₁-C₆ alkyl). In one embodiment, X² is N—(C₃-C₈ cycloalkyl). In one embodiment, X² is N—(C₁-C₆ alkoxy).

In one embodiment, X² is CH. In one embodiment, X² is N. In one embodiment, X² is O.

In one embodiment, X¹ is CR^{a1} and X² is CH. In one embodiment, X¹ is CR^{a1} and X² is N. In one embodiment, X¹ is CHR^{a1} and X² is O.

In one embodiment, X¹ is C—CH₃ and X² is CH. In one embodiment, X¹ is C—C₂H₅ and X² is CH. In one embodiment, X¹ is C—CH₃ and X² is N. In one embodiment, X¹ is C—C₂H₅ and X² is N. In one embodiment, X¹ is CH—CH₃ and X² is O. In one embodiment, X¹ is CH—C₂H₅ and X² is O.

In one embodiment, Y¹ is CR². In one embodiment, Y¹ is C-halogen. In one embodiment, Y¹ is C—(C₁-C₆ alkyl). In one embodiment, Y¹ is C—(C₁-C₆ alkoxy). In one embodiment, Y¹ is CH. In one embodiment, Y¹ is CF. In one embodiment, Y¹ is N.

In one embodiment, Y² is CR². In one embodiment, Y² is C-halogen. In one embodiment, Y² is C—(C₁-C₆ alkyl). In one embodiment, Y² is C—(C₁-C₆ alkoxy). In one embodiment, Y² is CH. In one embodiment, Y² is CF. In one embodiment, Y² is N.

In one embodiment, Y¹ is CR² and Y² is N. In one embodiment, Y¹ is N and Y² is CR². In one embodiment, Y¹ is CR² and Y² is CR². In one embodiment, Y¹ is N and Y² is N.

In one embodiment, Y¹ is CH and Y² is N. In one embodiment, Y¹ is CF and Y² is N. In one embodiment, Y¹ is N and Y² is CH. In one embodiment, Y¹ is N and Y² is CF.

In one embodiment, R is a halogen. In one embodiment, R is C₁-C₆ alkyl. In one embodiment, R is C₁-C₆ alkoxy. In one embodiment, R is —C(═O)—R³. In one embodiment, R is —C(═O)—OR³. In one embodiment, R is —S(O)₂—R³. In one embodiment, R is —NH—C(═O)—R³. In one embodiment, R is nitro. In one embodiment, R is —CN. In one embodiment, R is —C(═O)—NHR³. In one embodiment, R is —C(═O)—NHR³, and R³ is C₃-C₈ cycloalkyl or 4- to 10-membered heterocyclyl.

In one embodiment, R is —C(═O)—NH—(C₁-C₆ alkyl). In one embodiment, R is —C(═O)—NH—(C₃-C₈ cycloalkyl). In one embodiment, R is —C(═O)—NH—(C₃-C₆ cycloalkyl). In one embodiment, R is —C(═O)—NH—(4- to 10-membered heterocyclyl). In one embodiment, R is —C(═O)—NH—(4- to 6-membered heterocyclyl). In one embodiment, R is —C(═O)—NH—(4- to 6-membered oxygen-containing heterocyclyl). In one embodiment, R is —C(═O)—(C₁-C₆ alkyl). In one embodiment, R is —C(═O)—(C₃-C₈ cycloalkyl). In one embodiment, R is —C(═O)—O—(C₁-C₆ alkyl). In one embodiment, R is —C(═O)—O—(C₃-C₈ cycloalkyl). In one embodiment, R is —NH—C(═O)—(C₁-C₆ alkyl). In one embodiment, R is —NH—C(═O)—(C₃-C₈ cycloalkyl). In one embodiment, R is —NH—C(═O)-(4- to 10-membered heterocyclyl). In one embodiment, R is —NH—C(═O)-(4- to 6-membered heterocyclyl). In one embodiment, R is —S(O)₂—(C₁-C₆ alkyl). In one embodiment, R is —S(O)₂—(C₃-C₈ cycloalkyl). In one embodiment, R is C(═O)—NH₂. In one embodiment, R is C(═O)—NH—CH₃. In one embodiment, R is C(═O)—NH—CH₂CH₂OCH₃. In one embodiment, R is C(═O)—NH-cyclopropyl. In one embodiment, R is C(═O)—NH-cyclobutyl.

In one embodiment, R is selected from the group consisting of:

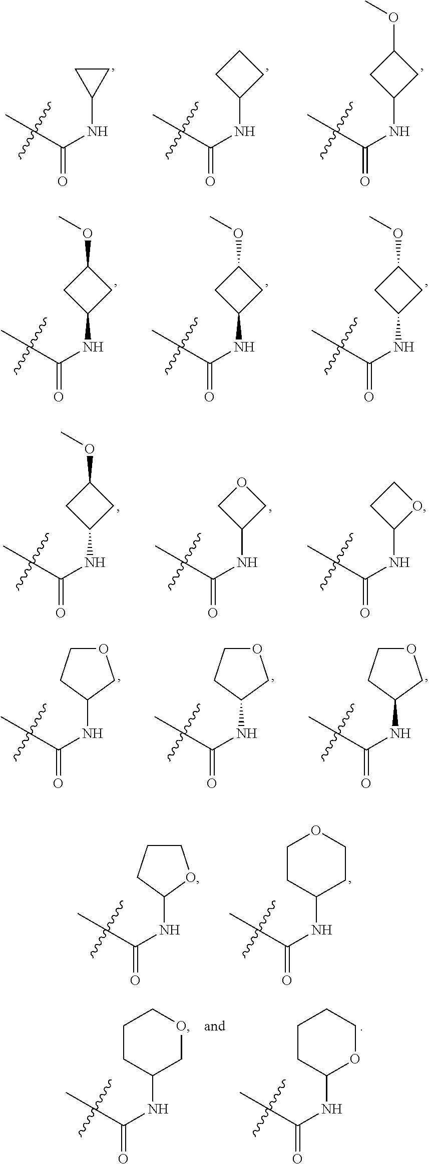

In one embodiment, provided herein is a compound of Formula (I-A):

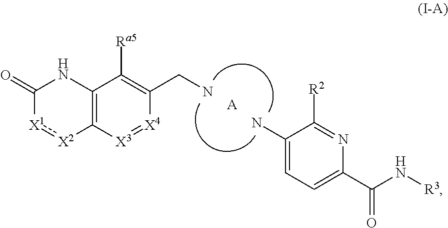

or a stereoisomer, or a mixture of stereoisomers thereof, or a pharmaceutically acceptable salt thereof.

In one embodiment, [symbol] is a double bond.

In one embodiment, [symbol] is a double bond and X¹ is CR^{a1}. In one embodiment, [symbol] is a double bond and X¹ is CH. In one embodiment, [symbol] is a double bond and X¹ is C—CH₃. In one embodiment, [symbol] is a double bond and X¹ is C—CH₂CH₃. In one embodiment, [symbol] is a double bond and X¹ is C—OCH₃.

In one embodiment, [symbol] is a double bond and X² is CR^{a2}. In one embodiment, [symbol] is a double bond and X² is CH. In one embodiment, [symbol] is a double bond and X² is C—CH₃. In one embodiment, [symbol] is a double bond and X² is C—CH₂CH₃. In one embodiment, [symbol] is a double bond and X² is N.

In one embodiment, [symbol] is a double bond, X¹ is CR^{a1} and X² is N. In one embodiment, [symbol] is a double bond, X¹ is CR^{a1} and X² is CR^{a2}. In one embodiment, [symbol] is a double bond, X¹ is N and X² is CR^{a2}.

In one embodiment, [symbol] is a double bond, X¹ is C—CH₃ and X² is CH. In one embodiment, [symbol] is a double bond, X¹ is C—C₂H₅ and X² is CH. In one embodiment, [symbol] is a double bond, X¹ is C—CH₃ and X² is N. In one embodiment, [symbol] is a double bond, X¹ is C—OCH₃ and X² is CH.

In one embodiment, [symbol] is a single bond.

In one embodiment, [symbol] is a single bond and X¹ is C(R^{a1})₂. In one embodiment, [symbol] is a single bond and X¹ is CHR^{a1}. In one embodiment, [symbol] is a single bond and X¹ is CH—CH₃. In one embodiment, [symbol] is a single bond and X¹ is CH—CH₂CH₃.

In one embodiment, [symbol] is a single bond and X¹ is NR^{a1}.

In one embodiment, [symbol] is a single bond and X² is O. In one embodiment, [symbol] is a single bond and X² is NR^{a2}.

In one embodiment, [symbol] is a single bond, X¹ is CH(R^{a1}) and X² is O. In one embodiment, [symbol] is a single bond, X¹ is CH(R^{a1}) and X² is NR^{a2}. In one embodiment, [symbol] is a single bond, X¹ is N(R^{a1}) and X² is CR^{a2}.

In one embodiment, [symbol] is a single bond, X¹ is CH—C₂H₃ and X² is O. In one embodiment, [symbol] is a single bond, X¹ is CH—CH₃ and X² is O.

In one embodiment, [symbol] is a single bond, X¹, X² together with R^{a1} and R^{a2} form a 3- to 6-membered Ring B. In one embodiment, [symbol] is a single bond, X¹, X² together with R^{a1} and R^{a2} form a 3- to 6-membered heteroaryl. In one embodiment, [symbol] is a single bond, X¹, X² together with R^{a1} and R^{a2} form a 3- to 6-membered heterocyclyl. In one embodiment, [symbol] is a single bond, X¹, X² together with R^{a1} and R^{a2} form a 5-membered heteroaryl. In one embodiment, [symbol] is a single bond, X¹, X² together with R^{a1} and R^{a2} form a 5-membered heterocyclyl.

In one embodiment, R^{a1} is hydrogen. In one embodiment, R^{a1} is C₁-C₆ alkyl. In one embodiment, R^{a1} is C₃-C₈ cycloalkyl. In one embodiment, R^{a1} is C₁-C₆ alkoxy.

In one embodiment, R^{a1} is C₁-C₄ alkyl. In one embodiment, R^{a1} is C₁-C₄ fluoroalkyl. In one embodiment, R^{a1} is methyl, ethyl, isopropyl, cyclopropyl, 1,1-difluoroethyl, 1-fluoroethyl, trifluoromethyl, difluoromethyl, or methoxy. In one embodiment, R^{a1} is methyl. In one embodiment, R^{a1} is ethyl. In one embodiment, R^{a1} is cyclopropyl. In one embodiment, R^{a1} is methoxy.

In one embodiment, R^{a2} is hydrogen. In one embodiment, R^{a2} is C₁-C₆ alkyl. In one embodiment, R^{a2} is C₃-C₈ cycloalkyl. In one embodiment, R^{a2} is cyclopropyl. In one embodiment, R^{a2} is C₁-C₆ alkoxy. In one embodiment, R^{a2} is methoxy. In one embodiment, R^{a1} is methyl and R^{a2} is hydrogen. In one embodiment, R^{a1} is ethyl and R^{a2} is hydrogen.

In one embodiment, R^{a1} and R^{a2} together with the atoms to which they are attached to form a 3- to 6-membered Ring B.

In one embodiment, the compound is a compound of Formula (I-B), (I-C), or (I-D):

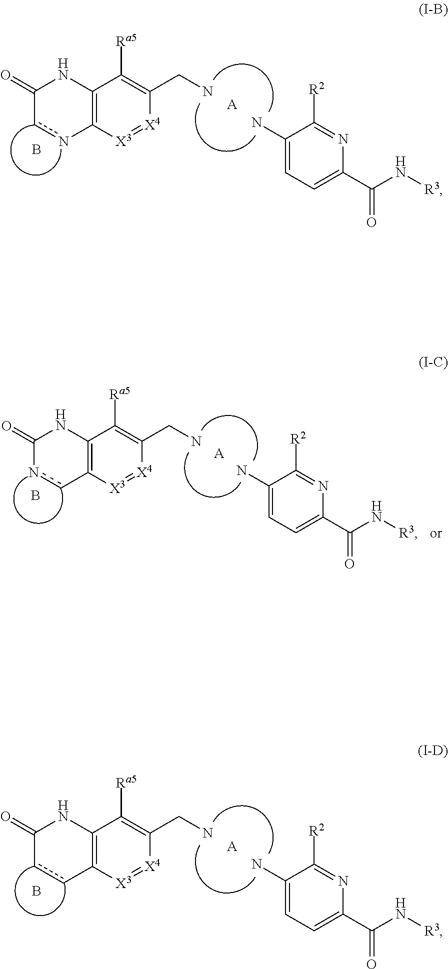

or a stereoisomer, a mixture of stereoisomers, or a pharmaceutically acceptable salt thereof.

In one embodiment, the compound is a compound of Formula (I-B-1), (I-C-1), or (I-D-1):

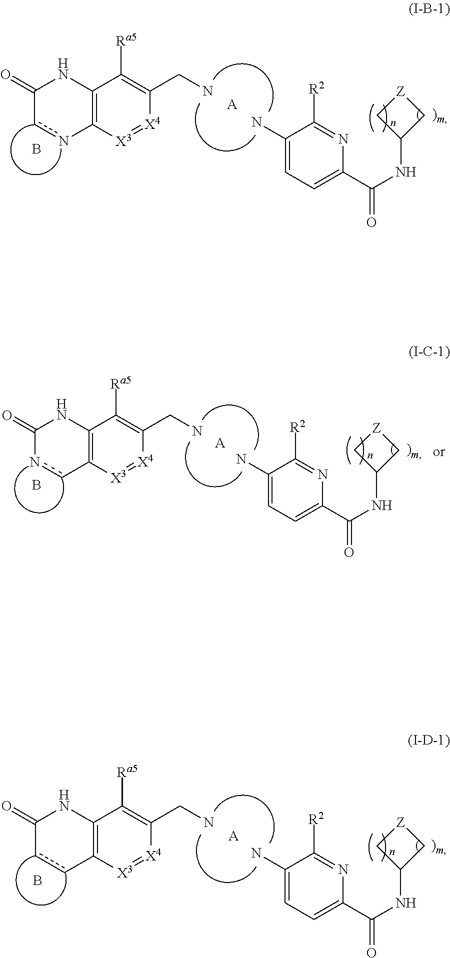

or a stereoisomer, a mixture of stereoisomers, or a pharmaceutically acceptable salt thereof, wherein:
m is 0, 1, 2, 3, or 4;
n is 1, 2, 3, or 4;
Z is O or C(R⁴)₂; and
each R⁴ is independently hydrogen, C₁-C₆ alkyl, or C₁-C₆ alkoxy.

In one embodiment, Ring B is a phenyl. In one embodiment, Ring B is a 5- to 6-membered heteroaryl. In one embodiment, Ring B is a 3- to 6-membered cycloalkyl. In one embodiment, Ring B is a 3- to 6-membered heterocyclyl. In one embodiment, Ring B is a 4- to 6-membered heterocyclyl. In one embodiment, Ring B is a 4- to 5-membered heterocyclyl. In one embodiment, Ring B is a 4- to 6-membered oxygen-containing heterocyclyl.

In one embodiment, Ring B is a 3-membered heterocyclyl. In one embodiment, Ring B is a 4-membered heterocyclyl. In one embodiment, Ring B is a 5-membered heterocyclyl. In one embodiment, Ring B is a 6-membered heterocyclyl. In one embodiment, Ring B is a 4- to 5-membered heterocyclyl containing at least one ring nitrogen atom. In one embodiment, Ring B contains at least one ring oxygen atom. In one embodiment, Ring B contains only one ring oxygen atom. In one embodiment, Ring B is a 5-membered heterocyclyl containing one ring oxygen atom. In one embodiment, Ring B is a 6-membered heterocyclyl containing one ring oxygen atom.

In one embodiment, Ring B is a 5-membered heteroaryl. In one embodiment, Ring B is a 5-membered heteroaryl containing at least one ring nitrogen atom. In one embodiment, Ring B is a 5-membered heteroaryl containing at least one ring oxygen atom.

In one embodiment, Ring B is a 4- to 6-membered cycloalkyl. In one embodiment, Ring B is a 4- to 5-membered cycloalkyl. In one embodiment, Ring B is a 4-membered cycloalkyl. In one embodiment, Ring B is a 5-membered cycloalkyl. In one embodiment, Ring B is a 6-membered cycloalkyl.

In one embodiment, Ring B is a 4- to 6-membered cycloalkenyl. In one embodiment, Ring B is a 4- or 5-membered cycloalkenyl. In one embodiment, Ring B is a 4-membered cycloalkenyl. In one embodiment, Ring B is a 5-membered cycloalkenyl. In one embodiment, Ring B is a 6-membered cycloalkenyl.

In one embodiment, Ring B is a pyrrole ring. In one embodiment, Ring B is a pyrrolidine ring. In one embodiment, Ring B is an imidazole ring. In one embodiment, Ring B is a cyclopentane ring. In one embodiment, Ring B is a cyclopentene ring. In one embodiment, Ring B is a cyclohexane ring. In one embodiment, Ring B is a cyclohexene ring. In one embodiment, Ring B is a tetrahydrofuran ring. In one embodiment, Ring B is a dihydrofuran ring. In one embodiment, Ring B is a tetrahydropyran ring. In one embodiment, Ring B is a dihydropyran ring.

In one embodiment, Ring B is a substituted. In one embodiment, Ring B is substituted with one or more halogen, —CN, C₁-C₆ alkyl, C₁-C₆ alkoxy or nitro. In one embodiment, Ring B is substituted with fluoro. In one embodiment, Ring B is substituted with chloro. In one embodiment, Ring B is substituted with bromo. In one embodiment, Ring B is substituted with cyano. In one embodiment, Ring B is substituted with nitro. In one embodiment, Ring B is substituted with methyl. In one embodiment, Ring B is substituted with ethyl. In one embodiment, Ring B is substituted with methoxy. In one embodiment, Ring B is substituted with ethoxy.

In one embodiment, Ring B is unsubstituted.

In one embodiment, X³ is CR^{a3}. In one embodiment, X³ is C—(C₁-C₆ alkyl). In one embodiment, X³ is C-halogen. In one embodiment, X³ is CH. In one embodiment, X³ is N.

In one embodiment, X⁴ is CR^{a4}. In one embodiment, X⁴ is C—(C₁-C₆ alkyl). In one embodiment, X⁴ is C-halogen. In one embodiment, X⁴ is CH. In one embodiment, X⁴ is N.

In one embodiment, X³ is N and X⁴ is CR^{a4}. In one embodiment, X³ is CR^{a3} and X⁴ is CR^{a4}. In one embodiment, X³ is CR^{a3} and X⁴ is N.

In one embodiment, X¹ is CR^{a1}, X² is CR^{a2} and X³ is N. In one embodiment, X¹ is CR^{a1}, X² is N and X³ is CR^{a3}. In one embodiment, X¹ is CHR^{a1}, X² is O and X³ is CR^{a3}. In one embodiment, X¹ is CHR^{a1}, X² is N and X³ is CR^{a3}. In one embodiment, X¹ is CR^{a1}, X² is CR^{a2} and X³ is CR^{a3}. In one embodiment, X¹ is NR^{a1}, X² is CR^{a2} and X³ is N. In one embodiment, X¹ is NR^{a1}, X² is CR^{a2} and X³ is CR^{a3}.

In one embodiment, X¹ is CR^{a1}, X² is CR^{a2} and X⁴ is N. In one embodiment, X¹ is CR^{a1}, X² is N and X⁴ is CR^{a4}. In one embodiment, X¹ is CHR^{a1}, X² is O and X⁴ is CR^{a4} In one embodiment, X¹ is CHR^{a1}, X² is N and X⁴ is CR^{a4}. In one embodiment, X¹ is CR^{a1}, X² is CR^{a2} and X⁴ is CR^{a4}. In one embodiment, X¹ is NR^{a1}, X² is CR^{a2} and X⁴ is N. In one embodiment, X¹ is NR^{a1}, X² is CR^{a2} and X⁴ is CR^{a4}.

In one embodiment, X¹ is CR^{a1}, X² is CR^{a2}, X³ is N and X⁴ is CR^{a4}. In one embodiment, X¹ is CR^{a1}, X² is N, X³ is CR^{a3} and X⁴ is CR^{a4}. In one embodiment, X¹ is CR^{a1}, X² is N, X³ is N and X⁴ is CR^{a4}. In one embodiment, X¹ is CHR^{a1}, X² is O, X³ is N and X⁴ is CR^{a4}. In one embodiment, X¹ is CHR^{a1}, X² is O, X³ is CR^{a3} and X⁴ is CR^{a4}. In one embodiment, X¹ is CHR^{a1}, X² is N, X³ is CR^{a3} and X⁴ is N. In one embodiment, X¹ is CR^{a1}, X² is CR^{a2}, X³ is CR^{a3} and X⁴ is N. In one embodiment, X¹ is NR^{a1}, X² is CR^{a2}, X³ is N and X⁴ is CR^{a4}. In one embodiment, X¹ is NR^{a1}, X² is CR^{a2}, X³ is CR^{a3} and X⁴ is CR^{a4}.

In one embodiment, the compound is a compound of Formula (II-A), (II-B), (II-C), (II-D), (II-E), (II-F), (II-G), (II-H), (II-I), (II-J), (II-K), (II-L), (II-M), (II-N), (II-O), (II-P), (II-Q), (II-R), (II-S), (II-T), (II-U), (II-V), (II-W), (II-X), (II-Y), (II-Z), (II-AA), (II-AB), (II-AC), (II-AD), (II-AE), (II-AF), (II-AG), (II-AH), (II-AI), or (II-AJ):

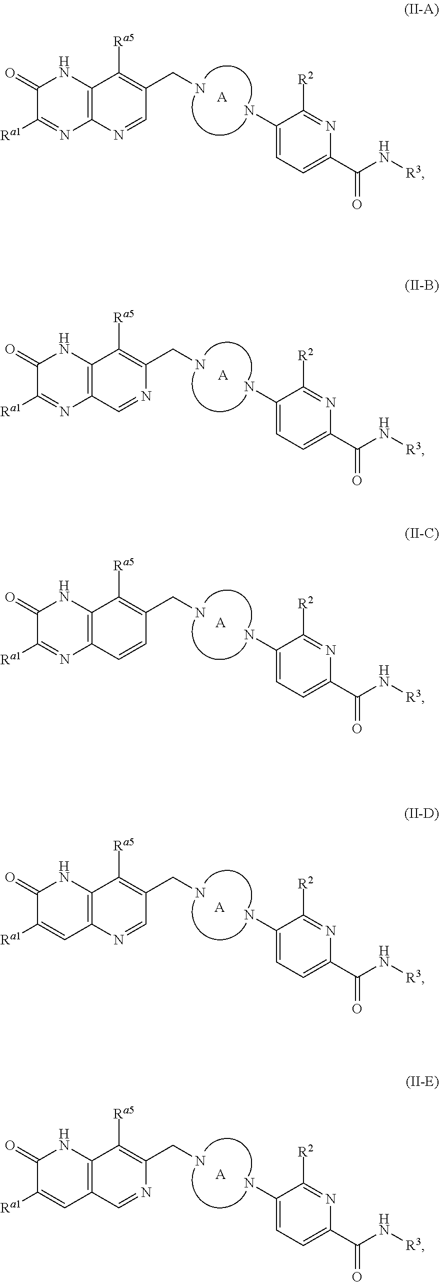

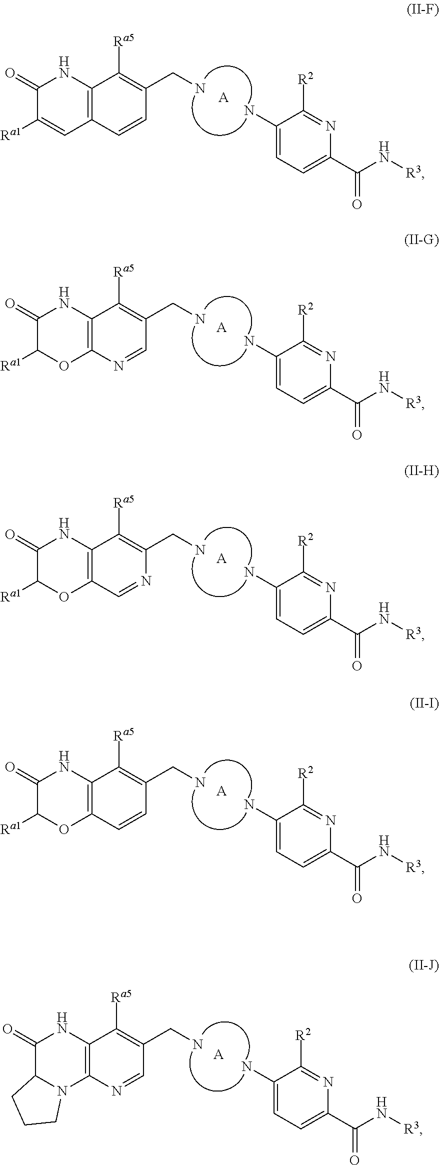

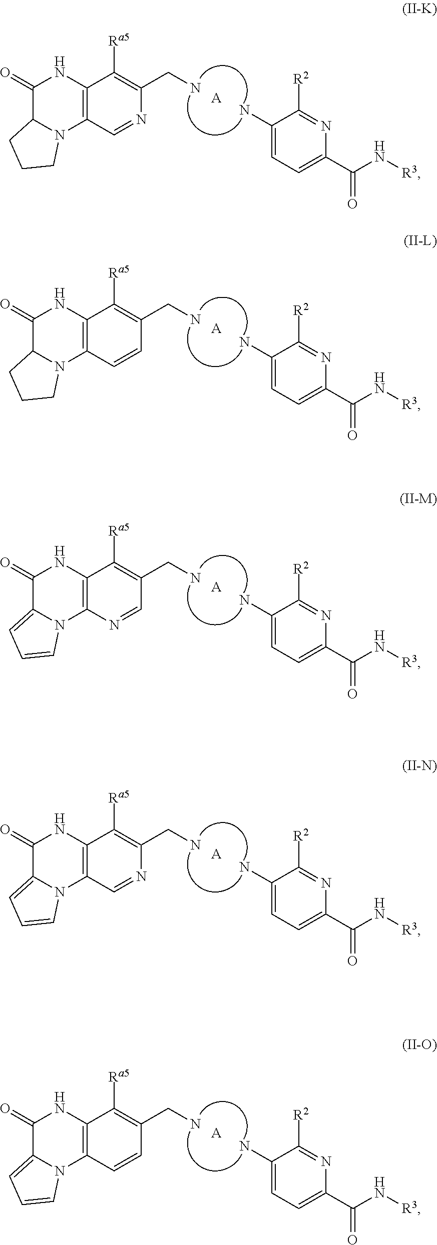

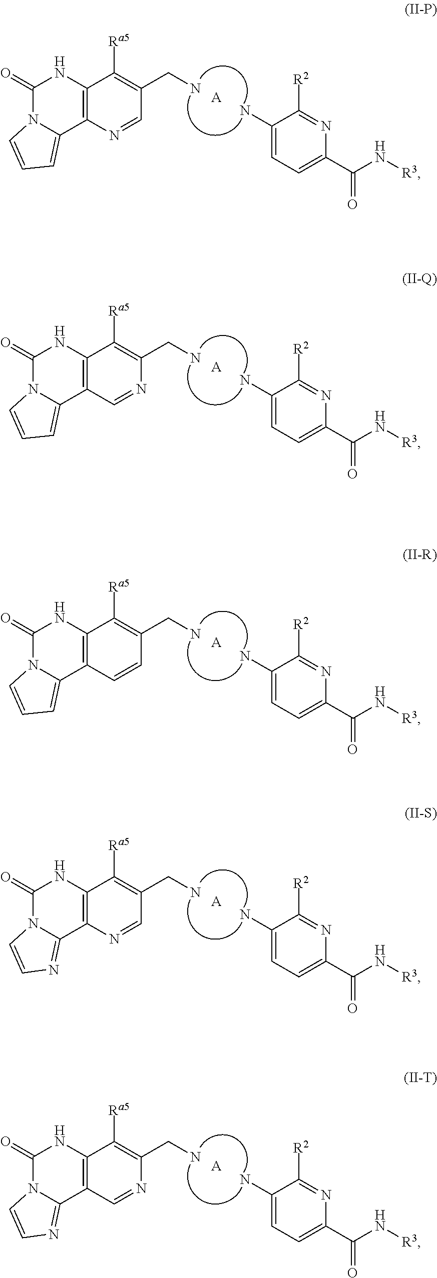

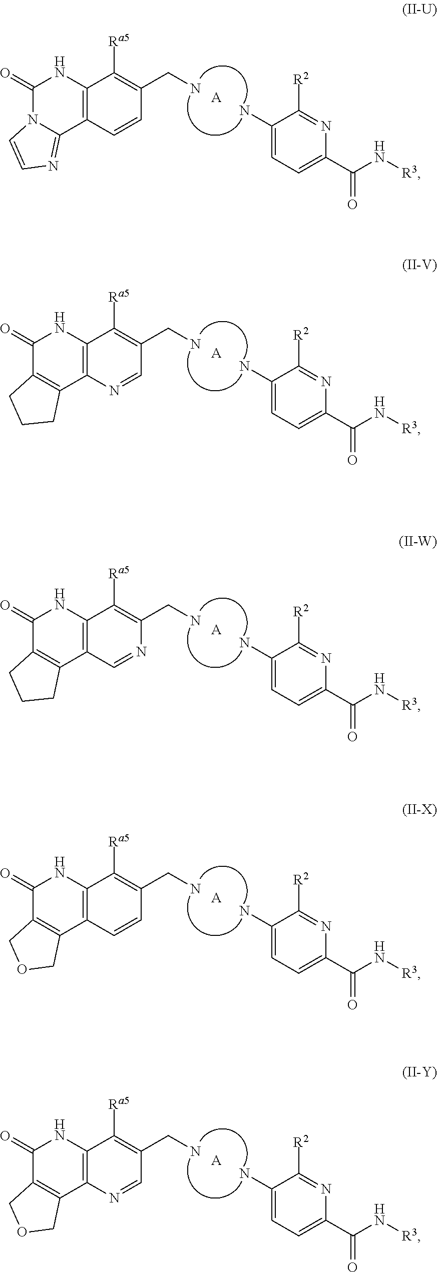

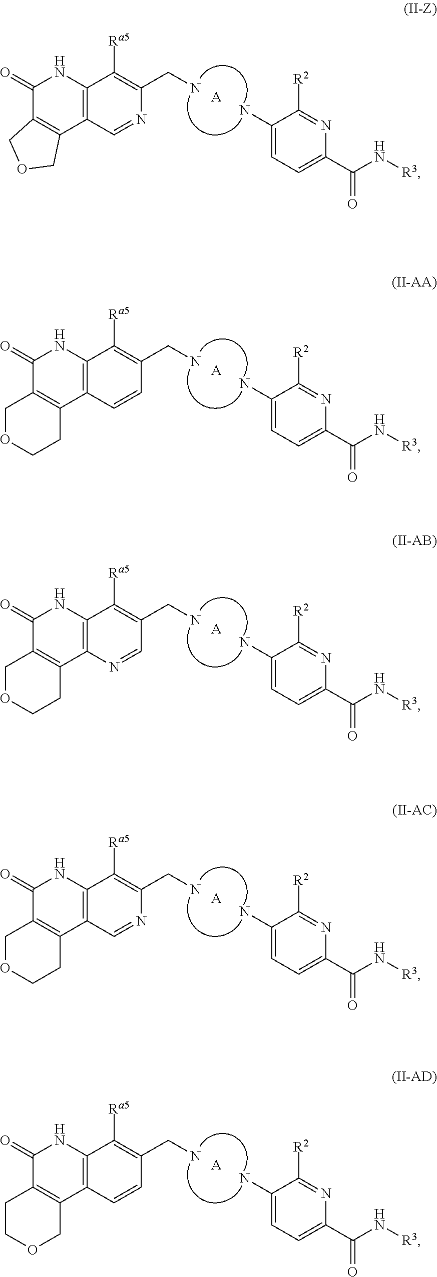

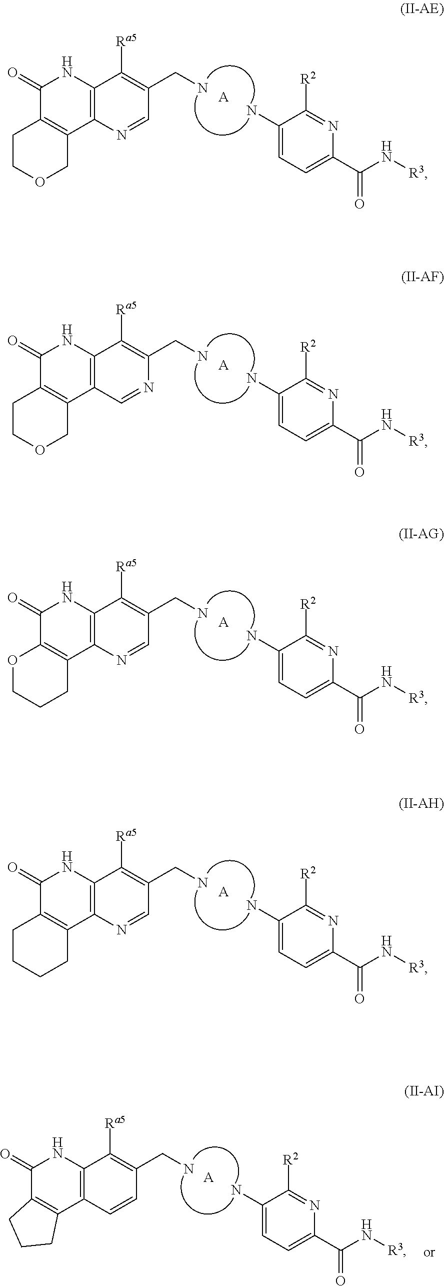

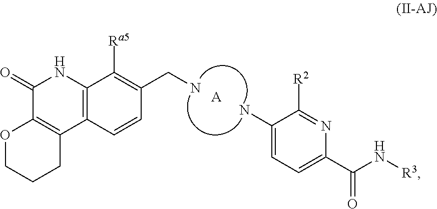

or a stereoisomer, a mixture of stereoisomers, or a pharmaceutically acceptable salt thereof.

In one embodiment, the compound is a compound of Formula (II-V-1), (II-AA-1), (II-AB-1), or (II-AI-1):

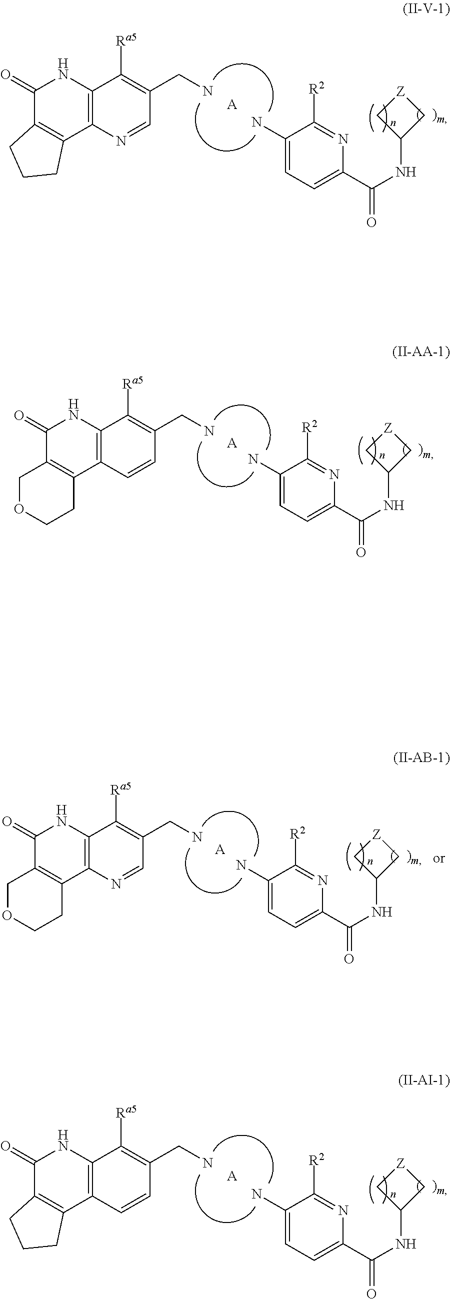

or a stereoisomer, a mixture of stereoisomers, or a pharmaceutically acceptable salt thereof, wherein:
m is 0, 1, 2, 3, or 4;
n is 1, 2, 3, or 4;
Z is O or C(R⁴)₂; and
each R⁴ is independently hydrogen, C₁-C₆ alkyl, or C₁-C₆ alkoxy.

In one embodiment of Formula (II-D)(or a sub-formula thereof), Ring A is

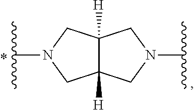 [SMILES: *N1C[C@H]2CN(*)C[C@@H]2C1]

wherein * is toward the direction of the methylene group linking Ring A and the ring containing X⁴. In one embodiment of Formula (II-D)(or a sub-formula thereof), Ring A is

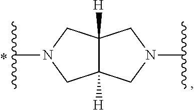 [SMILES: *N1C[C@H]2CN(*)C[C@@H]2C1]

wherein * is toward the direction of the methylene group linking Ring A and the ring containing X⁴.

In one embodiment, X¹ is CR^{a1}, X² is CR^{a2} and Y¹ is CR². In one embodiment, X¹ is CR^{a1}, X² is N and Y¹ is CR². In one embodiment, X¹ is CHR^{a1}, X² is O and Y¹ is CR². In one embodiment, X¹ is CHR^{a1}, X² is N and Y¹ is CR². In one embodiment, X¹ is CR^{a1}, X² is CR^{a2} and Y¹ is CR². In one embodiment, X¹ is NR^{a1}, X² is CR^{a2} and Y¹ is CR². In one embodiment, X¹ is NR^{a1}, X² is CR^{a2} and Y¹ is CR².

In one embodiment, X¹ is CR^{a1}, X² is CR^{a2}, Y¹ is CR² and Y² is N. In one embodiment, X¹ is CR^{a1}, X² is N, Y¹ is CR² and Y² is N. In one embodiment, X¹ is CHR^{a1}, X² is O, Y¹ is CR² and Y² is N. In one embodiment, X¹ is CHR^{a1}, X² is N, Y¹ is CR² and Y² is N. In one embodiment, X¹ is CR^{a1}, X² is CR^{a2}, Y¹ is CR² and Y² is N. In one embodiment, X¹ is NR^{a1}, X² is CR^{a2}, Y¹ is CR² and Y² is N. In one embodiment, X¹ is NR^{a1}, X² is CR^{a2}, Y¹ is CR² and Y² is N.

In one embodiment, X¹ is CR^{a1}, X² is CR^{a2}, X⁴ is N, Y¹ is CR² and Y² is N. In one embodiment, X¹ is CR^{a1}, X² is N, X⁴ is CR^{a4}, Y¹ is CR² and Y² is N. In one embodiment, X¹ is CHR^{a1}, X² is O, X⁴ is CR^{a4}, Y¹ is CR² and Y² is N. In one embodiment, X¹ is CHR^{a1} X² is N, X⁴ is CR^{a4}, Y¹ is CR² and Y² is N. In one embodiment, X¹ is CR^{a1}, X² is CR^{a2}, X⁴ is CR^{a4}, Y¹ is CR² and Y² is N. In one embodiment, X¹ is NR^{a1}, X² is CR^{a2}, X⁴ is N, Y¹ is CR² and Y² is N. In one embodiment, X¹ is NR^{a1}, X² is CR^{a2}, X⁴ is CR^{a4} and Y¹ is CR² and Y² is N.

In one embodiment, X¹ is CR^{a1}, X² is CR^{a2}, X³ is N, X⁴ is CR^{a4}, Y¹ is CR² and Y² is N. In one embodiment, X¹ is CR^{a1}, X² is N, X³ is CR^{a3}, X⁴ is CR^{a4}, Y¹ is CR² and Y² is N. In one embodiment, X¹ is CR^{a1}, X² is N, X³ is N, X⁴ is CR^{a4}, Y¹ is CR² and Y² is N. In one embodiment, X¹ is CHR^{a1}, X² is O, X³ is N, X⁴ is CR^{a4}, Y¹ is CR² and Y² is N. In one embodiment, X¹ is CHR^{a1}, X² is O, X³ is CR^{a3}, X⁴ is CR^{a4}, Y¹ is CR² and Y² is N. In one embodiment, X¹ is CHR^{a1}, X² is N, X³ is CR^{a3}, X⁴ is N, Y¹ is CR² and Y² is N. In one embodiment, X¹ is CR^{a1}, X² is CR^{a2}, X³ is CR^{a3}, X⁴ is N, Y¹ is CR² and Y² is N. In one embodiment, X¹ is NR^{a1}, X² is CR^{a2}, X³ is N, X⁴ is CR^{a4}, Y¹ is CR² and Y² is N. In one embodiment, X¹ is NR^{a1}, X² is CR^{a2}, X³ is CR^{a3}, X⁴ is CR^{a4}, Y¹ is CR² and Y² is N.

In one embodiment, R^{a5} is C₁-C₆ alkyl. In one embodiment, R^{a5} is methyl. In one embodiment, R^{a5} is ethyl. In one embodiment, R^{a5} is n-propyl or isopropyl. In one embodiment, R^{a5} is n-butyl, iso-butyl or tert-butyl. In one embodiment, R^{a5} is C₅ alkyl. In one embodiment, R^{a5} is C₆ alkyl. In one embodiment, R^{a5} is halogen. In one embodiment, R^{a5} is fluoro. In one embodiment, R^{a5} is chloro. In one embodiment, R^{a5} is bromo. In one embodiment, R^{a5} is hydrogen.

In one embodiment, Ring A is a fused heterocyclyl. In one embodiment, Ring A is a 6- to 12-membered fused heterocyclyl. In one embodiment, Ring A is a 6-membered fused heterocyclyl. In one embodiment, Ring A is a 7-membered fused heterocyclyl. In one embodiment, Ring A is an 8-membered fused heterocyclyl. In one embodiment, Ring A is a 9-membered fused heterocyclyl. In one embodiment, Ring A is a 10-membered fused heterocyclyl. In one embodiment, Ring A is a 11-membered fused heterocyclyl. In one embodiment, Ring A is a 12-membered fused heterocyclyl. In one embodiment, Ring A is a fused bicyclic heterocyclyl.

In one embodiment, Ring A is a bridged heterocyclyl. In one embodiment, Ring A is a 6- to 12-membered bridged heterocyclyl. In one embodiment, Ring A is a 6-membered bridged heterocyclyl. In one embodiment, Ring A is a 7-membered bridged heterocyclyl. In one embodiment, Ring A is an 8-membered bridged heterocyclyl. In one embodiment, Ring A is a 9-membered bridged heterocyclyl. In one embodiment, Ring A is a 10-membered bridged heterocyclyl. In one embodiment, Ring A is a 11-membered bridged heterocyclyl. In one embodiment, Ring A is a 12-membered bridged heterocyclyl. In one embodiment, Ring A is a bridged bicyclic heterocyclyl.

In one embodiment, Ring A is a spiro heterocyclyl. In one embodiment, Ring A is a 6- to 12-membered spiro heterocyclyl. In one embodiment, Ring A is a 6-membered spiro heterocyclyl. In one embodiment, Ring A is a 7-membered spiro heterocyclyl. In one embodiment, Ring A is a 8-membered spiro heterocyclyl. In one embodiment, Ring A is a 9-membered spiro heterocyclyl. In one embodiment, Ring A is a 10-membered spiro heterocyclyl. In one embodiment, Ring A is a 11-membered spiro heterocyclyl. In one embodiment, Ring A is a 12-membered spiro heterocyclyl. In one embodiment, Ring A is a spiro bicyclic heterocyclyl.

In one embodiment, Ring A contains at least two heteroatoms. In one embodiment, Ring A contains at least two heteroatoms that are both nitrogen. In one embodiment, Ring A contains only two heteroatoms that are both nitrogen.

In one embodiment, Ring A is

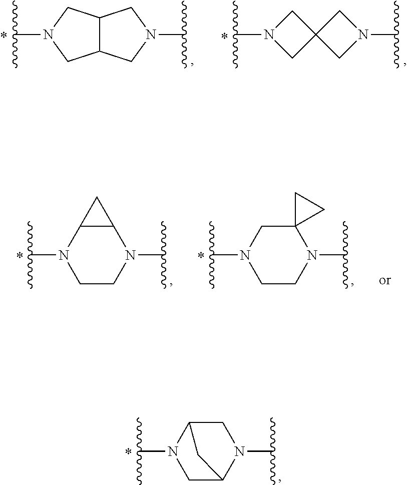 [SMILES: *N1CC2CN(*)CC2C1 ; *N1CC2(C1)CN(*)C2 ; *N1CCN(*)C2CC21 ; *N1CCN(*)C2(CC2)C1 ; *N1CC2CC1CN2*]

wherein * is toward the direction of the methylene group linking Ring A and the ring containing X⁴.

In one embodiment, Ring A is

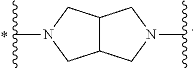 [SMILES: *N1CC2CN(*)CC2C1]

In one embodiment, Ring A is

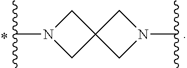 [SMILES: *N1CC2(C1)CN(*)C2]

In one embodiment, Ring A is

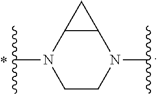 [SMILES: *N1CCN(*)C2CC21]

In one embodiment, Ring A is

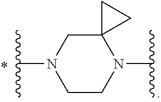 [SMILES: *N1CCN(*)C2(CC2)C1]

In one embodiment, Ring A is

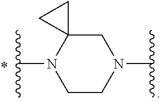 [SMILES: *N1CCN(*)C2(CC2)C1]

In one embodiment, Ring A is

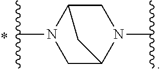 [SMILES: *N1CC2CC1CN2*]

In one embodiment, Ring A is

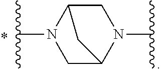 [SMILES: *N1CC2CC1CN2*]

In one embodiment, Ring A is octahydropyrrolo[3,4-c]pyrrole. In one embodiment, Ring A is 2,6-diazaspiro[3.3]heptane. In one embodiment, Ring A is 2,5-diazabicyclo[4.1.0]heptane. In one embodiment, Ring A is 2,5-diazabicyclo[2.2.1]heptane. In one embodiment, Ring A is 4,7-diazaspiro[2.5]octane.

In one embodiment, Ring A is (3ar,6ar)-octahydropyrrolo[3,4-c]pyrrole. In one embodiment, Ring A is (3as,6as)-octahydropyrrolo[3,4-c]pyrrole.

In one embodiment, X² is N, Ring A is

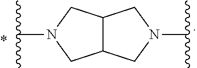 [SMILES: *N1CC2CN(*)CC2C1]

In one embodiment, X³ is N and Ring A is

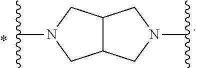 [SMILES: *N1CC2CN(*)CC2C1]

In one embodiment, X⁴ is N and Ring A is

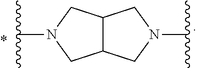 [SMILES: *N1CC2CN(*)CC2C1]

In one embodiment, X² is N, X³ is N and Ring A is

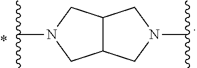 [SMILES: *N1CC2CN(*)CC2C1]

In one embodiment, X² is N, X⁴ is N and Ring A is

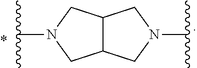 [SMILES: *N1CC2CN(*)CC2C1]

In one embodiment, X² is O, X³ is N and Ring A is

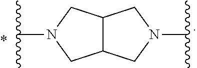 [SMILES: *N1CC2CN(*)CC2C1]

In one embodiment, X² is O, X⁴ is N and Ring A is

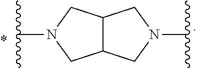 [SMILES: *N1CC2CN(*)CC2C1]

In one embodiment, X² is O and Ring A is

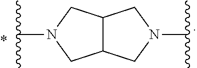 [SMILES: *N1CC2CN(*)CC2C1]

In one embodiment, X² is N, X³ is N and Ring A is

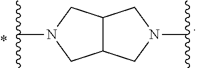 [SMILES: *N1CC2CN(*)CC2C1]

In one embodiment, Ring A is

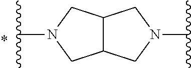 [SMILES: *N1CC2CN(*)CC2C1]

and Ring B is a heteroaryl, wherein * is toward the direction of the methylene group linking Ring A and the ring containing X⁴.

In one embodiment, Ring A is substituted. In one embodiment, Ring A is substituted with one or more groups selected from the group consisting of halogen, CN, C₁-C₆ alkyl, C₁-C₆ alkoxy and nitro.

In one embodiment, Ring A is unsubstituted.

In one embodiment, the compound is a compound of Formula (III-A), (III-B), (III-C), (III-D), (III-E), (III-F), (III-G), (III-H), (III-I), (III-J), (III-K), (III-L), (III-M), (III-N), (III-O), (III-P), (III-Q), (III-R), (III-S), (III-T), (III-U), (III-V), (III-W), (III-X), (III-Y), (III-Z), (III-AA), (III-AB), (III-AC), (III-AD), (III-AE), (III-AF), (III-AG), (III-AH), (III-AI), or (III-AJ):

[chemical structure]

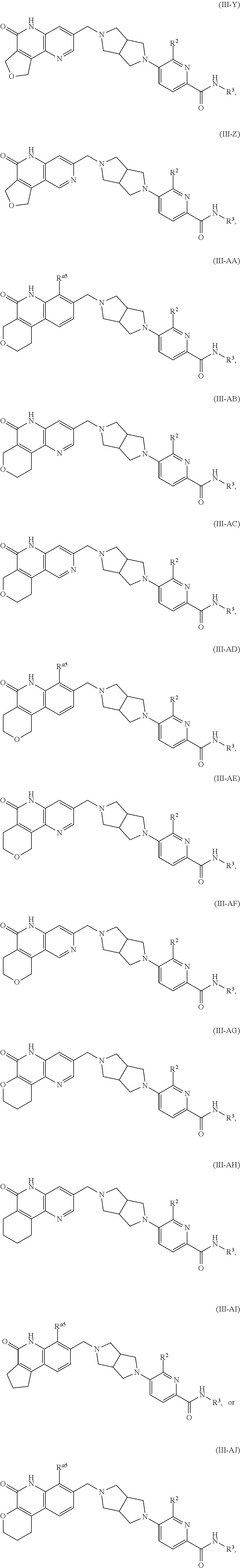

or a stereoisomer, a mixture of stereoisomers, or a pharmaceutically acceptable salt thereof.

In one embodiment of Formula (III-D)(or a sub-formula thereof), Ring A is

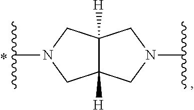 [SMILES: *N1C[C@H]2CN(*)C[C@@H]2C1]

wherein * is toward the direction of the methylene group linking Ring A and the ring containing X⁴. In one embodiment of Formula (III-D)(or a sub-formula thereof), Ring A is

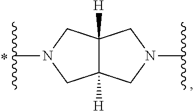 [SMILES: *N1C[C@H]2CN(*)C[C@@H]2C1]

wherein * is toward the direction of the methylene group linking Ring A and the ring containing X⁴.

In one embodiment, Ring A is cis-

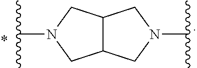 [SMILES: *N1CC2CN(*)CC2C1]

In one embodiment, Ring A is trans-

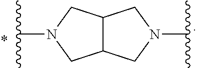 [SMILES: *N1CC2CN(*)CC2C1]

In one embodiment, Ring A is

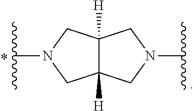 [SMILES: *N1C[C@H]2CN(*)C[C@@H]2C1]

In one embodiment, Ring A is

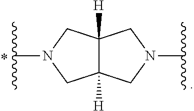 [SMILES: *N1C[C@H]2CN(*)C[C@@H]2C1]

In one embodiment, without being bound by a particular theory, a compound with trans-

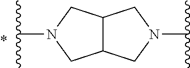 [SMILES: *N1CC2CN(*)CC2C1]

(or a stereoisomer such as

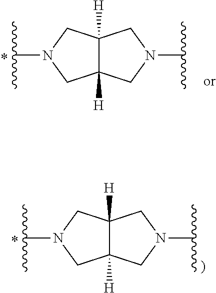 [SMILES: *N1C[C@H]2CN(*)C[C@@H]2C1 ; *N1C[C@H]2CN(*)C[C@@H]2C1]

as Ring A exhibits better potency and/or efficacy than the corresponding compound with cis-

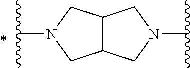 [SMILES: *N1CC2CN(*)CC2C1]

as Ring A. In one embodiment, without being bound by a particular theory, one of the stereoisomer exhibits better potency and/or efficacy than the other stereoisomer.

In one embodiment, Ring A is cis-

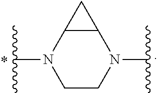 [SMILES: *N1CCN(*)C2CC21]

In one embodiment, Ring A is trans-

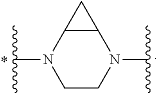 [SMILES: *N1CCN(*)C2CC21]

In one embodiment, Ring A is

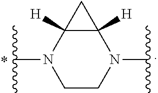 [SMILES: *N1CCN(*)[C@H]2C[C@H]21]

In one embodiment, Ring A is

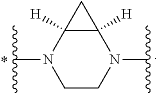 [SMILES: *N1CCN(*)[C@H]2C[C@H]21]

In one embodiment, Ring A is

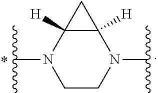 [SMILES: *N1CCN(*)[C@@H]2C[C@H]21]

In one embodiment, Ring A is

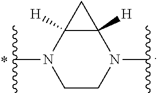 [SMILES: *N1CCN(*)[C@H]2C[C@@H]21]

In one embodiment, X³ is N and Ring A is

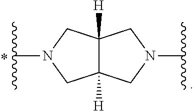 [SMILES: *N1C[C@H]2CN(*)C[C@@H]2C1]

In one embodiment, X³ is N and Ring A is

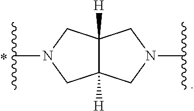 [SMILES: *N1C[C@H]2CN(*)C[C@@H]2C1]

In one embodiment, X³ is N and Ring A is

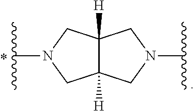 [SMILES: *N1C[C@H]2CN(*)C[C@@H]2C1]

In one embodiment, X¹ is CR^{a1}, X² is CR^{a2}, X³ is N, X⁴ is CR^{a4} and Ring A is

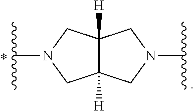 [SMILES: *N1C[C@H]2CN(*)C[C@@H]2C1]

In one embodiment, X¹ is CR^{a1}, X² is CR^{a2}, X³ is N, X⁴ is CR^{a4} and Ring A is

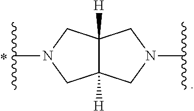 [SMILES: *N1C[C@H]2CN(*)C[C@@H]2C1]

In one embodiment, X¹ is CR^{a1}, X² is CR^{a2}, X³ is N, X⁴ is CR^{a4} and Ring A is

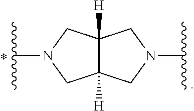 [SMILES: *N1C[C@H]2CN(*)C[C@@H]2C1]

In one embodiment, X² is N, Ring A is

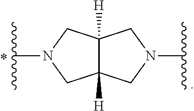 [SMILES: *N1C[C@H]2CN(*)C[C@@H]2C1]

In one embodiment, X³ is N and Ring A is

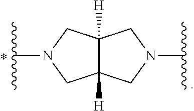 [SMILES: *N1C[C@H]2CN(*)C[C@@H]2C1]

In one embodiment, X⁴ is N and Ring A is

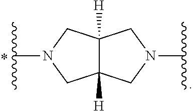 [SMILES: *N1C[C@H]2CN(*)C[C@@H]2C1]

In one embodiment, X² is N, X³ is N and Ring A is

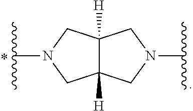 [SMILES: *N1C[C@H]2CN(*)C[C@@H]2C1]

In one embodiment, X² is N, X⁴ is N and Ring A is

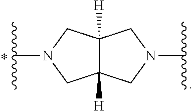 [SMILES: *N1C[C@H]2CN(*)C[C@@H]2C1]

In one embodiment, X² is O, X³ is N and Ring A is

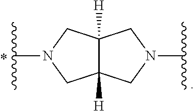 [SMILES: *N1C[C@H]2CN(*)C[C@@H]2C1]

In one embodiment, X² is O, X⁴ is N and Ring A is

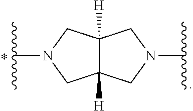 [SMILES: *N1C[C@H]2CN(*)C[C@@H]2C1]

In one embodiment, X² is O and Ring A is

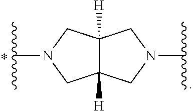 [SMILES: *N1C[C@H]2CN(*)C[C@@H]2C1]

In one embodiment, X² is N, X³ is N and Ring A is

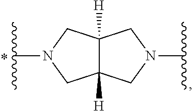 [SMILES: *N1C[C@H]2CN(*)C[C@@H]2C1]

In one embodiment, Ring A is

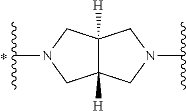 [SMILES: *N1C[C@H]2CN(*)C[C@@H]2C1]

and Ring B is a heteroaryl.

In one embodiment, the compound is a compound of Formula (IV-A1), (IV-B1), (IV-C1), (IV-D1), (IV-E1), (IV-F1), (IV-G1), (IV-H1), (IV-11), (IV-J1), (IV-K1), (IV-L1), (IV-M1), (IV-N1), (IV-O1), (IV-P1), (IV-Q1), (IV-R1), (IV-S1), (IV-T1), (IV-U1), (IV-V1), (IV-W1), (IV-X1), (IV-Y1), (IV-Z1), (IV-AA1), (IV-AB1), (IV-AC1), (IV-AD1), (IV-AE1), (IV-AF1), (IV-AG1), (IV-AH1), (IV-AI1), or (IV-AJ1):

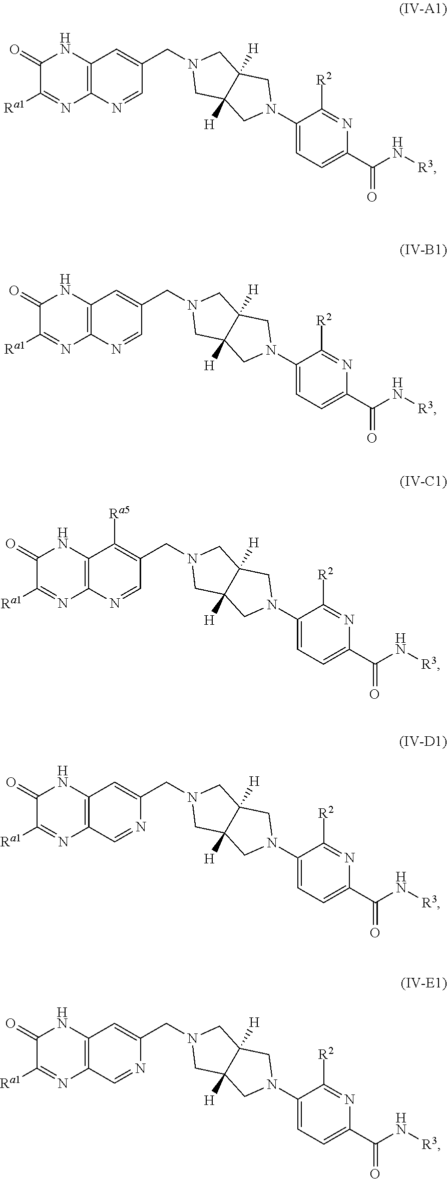

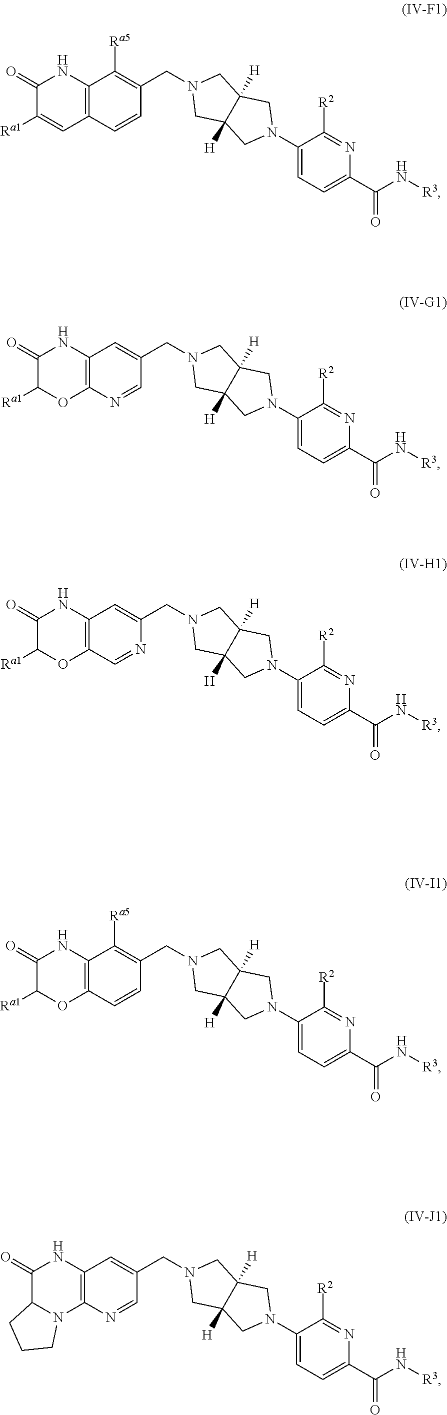

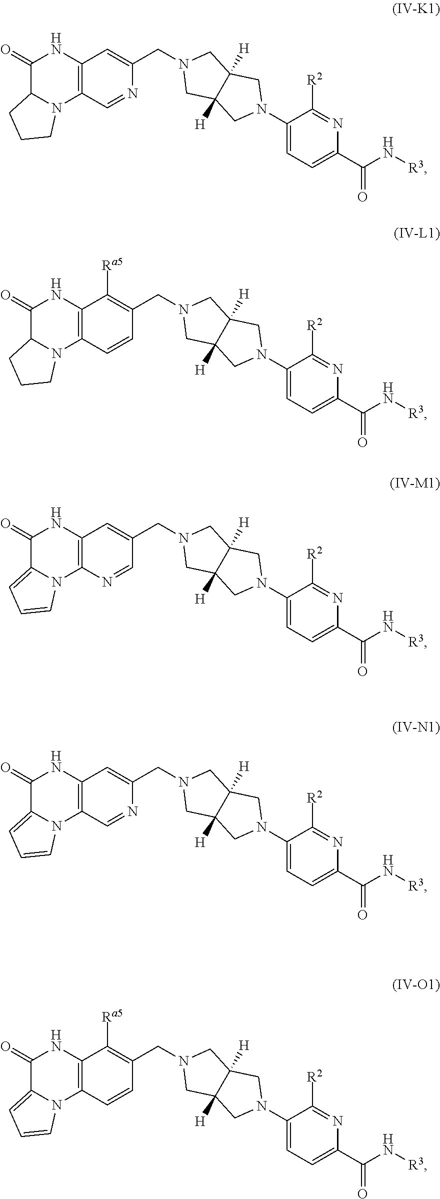

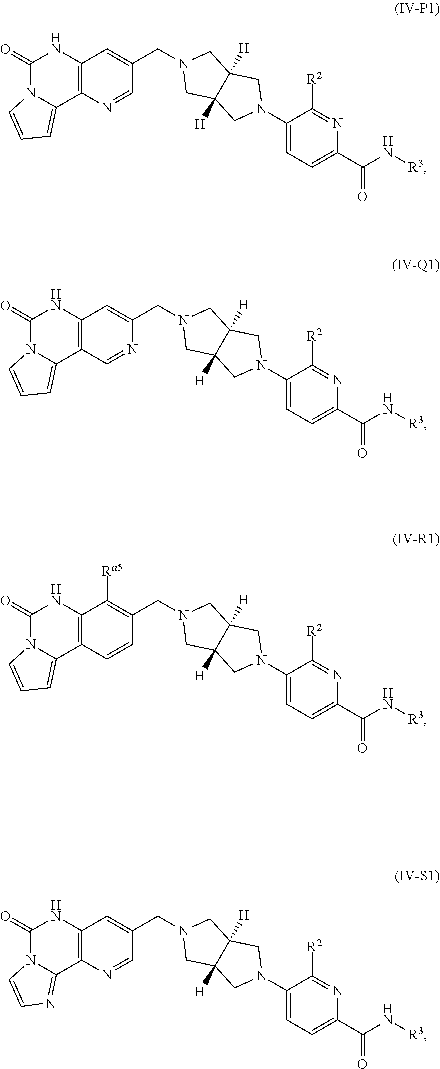

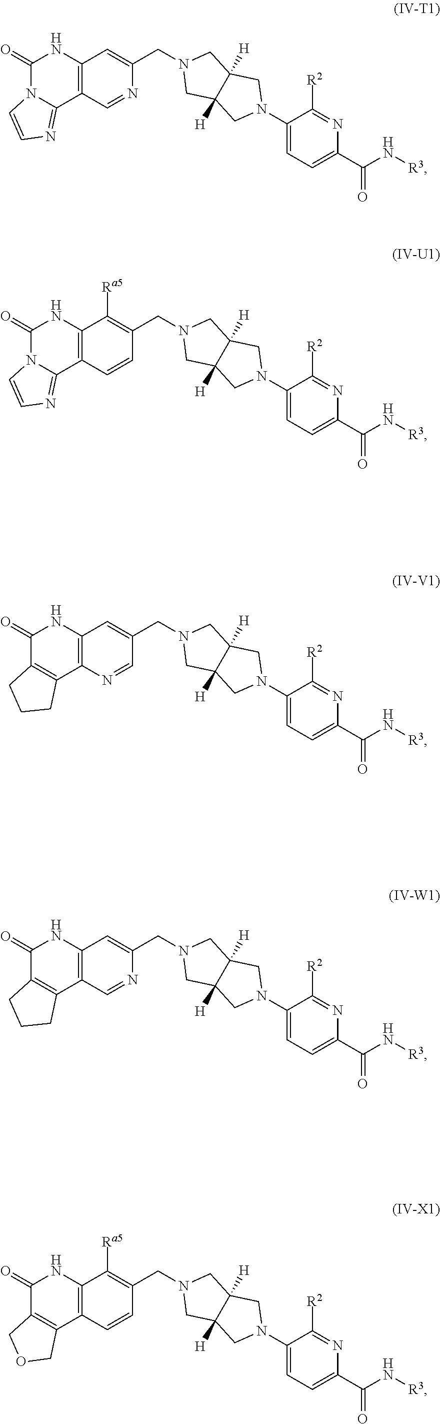

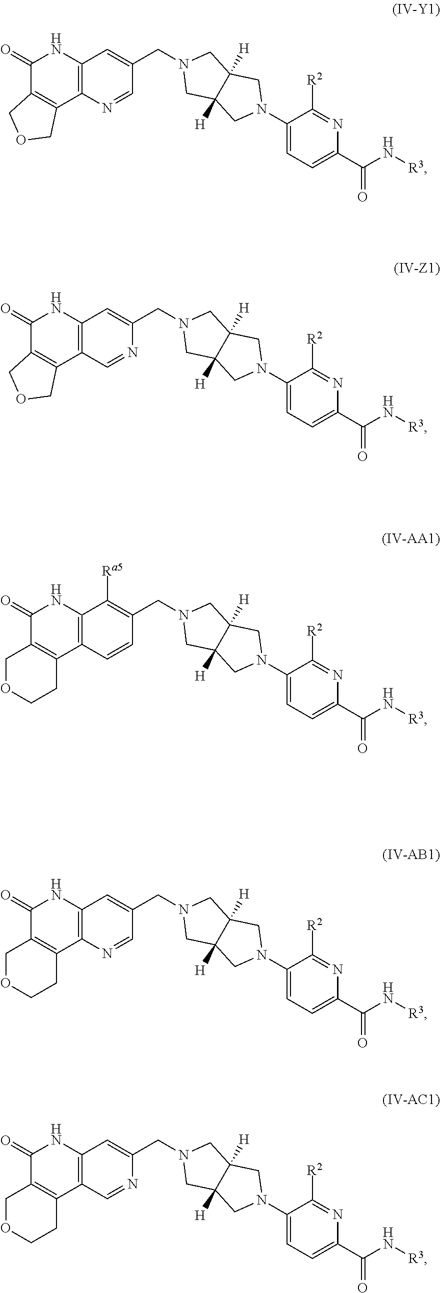

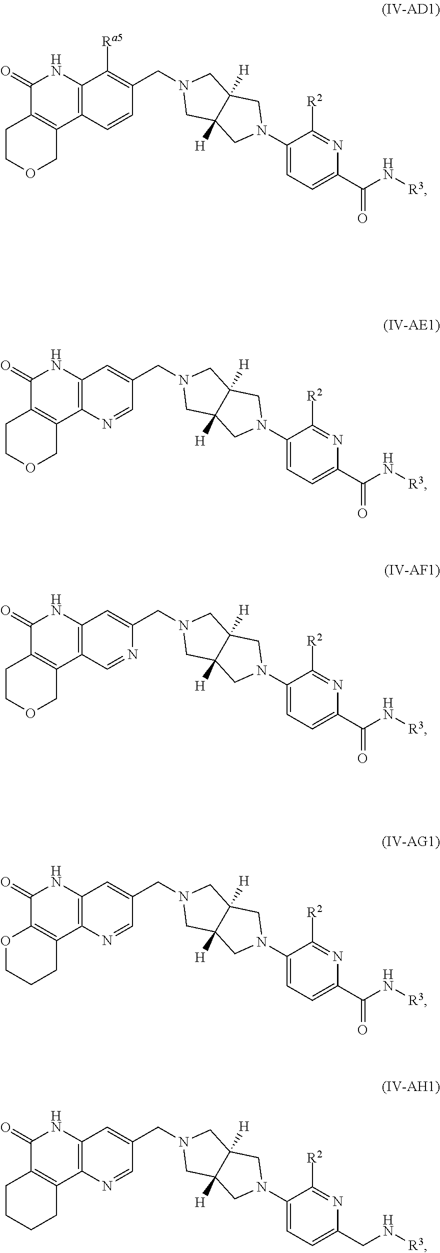

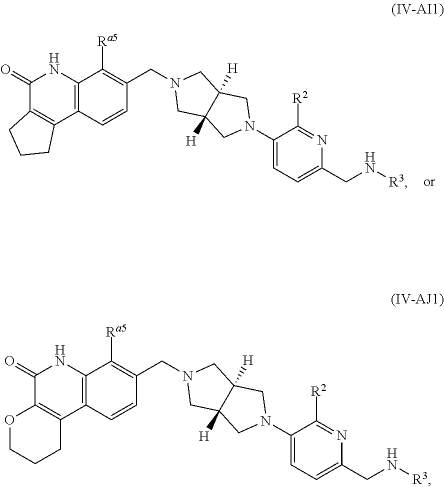

or a stereoisomer, a mixture of stereoisomers, or a pharmaceutically acceptable salt thereof.

In one embodiment, the compound is a compound of Formula (IV-V1-1), (IV-AA1-1), (IV-AB1-1), or (IV-AI1-1):

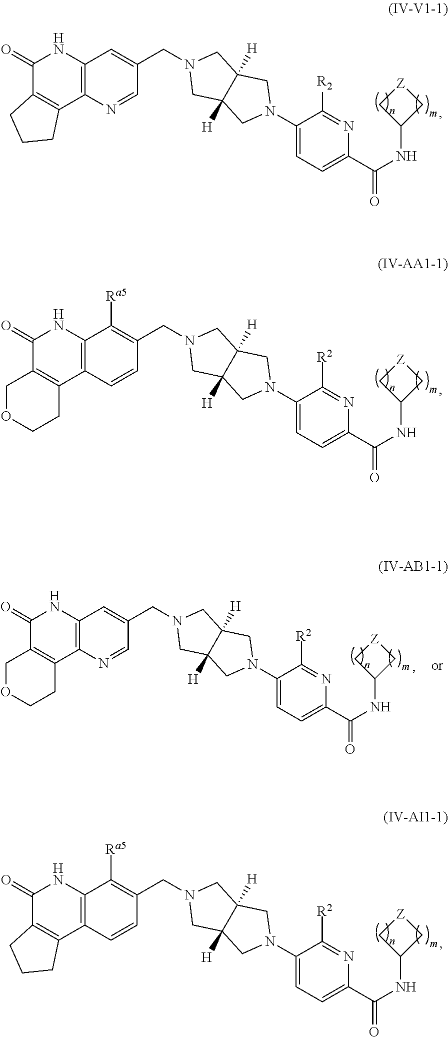

or a stereoisomer, a mixture of stereoisomers, or a pharmaceutically acceptable salt thereof, wherein:
m is 0, 1, 2, 3, or 4;
n is 1, 2, 3, or 4;
Z is O or C(R⁴)₂; and
each R⁴ is independently hydrogen, C₁-C₆ alkyl, or C₁-C₆ alkoxy.

In one embodiment, the compound is a compound of Formula (IV-A2), (IV-B2), (IV-C2), (IV-D2), (IV-E2), (IV-F2), (IV-G2), (IV-H2), (IV-12), (IV-J2), (IV-K2), (IV-L2), (IV-M2), (IV-N2), (IV-02), (IV-P2), (IV-Q2), (IV-R2), (IV-S2), (IV-T2), (IV-U2), (IV-V2), (IV-W2), (IV-X2), (IV-Y2), (IV-Z2), (IV-AA2), (IV-AB2), (IV-AC2), (IV-AD2), (IV-AE2), (IV-AF2), (IV-AG2), (IV-AH2), (IV-AI2), or (IV-AJ2):

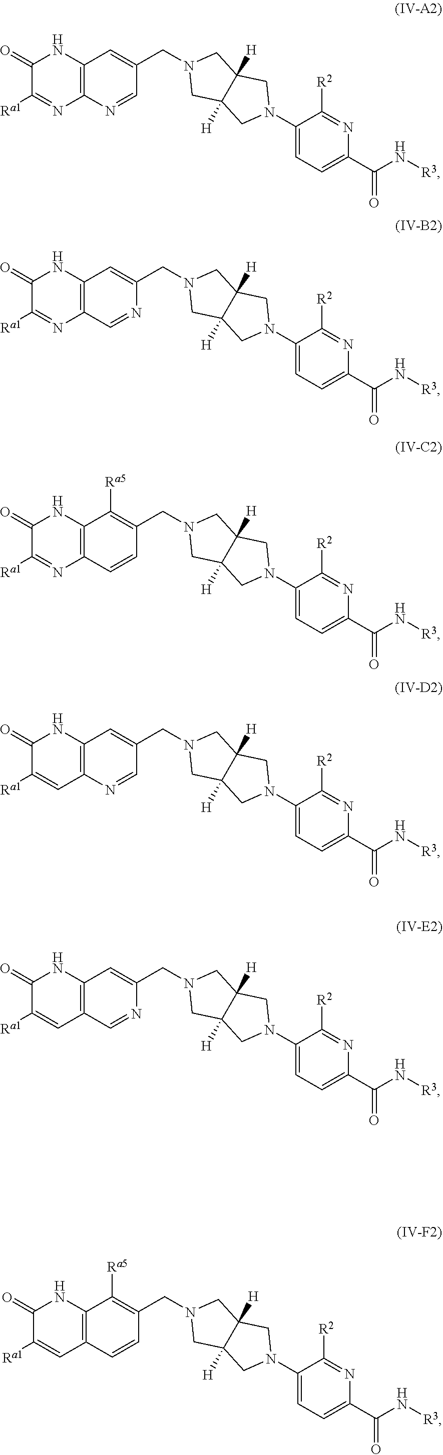

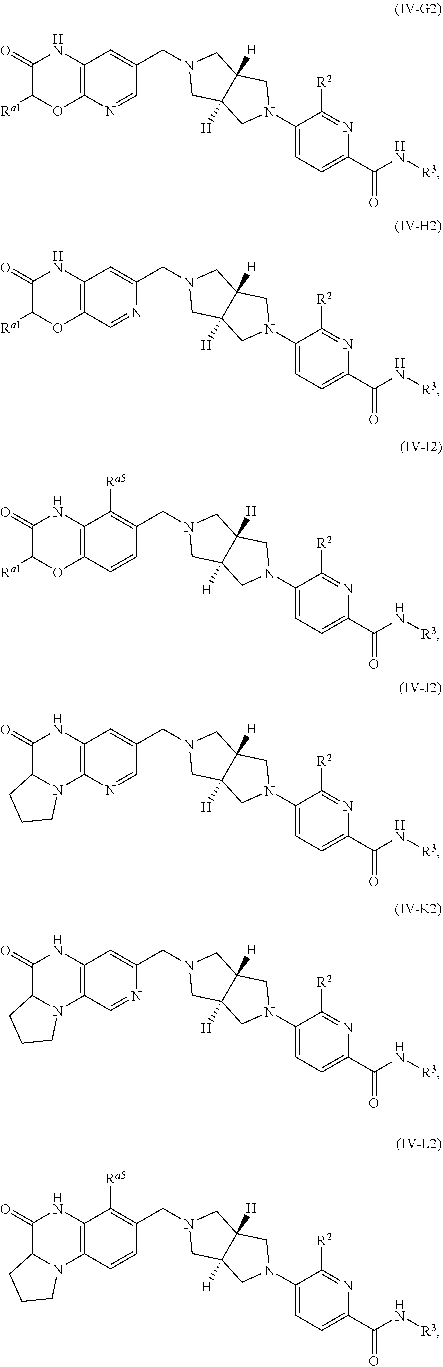

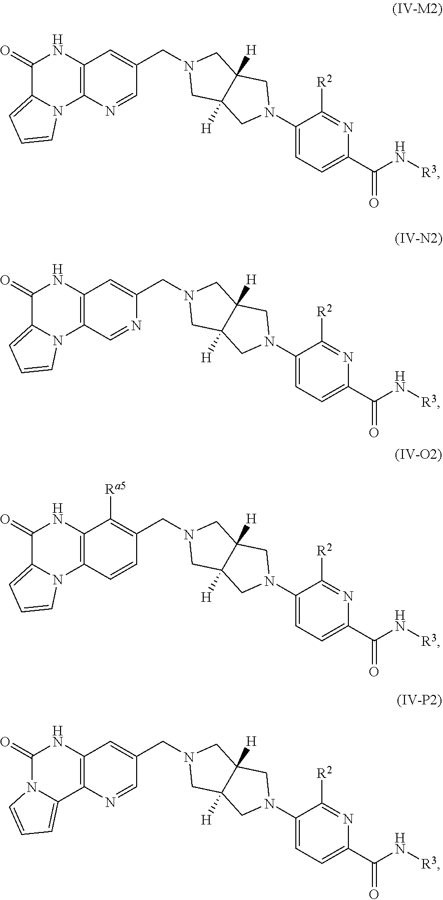

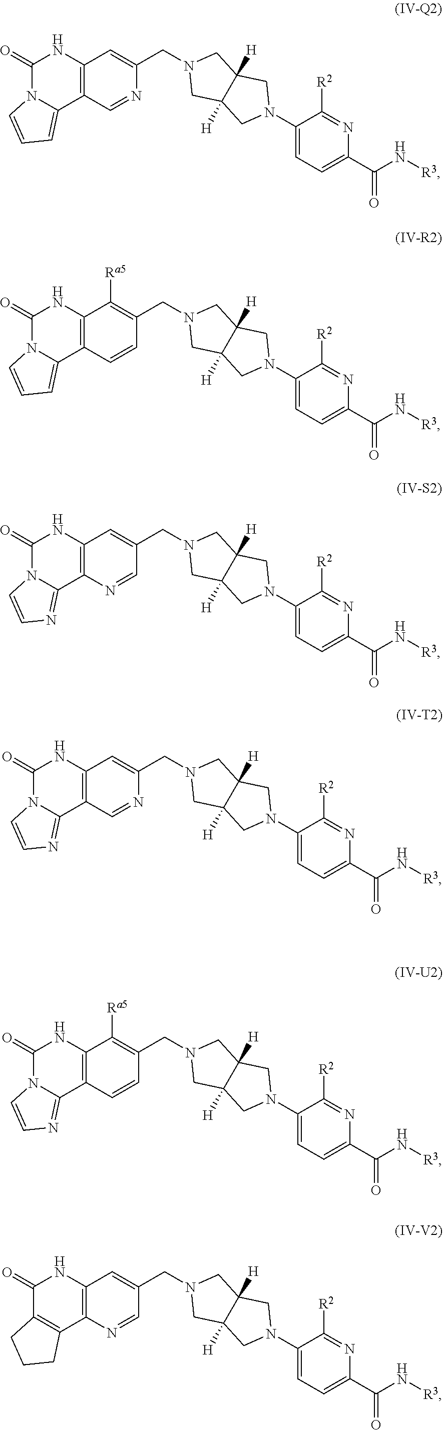

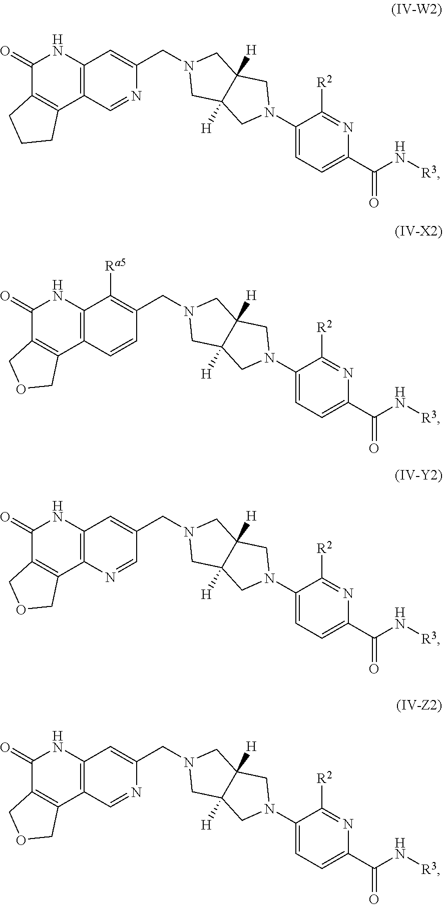

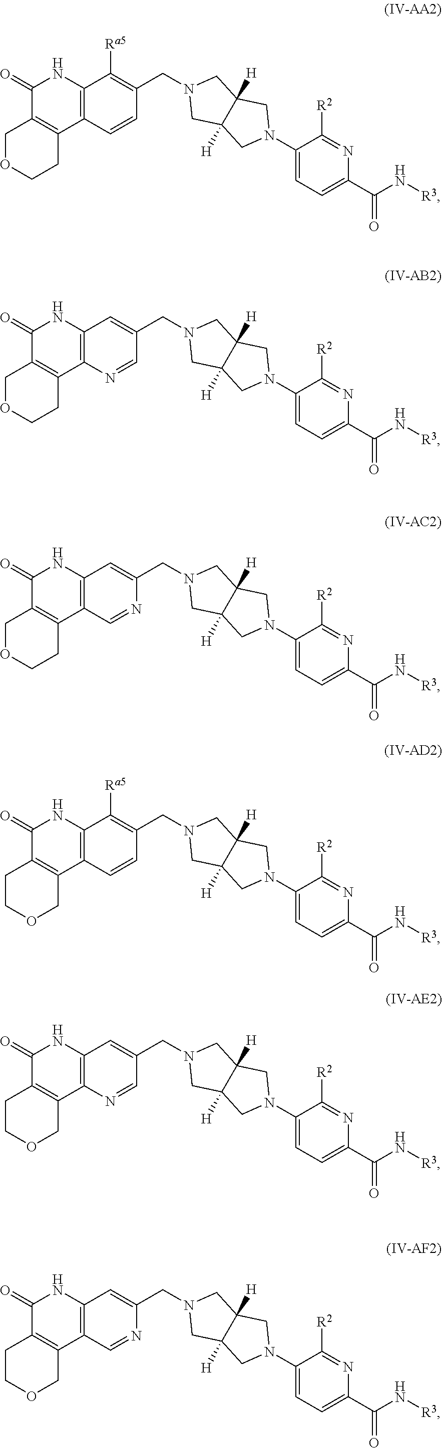

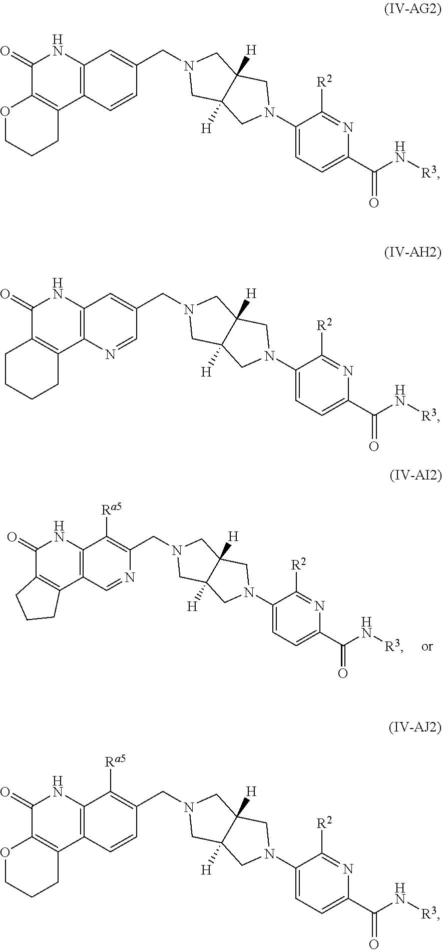

or a stereoisomer, a mixture of stereoisomers, or a pharmaceutically acceptable salt thereof.

In one embodiment, the compound is a compound of Formula (IV-V2-1), (IV-AA2-1), (IV-AB2-1), or (IV-AI2-1):

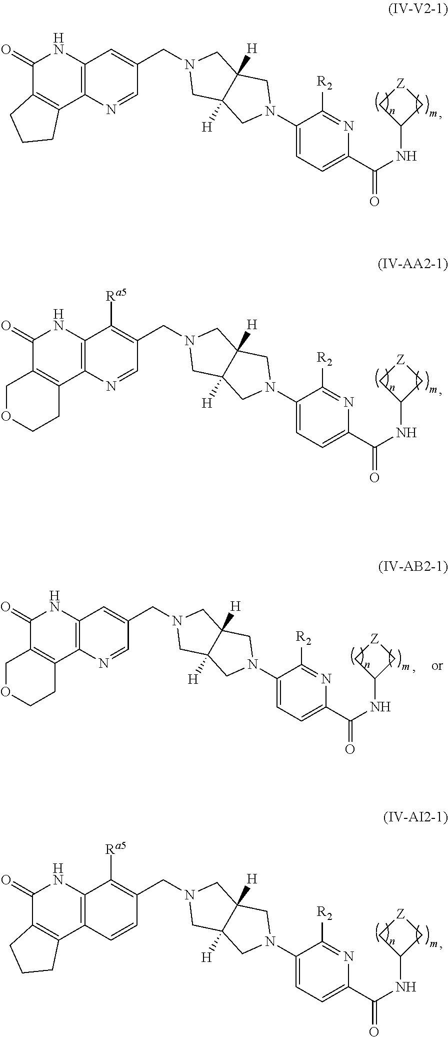

or a stereoisomer, a mixture of stereoisomers, or a pharmaceutically acceptable salt thereof, wherein:
m is 0, 1, 2, 3, or 4;
n is 1, 2, 3, or 4;
Z is O or C(R⁴)₂; and
each R⁴ is independently hydrogen, C₁-C₆ alkyl, or C₁-C₆ alkoxy.

In one embodiment, R² is hydrogen.

In one embodiment, R² is halogen. In one embodiment, R² is fluoro. In one embodiment, R² is chloro. In one embodiment, R² is bromo.

In one embodiment, R² is C₁-C₆ alkyl. In one embodiment, R² is C₁-C₄ alkyl. In one embodiment, R² is C₁-C₆ haloalkyl. In one embodiment, R² is C₁-C₄ fluoroalkyl. In one embodiment, R² is methyl. In one embodiment, R² is ethyl. In one embodiment, R² is propyl or isopropyl. In one embodiment, R² is n-butyl, iso-butyl or tert-butyl. In one embodiment, R² is fluoromethyl. In one embodiment, R² is bromomethyl. In one embodiment, R² is chloromethyl. In one embodiment, R² is difluoromethyl. In one embodiment, R² is trifluoromethyl.

In one embodiment, R² is C₁-C₆ alkoxy. In one embodiment, R² is methoxy. In one embodiment, R² is ethoxy.

In one embodiment, R² is C₃-C₈ cycloalkyl. In one embodiment, R² is C₃-C₆ cycloalkyl. In one embodiment, R² is cyclopropyl. In one embodiment, R² is cyclobutyl. In one embodiment, R² is cyclopentyl. In one embodiment, R² is cyclohexyl. In one embodiment, the cycloalkyl is optionally substituted.

In one embodiment, R² is hydrogen, halogen, C₁-C₄ alkyl, C₁-C₄ fluoroalkyl, or C₃-C₆ cycloalkyl. In one embodiment, R² is hydrogen, chloro, fluoro, methyl, difluoromethyl, trifluoromethyl, or cyclopropyl.

In one embodiment, R³ is hydrogen.

In one embodiment, R³ is C₁-C₆ alkyl. In one embodiment, R³ is C₁-C₄ alkyl. In one embodiment, R³ is methyl. In one embodiment, R³ is ethyl. In one embodiment, R³ is n-propyl or iso-propyl. In one embodiment, R³ is n-butyl, iso-butyl or tert-butyl. In one embodiment, R³ is C₅ alkyl. In one embodiment, R³ is C₆ alkyl. In one embodiment, R³ is C₃-C₈ cycloalkyl. In one embodiment, R³ is C₃-C₆ cycloalkyl. In one embodiment, R³ is cyclopropyl. In one embodiment, R³ is cyclobutyl. In one embodiment, R³ is cyclopentyl. In one embodiment, R³ is cyclohexyl.

In one embodiment, R³ is a 4- to 10-membered heterocyclyl. In one embodiment, R³ is a 4- to 8-membered heterocyclyl. In one embodiment, R³ is a 4- to 6-membered heterocyclyl. In one embodiment, R³ is a 4-membered heterocyclyl. In one embodiment, R³ is a 5-membered heterocyclyl. In one embodiment, R³ is a 6-membered heterocyclyl. In one embodiment, R³ is a 4- to 8-membered oxygen-containing heterocyclyl. In one embodiment, R³ is a 4- to 6-membered oxygen-containing heterocyclyl. In one embodiment, R³ is a 4- to 8-membered nitrogen-containing heterocyclyl. In one embodiment, R³ is a 4-membered oxygen-containing heterocyclyl. In one embodiment, R³ is a 5-membered oxygen-containing heterocyclyl. In one embodiment, R³ is a 6-membered oxygen-containing heterocyclyl. In one embodiment, R³ is oxetan-3-yl. In one embodiment, R³ is tetrahydrofuran-3-yl. In one embodiment, R³ is (R)-tetrahydrofuran-3-yl. In one embodiment, R³ is (S)-tetrahydrofuran-3-yl. In one embodiment, R³ is tetrahydro-2H-pyran-4-yl.

In one embodiment, R³ is

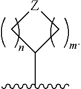

In one embodiment, R³ is

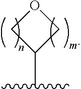

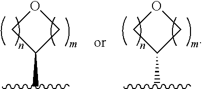

In one embodiment, R³ is

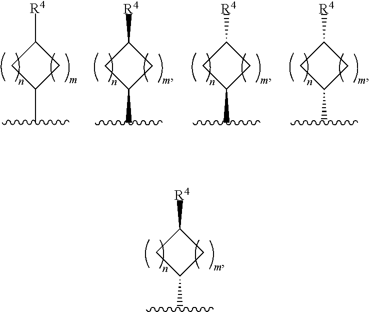

and R⁴ is C₁-C₃ alkoxy (e.g., methoxy).

In one embodiment, when ring B does not contain an oxygen ring atom (e.g., when Ring B is a carbocycle, e.g., Formula (II-V-1), Formula (II-AI-1), Formula (IV-V1-1), Formula (IV-AI1-1), Formula (IV-V2-1), Formula (IV-AI2-1)), R³ is

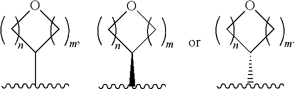

In one embodiment, when ring B contains an oxygen ring atom (e.g., when Ring B is an oxygen containing heterocyclyl, e.g., Formula (II-AA-1), Formula (II-AB-1), Formula (IV-AA1-1), Formula (IV-AB1-1), Formula (IV-AA2-1), Formula (IV-AB2-1)), R³ is

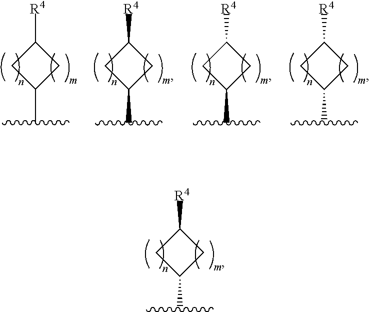

and R⁴ is C₁-C₃ alkoxy (e.g., methoxy). In another embodiment, R⁴ is hydrogen or C₁-C₃ alkyl (instead of C₁-C₃ alkoxy).

In one embodiment, m+n=1. In one embodiment, m+n=2. In one embodiment, m+n=3. In one embodiment, m+n=4. In one embodiment, m is 0, and n is 1. In one embodiment, m is 0, and n is 2. In one embodiment, m is 0, and n is 3. In one embodiment, m is 0, and n is 4. In one embodiment, m is 1, and n is 1. In one embodiment, m is 1, and n is 2. In one embodiment, m is 1, and n is 3. In one embodiment, m is 2, and n is 2.

In one embodiment, Z is O. In one embodiment, Z is C(R⁴)₂. In one embodiment, Z is CHR⁴. In one embodiment, Z is CH₂.

In one embodiment, R⁴ is hydrogen. In one embodiment, R⁴ is C₁-C₆ alkyl. In one embodiment, R⁴ is C₁-C₃ alkyl. In one embodiment, R⁴ is methyl. In one embodiment, R⁴ is ethyl. In one embodiment, R⁴ is n-propyl or iso-propyl. In one embodiment, R⁴ is C₄ alkyl. In one embodiment, R⁴ is C₅ alkyl. In one embodiment, R⁴ is C₆ alkyl. In one embodiment, R⁴ is C₁-C₆ alkoxy. In one embodiment, R⁴ is C₁-C₃ alkoxy. In one embodiment, R⁴ is methoxy. In one embodiment, R⁴ is ethoxy.

In one embodiment, R³ is unsubstituted.

In one embodiment, R³ is C₁-C₆ alkyl substituted with alkoxy. In one embodiment, R³ is C₁-C₆ alkyl substituted with C₁-C₆ alkoxy. In one embodiment, R³ is (C₁-C₆ alkoxy)-(C₁-C₆ alkyl). In one embodiment, R³ is (C₁-C₄ alkoxy)-(C₁-C₄ alkyl). In one embodiment, R³ is C₁-C₆ alkyl substituted with methoxy. In one embodiment, R³ is C₁-C₆ alkyl substituted with ethoxy. In one embodiment, R³ is 2-methoxymethyl. In one embodiment, R³ is 2-methoxyethyl. In one embodiment, R³ is 2-ethoxymethyl. In one embodiment, R³ is 2-ethoxyethyl. In one embodiment, R³ is C₁-C₆ alkyl substituted with halogen.

In one embodiment, R³ is C₃-C₈ cycloalkyl substituted with alkoxy. In one embodiment, R³ is C₃-C₈ cycloalkyl substituted with C₁-C₆ alkoxy. In one embodiment, R³ is (C₁-C₆ alkoxy)-(C₃-C₈ cycloalkyl). In one embodiment, R³ is (C₁-C₄ alkoxy)-(C₃-C₆ cycloalkyl). In one embodiment, R³ is (C₁-C₆ alkoxy)-(cyclopropyl). In one embodiment, R³ is (C₁-C₆ alkoxy)-(cyclobutyl). In one embodiment, R³ is C₃-C₈ cycloalkyl substituted with methoxy. In one embodiment, R³ is C₃-C₈ cycloalkyl substituted with ethoxy. In one embodiment, R³ is C₃-C₈ cycloalkyl substituted with halogen. In one embodiment, R³ is 3-methoxycyclobutyl. In one embodiment, R³ is (1S,3S)-3-methoxycyclobutyl. In one embodiment, R³ is (1R,3R)-3-methoxycyclobutyl.

In one embodiment, R³ is 4- to 10-membered heterocyclyl substituted with alkoxy. In one embodiment, R³ is 4- to 8-membered heterocyclyl substituted with C₁-C₆ alkoxy. In one embodiment, R³ is (C₁-C₄ alkoxy)-(4- to 6-membered heterocyclyl).

In one embodiment, when R³ has a chiral center, it has the S-configuration. In one embodiment, when R³ has a chiral center, it has the R-configuration.

In one embodiment, when a carbon at X¹ position is a chiral center, it has the S-configuration. In one embodiment, when a carbon at X¹ position is a chiral center, it has the R-configuration.

In one embodiment, the compounds provided herein are single enantiomers. In one embodiment, the compounds provided herein are single diastereoisomers. In one embodiment, the compounds provided herein are mixtures of enantiomers. In one embodiment, the compounds provided herein are mixtures of diastereoisomers. In one embodiment, the compounds provided herein are racemic compounds.

In some embodiment, the compound is a compound in Table 1, or a pharmaceutically acceptable salt thereof.

TABLE 1

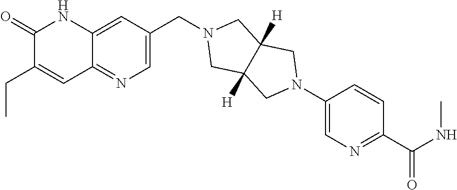 [SMILES: CCc1cc2ncc(CN3C[C@@H]4CN(c5ccc(C(=O)NC)nc5)C[C@@H]4C3)cc2[nH]c1=O]

Compound 1

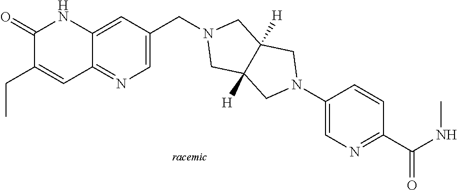 [SMILES: CCc1cc2ncc(CN3C[C@H]4CN(c5ccc(C(=O)NC)nc5)C[C@@H]4C3)cc2[nH]c1=O]

Compound 2

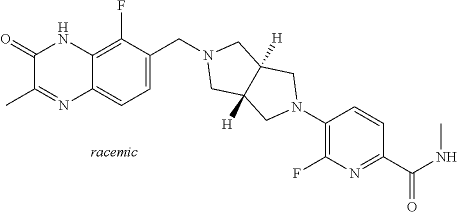 [SMILES: CNC(=O)c1ccc(N2C[C@@H]3CN(Cc4ccc5nc(C)c(=O)[nH]c5c4F)C[C@H]3C2)c(F)n1]

Compound 3

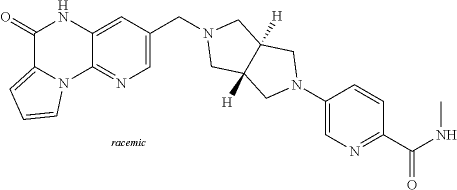 [SMILES: CNC(=O)c1ccc(N2C[C@@H]3CN(Cc4cnc5c(c4)[nH]c(=O)c4cccn45)C[C@H]3C2)cn1]

Compound 4

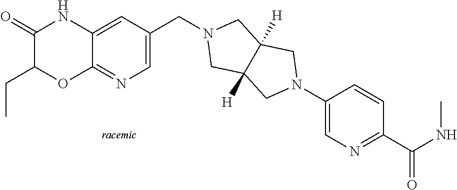 [SMILES: CCC1Oc2ncc(CN3C[C@H]4CN(c5ccc(C(=O)NC)nc5)C[C@@H]4C3)cc2NC1=O]

Compound 5

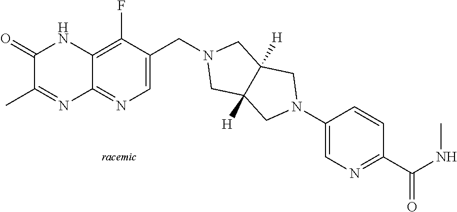 [SMILES: CNC(=O)c1ccc(N2C[C@@H]3CN(Cc4cnc5nc(C)c(=O)[nH]c5c4F)C[C@H]3C2)cn1]

Compound 6

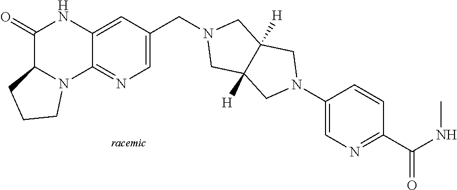 [SMILES: CNC(=O)c1ccc(N2C[C@@H]3CN(Cc4cnc5c(c4)NC(=O)[C@@H]4CCCN54)C[C@H]3C2)cn1]

Compound 7

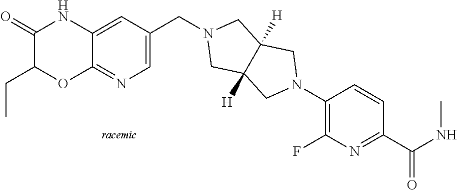 [SMILES: CCC1Oc2ncc(CN3C[C@H]4CN(c5ccc(C(=O)NC)nc5F)C[C@@H]4C3)cc2NC1=O]

Compound 8

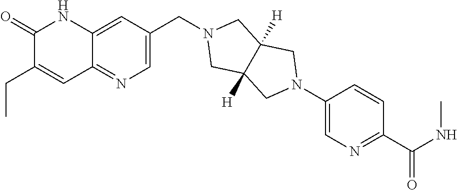 [SMILES: CCc1cc2ncc(CN3C[C@H]4CN(c5ccc(C(=O)NC)nc5)C[C@@H]4C3)cc2[nH]c1=O]

Compound 9

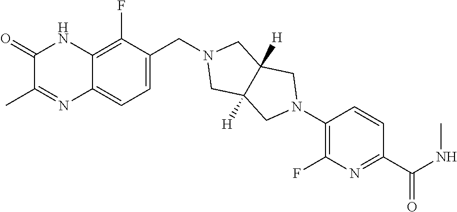 [SMILES: CNC(=O)c1ccc(N2C[C@H]3CN(Cc4ccc5nc(C)c(=O)[nH]c5c4F)C[C@@H]3C2)c(F)n1]

Compound 10

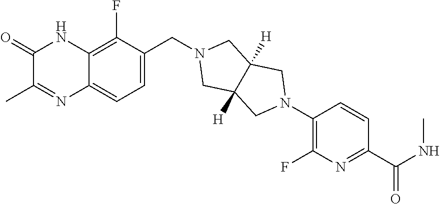 [SMILES: CNC(=O)c1ccc(N2C[C@@H]3CN(Cc4ccc5nc(C)c(=O)[nH]c5c4F)C[C@H]3C2)c(F)n1]

Compound 11

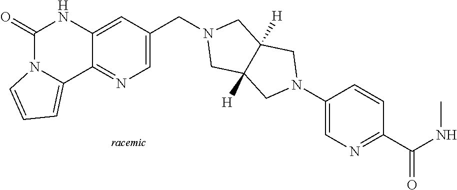 [SMILES: CNC(=O)c1ccc(N2C[C@@H]3CN(Cc4cnc5c(c4)[nH]c(=O)n4cccc54)C[C@H]3C2)cn1]

Compound 12

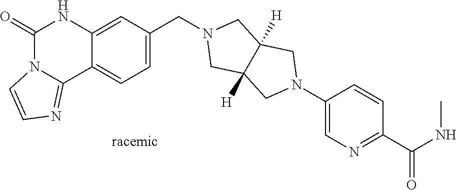 [SMILES: CNC(=O)c1ccc(N2C[C@@H]3CN(Cc4ccc5c(c4)[nH]c(=O)n4ccnc54)C[C@H]3C2)cn1]

Compound 13

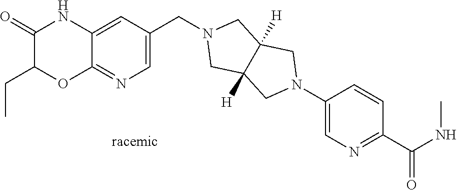 [SMILES: CCC1Oc2ncc(CN3C[C@H]4CN(c5ccc(C(=O)NC)nc5)C[C@@H]4C3)cc2NC1=O]

Compound 14

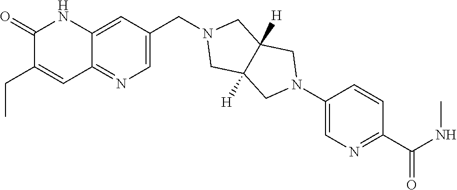 [SMILES: CCc1cc2ncc(CN3C[C@@H]4CN(c5ccc(C(=O)NC)nc5)C[C@H]4C3)cc2[nH]c1=O]

Compound 15

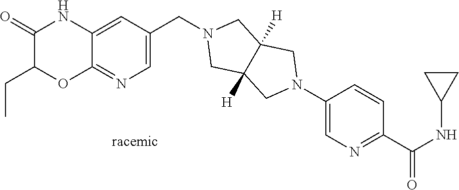 [SMILES: CCC1Oc2ncc(CN3C[C@H]4CN(c5ccc(C(=O)NC6CC6)nc5)C[C@@H]4C3)cc2NC1=O]

Compound 16

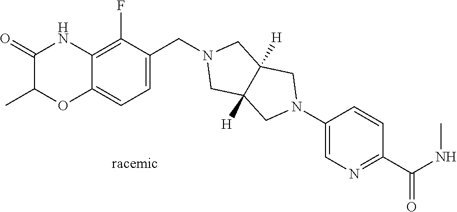 [SMILES: CNC(=O)c1ccc(N2C[C@@H]3CN(Cc4ccc5c(c4F)NC(=O)C(C)O5)C[C@H]3C2)cn1]

Compound 17

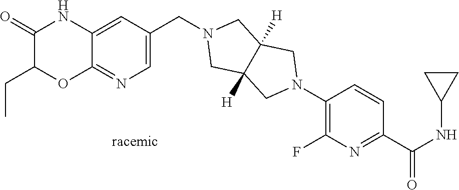 [SMILES: CCC1Oc2ncc(CN3C[C@H]4CN(c5ccc(C(=O)NC6CC6)nc5F)C[C@@H]4C3)cc2NC1=O]

Compound 18

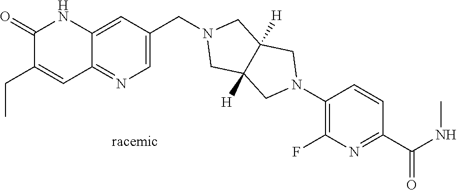 [SMILES: CCc1cc2ncc(CN3C[C@H]4CN(c5ccc(C(=O)NC)nc5F)C[C@@H]4C3)cc2[nH]c1=O]

Compound 19

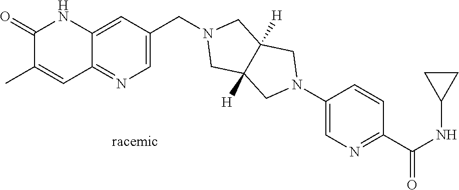 [SMILES: Cc1cc2ncc(CN3C[C@H]4CN(c5ccc(C(=O)NC6CC6)nc5)C[C@@H]4C3)cc2[nH]c1=O]

Compound 20

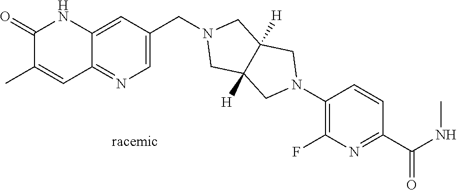 [SMILES: CNC(=O)c1ccc(N2C[C@@H]3CN(Cc4cnc5cc(C)c(=O)[nH]c5c4)C[C@H]3C2)c(F)n1]

Compound 21

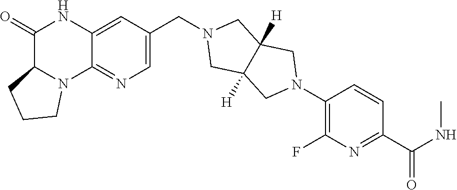 [SMILES: CNC(=O)c1ccc(N2C[C@H]3CN(Cc4cnc5c(c4)NC(=O)[C@@H]4CCCN54)C[C@@H]3C2)c(F)n1]

Compound 22

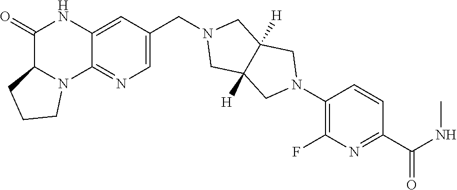 [SMILES: CNC(=O)c1ccc(N2C[C@@H]3CN(Cc4cnc5c(c4)NC(=O)[C@@H]4CCCN54)C[C@H]3C2)c(F)n1]

Compound 23

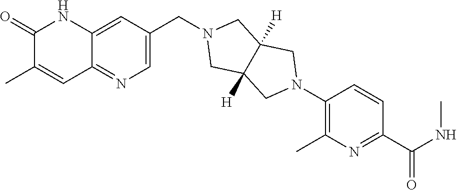 [SMILES: CNC(=O)c1ccc(N2C[C@@H]3CN(Cc4cnc5cc(C)c(=O)[nH]c5c4)C[C@H]3C2)c(C)n1]

Compound 24

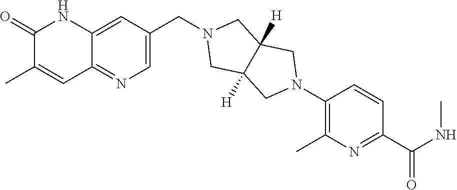 [SMILES: CNC(=O)c1ccc(N2C[C@H]3CN(Cc4cnc5cc(C)c(=O)[nH]c5c4)C[C@@H]3C2)c(C)n1]

Compound 25

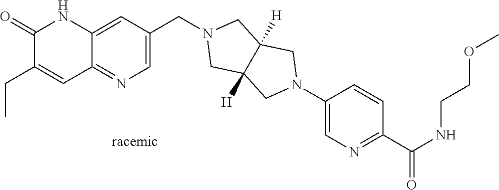 [SMILES: CCc1cc2ncc(CN3C[C@H]4CN(c5ccc(C(=O)NCCOC)nc5)C[C@@H]4C3)cc2[nH]c1=O]

Compound 26

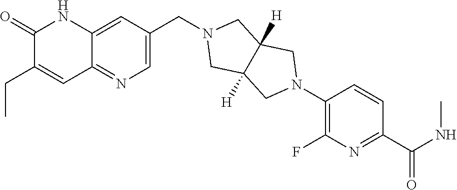 [SMILES: CCc1cc2ncc(CN3C[C@@H]4CN(c5ccc(C(=O)NC)nc5F)C[C@H]4C3)cc2[nH]c1=O]

Compound 27

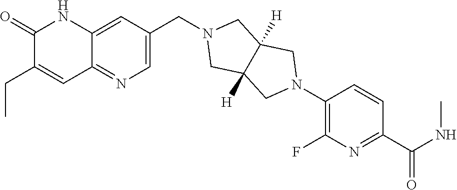 [SMILES: CCc1cc2ncc(CN3C[C@H]4CN(c5ccc(C(=O)NC)nc5F)C[C@@H]4C3)cc2[nH]c1=O]

Compound 28

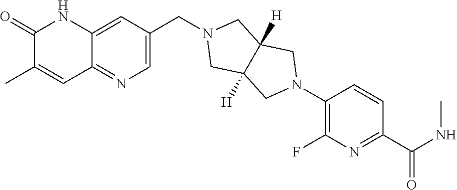 [SMILES: CNC(=O)c1ccc(N2C[C@H]3CN(Cc4cnc5cc(C)c(=O)[nH]c5c4)C[C@@H]3C2)c(F)n1]

Compound 29

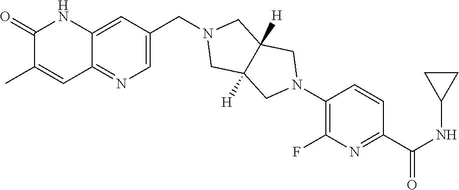 [SMILES: Cc1cc2ncc(CN3C[C@@H]4CN(c5ccc(C(=O)NC6CC6)nc5F)C[C@H]4C3)cc2[nH]c1=O]

Compound 30

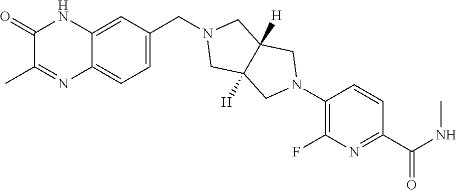 [SMILES: CNC(=O)c1ccc(N2C[C@H]3CN(Cc4ccc5nc(C)c(=O)[nH]c5c4)C[C@@H]3C2)c(F)n1]

Compound 31

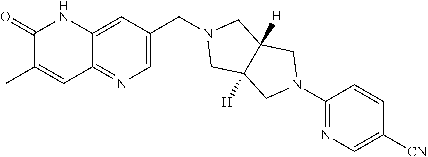 [SMILES: Cc1cc2ncc(CN3C[C@@H]4CN(c5ccc(C#N)cn5)C[C@H]4C3)cc2[nH]c1=O]

Compound 32

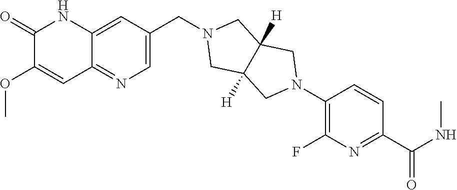 [SMILES: CNC(=O)c1ccc(N2C[C@H]3CN(Cc4cnc5cc(OC)c(=O)[nH]c5c4)C[C@@H]3C2)c(F)n1]

Compound 33

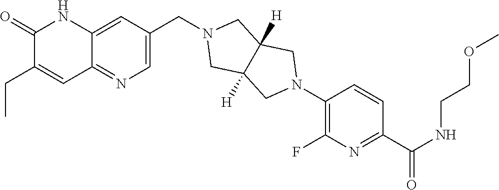 [SMILES: CCc1cc2ncc(CN3C[C@@H]4CN(c5ccc(C(=O)NCCOC)nc5F)C[C@H]4C3)cc2[nH]c1=O]

Compound 34

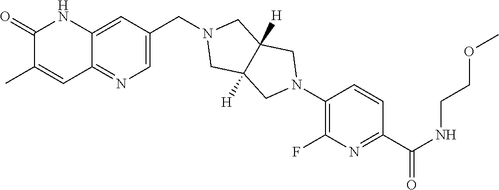 [SMILES: COCCNC(=O)c1ccc(N2C[C@H]3CN(Cc4cnc5cc(C)c(=O)[nH]c5c4)C[C@@H]3C2)c(F)n1]

Compound 35

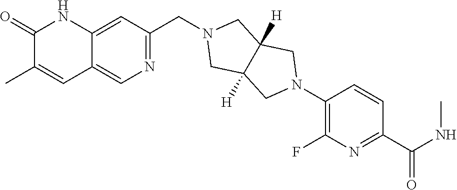 [SMILES: CNC(=O)c1ccc(N2C[C@H]3CN(Cc4cc5[nH]c(=O)c(C)cc5cn4)C[C@@H]3C2)c(F)n1]

Compound 36

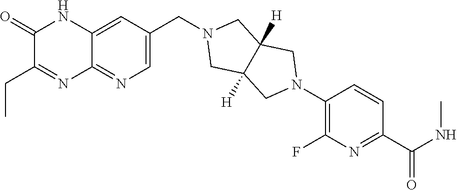 [SMILES: CCc1nc2ncc(CN3C[C@@H]4CN(c5ccc(C(=O)NC)nc5F)C[C@H]4C3)cc2[nH]c1=O]

Compound 37

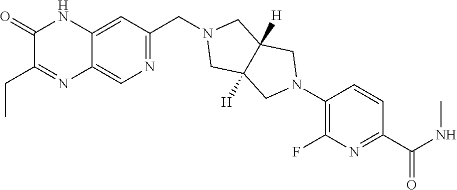 [SMILES: CCc1nc2cnc(CN3C[C@@H]4CN(c5ccc(C(=O)NC)nc5F)C[C@H]4C3)cc2[nH]c1=O]

Compound 38

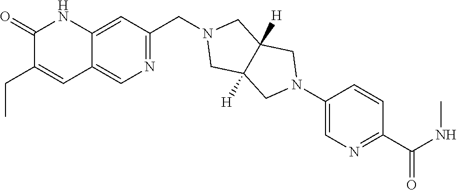 [SMILES: CCc1cc2cnc(CN3C[C@@H]4CN(c5ccc(C(=O)NC)nc5)C[C@H]4C3)cc2[nH]c1=O]

Compound 39

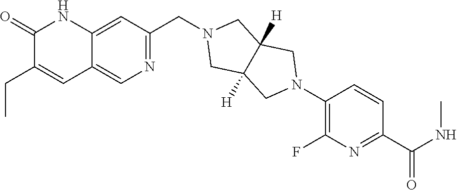 [SMILES: CCc1cc2cnc(CN3C[C@@H]4CN(c5ccc(C(=O)NC)nc5F)C[C@H]4C3)cc2[nH]c1=O]

Compound 40

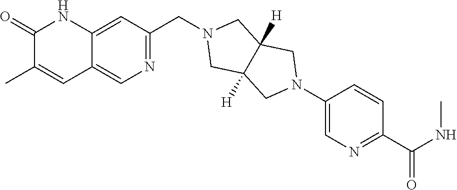 [SMILES: CNC(=O)c1ccc(N2C[C@H]3CN(Cc4cc5[nH]c(=O)c(C)cc5cn4)C[C@@H]3C2)cn1]

Compound 41

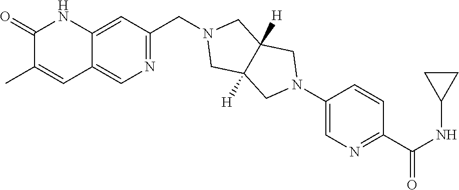 [SMILES: Cc1cc2cnc(CN3C[C@@H]4CN(c5ccc(C(=O)NC6CC6)nc5)C[C@H]4C3)cc2[nH]c1=O]

Compound 43

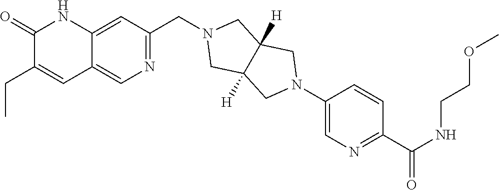 [SMILES: CCc1cc2cnc(CN3C[C@@H]4CN(c5ccc(C(=O)NCCOC)nc5)C[C@H]4C3)cc2[nH]c1=O]

Compound 44

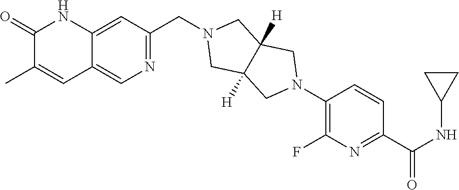 [SMILES: Cc1cc2cnc(CN3C[C@@H]4CN(c5ccc(C(=O)NC6CC6)nc5F)C[C@H]4C3)cc2[nH]c1=O]

Compound 45

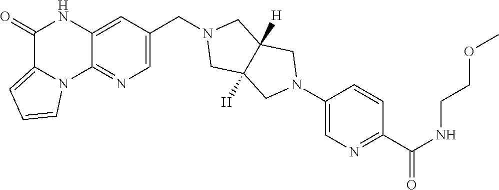 [SMILES: COCCNC(=O)c1ccc(N2C[C@H]3CN(Cc4cnc5c(c4)[nH]c(=O)c4cccn45)C[C@@H]3C2)cn1]

Compound 46

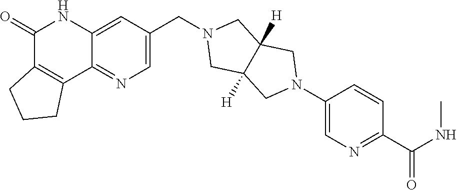 [SMILES: CNC(=O)c1ccc(N2C[C@H]3CN(Cc4cnc5c6c(c(=O)[nH]c5c4)CCC6)C[C@@H]3C2)cn1]

Compound 47

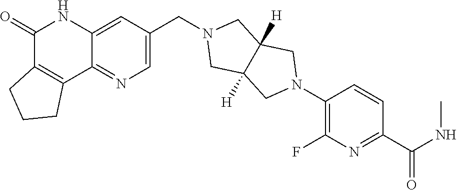 [SMILES: CNC(=O)c1ccc(N2C[C@H]3CN(Cc4cnc5c6c(c(=O)[nH]c5c4)CCC6)C[C@@H]3C2)c(F)n1]

Compound 48

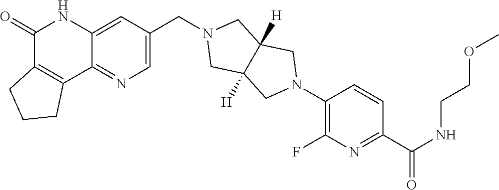 [SMILES: COCCNC(=O)c1ccc(N2C[C@H]3CN(Cc4cnc5c6c(c(=O)[nH]c5c4)CCC6)C[C@@H]3C2)c(F)n1]

Compound 49

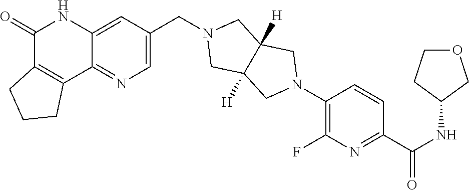 [SMILES: O=C(N[C@@H]1CCOC1)c1ccc(N2C[C@H]3CN(Cc4cnc5c6c(c(=O)[nH]c5c4)CCC6)C[C@@H]3C2)c(F)n1]

Compound 50

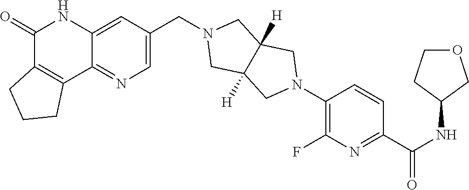 [SMILES: O=C(N[C@H]1CCOC1)c1ccc(N2C[C@H]3CN(Cc4cnc5c6c(c(=O)[nH]c5c4)CCC6)C[C@@H]3C2)c(F)n1]

Compound 51

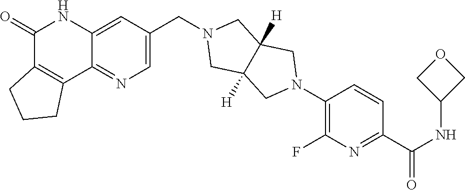 [SMILES: O=C(NC1COC1)c1ccc(N2C[C@H]3CN(Cc4cnc5c6c(c(=O)[nH]c5c4)CCC6)C[C@@H]3C2)c(F)n1]

Compound 52

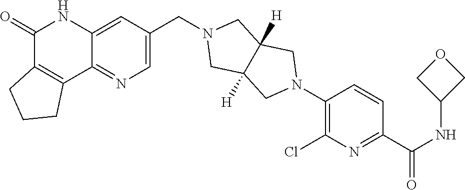 [SMILES: O=C(NC1COC1)c1ccc(N2C[C@H]3CN(Cc4cnc5c6c(c(=O)[nH]c5c4)CCC6)C[C@@H]3C2)c(Cl)n1]

Compound 53

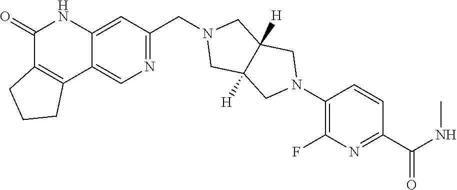 [SMILES: CNC(=O)c1ccc(N2C[C@H]3CN(Cc4cc5[nH]c(=O)c6c(c5cn4)CCC6)C[C@@H]3C2)c(F)n1]

Compound 54

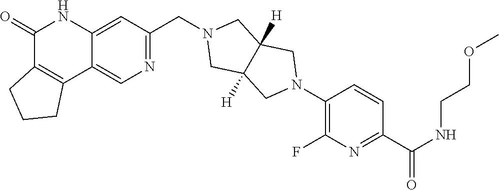 [SMILES: COCCNC(=O)c1ccc(N2C[C@H]3CN(Cc4cc5[nH]c(=O)c6c(c5cn4)CCC6)C[C@@H]3C2)c(F)n1]

Compound 55

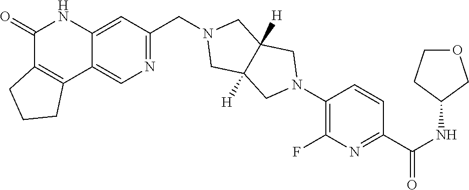 [SMILES: O=C(N[C@@H]1CCOC1)c1ccc(N2C[C@H]3CN(Cc4cc5[nH]c(=O)c6c(c5cn4)CCC6)C[C@@H]3C2)c(F)n1]

Compound 56

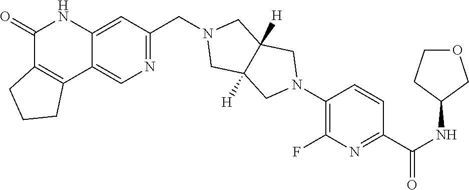 [SMILES: O=C(N[C@H]1CCOC1)c1ccc(N2C[C@H]3CN(Cc4cc5[nH]c(=O)c6c(c5cn4)CCC6)C[C@@H]3C2)c(F)n1]

Compound 57

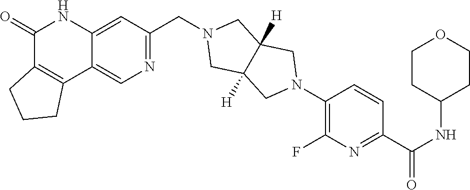 [SMILES: O=C(NC1CCOCC1)c1ccc(N2C[C@H]3CN(Cc4cc5[nH]c(=O)c6c(c5cn4)CCC6)C[C@@H]3C2)c(F)n1]

Compound 58

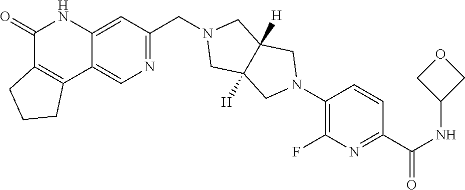 [SMILES: O=C(NC1COC1)c1ccc(N2C[C@H]3CN(Cc4cc5[nH]c(=O)c6c(c5cn4)CCC6)C[C@@H]3C2)c(F)n1]

Compound 59

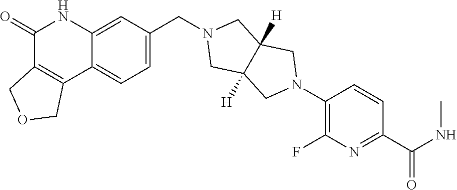 [SMILES: CNC(=O)c1ccc(N2C[C@H]3CN(Cc4ccc5c6c(c(=O)[nH]c5c4)COC6)C[C@@H]3C2)c(F)n1]

Compound 60

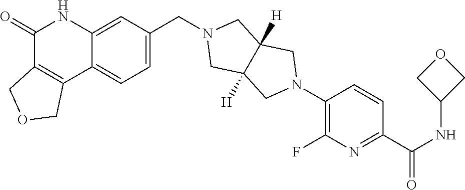 [SMILES: O=C(NC1COC1)c1ccc(N2C[C@H]3CN(Cc4ccc5c6c(c(=O)[nH]c5c4)COC6)C[C@@H]3C2)c(F)n1]

Compound 61

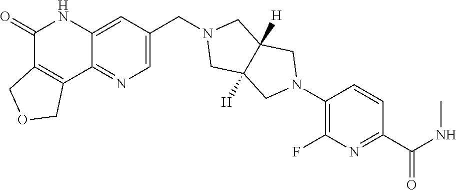 [SMILES: CNC(=O)c1ccc(N2C[C@H]3CN(Cc4cnc5c6c(c(=O)[nH]c5c4)COC6)C[C@@H]3C2)c(F)n1]

Compound 62

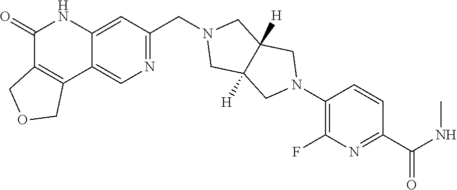 [SMILES: CNC(=O)c1ccc(N2C[C@H]3CN(Cc4cc5[nH]c(=O)c6c(c5cn4)COC6)C[C@@H]3C2)c(F)n1]

Compound 63

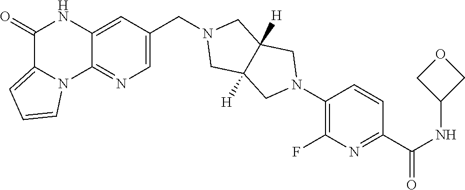 [SMILES: O=C(NC1COC1)c1ccc(N2C[C@H]3CN(Cc4cnc5c(c4)[nH]c(=O)c4cccn45)C[C@@H]3C2)c(F)n1]

Compound 64

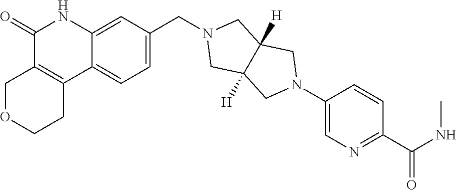 [SMILES: CNC(=O)c1ccc(N2C[C@H]3CN(Cc4ccc5c6c(c(=O)[nH]c5c4)COCC6)C[C@@H]3C2)cn1]

Compound 65

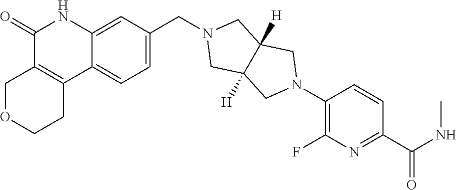 [SMILES: CNC(=O)c1ccc(N2C[C@H]3CN(Cc4ccc5c6c(c(=O)[nH]c5c4)COCC6)C[C@@H]3C2)c(F)n1]

Compound 66

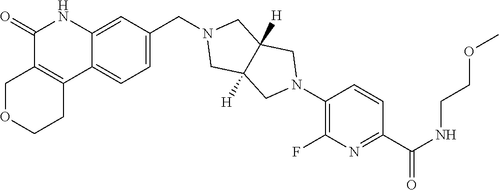 [SMILES: COCCNC(=O)c1ccc(N2C[C@H]3CN(Cc4ccc5c6c(c(=O)[nH]c5c4)COCC6)C[C@@H]3C2)c(F)n1]

Compound 67

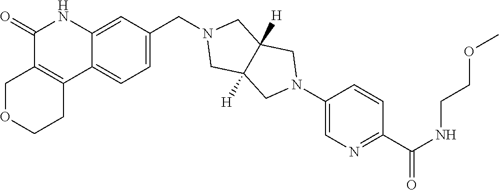 [SMILES: COCCNC(=O)c1ccc(N2C[C@H]3CN(Cc4ccc5c6c(c(=O)[nH]c5c4)COCC6)C[C@@H]3C2)cn1]

Compound 68

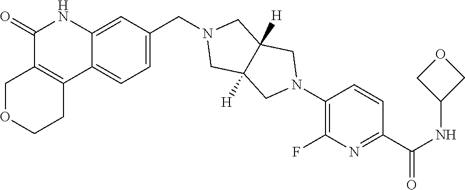 [SMILES: O=C(NC1COC1)c1ccc(N2C[C@H]3CN(Cc4ccc5c6c(c(=O)[nH]c5c4)COCC6)C[C@@H]3C2)c(F)n1]

Compound 69

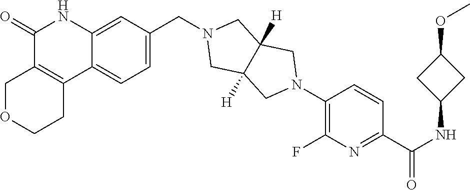 [SMILES: CO[C@H]1C[C@@H](NC(=O)c2ccc(N3C[C@H]4CN(Cc5ccc6c7c(c(=O)[nH]c6c5)COCC7)C[C@@H]4C3)c(F)n2)C1]

Compound 70

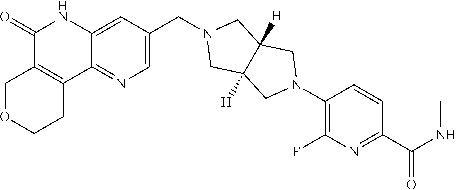 [SMILES: CNC(=O)c1ccc(N2C[C@H]3CN(Cc4cnc5c6c(c(=O)[nH]c5c4)COCC6)C[C@@H]3C2)c(F)n1]

Compound 71

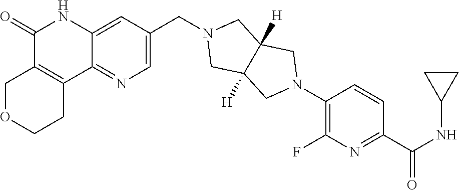 [SMILES: O=C(NC1CC1)c1ccc(N2C[C@H]3CN(Cc4cnc5c6c(c(=O)[nH]c5c4)COCC6)C[C@@H]3C2)c(F)n1]

Compound 72

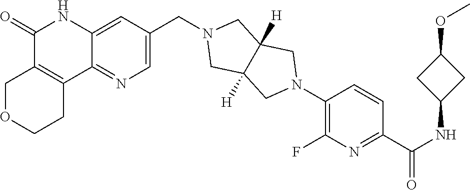 [SMILES: CO[C@H]1C[C@@H](NC(=O)c2ccc(N3C[C@H]4CN(Cc5cnc6c7c(c(=O)[nH]c6c5)COCC7)C[C@@H]4C3)c(F)n2)C1]

Compound 73

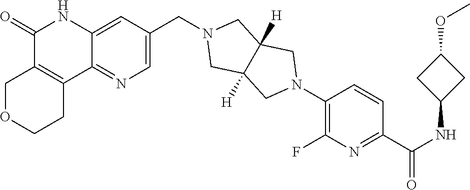 [SMILES: CO[C@H]1C[C@H](NC(=O)c2ccc(N3C[C@H]4CN(Cc5cnc6c7c(c(=O)[nH]c6c5)COCC7)C[C@@H]4C3)c(F)n2)C1]

Compound 74

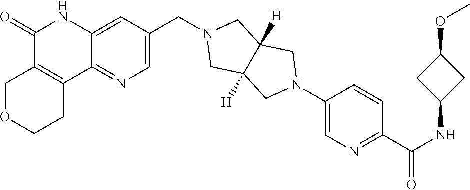 [SMILES: CO[C@H]1C[C@@H](NC(=O)c2ccc(N3C[C@H]4CN(Cc5cnc6c7c(c(=O)[nH]c6c5)COCC7)C[C@@H]4C3)cn2)C1]

Compound 75

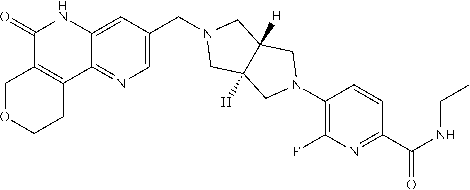 [SMILES: CCNC(=O)c1ccc(N2C[C@H]3CN(Cc4cnc5c6c(c(=O)[nH]c5c4)COCC6)C[C@@H]3C2)c(F)n1]

Compound 76

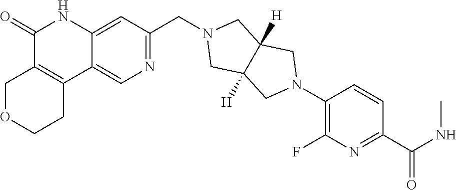 [SMILES: CNC(=O)c1ccc(N2C[C@H]3CN(Cc4cc5[nH]c(=O)c6c(c5cn4)CCOC6)C[C@@H]3C2)c(F)n1]

Compound 77

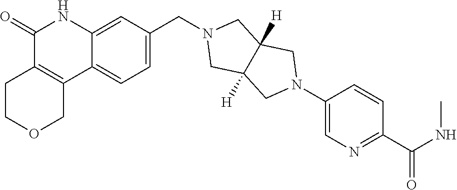 [SMILES: CNC(=O)c1ccc(N2C[C@H]3CN(Cc4ccc5c6c(c(=O)[nH]c5c4)CCOC6)C[C@@H]3C2)cn1]

Compound 78

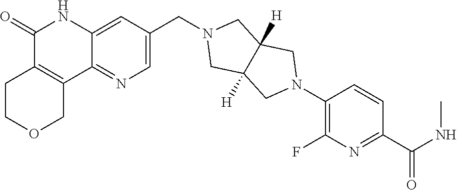 [SMILES: CNC(=O)c1ccc(N2C[C@H]3CN(Cc4cnc5c6c(c(=O)[nH]c5c4)CCOC6)C[C@@H]3C2)c(F)n1]

Compound 79

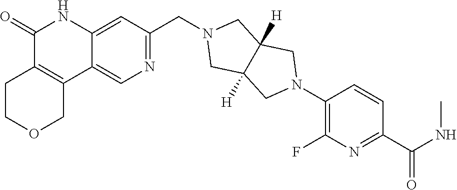 [SMILES: CNC(=O)c1ccc(N2C[C@H]3CN(Cc4cc5[nH]c(=O)c6c(c5cn4)COCC6)C[C@@H]3C2)c(F)n1]

Compound 80

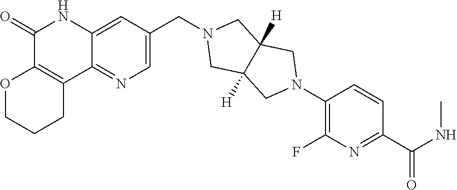 [SMILES: CNC(=O)c1ccc(N2C[C@H]3CN(Cc4cnc5c6c(c(=O)[nH]c5c4)OCCC6)C[C@@H]3C2)c(F)n1]

Compound 81

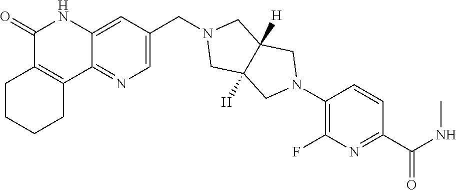 [SMILES: CNC(=O)c1ccc(N2C[C@H]3CN(Cc4cnc5c6c(c(=O)[nH]c5c4)CCCC6)C[C@@H]3C2)c(F)n1]

Compound 82

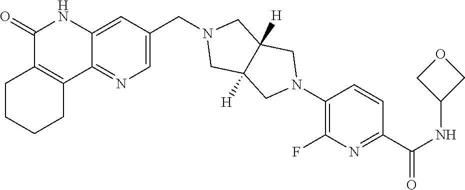 [SMILES: O=C(NC1COC1)c1ccc(N2C[C@H]3CN(Cc4cnc5c6c(c(=O)[nH]c5c4)CCCC6)C[C@@H]3C2)c(F)n1]

Compound 83

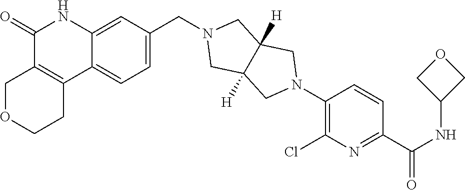 [SMILES: O=C(NC1COC1)c1ccc(N2C[C@H]3CN(Cc4ccc5c6c(c(=O)[nH]c5c4)COCC6)C[C@@H]3C2)c(Cl)n1]

Compound 84

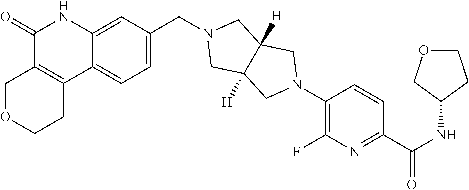 [SMILES: O=C(N[C@H]1CCOC1)c1ccc(N2C[C@H]3CN(Cc4ccc5c6c(c(=O)[nH]c5c4)COCC6)C[C@@H]3C2)c(F)n1]

Compound 85

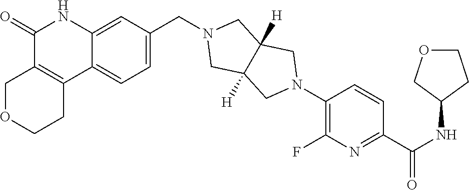 [SMILES: O=C(N[C@@H]1CCOC1)c1ccc(N2C[C@H]3CN(Cc4ccc5c6c(c(=O)[nH]c5c4)COCC6)C[C@@H]3C2)c(F)n1]

Compound 86

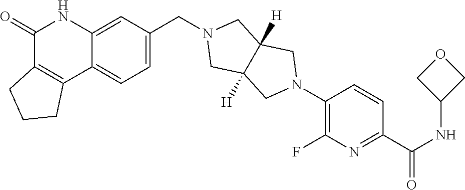 [SMILES: O=C(NC1COC1)c1ccc(N2C[C@H]3CN(Cc4ccc5c6c(c(=O)[nH]c5c4)CCC6)C[C@@H]3C2)c(F)n1]

Compound 87

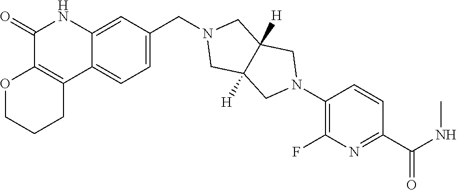 [SMILES: CNC(=O)c1ccc(N2C[C@H]3CN(Cc4ccc5c6c(c(=O)[nH]c5c4)OCCC6)C[C@@H]3C2)c(F)n1]

Compound 88

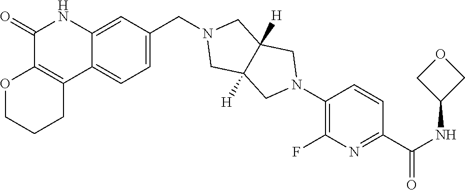

Compound 89

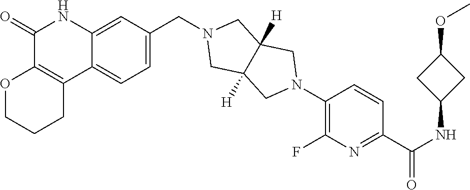 [SMILES: CO[C@H]1C[C@@H](NC(=O)c2ccc(N3C[C@H]4CN(Cc5ccc6c7c(c(=O)[nH]c6c5)OCCC7)C[C@@H]4C3)c(F)n2)C1]

Compound 90

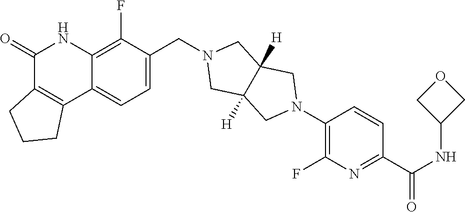 [SMILES: O=C(NC1COC1)c1ccc(N2C[C@H]3CN(Cc4ccc5c6c(c(=O)[nH]c5c4F)CCC6)C[C@@H]3C2)c(F)n1]

Compound 91

In one embodiment, without being bound by a particular theory, a compound provided herein is poly(ADP-ribose) polymerase 1 (PARP1) inhibitor that exhibits strong DNA trapping capability. In one embodiment, a compound provided herein has an IC₅₀ of less than 100 nM measured by an DNA trapping assay. In one embodiment, the IC₅₀ is less than 50 nM. In one embodiment, the IC₅₀ is less than 10 nM.

In one embodiment, without being bound by a particular theory, a compound provided herein is PARP1 inhibitor that exhibits good tumor penetration and retention capability (e.g., long residence time in tumor issue). In one embodiment, without being bound by a particular theory, the compound is able to achieve a higher concentration in tumor than in plasma (e.g., higher tumor/plasma concentration ratio). In one embodiment, without being bound by a particular theory, lower plasma concentration may lead to less side effects.

In one embodiment, the compounds provided herein are poly(ADP-ribose) polymerase 1 (PARP1) inhibitors that reduce the level of PARPI protein and/or inhibit or reduce at least one biological activity of PARP1 protein. In one embodiment, the expression level of the PARP1 protein is reduced by 10%, 20%, 30%, 40%, 50%, 60%, 70%, 80%, 90%, 95% or 99%. In one embodiment, the biological activity of the PARP1 protein is reduced by 10%, 20%, 30%, 40%, 50%, 60%, 70%, 80%, 90%, 95% or 99%.

In one embodiment, the compounds provided herein are capable of penetrating the blood brain barrier (BBB). In one embodiment, the ratio of compound that penetrates the BBB is >0.05, wherein 1 is complete BBB penetration, and 0 is no penetration. In one embodiment, the ratio of compound that penetrates the BBB is >0.1. In one embodiment, the ratio of compound that penetrates the BBB is >0.2. In one embodiment, the ratio of compound that penetrates the BBB is >0.3. In one embodiment, the ratio of compound that penetrates the BBB is >0.3.

In one embodiment, the compounds provided herein bind to a PARPI protein with an affinity in the range of about 1 μM to about 100 μM, about 1 μM to about 1 μM, about 1 μM to about 500 nM, or about 1 μM to about 100 nM. In some embodiment, the compounds provided herein bind to a PARP1 protein with an affinity of about 1 μM to about 100 μM, about 1 nM to about 100 μM, about 1 μM to about 100 μM, about 1 μM to about 50 μM, about 1 μM to about 40 μM, about 1 μM to about 30 μM, about 1 μM to about 20 μM, or about 1 μM to about 10 μM, about 1 μM, about 5 μM, about 10 μM, about 15 μM, about 20 μM, about 25 μM, about 30 μM, about 35 μM, about 40 μM, about 45 μM, about 50 μM, about 60 μM, about 70 μM, about 80 μM, about 90 μM, or about 100 μM. In some embodiment, the compounds provided herein bind to a PARP1 protein with an affinity of about 100 nM to about 1 μM, about 100 nM to about 900 nM, about 100 nM to about 800 nM, about 100 nM to about 700 nM, about 100 nM to about 600 nM, about 100 nM to about 500 nM, about 100 nM to about 400 nM, about 100 nM to about 300 nM, about 100 nM to about 200 nM, about 200 nM to about 1 μM, about 300 nM to about 1 μM, about 400 nM to about 1 μM, about 500 nM to about 1 μM, about 600 nM to about 1 μM, about 700 nM to about 1 μM, about 800 nM to about 1 μM, about 900 nM to about 1 μM, about 100 nM, about 200 nM, about 300 nM, about 400 nM, about 500 nM, about 600 nM, about 700 nM, about 800 nM, or about 900 nM. In some embodiment, the compounds provided herein bind to a PARP1 protein with an affinity of about 1 nM to about 100 nM, about 1 nM to about 90 nM, about 1 nM to about 80 nM, about 1 nM to about 70 nM, about 1 nM to about 60 nM, about 1 nM to about 50 nM, about 1 nM to about 40 nM, about 1 nM to about 30 nM, about 1 nM to about 20 nM, about 1 nM to about 10 nM, about 10 nM to about 100 nM, about 20 nM to about 100 nM, about 30 nM to about 100 nM, about 40 nM to about 100 nM, about 50 nM to about 100 nM, about 60 nM to about 100 nM, about 70 nM to about 100 nM, about 80 nM to about 100 nM, about 90 nM to about 100 nM, about 1 nM, about 2 nM, about 3 nM, about 4 nM, about 5 nM, about 6 nM, about 7 nM, about 8 nM, about 9 nM, about 10 nM, about 20 nM, about 30 nM, about 40 nM, about 50 nM, about 60 nM, about 70 nM, about 80 nM, about 90 nM, or about 100 nM. In some embodiment, the compounds provided herein bind to a PARP1 protein with an affinity of less than about 1 μM, less than about 500 nM, less than about 100 nM, less than about 10 nM, or less than about 1 nM. In one embodiment, the compounds provided herein bind to a PARP1 protein with an affinity of less than 1 nM.

In one embodiment, the compounds provided herein inhibit PARP1 activity with an IC₅₀ of about 1 pM to about 100 μM, or about 1 pM to about 1 μM, or about 1 pM to about 500 nM, or about 1 pM to about 100 nM. In one embodiment, the compounds provided herein inhibit PARP1 activity with an IC₅₀ of about 1 pM to about 100 μM, about 1 nM to about 100 μM, about 1 μM to about 100 μM, about 1 μM to about 50 μM, about 1 μM to about 40 μM, about 1 μM to about 30 μM, about 1 μM to about 20 μM, or about 1 μM to about 10 μM, about 1 μM, about 5 μM, about 10 μM, about 15 μM, about 20 μM, about 25 μM, about 30 μM, about 35 μM, about 40 μM, about 45 μM, about 50 μM, about 60 μM, about 70 μM, about 80 μM, about 90 μM, or about 100 μM. In some embodiment, the compounds provided herein inhibit PARP1 activity with an IC₅₀ of about 100 nM to about 1 μM, about 100 nM to about 900 nM, about 100 nM to about 800 nM, about 100 nM to about 700 nM, about 100 nM to about 600 nM, about 100 nM to about 500 nM, about 100 nM to about 400 nM, about 100 nM to about 300 nM, about 100 nM to about 200 nM, about 200 nM to about 1 μM, about 300 nM to about 1 μM, about 400 nM to about 1 μM, about 500 nM to about 1 μM, about 600 nM to about 1 μM, about 700 nM to about 1 μM, about 800 nM to about 1 μM, about 900 nM to about 1 μM, about 100 nM, about 200 nM, about 300 nM, about 400 nM, about 500 nM, about 600 nM, about 700 nM, about 800 nM, or about 900 nM. In some embodiment, the compounds provided herein inhibit PARP1 activity with an IC₅₀ of about 1 nM to about 100 nM, about 1 nM to about 90 nM, about 1 nM to about 80 nM, about 1 nM to about 70 nM, about 1 nM to about 60 nM, about 1 nM to about 50 nM, about 1 nM to about 40 nM, about 1 nM to about 30 nM, about 1 nM to about 20 nM, about 1 nM to about 10 nM, about 10 nM to about 100 nM, about 20 nM to about 100 nM, about 30 nM to about 100 nM, about 40 nM to about 100 nM, about 50 nM to about 100 nM, about 60 nM to about 100 nM, about 70 nM to about 100 nM, about 80 nM to about 100 nM, about 90 nM to about 100 nM, about 1 nM, about 2 nM, about 3 nM, about 4 nM, about 5 nM, about 6 nM, about 7 nM, about 8 nM, about 9 nM, about 10 nM, about 20 nM, about 30 nM, about 40 nM, about 50 nM, about 60 nM, about 70 nM, about 80 nM, about 90 nM, or about 100 nM. In one embodiment, the compounds provided herein inhibit PARPI activity with an IC₅₀ of less than 1 μM, less than 500 nM, less than 100 nM, less than 10 nM, or less than 1 nM. In one embodiment, the compounds provided herein inhibit PARP1 activity with an IC₅₀ of less than 1 nM.

### Methods of Use

In one embodiment, provided herein is a method of treating diseases or conditions by inhibiting the PARP1 protein comprising administering to a subject in need thereof a therapeutically effective amount of a compound provided herein or a pharmaceutical composition provided herein.

In one embodiment, provided herein is a method of treating a cancer, comprising administering to a subject having the cancer a therapeutically effective amount of a compound provided herein or a pharmaceutical composition provided herein.

In one embodiment, the cancer is deficient in Homologous Recombination (HR) dependent DNA double-strand DNA break (DSB) repair pathway.

In one embodiment, the cancer comprises one or more cancer cells having a reduced or abrogated ability to repair DNA DSB by HR relative to normal cells.

In one embodiment, the cancer is a cancer that comprises cancer cells that are heterozygous for a mutation in a gene encoding a component of HR dependent DNA DSB repair pathway. Without being bound by a particular theory, a cancer which is deficient in HR dependent DNA DSB repair may comprise one or more cancer cells which have a reduced or abrogated ability to repair DNA DSBs through that pathway compared with normal cells.

In one embodiment, provided herein is a method of treating cancer that is deficient in Homologous Recombination (HR) dependent DNA DSB repair activity. Without being bound by a particular theory, The HR dependent DNA DSB repair pathway repairs double-strand breaks (DSBs) in DNA via homologous mechanisms to reform a continuous DNA helix (K. K. Khanna and S. P. Jackson, Nat. Genet. 27 (3): 247-254 (2001)). The components of the HR dependent DNA DSB repair pathway include, but are not limited to, ATM (NM_000051), RAD51 (NM_002875), RAD51 L1 (NM_002877), RAD51 C (NM_002876), RAD51 L3 (NM_002878), DMC1 (NM_007068), XRCC2 (NM_005431), XRCC3 (NM_005432), RAD52 (NM_002879), RAD54L (NM_003579), RAD54B (NM_012415), BRCA1 (NM_007295), BRCA2 (NM_000059), RAD50 (NM_005732), MRE11A (NM_005590) and NBS1 (NM_002485).

In one embodiment, the cancer cells have a breast cancer type 1 (BRCA1) or breast cancer type 2 (BRCA2) deficient phenotype.

In one embodiment, the cancer cells are deficient in BRCA1. In one embodiment, the cancer cells are deficient in BRCA2. In one embodiment, the cancer cells are deficient in both BRCA1 and BRCA2.

In one embodiment, the cancer is a cancer that comprises cancer cells that is heterozygous for a mutation in BRCA1 and/or BRCA2.

In one embodiment, a compound provided herein is used to treat a cancer, wherein the cancer is a BRCA1 mutant cancer. In one embodiment, a compound provided herein is used to treat a cancer, wherein the cancer is a BRCA2 mutant cancer. In one embodiment, a compound provided herein is used to treat a cancer, wherein the cancer is a BRCA1 mutant cancer and a BRCA2 mutant cancer. In one embodiment, the cancer is not a BRCA1 mutant cancer or a BRCA2 mutant cancer. In one embodiment, the cancer is a BRCA1 deficient cancer. In one embodiment, the cancer is a BRCA2 deficient cancer. In one embodiment, the cancer is a BRCA1 deficient cancer and a BRCA2 deficient cancer.

In one embodiment, the cancer is breast cancer, ovarian cancer, pancreas cancer, prostate cancer, hematological cancer, gastrointestinal cancer, lung cancer, or brain cancer. In one embodiment, the brain cancer is a glioma or glioblastoma. In one embodiment, the brain cancer is a metastatic cancer arising from a tumor elsewhere in the body such as breast, ovary, pancreas, prostate, hematological, gastrointestinal such as gastric and colorectal, or lung cancer such as small cell or non-small cell lung cancer.

In one embodiment, provided herein is a method of inhibiting PARPI protein, comprising contacting the PARP1 protein with an effective amount of a compound provided herein or a pharmaceutical composition provided herein. In one embodiment, the inhibition occurs in a subject suffering from a PARP1 mediated disease or condition.

In one embodiment, the PARP1 mediated disease or condition is cancer.

In one embodiment, the cancer is breast cancer. In one embodiment, the cancer is ovarian cancer. In one embodiment, the cancer is pancreas cancer. In one embodiment, the cancer is prostate cancer. In one embodiment, the cancer is hematological cancer. In one embodiment, the cancer is gastrointestinal cancer. In one embodiment, the cancer is lung cancer. In one embodiment, the cancer is brain cancer.

In some embodiment, such methods comprise (a) identifying a cancer in a subject as a PARP1 inhibitor-sensitive cancer, and then (b) administering a therapeutically effective amount of a compound provided herein to the subject.

In some embodiment, compounds provided herein are provided for use as a medicament or are provided for use in preparing a medicament, e.g., for the treatment of cancer. In some embodiment, compounds provided herein are provided for use in a method for the treatment of cancer.

In one embodiment, compounds provided herein are provided for use in a method for the treatment of diseases or conditions by inhibiting PARP1 protein.

### Pharmaceutical Compositions

Also provided herein are pharmaceutical compositions comprising a compound provided herein and a pharmaceutically acceptable excipient.

The pharmaceutical composition provided herein may be administered by various routes to mammals, including rodents and humans. In one embodiment, the administration is intranasal, intravenous, intraperitoneal, intramuscular, intraarticular, intralesional, intratracheal, subcutaneous, or intradermal administration. In one embodiment, the administration is intravenous administration. In one embodiment, the administration is intramuscular administration.

In one embodiment, a pharmaceutical composition provided herein may be orally administered in any orally acceptable dosage form including capsules, tablets, aqueous suspensions or solutions.

In one embodiment, compounds provided herein are administered to a mammal in the form of a raw chemical without any other components present. In one embodiment, compounds provided herein are administered to a mammal as part of a pharmaceutical composition containing the compound combined with a suitable pharmaceutically acceptable carrier (see, for example, Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)). Non-limiting examples of pharmaceutically suitable carriers include solids and/or liquids such as water, alcohol and glycerol. The amount of carrier in the treatment composition can range from about 5 to about 99 wt % based on the total weight of the treatment composition or therapeutic combination. Pharmaceutically acceptable excipients and diluents include, but are not limited to buffers, preservatives, binders, fillers, disintegrants, lubricants, wetting agents, antioxidants, flavorings, thickeners, coloring agents, emulsifiers, suspending agents and the like. Non-limiting examples of excipients and diluents also include sucrose, lactose, dextrose, sorbitol, mannitol, erythritol, maltitol, starch, alginate, gelatin, calcium phosphate, calcium silicate, cellulose, polyvinyl pyrrolidone, water, methylhydroxy benzoate, propylhydroxy benzoate, talc, magnesium stearate and mineral oil.

In one embodiment, a pharmaceutical composition provided herein may be prepared as liquid suspensions or solutions using a liquid, such as an oil, water, an alcohol, and combinations of these.

In one embodiment, a pharmaceutical composition provided herein may be prepared as a sterile injectable, which may be aqueous or oleaginous suspensions. The suspension may be formulated according to techniques known in the art using suitable dispersing or wetting agents (e.g., Polysorbate). The sterile injectable formulation may be also a sterile injectable solution or suspension in a diluent or solvent. In addition, sterile fixed oils are conventionally employed as a solvent or suspending medium. Pharmaceutically acceptable natural oils or fatty acids may also be used in the preparation of injectable formulations as well as.

In one embodiment, a pharmaceutical composition provided herein may be administered in the form of suppositories for rectal administration.

In one embodiment, a pharmaceutical composition provided herein may also be administered topically, especially when the target of treatment includes areas or organs readily accessible by topical application, including diseases of the eye, the skin, or the lower intestinal tract. Topical application for the lower intestinal tract may be effected in a rectal suppository formulation or in a suitable enema formulation. Topically-transdermal patches may also be used. For topical applications, the pharmaceutical compositions may be formulated in a suitable ointment, lotion, or cream containing the active component suspended or dissolved in one or more carriers.

In one embodiment, a pharmaceutical composition provided herein may also be administered ophthalmically and formulated as micronized suspensions in isotonic, pH adjusted sterile saline, or, solutions in isotonic, pH adjusted sterile saline, either with or without a preservative such as benzyl alkonium chloride. In one embodiment, for ophthalmic uses, the pharmaceutical compositions may be formulated in an ointment such as petrolatum.

In one embodiment, a pharmaceutical composition provided herein may also be administered by nasal aerosol or inhalation. Such compositions are prepared according to techniques well known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In one embodiment, the pharmaceutical compositions to be used for in vivo administration can be sterile. In one embodiment, this is accomplished by filtration through, e.g., sterile filtration membranes.

In one embodiment, pharmaceutical compositions provided herein include all compositions where a compound provided herein is combined with one or more pharmaceutically acceptable carriers. In one embodiment, the compound provided herein is present in the composition in an amount that is effective to achieve its intended therapeutic purpose.

In one embodiment, a pharmaceutical composition provided herein can be administered to any patient that may experience the beneficial effects of a compound provided herein. In one embodiment, the patients are mammals, e.g., humans and companion animals. In one embodiment, the patient is a human.

In one embodiment, also provided herein are kits which comprise a compound provided herein (or a composition comprising a compound provided herein) packaged in a manner that facilitates their use to practice methods provided herein. In one embodiment, the kit includes a compound provided herein (or a composition comprising a compound provided herein) packaged in a container, such as a sealed vial, with a label affixed to the container or included in the kit that describes use of the compound or composition to practice the method provided herein. In one embodiment, the compound or composition is packaged in a unit dosage form. In one embodiment, the kit further includes a device suitable for administering the compound or composition according to the intended route of administration. In one embodiment, the kit comprises a compound provided herein, and instructions for administering the compound to a patient having cancer.

### Examples

### Example 1: Process of Preparation

In one embodiment, provided herein is a process (Method 1) for the preparation of a compound provided herein comprising the following steps:

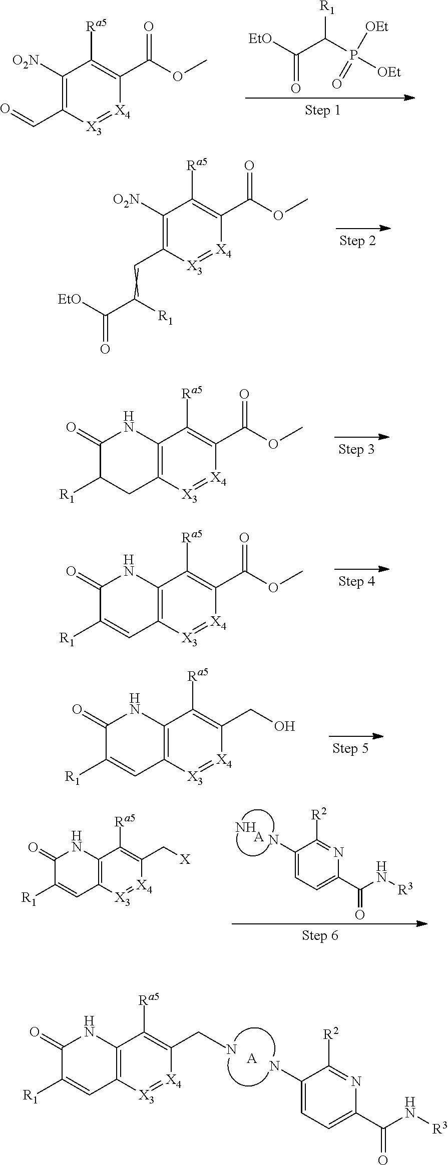

wherein X is a halogen, such as Br and Cl.

Step 1: at a suitable temperature such as from about −78 to 25° C., in the presence of a suitable inorganic base such as sodium hydride in a suitable solvent such as THF.

Step 2: at a suitable temperature such as 25 to 80° C., in the presence of a suitable hydrogenation catalyst such as palladium on carbon, in the presence of hydrogen at a suitable pressure such as 15-100 psi in a suitable solvent such as EtOAc or EtOH.

Step 3: at a suitable temperature such as from room temperature to about 120° C., in the presence of a suitable oxidant such as DDQ or MnO₂ in a suitable solvent such as THF or dioxane.

Step 4: at a suitable temperature such as from 0 to 25° C., in the presence of a suitable reductant such as LiAlH₄ in a suitable solvent such as THF.

Step 5: at a suitable temperature such as from 25 to 80° C., in the presence of a suitable halogenation reagent such as SOCl₂ or HBr in a suitable solvent such as DCM or AcOH.

Step 6: at a suitable temperature such as from 25 to 80° C., in the presence of a suitable base such as DIEA or TEA, in the presence of an additive such as potassium iodide in a suitable solvent such as MeCN.

In one embodiment, provided herein is a process (Method 2) for the preparation of a compound provided herein comprising the following steps:

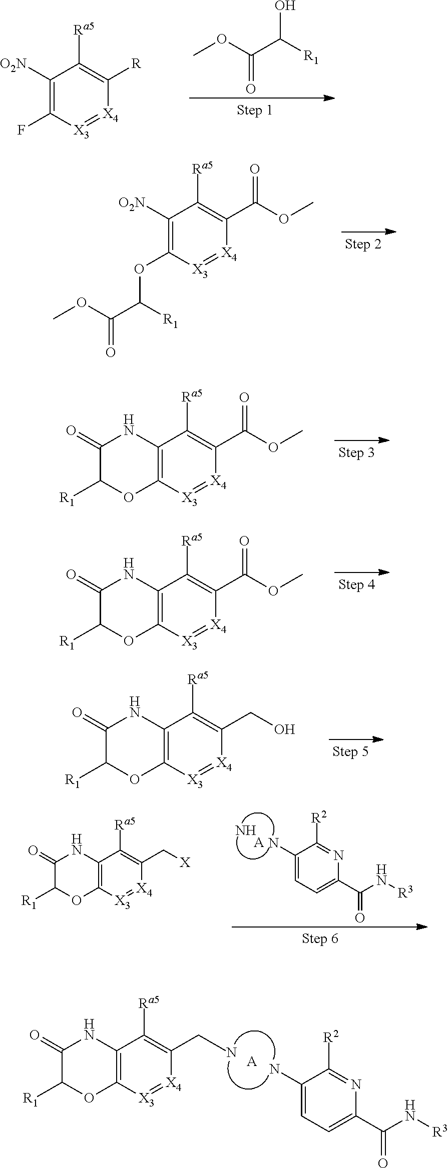

wherein X is a halogen such as Br and Cl.

Step 1: at a suitable temperature such as from about −78 to 25° C., in the presence of a suitable base such as DIEA or TEA in a suitable solvent such as THE or MeCN.

Step 2: at a suitable temperature such as 25 to 80° C., in the presence of a suitable hydrogenation catalyst such as palladium on carbon, in the presence of hydrogen at a suitable pressure such as 15-100 psi in a suitable solvent such as EtOAc or EtOH.

Step 3: at a suitable temperature such as from room temperature to about 120° C., in the presence of a suitable oxidant such as DDQ or MnO₂ in a suitable solvent such as THE or dioxane.

Step 4: at a suitable temperature such as from 0 to 25° C., in the presence of a suitable reductant such as LiAlH₄ in a suitable solvent such as THF.

Step 5: at a suitable temperature such as from 25 to 80° C., in the presence of a suitable halogenation reagent such as SOCl₂ or HBr in a suitable solvent such as DCM or AcOH.

Step 6: at a suitable temperature such as from 25 to 80° C., in the presence of a suitable base such as DIEA or TEA, in the presence of an additive such as potassium iodide in a suitable solvent such as MeCN.

In one embodiment, provided herein is a process (Method 3) for the preparation of a compound provided herein comprising the following steps:

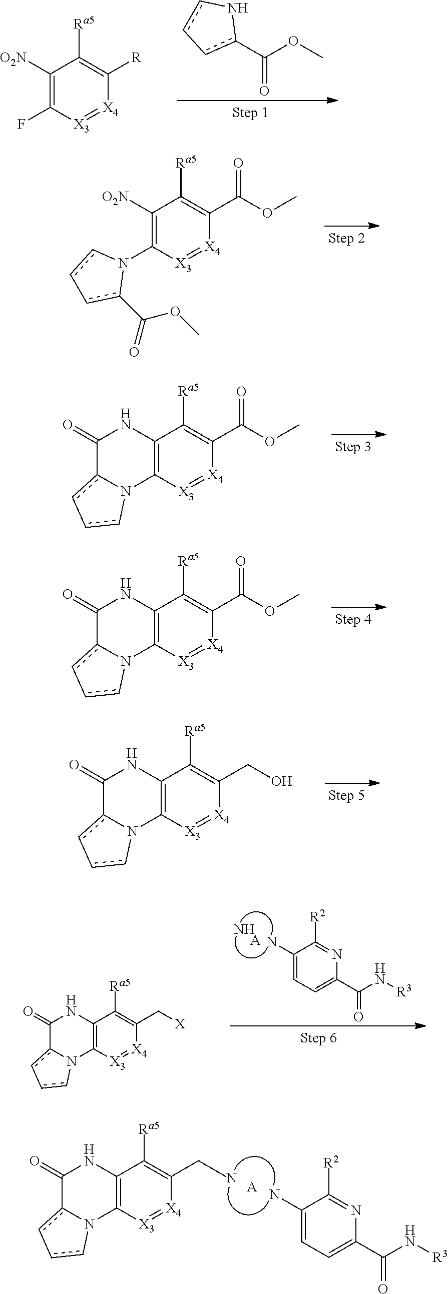

wherein X is a halogen such as Br and Cl.

Step 1: at a suitable temperature such as from about −78 to 25° C., in the presence of a suitable base such as DIEA or sodium hydride in a suitable solvent such as THF or MeCN.

Step 2: at a suitable temperature such as 25 to 80° C., in the presence of a suitable hydrogenation catalyst such as palladium on carbon, in the presence of hydrogen at a suitable pressure such as 15-100 psi in a suitable solvent such as EtOAc or EtOH.

Step 3: at a suitable temperature such as from room temperature to about 120° C., in the presence of a suitable oxidant such as DDQ or MnO₂ in a suitable solvent such as THF or dioxane.

Step 4: at a suitable temperature such as from 0 to 25° C., in the presence of a suitable reductant such as LiAlH₄ in a suitable solvent such as THF.

Step 5: at a suitable temperature such as from 25 to 80° C., in the presence of a suitable halogenation reagent such as SOCl₂ or HBr in a suitable solvent such as DCM or AcOH.

Step 6: at a suitable temperature such as from 25 to 80° C., in the presence of a suitable base such as DIEA or TEA, in the presence of an additive such as potassium iodide in a suitable solvent such as MeCN.

In one embodiment, provided herein is a process (Method 4) for the preparation of a compound provided herein comprising the following steps:

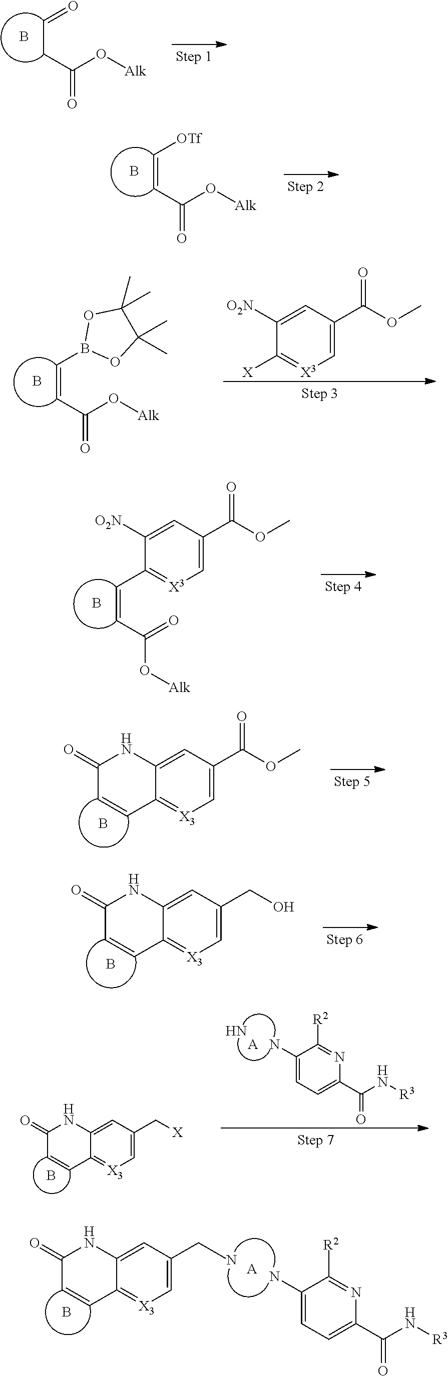

wherein X is a halogen such as Br and Cl; wherein Alk is methyl or ethyl.

Step 1: at a suitable temperature such as from −78° C. to room temperature, in the presence of a suitable base such as DIEA, sodium hydride, or 2,6-di-tert-butyl-4-methylpyridine, in the presence of a suitable triflation reagent such as trifluoromethanesulfonic anhydride, in a suitable solvent such as DCM.

Step 2: at a suitable temperature such as from room temperature to 120° C., in the presence of a suitable base such as KOAc, in the presence of a suitable palladium catalyst such as Pd(dppf)Cl₂, in a suitable solvent such as dioxane.

Step 3: at a suitable temperature such as from 60 to 120° C., in the presence of a suitable inorganic base such as K₂CO₃ or Cs₂CO₃, in the presence of a suitable palladium catalyst such as Pd(dppf)Cl₂ or Pd(PPh₃)₄, in a suitable solvent or mixed solvent such as dioxane or dioxane/H₂O.

Step 4: at a suitable temperature such as from room temperature to 120° C., in the presence of a suitable inorganic additive such as NH₄Cl, in the presence of a suitable metal reductant such as Fe, in a suitable solvent mixture such as THE/MeOH/H₂O.

Step 5: at a suitable temperature such as from 0 to 25° C., in the presence of a suitable reductant such as LiAlH₄, in a suitable solvent such as THF.

Step 6: at a suitable temperature such as from 25 to 80° C., in the presence of a suitable halogenation reagent such as SOCl₂ or HBr, in a suitable solvent such as DCM or AcOH.

Step 7: at a suitable temperature such as from 25 to 80° C., in the presence of a suitable base such as DIEA or TEA, in the presence of an additive such as potassium iodide, in a suitable solvent such as MeCN.

In one embodiment, provided herein is a process (Method 5) for the preparation of a compound provided herein comprising the following steps:

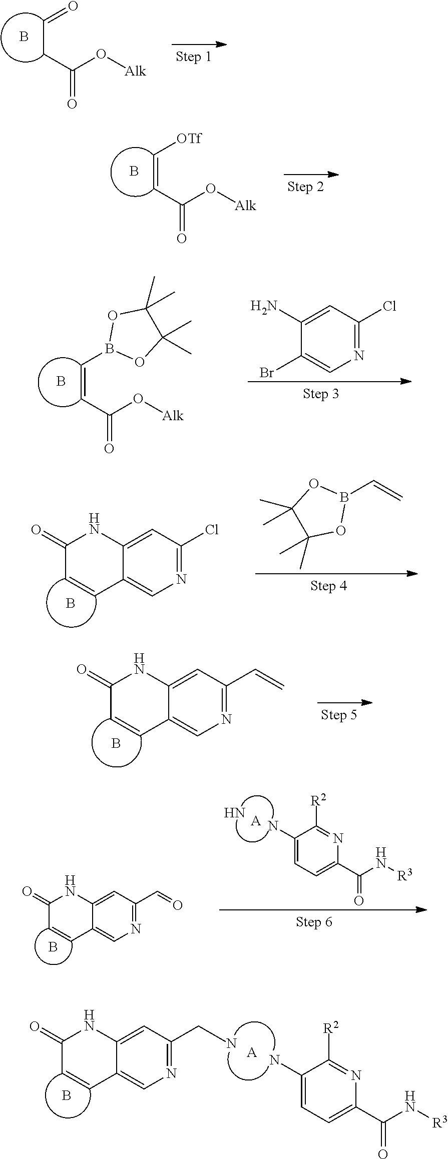

wherein Alk is methyl or ethyl.

Step 1: at a suitable temperature such as from −78° C. to room temperature, in the presence of a suitable base such as DIEA, sodium hydride, or 2,6-di-tert-butyl-4-methylpyridine, in the presence of a suitable triflation reagent such as trifluoromethanesulfonic anhydride, in a suitable solvent such as DCM.

Step 2: at a suitable temperature such as from room temperature to 120° C., in the presence of a suitable base such as KOAc, in the presence of a suitable palladium catalyst such as Pd(dppf)Cl₂, in a suitable solvent such as dioxane.

Step 3: at a suitable temperature such as from 60 to 120° C., in the presence of a suitable inorganic base such as K₂CO₃, in the presence of a suitable palladium catalyst such as Pd(dppf)Cl₂, in a suitable solvent or mixed solvent such as dioxane or dioxane/H₂O.

Step 4: at a suitable temperature such as from 60 to 120° C., in the presence of a suitable inorganic base such as K₂CO₃, in the presence of a suitable palladium catalyst such as Pd(dppf)Cl₂, in a suitable mixed solvent such as dioxane/H₂O.

Step 5: at a suitable temperature such as from room temperature to 60° C., in the presence of a suitable oxidation reagent combination such as K₂OsO₄/NaIO₄, in the presence of a suitable base such as 2,6-lutidine, in a suitable mixed solvent such as THF/H₂O.

Step 6: at a suitable temperature such as from room temperature to 60° C., in the presence of a suitable reductant such as NaBH₃CN or NaBH (OAc)₃, in the presence of a suitable acidic additive such as AcOH, in a suitable solvent such as DCM or MeOH.

Several methods for preparing the compounds provided herein are illustrated in the following examples. Unless otherwise noted, all starting materials were obtained from commercial suppliers and used without further purification, or alternatively can be synthesized by a skilled artisan in the field using well-known methods.

| Abbreviation | Meaning |
|---|---|
| ° C. | degree centigrade |
| AcOH | acetic acid |
| aq. | aqueous |
| CDI | carbonyldiimidazole |
| Cs₂CO₃ | cesium carbonate |
| DBU | 1,8-diazabicyclo[5,4,0]-undec-7-ene |
| DCE | dichloroethane |
| DCM | dichloromethane |
| DDQ | 2,3-dichloro-5,6-dicyano-1,4-benzoquinone |
| DIBAL-H | diisobutylaluminum hydride |
| DIEA/DIPEA | N,N-diisopropylethylamine |
| DMF | N,N-dimethylformamide |
| DMSO | dimethyl sulfoxide |
| Et₂O | diethyl ether |
| EtOAc/EA | ethyl acetate |
| EtOH | ethanol |
| FA | formic acid |
| Fe | iron |
| g | gram(s) |
| h/hr | hour |
| H₂ | hydrogen |
| H₂O | water |
| H₂SO₄ | sulfuric acid |
| HATU | (1-[bis(dimethylamino)methylene]-1H-1,2,3-triazolo |
| | [4,5-b]pyridinium 3-oxide hexafluorophosphate |
| hex | hexane |
| HBr | hydrobromic acid |
| HCl | hydrochloric acid |
| HPLC | high performance liquid chromatography |
| K₂CO₃ | potassium carbonate |
| K₃PO₄ | potassium phosphate |
| KOAc | potassium acetate |
| KOH | potassium hydroxide |
| LiAlH₄/LAH | lithium aluminum hydride |
| Li-HMDS | lithium bis(trimethylsilyl)amide |
| M | mol/L |
| MeCN/ACN | acetonitrile |
| MeOH | methanol |
| mg | milligram(s) |
| MgSO₄ | magnesium sulfate |
| min | minute(s) |
| mL | milliliters |
| mmol | millimoles |
| MnO₂ | manganese dioxide |
| N₂ | nitrogen |
| Na₂CO₃ | sodium carbonate |
| NaH | Sodium hydride |
| Na₂SO₄ | sodium sulfate |
| NaHCO₃ | sodium bicarbonate |
| NaOAc | sodium acetate |
| NaOH | sodium hydroxide |
| NBS | N-Bromosuccinimide |
| NH₃•H₂O | ammonium hydroxide |
| NH₄Cl | ammonium chloride |
| NH₄HCO₃ | ammonium bicarbonate |
| Pd(dppf)Cl₂ | [1,1′-bis(diphenylphosphino) |
| | ferrocene]dichloropalladium(II) |
| Pd/C | palladium on carbon |
| PE | petroleum ether |
| prep | preparative |
| r.t./rt | room temperature |
| RT | retention time |
| SeO₂ | Selenium oxide |
| SOCl₂ | thionyl chloride |
| TEA | triethylamine |
| Temp | temperature |
| TFA | trifluoroacetic acid |
| THF | tetrahydrofuran |
| TLC | thin layer chromatography |
| UV | ultraviolet |
| wt. | weight |
| Zn | Zinc |

### Example 2: Preparation of Intermediates

For intermediates that were used in the next reaction step as a crude or as a partially purified intermediate, in some cases no molar amounts are mentioned for such intermediate in the next reaction step or alternatively estimated molar amounts or theoretical molar amounts for such intermediate in the next reaction step are indicated in the reaction protocols described below.

### Preparation of Intermediate 1

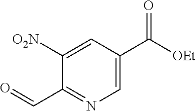 [SMILES: CCOC(=O)c1cnc(C=O)c([N+](=O)[O-])c1]

To a solution of ethyl ethyl 6-methyl-5-nitronicotinate (10 g, 47.58 mmol) in dioxane (200 mL) was added SeO₂ (7.92 g, 71.38 mmol). The mixture was stirred at 110° C. for 16 hr. The reaction mixture was cooled to room temperature, filtered through a pad of celite and the celite was washed with ethyl acetate. The combined filtrate was concentrated, and the residue was purified by silica gel chromatography to afford intermediate 1 (9.3 g, yield: 87%) as a yellow oil.

### Preparation of Intermediate 2

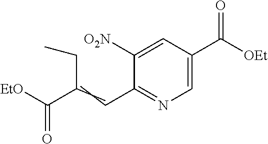 [SMILES: CCOC(=O)C(=Cc1ncc(C(=O)OCC)cc1[N+](=O)[O-])CC]

To a stirred solution of NaH (6.6 g, 165.10 mmol) in anhydrous THF (100 mL) was added dropwise ethyl 2-(diethoxyphosphoryl) butanoate (41.64 g, 165.10 mmol) in anhydrous THF (100 mL) at 0° C. The resulting mixture was stirred at 0° C. for 10 min then warmed to room temperature over 10 min and stirred at 40° C. for 5 min. And then the reaction mixture was cooled to −78° C., a solution of intermediate 1 (15.478 g, 68.79 mmol) in anhydrous THF (150 mL) was slowly added. The mixture was stirred at −78° C. for 30 min. The mixture was quenched with saturated ammonium chloride aqueous solution THF (20 mL), extracted with ethyl acetate (200×2 mL). The combined the organic layers were washed with brine, dried over Na₂SO₄, filtered and concentrated, the resulting residue was purified by silica gel chromatography to afford intermediate 2 (mixture of E/Z isomers=1:1) (13.7 g, yield: 62%) as a yellow oil.

The following intermediate was synthesized by an analogous method as described above for intermediate 2.

| Intermediate No | Structure | Starting Materials |
|---|---|---|
| 37 | 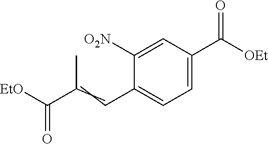 [SMILES: CCOC(=O)C(C)=Cc1ccc(C(=O)OCC)cc1[N+](=O)[O-]] | Intermediate 1 & ethyl 2- (diethoxyphosphoryl)propanoate |

### Preparation of Intermediate 3

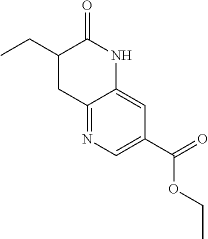 [SMILES: CCOC(=O)c1cnc2c(c1)NC(=O)C(CC)C2]

To a mixture of intermediate 2 (13.7 g, 42.51 mmol)(mixture of E/Z isomers=1:1) in EtOH (200 mL) was added 10% wt. Pd/C (7 g), and the mixture was stirred at room temperature overnight under H₂ atmosphere. The mixture was filtered through a celite bed and the celite bed was washed with ethanol. After concentration, 4M HCl in dioxanes (100 ml) was added to the resulting residue and the mixture was stirred at room temperature for 1 h. The mixture was diluted with Et₂O, and the precipitate was filtered, washed with diethyl ether twice to afford intermediate 3 (6.7 g, yield: 63.6%) as a white solid.

The following intermediate was synthesized by an analogous method as described above for intermediate 3.

| Intermediate No | Structure | Starting Materials |
|---|---|---|
| 38 | 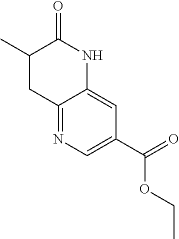 [SMILES: CCOC(=O)c1cnc2c(c1)NC(=O)C(C)C2] | Intermediate 37 |

### Preparation of

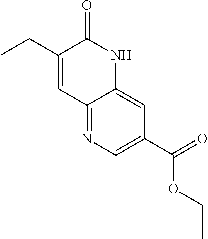 [SMILES: CCOC(=O)c1cnc2cc(CC)c(=O)[nH]c2c1]

To a solution of intermediate 3 (6.7 g, 26.99 mmol) in 1,4-dioxane (120 mL) was added DDQ (6.738 g, 29.68 mmol). The mixture was stirred for 3 h under reflux. The solvent was removed under reduced pressure, then saturated sodium hydrogen carbonate aqueous solution was added to the residue and the resulting mixture was stirred at rt for 1 h. The precipitate was filtered to afford intermediate 4 (4 g, yield: 60%) as a gray solid.

The following intermediates were synthesized by an analogous method as described above for intermediate 4.

| Intermediate No | Structure | Starting Materials |
|---|---|---|
| 39 | 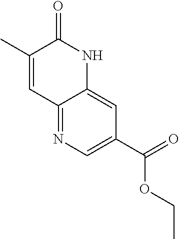 [SMILES: CCOC(=O)c1cnc2cc(C)c(=O)[nH]c2c1] | Intermediate 38 |
| 50 | 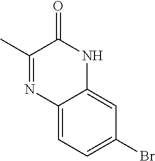 | Intermediate 49 |

### Preparation of Intermediate 5

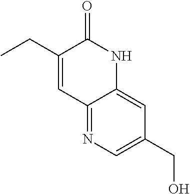

To a solution of intermediate 4 (4 g, 16.26 mmol) in THF (20 mL) was added dropwise LiAlH₄ (32.52 mL, 32.52 mmol, 1 M in THF) at 0° C. over a period of 45 min under nitrogen. The resulting mixture was stirred at 0° C. for 1.5 h. The reaction mixture was quenched with 1 M HCl (aq.)(15 mL). The reaction mixture was concentrated and the solid was diluted with water (~70 mL) and 15 mL of 1M HCl solution gave a yellow suspension. The solid was collected by filtration, washed with water, diethyl ether and dried to afford the crude product as a yellow solid (contaminated by some inorganic salt). This solid was suspended in a mixture of methanol and DCM (2:1)(100 mL) and heated to reflux for 1 hr. The solid was filtered off. This solid was resuspended in methanol/DCM mixture and repeated this procedure for 5 times to extract most of the product from the solid mixture. The combined filtrate was then concentrated to about 40 mL and the solid was collected by filtration, washed with ether, dried under vacuum to afford intermediate 5 (2.67 g, yield: 80.39%) as a yellow solid.

The following intermediates were synthesized by an analogous method as described above for intermediate 5.

| Intermediate No | Structure | Starting Materials |
|---|---|---|
| 15 | 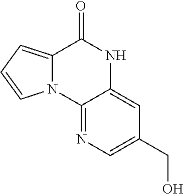 [SMILES: O=c1[nH]c2cc(CO)cnc2n2cccc12] | Intermediate 14 |
| 27 | 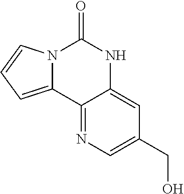 [SMILES: O=c1[nH]c2cc(CO)cnc2c2cccn12] | Intermediate 26 |
| 30 | 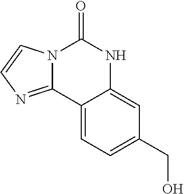 [SMILES: O=c1[nH]c2cc(CO)ccc2c2nccn12] | Intermediate 29 |
| 40 | 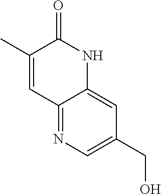 | Intermediate 39 |

### Preparation of Intermediate 6

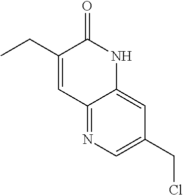

To a suspension of intemediate 6 (204 mg, 1.00 mmol) in DCM (4 mL) was added SOCl₂ (714 mg, 6.00 mmol) and one drop of DMF at 0° C. The resulting mixture was stirred at rt for 6 hours. The mixture was concentrated to give crude intermdiate 6 (222 mg, yield: 100%), which was used directly for next step.

The following intermediates were synthesized by an analogous method as described above for intermediate 6.

| Intermediate No | Structure | Starting Materials |
|---|---|---|
| 16 | 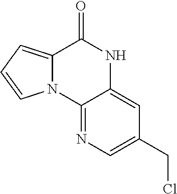 [SMILES: O=c1[nH]c2cc(CCl)cnc2n2cccc12] | Intermediate 15 |
| 20 | 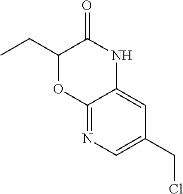 | Intermediate 19 |
| 24 | 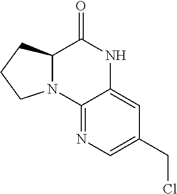 [SMILES: O=C1Nc2cc(CCl)cnc2N2CCC[C@@H]12] | Intermediate 23 |
| 28 | 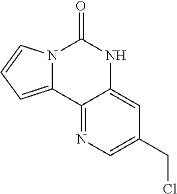 [SMILES: O=c1[nH]c2cc(CCl)cnc2c2cccn12] | Intermediate 27 |
| 31 | 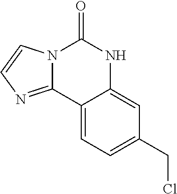 [SMILES: O=c1[nH]c2cc(CCl)ccc2c2nccn12] | Intermediate 30 |
| 36 | 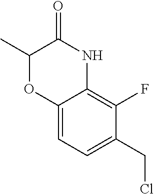 | Intermediate 35 |
| 41 | 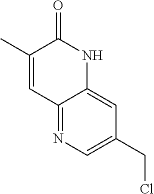 | Intermediate 40 |
| 47 | 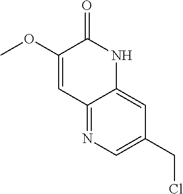 | Intermediate 46 |

### Preparation of Intermediate 7

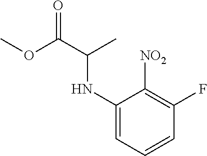 [SMILES: COC(=O)C(C)Nc1cccc(F)c1[N+](=O)[O-]]

To a solution of 1,3-difluoro-2-nitrobenzene (23.28 mL, 220 mmol) and methyl 2-aminopropanoate (27.22 g, 264 mmol) in DMF (120 mL) and DIPEA (85.30 g, 660 mmol) was dropwise. The mixture was heated to 60° C. stirred for 2 hrs. The solution was quenched with H₂O and then extracted with EtOAc. The organic layer was washed with brine, dried over Na₂SO₄, filtered and concentrated, chromatographed on silica gel (PE/EA 20:1 to 10:1 to 8:1) to give the intermediate 7 (38.7 g, yield: 72.6%) as a yellow solid.

The following intermediate was synthesized by an analogous method as described above for intermediate 7.

| Intermediate No | Structure | Starting Materials |
|---|---|---|
| 48 | 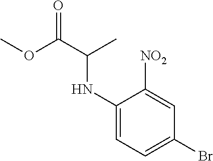 [SMILES: COC(=O)C(C)Nc1ccc(Br)cc1[N+](=O)[O-]] | 4-bromo-1-fluoro-2- nitrobenzene |

### Preparation of Intermediate 8

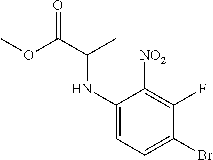 [SMILES: COC(=O)C(C)Nc1ccc(Br)c(F)c1[N+](=O)[O-]]

To a solution of intermediate 7 (4.4 g, 18.1 mmol) in DMF (50 mL) was added NBS (3.23 g, 18.1 mmol). The mixture was stirred at rt for 18 h under air atmosphere. The reaction mixture was poured into water (200 mL) and extracted with EtOAc (100 mL×2). The combined organic layers were dried over Na₂SO₄ and filtered. The filtrate was concentrated under reduced pressure to give a crude product, which was used for the next step without further purification.

### Preparation of Intermediate 9

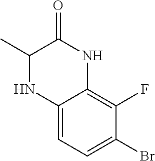

A mixture of intermediate 8 (9.7 g crude, 30.2 mmol), Zn (11.8 g, 181 mmol) and NH₄Cl (16.2 g, 53.5 mmol) in THF (50 mL)/MeOH (50 mL)/H₂O (25 mL) was heated to 80° C. stirred for 3 hrs. The mixture was filtered, and the filtrate was concentrated as a crude product, which was chromatographed on silica gel (PE/EA 20:1 to 10:1 to 5:1) to give intermediate 9 (7.1 g, yield: 90.7%).

### Preparation of Intermediate 10

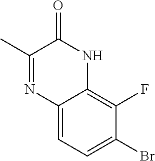

To a solution of intermediate 9 (620 mg, 2.39 mmol) in DCM (10 mL) was added a solution of DDQ (651 mg, 2.87 mmol) in DCM was dropwise. The mixture was stirred for 2 hr at r.t. and concentrated to give a crude product which was chromatographed on silica gel (PE/EA 10:1 to 7:1 to 5:1) to give the intermediate 10 (500 mg, yield 81.2%) as a while solid.

### Preparation of Intermediate 11

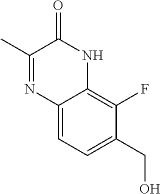

To a solution of intermediate 10 (1 g, 3.8 mmol) in dioxane (40 mL) was added (tributylstannyl) methanol (1.50 g, 4.6 mmol) and Xphos Pd G2 (0.15 g, 0.195 mmol). The mixture was stirred at 80° C. for 18 h under N₂ atmosphere. The reaction mixture was poured into water (100 mL) and extracted with ethyl acetate (100 mL×2). The combined organic layers were dried over Na₂SO₄ and filtered. The filtrate was concentrated under reduced pressure to give a residue, which was purified by column chromatography on silica gel eluted with DCM/MeOH (from 1/0 to 10/1) to afford intermediate 11 (600 mg, yield: 74%).

The following intermediates were synthesized by an analogous method as described above for intermediate 11.

| Intermediate No | Structure | Starting Materials |
|---|---|---|
| 23 | 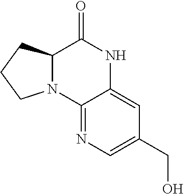 [SMILES: O=C1Nc2cc(CO)cnc2N2CCC[C@@H]12] | Intermediate 22 |
| 34 | 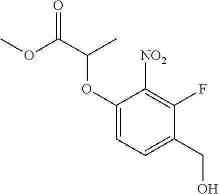 [SMILES: COC(=O)C(C)Oc1ccc(CO)c(F)c1[N+](=O)[O-]] | Intermediate 33 |
| 46 | 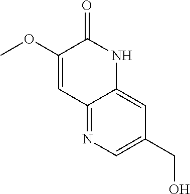 | Intermediate 45 |
| 51 | 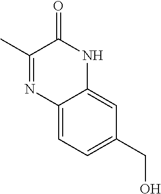 | Intermediate 50 |

### Preparation of Intermediate 12

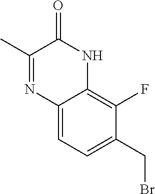

To a solution of intermediate 11 (200 mg, 0.961 mmol) in AcOH (8 mL) and H₂O (4 mL) was added HBr (9.3 g, 115.2 mmol). The mixture was stirred at 80° C. for 3 hr. The resulting mixture was poured into saturated NaHCO₃ (100 mL) and extracted with EtOAc (100 mL×2). The combined organic layers were dried over anhydrous Na₂SO₄ and filtered. The filtrate was concentrated under reduced pressure to give a residue, which was purified by column chromatography on silica gel eluted with PE/EA (5/1 to 1/1) and concentrated under reduced pressure to afford intermediate 12 (105 mg, yield: 40%).

The following intermediate was synthesized by an analogous method as described above for intermediate 12.

| Intermediate No | Structure | Starting Materials |
|---|---|---|
| 52 | 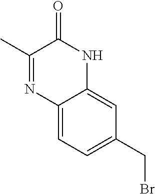 | Intermediate 51 |

### Preparation of Intermediate 13

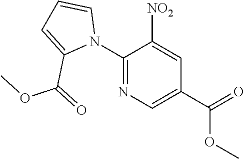

To a solution of methyl 6-chloro-5-nitropyridine-3-carboxylate (1.5 g, 6.92 mmol) in DMF (5 mL) were added methyl 1H-pyrrole-2-carboxylate (1.04 g, 8.31 mmol) and Cs₂CO₃ (6.77 g, 20.7 mmol) and the reaction was stirred at 60° C. for 30 min. The reaction was quenched with water and extracted with EtOAc. The organic layer was washed with brine, dried over Na₂SO₄, filtered, and concentrated in vacuo. The residue was purified using silica gel column chromatography eluting with ethyl acetate in petroleum ether (1:1) to afford intermediate 13 (900 mg, yield: 40.4%) as a yellow oil.

### Preparation of Intermediate 14

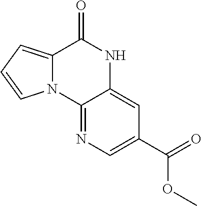 [SMILES: COC(=O)c1cnc2c(c1)[nH]c(=O)c1cccn12]

To a solution of intermediate 13 (700 mg, 2.29 mmol) in AcOH (5 mL), H₂O (5 mL) and THF (5 mL) was added Zn (1.8 g, 27.5 mmol). The reaction mixture was stirred at 80° C. for 3 h. The reaction mixture was filtered and washed with DCM/MeOH (1/1). The filtrate was concentrated under reduced pressure to give a crude product, which was used for the next step without further purification (500 mg, yield: 89.6%).

### Preparation of Intermediate 17

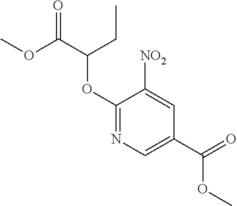

To methyl 6-chloro-5-nitropyridine-3-carboxylate (1 g, 4.6 mmol) and methyl 2-hydroxybutanoate (0.65 g, 5.5 mmol) in anhydrous tetrahydrofuran (15 mL) under nitrogen at 0° C. was added DBU (1.03 mL, 6.92 mmol). The reaction mixture was stirred at 0° C. for 30 minutes, then allowed to stir at ambient temperature for one hour. A solid precipitated out of solution. Next, the reaction mixture was diluted with ethyl acetate (15 mL), solids were removed by filtration, and the filtrate was concentrated in the presence of silica to provide a crude product, which was purified by column chromatography eluting with a gradient of 10-50% ethyl acetate in hexanes to provide intermediate 17 (1.2 g, yield: 87.1%) as a white solid.

### Preparation of Intermediate 18

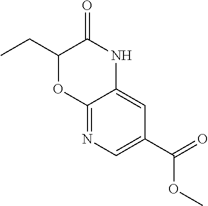 [SMILES: CCC1Oc2ncc(C(=O)OC)cc2NC1=O]

To a solution of intermediate 17 (1.2 g, 4.02 mmol) in glacial acetic acid (10 mL) was added powdered iron (1.12 g, 20.1 mmol). The resulting suspension was heated to 80° C. for 2 hours. Then, the resulting reaction mixture was cooled down to r.t., filtered through celite, and washed with ethyl acetate. The filtrates were washed with water and brine, dried with sodium sulfate, filtered, and concentrated in vacuo to afford intermediate 18 (0.8 g, yield: 84.2%) as a crude product, which was directly used in the next step without further purification.

The following intermediate was synthesized by an analogous method as described above for intermediate 18.

| Intermediate | | Starting |
|---|---|---|
| No | Structure | Materials |
| 49 | 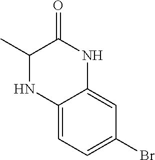 | Intermediate 48 |

### Preparation of Intermediate 19

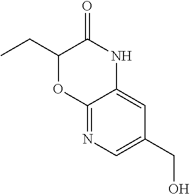

To a solution of intermediate 18 (500 mg, 2.1 mmol) in dry THF (10 mL) at 0° C. was added DIBAL-H (4.2 mL, 1 M in toluene) over 1 h. After the addition, the reaction mixture was warmed up to r.t. After the reaction was complete as indicated by TLC analysis, the reaction was quenched with a saturated NH₄Cl solution (20 mL) and extracted with EtOAc (100 ml×3). The combined organic layers were dried over anhydrous Na₂SO₄, filtered and concentrated. The crude product was purified by silica gel chromatography (DCM: MeOH=20:1 to 10:1) to give intermediate 19. (150 mg, yield: 34.1%).

### Preparation of Intermediate 21

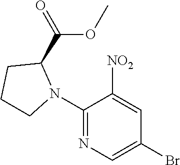 [SMILES: COC(=O)[C@@H]1CCCN1c1ncc(Br)cc1[N+](=O)[O-]]

To a solution of 5-bromo-2-chloro-3-nitropyridine (8 g, 33.69 mmol) in DMF (100 mL) was added methyl(2R)-pyrrolidine-2-carboxylate hydrochloride (6.14 g, 37.0 mmol) and DIEA (16.7 mL, 101.0 mmol). The resulting reaction mixture was stirred at 80° C. for 3 h. The reaction was diluted with water and extracted with EtOAc. The combined organic layer was washed with brine, dried over Na₂SO₄, filtered, and concentrated in vacuo to afford intermediate 21 (10 g, yield: 80.9%) as a yellow oil.

### Preparation of Intermediate 22

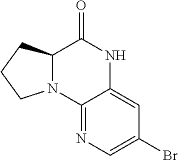 [SMILES: O=C1Nc2cc(Br)cnc2N2CCC[C@@H]12]

To a solution of intermediate 21 (10.0 g, 27.2 mmol) in MeOH (100 mL) and H₂O (20 mL) was added Fe (7.6 g, 136.3 mmol) and NH₄Cl (14.6 g, 272.6 mmol). The resulting reaction mixture was stirred at 85° C. for 1 h. The reaction was filtered, and the filtrate was diluted with water and extracted with EtOAc. The combined organic layer was dried over Na₂SO₄, filtered, and concentrated in vacuo. The residue was purified by silica gel column chromatography to afford intermediate 22 (2 g, yield: 24.6%) as a yellow solid.

### Preparation of Intermediate 25

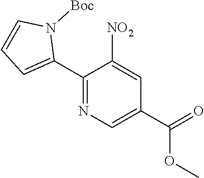 [SMILES: COC(=O)c1cnc(-c2cccn2C(=O)OC(C)(C)C)c([N+](=O)[O-])c1]

A mixture of methyl 6-chloro-5-nitropyridine-3-carboxylate (2 g, 9.23 mmol), {1-[(tert-butoxy)carbonyl]-1H-pyrrol-2-yl}boronic acid (2.34 g, 11.0 mmol), Pd(dppf)Cl₂ (0.68 g, 0.92 mmol) and Na₂CO₃ (2.94 g, 27.7 mmol) in 1,4-dioxane (60 mL) and H₂O (15 mL) was stirred for 1 hours at 100° C. under N₂ atmosphere overnight. The reaction was diluted water and extracted with EtOAc. The organic layer was separated, washed with brine, dried over Na₂SO₄, filtered, and concentrated in vacuo. The residue was purified by silica gel column chromatography eluting with ethyl acetate in petroleum ether (5:1) to intermediate 25 (1.1 g, yield: 30.8%) as a red oil.

### Preparation of Intermediate 26

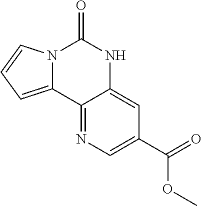 [SMILES: COC(=O)c1cnc2c(c1)[nH]c(=O)n1cccc21]

To a solution of intermediate 25 (1 g, 2.59 mmol) in THF (15 mL) and H₂O (15 mL) was added Zn (1.65 g, 25.9 mmol) and NH₄Cl (2.8 g, 51.8 mmol). The reaction was stirred at 80° C. for 6 hr. The reaction was diluted with EtOAc and brine. The organic layer was concentrated in vacuo. The residue was purified by silica gel column chromatography eluting with methanol in DCM to afford intermediate 26 (300 mg, yield: 45.2%) as a white solid.

### Preparation of Intermediate 29

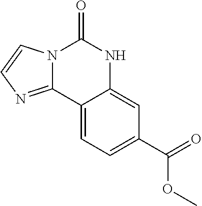 [SMILES: COC(=O)c1ccc2c(c1)[nH]c(=O)n1ccnc21]

To a solution of tert-butyl 2-bromo-1H-imidazole-1-carboxylate (1.27 g, 5.12 mmol) and [4-(methoxycarbonyl)-2-nitrophenyl]boronic acid (900 mg, 4.0 mmol) in dioxane (20 mL) and water (10 mL) was added Pd(dppf)Cl₂ (0.38 g, 0.51 mmol) and Cs₂CO₃ (3.34 g, 10.2 mmol). The mixture was stirred at 100° C. for 3 h. The reaction mixture was poured into water (100 mL) and extracted with EtOAc (100 mL×2). The combined organic layers were dried over Na₂SO₄ and filtered. The filtrate was concentrated under reduced pressure to give a residue, which was purified by column chromatography on silica gel eluted with PE/EA (from 1/0 to 1/2) to give intermediate 29 (170 mg, yield: 13.6%).

### Preparation of Intermediate 32

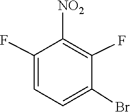 [SMILES: O=[N+]([O-])c1c(F)ccc(Br)c1F]

To a solution of 1,3-difluoro-2-nitrobenzene (5.00 g, 31.4 mmol) in H₂SO₄ (15 mL) was added NBS (5.60 g, 31.4 mmol). The mixture was stirred at 80° C. for 18 h. The reaction mixture was poured into ice water (600 mL) and extracted with EtOAc (200 mL). The organic layer was washed with saturated NaHCO₃ (100 mL) and dried over anhydrous Na₂SO₄ and filtered. The filtrate was concentrated under vacuum to give a residue, which was purified by column chromatography on silica gel to give intermediate 32 (6.9 g, yield: 92.6%) as a brown oil.

### Preparation of Intermediate 33

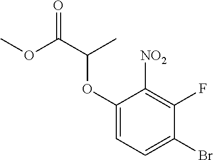 [SMILES: COC(=O)C(C)Oc1ccc(Br)c(F)c1[N+](=O)[O-]]

To a solution of intermediate 32 (4.75 g, 19.9 mmol) and methyl 2-hydroxypropanoate (2.7 g, 25.9 mmol) in DMF (50 mL) was added Cs₂CO₃ (13 g, 39.9 mmol). The mixture was stirred at r.t. for 18 hr. The reaction was poured into water (100 mL) and extracted with EtOAc (100 mL×2). The combined organic layers were washed by brine (100 mL×3). The organic layer was dried over Na₂SO₄ and filtered. The filtrate was concentrated under reduced pressure to give a residue, which was purified by column chromatography on silica gel to give intermediate 33 (500 mg, yield: 7.78%) as a brown solid.

### Preparation of Intermediate 35

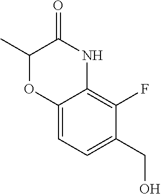

To a solution of intermediate 34 (180 mg, 0.66 mmol) in MeOH (4 mL), THF (2 mL), and H₂O (2 mL) was added Zn (259 mg, 3.9 mmol) and NH₄Cl (352 mg, 6.60 mmol). The mixture was stirred at 80° C. for 3 h. The reaction was filtered, and filtrate was extracted with EtOAc (50 mL×2). The combined organic layers were dried over Na₂SO₄ and filtered. The filtrate was concentrated under reduced pressure to give a residue, which was purified by column chromatography on silica gel to give intermediate 35 (50 mg, yield: 35.9%) as a yellow oil.

### Preparation of Intermediate 42

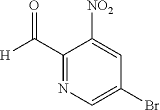 [SMILES: O=Cc1ncc(Br)cc1[N+](=O)[O-]]

A mixture of 5-bromo-2-methyl-3-nitropyridine (1 g, 4.60 mmol) and SeO₂ (0.77 g, 6.9 mmol) in dioxane (5 mL) was stirred at 110° C. for 48 h. The reaction mixture was cooled down to room temperature, filtered through a pad of celite and the celite was washed with ethyl acetate. The combined filtrate was concentrated, and the resulting residue was purified by flash silica gel chromatography, elution gradient 0 to 70% ethyl acetate in hexanes to afford intermediate 42 (600 mg, yield: 56.3%) as a brown oil.

### Preparation of Intermediate 43

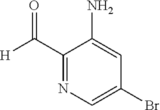

To a solution of intermediate 42 (5.1 g, 22.0 mmol) in THF (45 mL) was added Fe (6.17 g, 110.3 mmol) and AcOH (5 mL). The reaction mixture was stirred for 1 h at 25° C. and filtered through Celite and washed with EtOAc. The filtrate was diluted with water and extracted with EtOAc. The organic layer was washed with brine and dried over Na₂SO₄, filtered, and concentrated in vacuo to give a crude product which was purified by column chromatography on silica gel using 0-5% EtOAc in hexane to give intermediate 43 (4 g, yield: 90.1%) as a yellow solid.

### Preparation of Intermediate 44

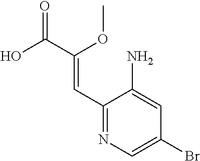

At −78° C., ethyl 2-methoxyacetate (1.763 mL, 14.9 mmol) was added to a solution of Li-HMDS in THF (14.9 mL, 14.9 mmol). After 20 minutes, a solution intermediate 43 (3 g, 14.9 mmol) in THF (20 mL) was added dropwise from a syringe. The resulting mixture was allowed to warm up slowly to room temperature. After 18 hours, the reaction was quenched with 6N HCl (1.1 mL, 6.6 mmol), providing a precipitate. The mixture was heated up to reflux for 2 hours. The reaction was then concentrated in vacuo, and the residue was triturated with dichloromethane and methanol. A solid was removed by filtration, and the filtrate was concentrated in vacuo. The combined filtrate was concentrated, and the resulting residue was purified by flash silica chromatography (eluant, 100% dichloromethane to 100:10:1 dichloromethane:methanol; triethylamine), yielding intermediate 44 (2.5 g, yield: 61.34%) a s a white solid.

### Preparation of Intermediate 45

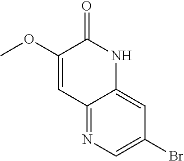

A mixture of intermediate 44 (2.5 g, 9.15 mmol), CDI (4.45 g, 27.4 mmol) in MeCN (30 mL) was degassed and stirred at 50° C. for overnight. The mixture was filtered through a Celite and the Celite washed with EtOAc and EtOH. The mixture was diluted with ether and the solid was filtered off, washed with diethyl ether, and dried under vacuum to afford intermediate 45 (1.5 g, yield 64.2%) as a white solid.

### Preparation of Intermediate 100

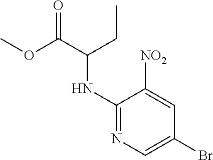 [SMILES: CCC(Nc1ncc(Br)cc1[N+](=O)[O-])C(=O)OC]

To a solution of 5-bromo-2-chloro-3-nitropyridine (10 g, 42.1 mmol) in DMF (100 mL) was added methyl 2-aminobutanoate hydrochloride (7.76 g, 50.5 mmol) and DIEA (20.8 mL, 126.3 mmol). The mixture was stirred at 80° C. for 3 hr. The reaction mixture was poured into water (200 mL) and extracted with EtOAc (100 mL×2). The combined organic layers were washed with brine (100 mL×3). The organic layer was dried over Na₂SO₄ and filtered. The filtrate was concentrated under reduced pressure to give a residue, which was purified by column chromatography on silica gel eluted with PE/EA (10/1) to give intermediate 100 (11.2 g, 84% yield).

### Preparation of Intermediate 101

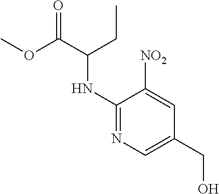 [SMILES: CCC(Nc1ncc(CO)cc1[N+](=O)[O-])C(=O)OC]

To a solution of intermediate 100 (3 g, 9.430 mmol) and (tributylstannyl) methanol (3.63 g, 11.3 mmol) in dioxane (60 mL) was added XPhos Pd G2 (24.64 mg, 0.031 mmol). The mixture was stirred at 80° C. for 18 hr under N₂ atmosphere. The reaction was concentrated under reduced pressure to give a residue, which was purified by column chromatography on silica gel eluted with PE/EA (10/1 to 3/1) to give intermediate 101 (2.3 g, 90% yield).

### Preparation of Intermediate 102

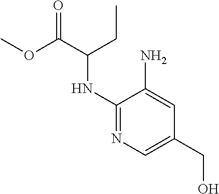 [SMILES: CCC(Nc1ncc(CO)cc1N)C(=O)OC]

To a solution of intermediate 101 (1.2 g, 4.4 mmol) in MeOH (20 mL) was added Pd/C 10% (0.47 g). The mixture was stirred at r.t. for 18 hr under H₂ atmosphere. The reaction mixture was filtered. The filtrate was concentrated under reduced pressure to give intermediate 102 as a crude product, which was used for the next step without further purification.

### Preparation of Intermediate 103

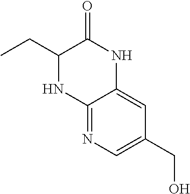

To a solution of intermediate 102 (1.0 g, 4.1 mmol) in DMF (20 mL) was added K₂CO₃ (1.16 g, 8.3 mmol). The mixture was stirred at 80° C. for 18 hr. The reaction mixture was filtered, and filtrate was concentrated under reduced pressure to give a residue, which was triturated in EtOAc (50 mL) and filtered. The filter pellet was dried under reduced pressure to give intermediate 103 as a crude product, which was used for the next step without further purification.

### Preparation of Intermediate 104

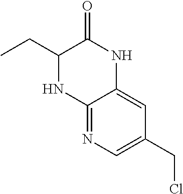

To a suspension of intermediate 103 (200 mg, 0.96 mmol) in DCM (8 mL) was added SOCl₂ (0.070 mL, 0.96 mmol). The mixture was stirred at r.t. for 18 hr. The reaction was concentrated under reduced pressure to give a residue, which was triturated in EtOAc (30 mL) and filtered. The filtrate was dried under reduced pressure to give intermediate 104 (100 mg, 46% yield) as a crude product, which was used for the next step without further purification.

### Preparation of Intermediate 105

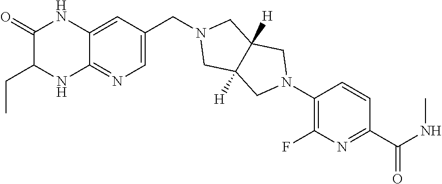 [SMILES: CCC1Nc2ncc(CN3C[C@@H]4CN(c5ccc(C(=O)NC)nc5F)C[C@H]4C3)cc2NC1=O]

To a solution of intermediate 104 (100 mg, crude) in CH₃CN (6 mL) were added intermediate 67 (100 mg, 0.33 mmol), DIEA (128.5 mg, 0.99 mmol) and potassium iodide (5.5 mg, 0.03 mmol). The reaction mixture was stirred at 80° C. for 2 h. It was concentrated under reduced pressure to give a residue, which was purified by column chromatography on silica gel eluted with DCM/MeOH (10/1) to give intermediate 105 (43 mg, yield: 28.5%) as a brown solid.

### Preparation of Intermediate 106

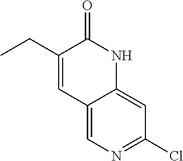

To a solution of methyl butanoate (10.9 mL, 95.8 mmol) in THF (50 mL) was added LDA (2N, 49.4 mL, 98.9 mmol) at −75° C. After stirred at −78° C. for 1 hr, 4-amino-6-chloropyridine-3-carbaldehyde (5 g, 31.935 mmol) was added. The mixture was warmed up to r.t. and stirred overnight. The mixture was quenched by saturated ammonium chloride aqueous solution and extracted with EtOAc. The organic layers were washed with brine, dried over Na₂SO₄, filtered, and concentrated to give crude product, which was purified by silica gel column chromatography (DCM:MeOH=20:1) to afford intermediate 106 (3.7 g, 56.4% yield) as white solid.

The following intermediate was synthesized by an analogous method as described above for intermediate 106.

| Intermediate | | Starting |
|---|---|---|
| No | Structure | Materials |
| 109 | 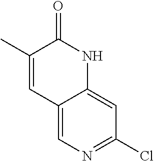 | methyl propionate |

### Preparation of Intermediate 107

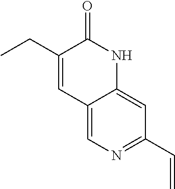

Pd(dppf)Cl₂ (245.4 mg, 0.33 mmol) was added to a stirred mixture of intermediate 106 (700 mg, 3.3 mmol), 2-ethenyl-4,4,5,5-tetramethyl-1,3,2-dioxaborolane (0.68 mL, 4.0 mmol) and K₂CO₃ (1.39 g, 10.0 mmol) in 1,4-dioxane (5 mL)/water (1 mL) and the resulting mixture was stirred at 90° C. overnight under N₂. The reaction mixture was diluted with water and extracted with ethyl acetate. The organic layers were combined, dried over sodium sulphate, filtered, and concentrated to give crude product. The residue was purified by flash silica chromatography, elution gradient 0 to 20% MeOH in DCM. Product fractions were concentrated under reduced pressure to dryness to afford intermediate 107 (600 mg, 89.3% yield) as a yellow solid.

The following intermediates were synthesized by an analogous method as described above for intermediate 107.

| Intermediate | | Starting |
|---|---|---|
| No | Structure | Materials |
| 110 | 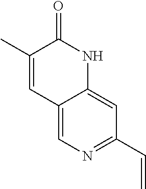 | Intermediate 109 |
| 119 | 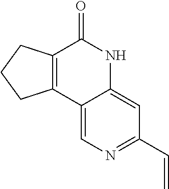 [SMILES: C=Cc1cc2[nH]c(=O)c3c(c2cn1)CCC3] | Intermediate 118 |
| 136 | 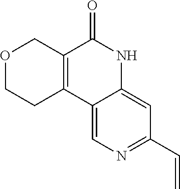 [SMILES: C=Cc1cc2[nH]c(=O)c3c(c2cn1)CCOC3] | Intermediate 135 |
| 149 | 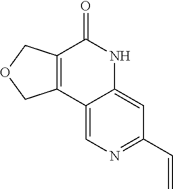 [SMILES: C=Cc1cc2[nH]c(=O)c3c(c2cn1)COC3] | Intermediate 148 |
| 157 | 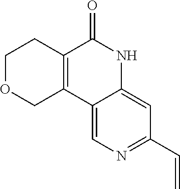 [SMILES: C=Cc1cc2[nH]c(=O)c3c(c2cn1)COCC3] | Intermediate 156 |

### Preparation of Intermediate 108

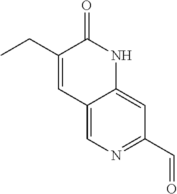

A mixture of potassium osmate (VI) (184 mg, 0.49 mmol) in H₂O (3 mL) was added to a solution of intermediate 107 (500 mg, 2.49 mmol), 2,6-lutidine (0.58 mL, 4.9 mmol) and NaIO₄ (2136 mg, 9.9 mmol) in THF (6 mL) and the resulting mixture was stirred at r.t. overnight. The reaction mixture was diluted with water and extracted with ethyl acetate. The organic layer was combined and concentrated to dryness. The resulting residue was purified by flash silica chromatography, elution gradient 0 to 15% MeOH in DCM. Product fractions were concentrated under reduced pressure to afford intermediate 108 (250 mg, 49.5% yield) as dark-yellow foam.

The following intermediates were synthesized by an analogous method as described above for intermediate 108.

| Intermediate | | Starting |
|---|---|---|
| No | Structure | Materials |
| 111 | 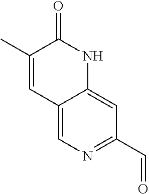 | Intermediate 110 |
| 120 | 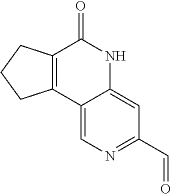 [SMILES: O=Cc1cc2[nH]c(=O)c3c(c2cn1)CCC3] | Intermediate 119 |
| 137 | 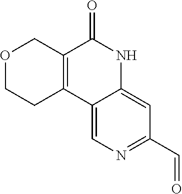 [SMILES: O=Cc1cc2[nH]c(=O)c3c(c2cn1)CCOC3] | Intermediate 136 |
| 150 | 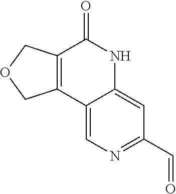 [SMILES: O=Cc1cc2[nH]c(=O)c3c(c2cn1)COC3] | Intermediate 149 |
| 158 | 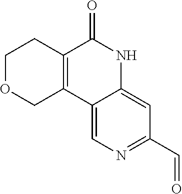 [SMILES: O=Cc1cc2[nH]c(=O)c3c(c2cn1)COCC3] | Intermediate 157 |

### Preparation of Intermediate 112

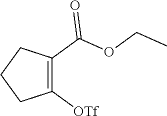 [SMILES: CCOC(=O)C1=C(OS(=O)(=O)C(F)(F)F)CCC1]

To a solution of methyl 2-oxocyclopentane-1-carboxylate (2 g, 14.07 mmol) in DCM (40 mL) was added NaH (0.68 g, 16.88 mmol) at 0° C. The reaction mixture was stirred for 0.5 hr and trifluoromethanesulfonic anhydride (4.76 g, 16.88 mmol) was added. The reaction mixture was warmed up and stirred at r.t. for 18 hr. The reaction was quenched with saturated NH₄Cl (100 mL) and extracted with DCM (50 mL×2). The combined organic layers were dried over Na₂SO₄ and filtered. The filtrate was concentrated under reduced pressure to give a residue, which was purified by column chromatography on silica gel eluted with PE/EA (10/1) to provide intermediate 112 (750 mg, yield: 19.44%) as a yellow oil.

### Preparation of Intermediate 113

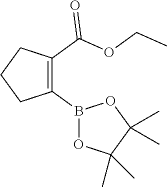 [SMILES: CCOC(=O)C1=C(B2OC(C)(C)C(C)(C)O2)CCC1]

To a solution of intermediate 112 (750 mg, 2.73 mmol) and 4,4,5,5-tetramethyl-2-(tetramethyl-1,3,2-dioxaborolan-2-yl)-1,3,2-dioxaborolane (792.9 mg, 3.12 mmol) in dioxane (10 mL) were added KOAc (510.7 mg, 5.2 mmol) and Pd(dppf)Cl₂ (190.3 mg, 0.26 mmol). The reaction mixture was stirred at 80° C. for 12 h under N₂ atmosphere. After cooling down to rt, the reaction mixture was poured into water (100 mL) and extracted with EtOAc (100 mL×2). The combined organic layers were dried over Na₂SO₄ and filtered. The filtrate was concentrated under reduced pressure to give a residue, which was purified by column chromatography on silica gel eluted with PE/EA (10/1) to provide intermediate 113 (600 mg, yield: 86.64%) as a white solid.

The following intermediates were synthesized by an analogous method as described above for intermediate 113.

| Intermediate | | Starting |
|---|---|---|
| No | Structure | Materials |
| 131 | 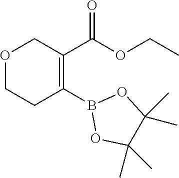 [SMILES: CCOC(=O)C1=C(B2OC(C)(C)C(C)(C)O2)CCOC1] | Intermediate 130 |
| 143 | 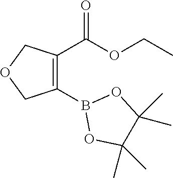 [SMILES: CCOC(=O)C1=C(B2OC(C)(C)C(C)(C)O2)COC1] | Intermediate 142 |
| 152 | 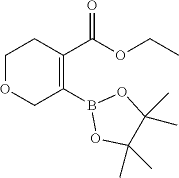 [SMILES: CCOC(=O)C1=C(B2OC(C)(C)C(C)(C)O2)COCC1] | Intermediate 151 |

### Preparation of Intermediate 114

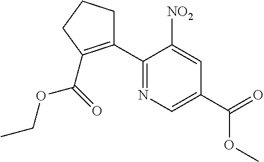 [SMILES: CCOC(=O)C1=C(c2ncc(C(=O)OC)cc2[N+](=O)[O-])CCC1]

To a solution of intermediate 113 (600 mg, 2.25 mmol) and methyl 6-chloro-5-nitropyridine-3-carboxylate (400 mg, 1.84 mmol) in dioxane (10 mL) and H₂O (2 mL) were added Pd(dppf)Cl₂ (135.14 mg, 0.185 mmol) and K₂CO₃ (510.52 mg, 3.694 mmol). The reaction mixture was stirred at 100° C. for 3 hr under N₂ atmosphere. The reaction mixture was poured into water (100 mL) and extracted with EtOAc (100 mL×2). The combined organic layers were dried over Na₂SO₄ and filtered. The filtrate was concentrated under reduced pressure to give a residue, which was purified by column chromatography on silica gel eluted with PE/EA (10/1) to provide intermediate 114 (400 mg, yield: 67.62%) as a yellow solid.

The following intermediate was synthesized by an analogous method as described above for intermediate 114.

| Intermediate | | Starting |
|---|---|---|
| No | Structure | Materials |
| 144 | 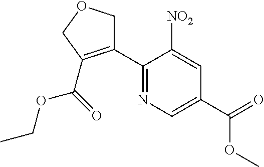 [SMILES: CCOC(=O)C1=C(c2ncc(C(=O)OC)cc2[N+](=O)[O-])COC1] | Intermediate 143 |

### Preparation of Intermediate 115

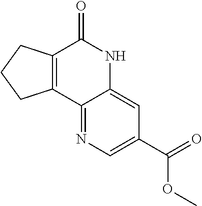 [SMILES: COC(=O)c1cnc2c3c(c(=O)[nH]c2c1)CCC3]

To a solution of intermediate 114 (400 mg, 1.24 mmol) in MeOH (6 mL), THF (3 mL) and H₂O (3 mL) was added NH₄Cl (668 mg, 12.4 mmol) and Fe (350 mg, 6.2 mmol). The mixture was stirred at 80° C. for 2 h. The reaction mixture was poured into water (100 mL) and extracted with DCM/MeOH (10/1, 100 mL×2). The combined organic layers were dried over Na₂SO₄ and filtered. The filtrate was concentrated under reduced pressure to give a residue, which was purified by column chromatography on silica gel eluted with DCM/MeOH (3.6% MeOH) to provide intermediate 115 (80 mg, yield: 26.23%) as a yellow solid.

The following intermediate was synthesized by an analogous method as described above for intermediate 115.

| Intermediate | Structure | Starting |
|---|---|---|
| No | | Materials |
| 145 | 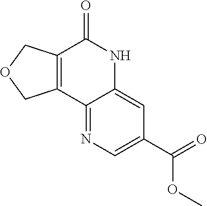 [SMILES: COC(=O)c1cnc2c3c(c(=O)[nH]c2c1)COC3] | Intermediate 144 |

### Preparation of Intermediate 116

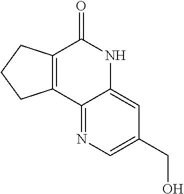 [SMILES: O=c1[nH]c2cc(CO)cnc2c2c1CCC2]

To a solution of intermediate 115 (80 mg, 0.32 mmol) in THF (6 mL) was added LiAlH₄ (0.98 mL, 1N in THF, 0.98 mmol) at 0° C. The mixture was stirred at 0° C. for 1 hr. The reaction mixture was quenched with MeOH and concentrated under reduced pressure to provide intermediate 116 (70 mg, yield: 98.83%) as a white solid, which was used directly in next step without further purification.

The following intermediates were synthesized by an analogous method as described above for intermediate 116.

| Intermediate | | Starting |
|---|---|---|
| No | Structure | Materials |
| 124 | 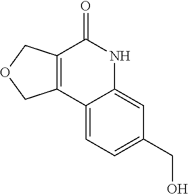 [SMILES: O=c1[nH]c2cc(CO)ccc2c2c1COC2] | Intermediate 123 |
| 128 | 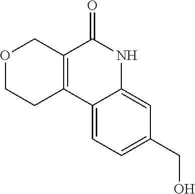 [SMILES: O=c1[nH]c2cc(CO)ccc2c2c1COCC2] | Intermediate 127 |
| 133 | 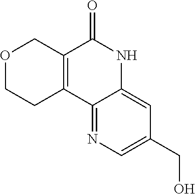 [SMILES: O=c1[nH]c2cc(CO)cnc2c2c1COCC2] | Intermediate 132 |
| 140 | 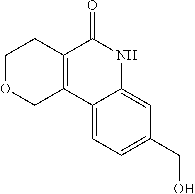 [SMILES: O=c1[nH]c2cc(CO)ccc2c2c1CCOC2] | Intermediate 139 |
| 146 | 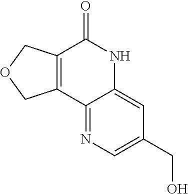 [SMILES: O=c1[nH]c2cc(CO)cnc2c2c1COC2] | Intermediate 145 |
| 154 | 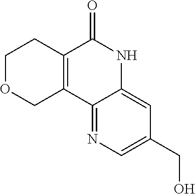 [SMILES: O=c1[nH]c2cc(CO)cnc2c2c1CCOC2] | Intermediate 153 |
| 180 | 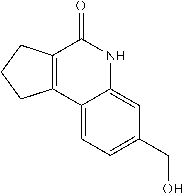 [SMILES: O=c1[nH]c2cc(CO)ccc2c2c1CCC2] | Intermediate 179 |
| 187 | 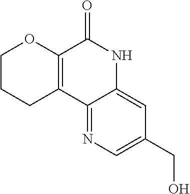 [SMILES: O=c1[nH]c2cc(CO)cnc2c2c1OCCC2] | Intermediate 186 |
| 192 | 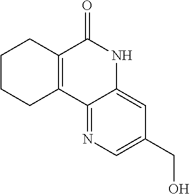 [SMILES: O=c1[nH]c2cc(CO)cnc2c2c1CCCC2] | Intermediate 191 |
| 201 | 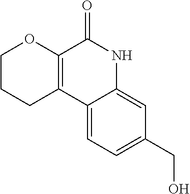 [SMILES: O=c1[nH]c2cc(CO)ccc2c2c1OCCC2] | Intermediate 200 |
| 205 | 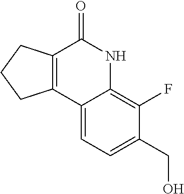 [SMILES: O=c1[nH]c2c(F)c(CO)ccc2c2c1CCC2] | Intermediate 204 |

### Preparation of Intermediate 117

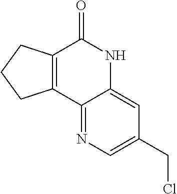 [SMILES: O=c1[nH]c2cc(CCl)cnc2c2c1CCC2]

To a solution of intermediate 116 (65 mg, 0.30 mmol) in DCM (10 mL) was added SOCl₂ (107.2 mg, 0.90 mmol). The mixture was stirred at r.t. for 2 h. The reaction mixture was concentrated under reduced pressure to give a residue, which was purified by column chromatography on silica gel eluted with DCM/MeOH (10/1) to provide intermediate 117 (70 mg, yield: 99.22%) as a white solid.

The following intermediates were synthesized by an analogous method as described above for intermediate 117.

| Intermediate | | Starting |
|---|---|---|
| No | Structure | Materials |
| 125 | 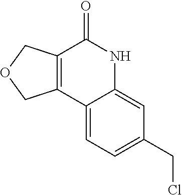 [SMILES: O=c1[nH]c2cc(CCl)ccc2c2c1COC2] | Intermediate 124 |
| 129 | 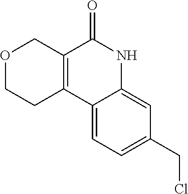 [SMILES: O=c1[nH]c2cc(CCl)ccc2c2c1COCC2] | Intermediate 128 |
| 134 | 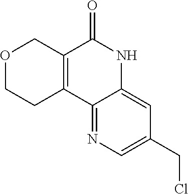 [SMILES: O=c1[nH]c2cc(CCl)cnc2c2c1COCC2] | Intermediate 133 |
| 141 | 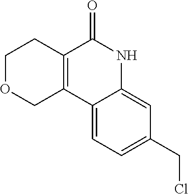 [SMILES: O=c1[nH]c2cc(CCl)ccc2c2c1CCOC2] | Intermediate 140 |
| 147 | 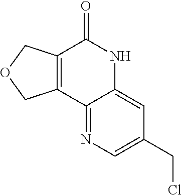 [SMILES: O=c1[nH]c2cc(CCl)cnc2c2c1COC2] | Intermediate 146 |
| 155 | 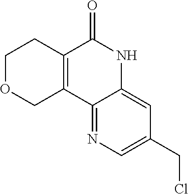 [SMILES: O=c1[nH]c2cc(CCl)cnc2c2c1CCOC2] | Intermediate 154 |
| 181 | 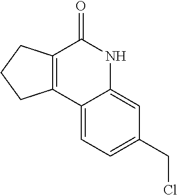 [SMILES: O=c1[nH]c2cc(CCl)ccc2c2c1CCC2] | Intermediate 180 |
| 188 | 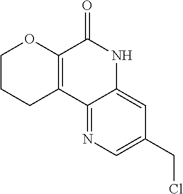 [SMILES: O=c1[nH]c2cc(CCl)cnc2c2c1OCCC2] | Intermediate 187 |
| 193 | 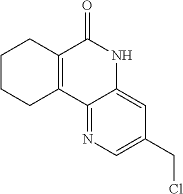 [SMILES: O=c1[nH]c2cc(CCl)cnc2c2c1CCCC2] | Intermediate 192 |
| 202 | 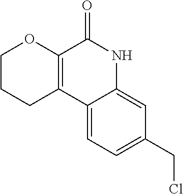 [SMILES: O=c1[nH]c2cc(CCl)ccc2c2c1OCCC2] | Intermediate 201 |
| 206 | 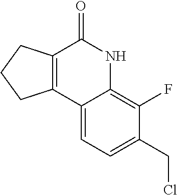 [SMILES: O=c1[nH]c2c(F)c(CCl)ccc2c2c1CCC2] | Intermediate 205 |

### Preparation of Intermediate 118

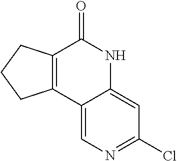 [SMILES: O=c1[nH]c2cc(Cl)ncc2c2c1CCC2]

To a mixture of 5-bromo-2-chloropyridin-4-amine (2.34 g, 11.2 mmol) in dioxane (30 mL) and water (5 mL) was added intermediate 113 (3.0 g, 11.27 mmol), Pd(dppf)Cl₂ (826 mg, 1.13 mmol) and K₂CO₃ (4.67 g, 33.82 mmol). The resulting mixture was stirred at 80° C. for 5 hr. After cooled down to rt, the reaction was diluted with water (20 mL) and extracted with DCM (100 mL×3). The combined organics were dried over anhydrous Na₂SO₄, filtered and concentrated in vacuo. The residue was purified by silica gel column chromatography (MeOH:DCM=1:10) to provide intermediate 118 (1.8 g, yield: 72.37%) as a white solid.

The following intermediates were synthesized by an analogous method as described above for intermediate 118.

| Intermediate | | Starting |
|---|---|---|
| No | Structure | Materials |
| 148 | 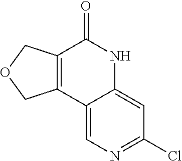 [SMILES: O=c1[nH]c2cc(Cl)ncc2c2c1COC2] | Intermediate 143 |
| 156 | 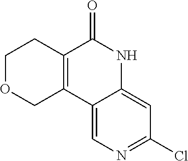 [SMILES: O=c1[nH]c2cc(Cl)ncc2c2c1CCOC2] | Intermediate 152 |

### Preparation of Intermediate 121

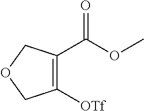 [SMILES: COC(=O)C1=C(OS(=O)(=O)C(F)(F)F)COC1]

To a solution of methyl 4-oxotetrahydrofuran-3-carboxylate (2 g, 13.8 mmol) in DCM (5 mL) was added DIEA (3.59 g, 27.7 mmol) in DCM (30 mL) at −78° C. under nitrogen atmosphere, and then trifluoromethanesulfonic anhydride (3.5 mL, 20.82 mmol) in DCM (5 mL) was added dropwise at −70° C. for 15 mins. The reaction mixture was stirred at 0° C. for 3 hr and quenched by cold water (30 mL). The mixture was extracted with EtOAc and washed with brine, dried over Na₂SO₄, filtered and concentrated to get a residue, which was purified by silica gel column chromatography (petroleum ether:ethyl acetate=20:1) to afford intermediate 121 (3.2 g, yield: 83.50%) as a yellow oil.

The following intermediate was synthesized by an analogous method as described above for intermediate 121.

| Intermediate | | Starting |
|---|---|---|
| No | Structure | Materials |
| 142 | 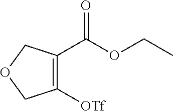 [SMILES: CCOC(=O)C1=C(OS(=O)(=O)C(F)(F)F)COC1] | ethyl 4-oxotetra- hydrofuran-3- carboxylate |

### Preparation of Intermediate 122

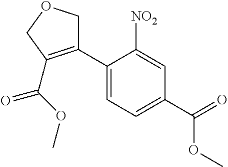 [SMILES: COC(=O)C1=C(c2ccc(C(=O)OC)cc2[N+](=O)[O-])COC1]

To a solution of methyl intermediate 121 (1.8 g, 6.52 mmol) in dioxane (20 mL) was added (4-(methoxycarbonyl)-2-nitrophenyl) boronic acid (1.47 g, 6.52 mmol), Pd(PPh₃)₄ (0.75 g, 0.65 mmol) and Cs₂CO₃ (4.25 g, 13.04 mmol). The resulting reaction mixture was stirred at 100° C. for 3 h under nitrogen atmosphere. The mixture was concentrated and purified by silica gel column chromatography (petroleum ether: ethyl acetate=1:1) to afford intermediate 122 (1.6 g, yield: 79.90%) as a white solid.

### Preparation of Intermediate 123

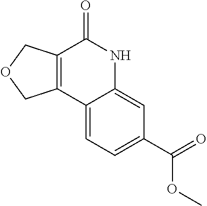 [SMILES: COC(=O)c1ccc2c3c(c(=O)[nH]c2c1)COC3]

To a solution of intermediate 122 (1 g, 3.26 mmol) in EtOH (20 mL)/Water (10 mL) were added Fe (0.91 g, 16.28 mmol) and NH₄Cl (0.87 g, 16.28 mmol). The resulting reaction mixture was stirred at 80° C. for 2 hr under nitrogen atmosphere. After the mixture was cooled down to rt, it was filtered through Celite and the filtrate was concentrated to give intermediate 13 (crude, 600 mg, 75.18%) as a yellow solid, which was used directly in next step without further purification.

### Preparation of Intermediate 126

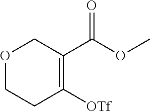 [SMILES: COC(=O)C1=C(OS(=O)(=O)C(F)(F)F)CCOC1]

To a stirred solution of methyl 4-oxooxane-3-carboxylate (2 g, 12.65 mmol) in DCM (8 mL) was added a solution of 2,6-di-tert-butyl-4-methylpyridine (2.60 mL, 12.65 mmol) in DCM (2 mL) at 0° C. under nitrogen atmosphere, and then trifluoromethanesulfonic anhydride (2.10 mL, 12.65 mmol) was added dropwise at 0° C. under nitrogen. The resulting reaction mixture was stirred at r.t. for 16 hours. The solid was filtered out through a pad of Celite, and the filtrate was evaporated under reduced pressure to afford crude intermediate 126 (3 g, 10.34 mmol) as a yellow oil, which was used in the next step directly without further purification.

The following intermediates were synthesized by an analogous method as described above for intermediate 126.

| Intermediate | | Starting |
|---|---|---|
| No | Structure | Materials |
| 130 | 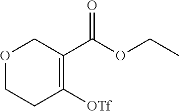 [SMILES: CCOC(=O)C1=C(OS(=O)(=O)C(F)(F)F)CCOC1] | ethyl 4-oxo- oxane-3- carboxylate |
| 138 | 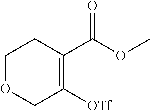 [SMILES: COC(=O)C1=C(OS(=O)(=O)C(F)(F)F)COCC1] | methyl 3-oxo- oxane-4- carboxylate |
| 151 | 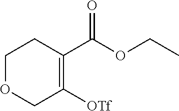 [SMILES: CCOC(=O)C1=C(OS(=O)(=O)C(F)(F)F)COCC1] | ethyl 3-oxo- oxane-4- carboxylate |

### Preparation of Intermediate 127

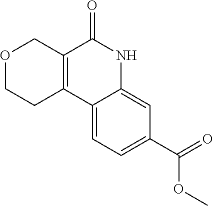 [SMILES: COC(=O)c1ccc2c3c(c(=O)[nH]c2c1)COCC3]

To a mixture of [2-amino-4-(methoxycarbonyl)phenyl]boronic acid (2.02 g, 10.34 mmol) and intermediate 126 (3 g, 10.34 mmol) in 1,4-dioxane (4 mL) and water (1 mL) was added PdCl₂(dppf) (0.75 g, 1.03 mmol) and K₂CO₃ (4.29 g, 31.01 mmol). The resulting mixture was stirred at 100° C. for 5 hours. After cooled down to room temperature, the reaction mixture was diluted with water (20 mL) and extracted with DCM (100 mL×3). The combined organics were dried with anhydrous Na₂SO₄, filtered, and concentrated in vacuo. The residue was purified by silica gel chromatography (MeOH:DCM=1:10) to provide intermediate 127 (1.5 g, yield: 55.97%) as a white solid.

The following intermediate was synthesized by an analogous method as described above for intermediate 127.

| Intermediate | | Starting |
|---|---|---|
| No | Structure | Materials |
| 139 | 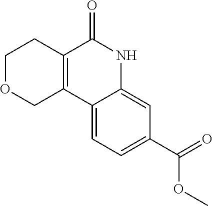 [SMILES: COC(=O)c1ccc2c3c(c(=O)[nH]c2c1)CCOC3] | Intermediate 138 |
| 179 | 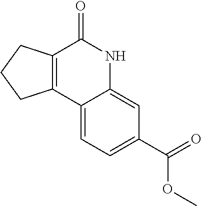 [SMILES: COC(=O)c1ccc2c3c(c(=O)[nH]c2c1)CCC3] | Intermediate 113 |

### Preparation of Intermediate 132

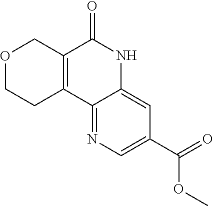 [SMILES: COC(=O)c1cnc2c3c(c(=O)[nH]c2c1)COCC3]

To a solution of methyl 5-amino-6-chloronicotinate (280 mg, 1.50 mmol) and intermediate 131 (482.79 mg, 1.80 mmol) in dioxane/water (4/1 ratio, 6 mL) was added K₂CO₃ (414.78 mg, 3.00 mmol) and Pd(dppf)Cl₂ (109.79 mg, 0.15 mmol). The resulting reaction mixture was stirred at 100° C. for 2 h under N₂ atmosphere. The reaction mixture was concentrated under reduced pressure to give a residue, which was purified by silica gel column chromatography eluted with DCM/MeOH (25/1) to give intermediate 132 (150 mg, yield: 38.41%) as a brown solid.

The following intermediate was synthesized by an analogous method as described above for intermediate 132.

| Intermediate | | Starting |
|---|---|---|
| No | Structure | Materials |
| 153 | 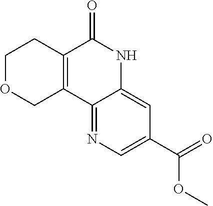 [SMILES: COC(=O)c1cnc2c3c(c(=O)[nH]c2c1)CCOC3] | Intermediate 152 |

### Preparation of Intermediate 135

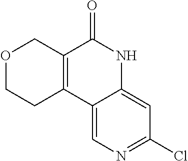 [SMILES: O=c1[nH]c2cc(Cl)ncc2c2c1COCC2]

To a solution of 5-bromo-2-chloropyridin-4-amine (1 g, 4.82 mmol) and intermediate 130 (1.55 g, 5.78 mmol) in dioxane and water (4/1, 22 mL) were added K₂CO₃ (1.33 g, 9.64 mmol) and Pd(dppf)Cl₂ (0.35 g, 0.48 mmol). The resulting reaction mixture was stirred at 100° C. for 2 h under N₂ atmosphere. The reaction mixture was concentrated under reduced pressure to give a residue, which was purified by silica gel column chromatography eluted with DCM/MeOH (25/1) to give intermediate 135 (680 mg, yield: 59.61%) as a yellow solid.

### Preparation of Intermediate 53

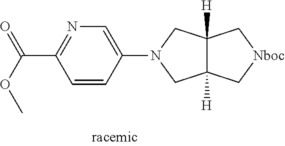

A solution of methyl 5-bromopicolinate (300 mg, 1.39 mmol), (3aRS,6aRS)-tert-butyl hexahydropyrrolo[3,4-c]pyrrole-2(1H)-carboxylate (295 mg, 1.39 mmol), Cs₂CO₃ (893 mg, 2.74 mmol), RuPhos Pd G3 (117 mg, 0.14 mmol) in dioxane (20 mL) was stirred at 100° C. for 3 hours under nitrogen atmosphere. The solvent was removed, and residue was diluted with 30 mL water and extracted with ethyl acetate (20 mL×2). The combined organic layer was dried over anhydrous sodium sulphate. The mixture was filtered and concentrated. The residue was purified by C18 column (acetonitrile:water (0.1% ammonium bicarbonate)=5% to 60%) to give intermediate 53 (350 mg, yield: 72.56%) as a yellow solid.

The following intermediate was synthesized by an analogous method as described above for intermediate 53.

| Intermediate | | Starting |
|---|---|---|
| No | Structure | Materials |
| 95 | 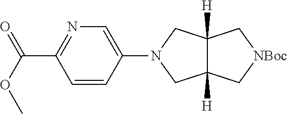 [SMILES: COC(=O)c1ccc(N2C[C@H]3CN(C(=O)OC(C)(C)C)C[C@H]3C2)cn1] | cis-2-Boc-hexa- hydropyrrolo [3,4-c]pyrrole |

### Preparation of Intermediate 54

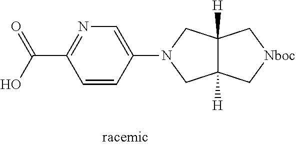

To a solution of intermediate 53 (350 mg, 1.01 mmol) in THF/MeOH (10 mL/2 mL) was added a solution of lithium hydroxide in water (3 mL, 1.0 mmol). The mixture was stirred at 20° C. for 3 hours. The mixture was concentrated, diluted with water, acidified to pH=5 with 1M HCl (aq.) and exacted with ethyl acetate. The organic layer was dried over Na₂SO₄, filtered, and concentrated to give intermediate 54 (280 mg, crude) as a yellow oil, which was used directly for next step.

The following intermediates were synthesized by an analogous method as described above for intermediate 54.

| Intermediate No | Structure | Starting Materials |
|---|---|---|
| 79 | 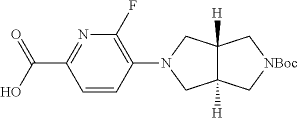 [SMILES: CC(C)(C)OC(=O)N1C[C@H]2CN(c3ccc(C(=O)O)nc3F)C[C@@H]2C1] | Intermediate 69 |
| | racemic | |
| 90 | 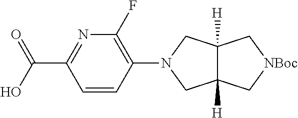 [SMILES: CC(C)(C)OC(=O)N1C[C@@H]2CN(c3ccc(C(=O)O)nc3F)C[C@H]2C1] | Intermediate 65 |
| 96 | 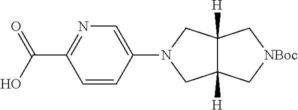 [SMILES: CC(C)(C)OC(=O)N1C[C@@H]2CN(c3ccc(C(=O)O)nc3)C[C@@H]2C1] | Intermediate 95 |
| 159 | 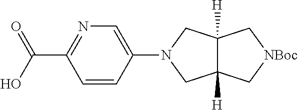 [SMILES: CC(C)(C)OC(=O)N1C[C@@H]2CN(c3ccc(C(=O)O)nc3)C[C@H]2C1] | Intermediate 62 |

### Preparation of Intermediate 55

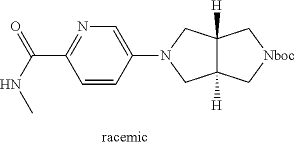

To a solution of intermediate 54 (260 mg, crude) in N,N-dimethylformamide (5 mL) at 25° C. were added DIEA (202 mg, 1.56 mmol), HATU (445 mg, 1.17 mmol) and methanamine hydrochloride (79 mg, 1.17 mmol). The resulting mixture was stirred for 5 h at rt. The solvent was removed, and residue was diluted with 10 mL of water and extracted with ethyl acetate (10 mL×3). The combined organic layer was dried with over anhydrous sodium sulphate, filtered and concentrated. The residue was purified by silica column chromatography (petroleum ether:ethyl acetate=1:1) to give intermediate 55 (180 mg, yield: 66.91%) as a yellow solid.

The following intermediates were synthesized by an analogous method as described above for intermediate 55.

| Intermediate No | Structure | Starting Materials |
|---|---|---|
| 77 | 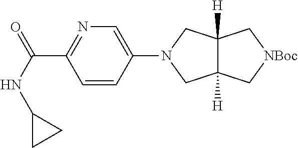 [SMILES: CC(C)(C)OC(=O)N1C[C@H]2CN(c3ccc(C(=O)NC4CC4)nc3)C[C@@H]2C1] | Intermediate 54 & cyclopropylamine |
| | racemic | |
| 80 | 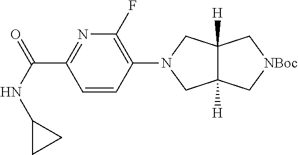 [SMILES: CC(C)(C)OC(=O)N1C[C@H]2CN(c3ccc(C(=O)NC4CC4)nc3F)C[C@@H]2C1] | Intermediate 79 & cyclopropylamine |
| | racemic | |
| 88 | 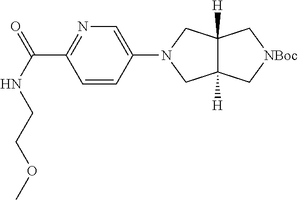 [SMILES: COCCNC(=O)c1ccc(N2C[C@@H]3CN(C(=O)OC(C)(C)C)C[C@H]3C2)cn1] | Intermediate 54 & 2- methoxyethan-1-amine |
| | racemic | |
| 91 | 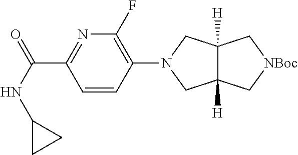 [SMILES: CC(C)(C)OC(=O)N1C[C@@H]2CN(c3ccc(C(=O)NC4CC4)nc3F)C[C@H]2C1] | Intermediate 90 & cyclopropylamine |
| 94 | 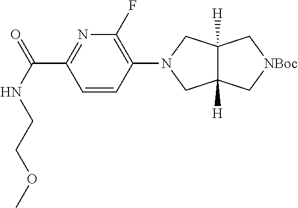 [SMILES: COCCNC(=O)c1ccc(N2C[C@H]3CN(C(=O)OC(C)(C)C)C[C@@H]3C2)c(F)n1] | Intermediate 90 & methoxyethan-1-amine |
| 97 | 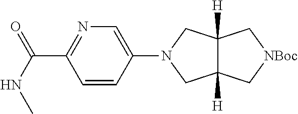 [SMILES: CNC(=O)c1ccc(N2C[C@H]3CN(C(=O)OC(C)(C)C)C[C@H]3C2)cn1] | Intermediate 96 & methanamine hydrochloride |
| 160 | 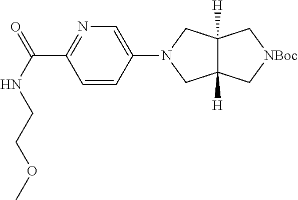 [SMILES: COCCNC(=O)c1ccc(N2C[C@H]3CN(C(=O)OC(C)(C)C)C[C@@H]3C2)cn1] | Intermediate 159 & methoxyethan-1-amine |
| 163 | 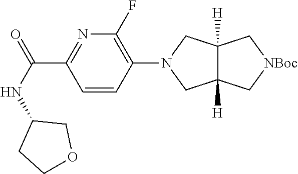 [SMILES: CC(C)(C)OC(=O)N1C[C@@H]2CN(c3ccc(C(=O)N[C@H]4CCOC4)nc3F)C[C@H]2C1] | Intermediate 90 & (S)- tetrahydrofuran-3-amine |
| 165 | 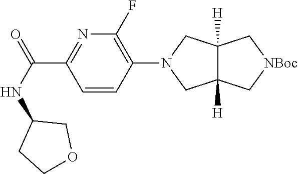 [SMILES: CC(C)(C)OC(=O)N1C[C@@H]2CN(c3ccc(C(=O)N[C@@H]4CCOC4)nc3F)C[C@H]2C1] | Intermediate 90 & (R)- tetrahydrofuran-3-amine |
| 167 | 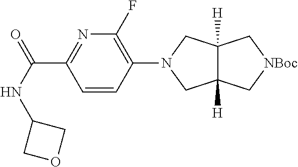 [SMILES: CC(C)(C)OC(=O)N1C[C@@H]2CN(c3ccc(C(=O)NC4COC4)nc3F)C[C@H]2C1] | Intermediate 90 & oxetan-3- amine |
| 169 | 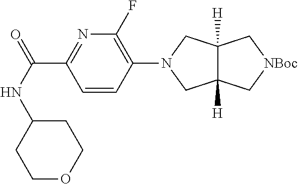 [SMILES: CC(C)(C)OC(=O)N1C[C@@H]2CN(c3ccc(C(=O)NC4CCOCC4)nc3F)C[C@H]2C1] | Intermediate 90 & tetrahydro-2H-pyran-4- amine |
| 171 | 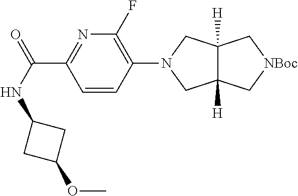 [SMILES: CO[C@H]1C[C@@H](NC(=O)c2ccc(N3C[C@H]4CN(C(=O)OC(C)(C)C)C[C@@H]4C3)c(F)n2)C1] | Intermediate 90 & (1s,3s)-3- methoxycyclobutan-1-amine |
| 175 | 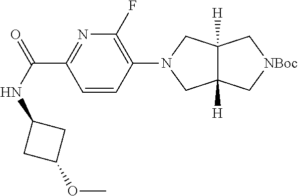 [SMILES: CO[C@H]1C[C@H](NC(=O)c2ccc(N3C[C@H]4CN(C(=O)OC(C)(C)C)C[C@@H]4C3)c(F)n2)C1] | Intermediate 90 & (1r,3r)-3- methoxycyclobutan-1-amine |
| 177 | 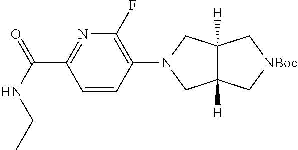 [SMILES: CCNC(=O)c1ccc(N2C[C@H]3CN(C(=O)OC(C)(C)C)C[C@@H]3C2)c(F)n1] | Intermediate 90 & ethylamine |

### Preparation of Intermediate 56

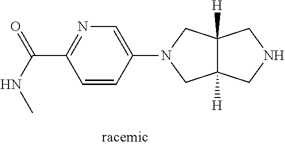 [SMILES: CNC(=O)c1ccc(N2C[C@@H]3CNC[C@H]3C2)cn1]

The mixture of intermediate 55 (180 mg, 0.52 mmol) in TFA (1 mL) and DCM (5 mL) was stirred at rt for 1 hr. The solvent was removed, the residue was diluted with EtOAc, basified with 1M of NaOH (aq.) and was extracted with EtOAc. The combined organic phase was dried over Na₂SO₄, filtered, and concentrated to give intermediate 56 (90 mg, crude) as a yellow oil, which was used directly in the next step.

The following intermediates were synthesized by an analogous method as described above for intermediate 56.

| Intermediate No | Structure | Starting Materials |
|---|---|---|
| 78 | 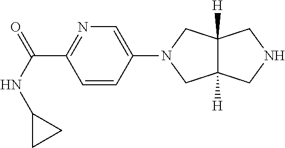 [SMILES: O=C(NC1CC1)c1ccc(N2C[C@@H]3CNC[C@H]3C2)cn1] | Intermediate 77 |
| | racemic | |
| 81 | 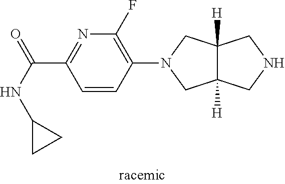 [SMILES: O=C(NC1CC1)c1ccc(N2C[C@@H]3CNC[C@H]3C2)c(F)n1] | Intermediate 80 |
| 89 | 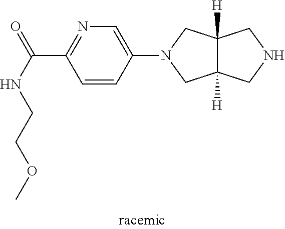 [SMILES: COCCNC(=O)c1ccc(N2C[C@@H]3CNC[C@H]3C2)cn1] | Intermediate 88 |
| 98 | 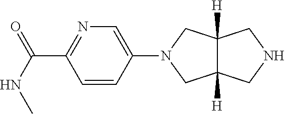 [SMILES: CNC(=O)c1ccc(N2C[C@H]3CNC[C@H]3C2)cn1] | Intermediate 97 |
| 161 | 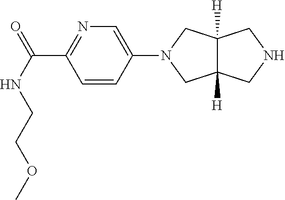 [SMILES: COCCNC(=O)c1ccc(N2C[C@H]3CNC[C@@H]3C2)cn1] | Intermediate 160 |
| 162 | 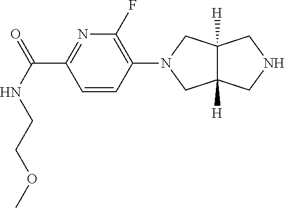 [SMILES: COCCNC(=O)c1ccc(N2C[C@H]3CNC[C@@H]3C2)c(F)n1] | Intermediate 94 |
| 164 | 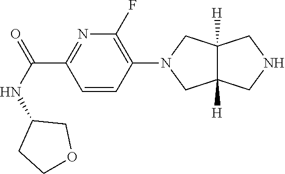 [SMILES: O=C(N[C@H]1CCOC1)c1ccc(N2C[C@H]3CNC[C@@H]3C2)c(F)n1] | Intermediate 163 |
| 166 | 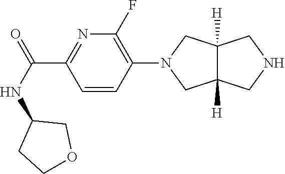 [SMILES: O=C(N[C@@H]1CCOC1)c1ccc(N2C[C@H]3CNC[C@@H]3C2)c(F)n1] | Intermediate 165 |
| 168 | 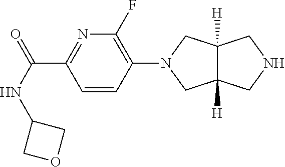 [SMILES: O=C(NC1COC1)c1ccc(N2C[C@H]3CNC[C@@H]3C2)c(F)n1] | Intermediate 167 |
| 170 | 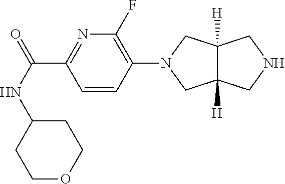 [SMILES: O=C(NC1CCOCC1)c1ccc(N2C[C@H]3CNC[C@@H]3C2)c(F)n1] | Intermediate 169 |
| 172 | 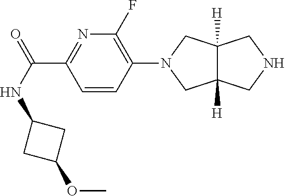 [SMILES: CO[C@H]1C[C@@H](NC(=O)c2ccc(N3C[C@H]4CNC[C@@H]4C3)c(F)n2)C1] | Intermediate 171 |
| 174 | 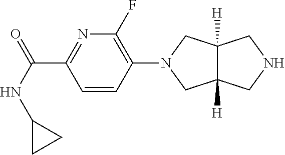 [SMILES: O=C(NC1CC1)c1ccc(N2C[C@H]3CNC[C@@H]3C2)c(F)n1] | Intermediate 91 |
| 176 | 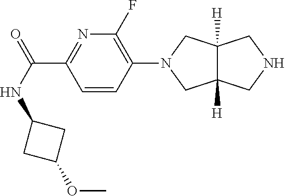 [SMILES: CO[C@H]1C[C@H](NC(=O)c2ccc(N3C[C@H]4CNC[C@@H]4C3)c(F)n2)C1] | Intermediate 175 |
| 178 | 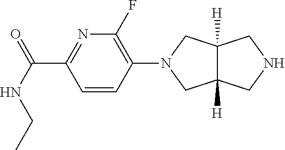 [SMILES: CCNC(=O)c1ccc(N2C[C@H]3CNC[C@@H]3C2)c(F)n1] | Intermediate 177 |

### Preparation of Intermediate 57

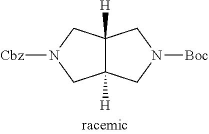 [SMILES: CC(C)(C)OC(=O)N1C[C@H]2CN(C(=O)OCc3ccccc3)C[C@@H]2C1]

To a solution of (3aRS,6aRS)-tert-butyl hexahydropyrrolo[3,4-c]pyrrole-2(1H)-carboxylate oxalate (3 g, 14.13 mmol), TEA (1.43 g, 14.13 mmol) in DCM (50 mL) at rt under N₂ was added slowly a solution of benzyl 2,5-dioxopyrrolidine-1-carboxylate (3.63 g, 15.55 mmol) in DCM (20 mL). The mixture was stirred at 20° C. for 12 hours. The mixture was diluted with 20 mL water and extracted with DCM (20 mL×2). The organic layer was washed with brine (20 mL×2), dried over anhydrous sodium sulphate, filtered, and concentrated. The residues were purified by silica column chromatography (petroleum ether:ethyl acetate=5:1) to afford intermediate 57 (4.3 g, yield: 83.4%) as a white solid.

### Preparation of Intermediate 58 & 59

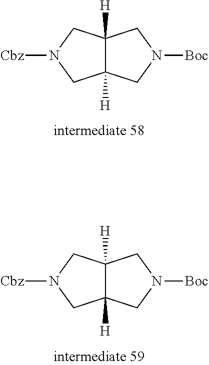 [SMILES: CC(C)(C)OC(=O)N1C[C@H]2CN(C(=O)OCc3ccccc3)C[C@@H]2C1 ; CC(C)(C)OC(=O)N1C[C@@H]2CN(C(=O)OCc3ccccc3)C[C@H]2C1]

Intermediate 57 (4.3 g, 11.79 mmol) was separated by chiral HPLC (separation condition: Column: IH-3.0 cm; Mobile Phase: Hex: EtOH=95:5, at 25 mL/min; Temp: 40° C.; Wavelength: 214 nm). The first fraction was collected as intermediate 58 (1.7 g, 100% e.e. at RT=10.72 min) as a white solid and the second fraction was collected as intermediate 59 (1.7 g, 97.65% e.e. at RT=12.43 min) as a white solid.

### Preparation of Intermediate 60

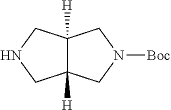 [SMILES: CC(C)(C)OC(=O)N1C[C@@H]2CNC[C@H]2C1]

To a solution of intermediate 59 (280 mg, 0.81 mmol) in MeOH (10 mL) was added Pd/C 10% (280 mg, 2.63 mmol). The mixture was stirred at r.t. for 18 h under H₂ atmosphere. The reaction mixture was filtered, and filtrate was concentrated under reduced pressure to give intermediate 60 (crude) as a white solid, which was used in the next step without further purification.

The following intermediate was synthesized by an analogous method as described above for intermediate 60.

| Intermediate | | |
|---|---|---|
| No | Structure | Starting Materials |
| 61 | 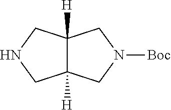 [SMILES: CC(C)(C)OC(=O)N1C[C@H]2CNC[C@@H]2C1] | Intermediate 58 |

### Preparation of Intermediate 62

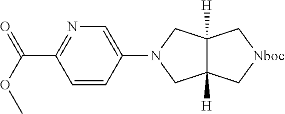

A solution of methyl 5-bromopicolinate (200 mg, 0.91 mmol), intermediate 60 (193 mg, 0.91 mmol), Cs₂CO₃ (591 mg, 1.82 mmol) and RuPhos Pd G3 (76 mg, 0.091 mmol) in dioxane (10 mL) was stirred at 100° C. for 5 hours under nitrogen atmosphere. The solvent was removed, and residue was diluted with water (30 mL) and extracted with ethyl acetate (20 mL×2). The combined organic layer was dried with over anhydrous sodium sulphate and concentrated. The residue was purified by C18 column (acetonitrile:water (0.1% ammonium bicarbonate)=5% to 60%) to give intermediate 62 (220 mg, yield: 62.8%) as yellow solid.

The following intermediates were synthesized by an analogous method as described above for intermediate 62.

| Intermediate No | Structure | Starting Materials |
|---|---|---|
| 74 | 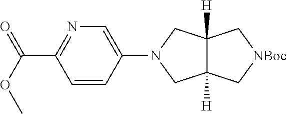 [SMILES: COC(=O)c1ccc(N2C[C@@H]3CN(C(=O)OC(C)(C)C)C[C@H]3C2)cn1] | Intermediate 61 |
| 92 | 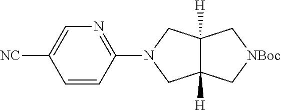 [SMILES: CC(C)(C)OC(=O)N1C[C@@H]2CN(c3ccc(C#N)cn3)C[C@H]2C1] | Intermediate 60 & 6- bromonicotinonitrile |

### Preparation of Intermediate 63

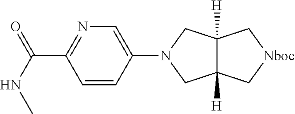

A solution of intermediate 62 (220 mg, 0.63 mmol) in 33% methylamine ethanol solution (2 mL) was stirred at 60° C. for 12 hours. The mixture was concentrated to give intermediate 63 (200 mg, yield: 82.1%) as a yellow oil, which was used in the next step without further purification.

The following intermediate was synthesized by an analogous method as described above for intermediate 63.

| Intermediate No | Structure | Starting Materials |
|---|---|---|
| 75 | 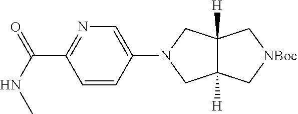 [SMILES: CNC(=O)c1ccc(N2C[C@@H]3CN(C(=O)OC(C)(C)C)C[C@H]3C2)cn1] | Intermediate 74 |

### Preparation of Intermediate 64

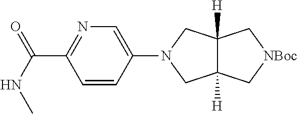 [SMILES: CNC(=O)c1ccc(N2C[C@@H]3CN(C(=O)OC(C)(C)C)C[C@H]3C2)cn1]

To a solution of intermediate 63 (200 mg, 0.58 mmol) in DCM (5 mL) was added TFA (0.4 mL, 5.77 mmol). The resulting reaction mixture was stirred at r.t. for 1 h. The solvent was removed, and the residue was diluted with EtOAc, basified with 1 M of NaOH (aq.) and extracted with EtOAc. The combined organic phase was dried over Na₂SO₄, filtered, and concentrated to give the crude intermediate 64 (120 mg, yield: 84.4%) as a yellow oil, which was used directly in the next step.

The following intermediate was synthesized by an analogous method as described above for intermediate 64.

| Intermediate No | Structure | Starting Materials |
|---|---|---|
| 76 | 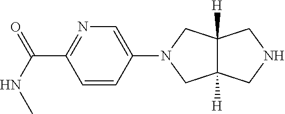 [SMILES: CNC(=O)c1ccc(N2C[C@@H]3CNC[C@H]3C2)cn1] | Intermediate 75 |
| 93 | 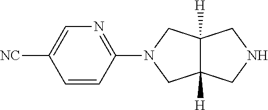 [SMILES: N#Cc1ccc(N2C[C@H]3CNC[C@@H]3C2)nc1] | Intermediate 92 |

### Preparation of Intermediate 65

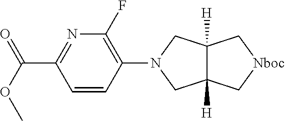

To a solution of intermediate 60 (120 mg, 0.57 mmol) and methyl 5-bromo-6-fluoropyridine-2-carboxylate (145 mg, 0.62 mmol) in dioxane (10 mL) was added Cs₂CO₃ (368 mg, 1.13 mmol) and RuPhos Pd G3 (12 mg, 0.014 mmol). The mixture was stirred at 80° C. for 3 h under N₂ atmosphere. The reaction was poured into water (50 mL) and extracted with EtOAc (50 mL×2). The combined organic layers were dried over anhydrous Na₂SO₄ and filtered. The filtrate was concentrated under reduced pressure to give a residue, which was purified by column chromatography on silica gel to give intermediate 65 (80 mg, yield: 38.73%) as a white solid.

The following intermediates were synthesized by an analogous method as described above for intermediate 65.

| Intermediate No | Structure | Starting Materials |
|---|---|---|
| 68 | 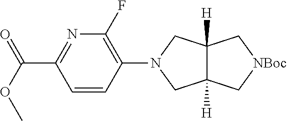 [SMILES: COC(=O)c1ccc(N2C[C@@H]3CN(C(=O)OC(C)(C)C)C[C@H]3C2)c(F)n1] | Intermediate 61 |
| 69 | 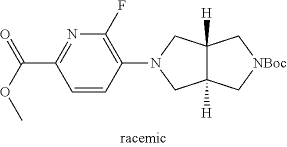 [SMILES: COC(=O)c1ccc(N2C[C@@H]3CN(C(=O)OC(C)(C)C)C[C@H]3C2)c(F)n1] | (3aRS,6aRS)-tert-butyl hexahydropyrrolo[3,4- c]pyrrole-2(1H)-carboxylate |
| 82 | 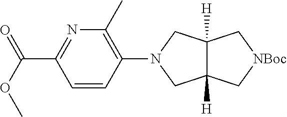 [SMILES: COC(=O)c1ccc(N2C[C@H]3CN(C(=O)OC(C)(C)C)C[C@@H]3C2)c(C)n1] | Intermediate 60 & methyl 5- bromo-6-methylpicolinate |
| 83 | 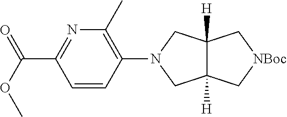 [SMILES: COC(=O)c1ccc(N2C[C@@H]3CN(C(=O)OC(C)(C)C)C[C@H]3C2)c(C)n1] | Intermediate 61 & methyl 5- bromo-6-methylpicolinate |

### Preparation of Intermediate 66

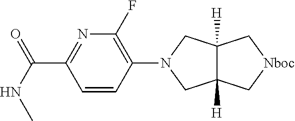

To a solution of intermediate 66 (80 mg, 0.22 mmol) in EtOH (8 mL) was added a solution of methanamine in EtOH (0.009 mL, 0.018 mmol). The resulting reaction mixture was stirred at 60° C. for 18 h. The reaction was concentrated under reduced pressure to give intermediate 66 (crude) as a white solid, which was used in the next step without further purification.

The following intermediates were synthesized by an analogous method as described above for intermediate 66.

| Intermediate No | Structure | Starting Materials |
|---|---|---|
| 70 | 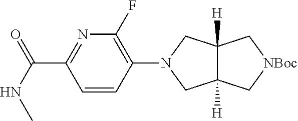 [SMILES: CNC(=O)c1ccc(N2C[C@@H]3CN(C(=O)OC(C)(C)C)C[C@H]3C2)c(F)n1] | Intermediate 68 |
| 71 | 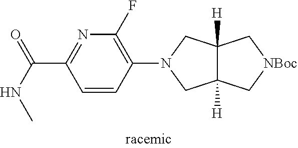 [SMILES: CNC(=O)c1ccc(N2C[C@@H]3CN(C(=O)OC(C)(C)C)C[C@H]3C2)c(F)n1] | Intermediate 69 |
| 84 | 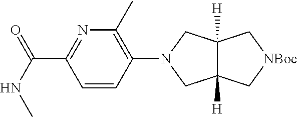 [SMILES: CNC(=O)c1ccc(N2C[C@H]3CN(C(=O)OC(C)(C)C)C[C@@H]3C2)c(C)n1] | Intermediate 82 |
| 85 | 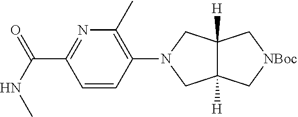 [SMILES: CNC(=O)c1ccc(N2C[C@@H]3CN(C(=O)OC(C)(C)C)C[C@H]3C2)c(C)n1] | Intermediate 83 |

### Preparation of Intermediate 67

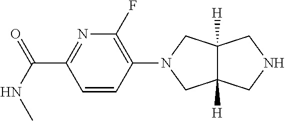 [SMILES: CNC(=O)c1ccc(N2C[C@H]3CNC[C@@H]3C2)c(F)n1]

A solution of intermediate 66 (79 mg, 0.22 mmol) in DCM was added HCl in EtOAc (6 mL, 4 N). The resulting reaction mixture was stirred at rt for 1 h. The reaction was concentrated under reduced pressure to give intermediate 67 (crude), which was used in the next step without further purification.

The following intermediates were synthesized by an analogous method as described above for intermediate 67.

| Intermediate No | Structure | Starting Materials |
|---|---|---|
| 72 | 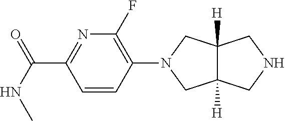 [SMILES: CNC(=O)c1ccc(N2C[C@@H]3CNC[C@H]3C2)c(F)n1] | Intermediate 70 |
| 73 | 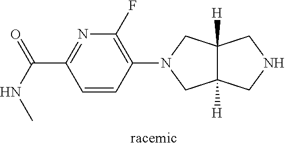 [SMILES: CNC(=O)c1ccc(N2C[C@@H]3CNC[C@H]3C2)c(F)n1] | Intermediate 71 |
| 86 | 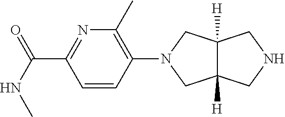 [SMILES: CNC(=O)c1ccc(N2C[C@H]3CNC[C@@H]3C2)c(C)n1] | Intermediate 84 |
| 87 | 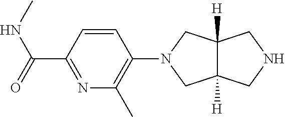 [SMILES: CNC(=O)c1ccc(N2C[C@@H]3CNC[C@H]3C2)c(C)n1] | Intermediate 85 |

### Preparation of Intermediate 99

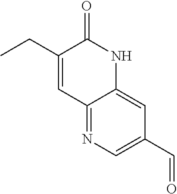

To a mixture of intermediate 5 (220 mg, 1.08 mmol) in DCM (20 mL) was added MnO₂ (938 mg, 10.80 mmol) at 0° C. The mixture was stirred at 30° C. under nitrogen atmosphere for 12 hrs. The mixture was filtered off and washed with DCM, the filtrate was concentrated under reduced pressure to give intermediate 99 (180 mg, crude) as white solid, which was used directly in the next step.

### Preparation of Intermediate 182

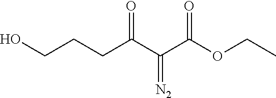

To a solution of ethyl 2-diazoacetate (5.96 g, 52.27 mmol) and oxolan-2-one (3.00 g, 34.85 mmol) in THF (180 mL) was added Li-HMDS (52.3 mL, 52.3 mmol) at −78° C. The reaction mixture was stirred at rt for 1.5 h under nitrogen atmosphere. The reaction mixture was poured into saturated NaHCO₃ (200 mL) and extracted with EtOAc. The combined organic layers were washed with brine. The organic layer was dried over Na₂SO₄ and filtered. The filtrate was concentrated under reduced pressure to give a residue, which was purified by column chromatography on silica gel eluted with PE/EtOAc (2/1) to afford intermediate 182 (3.6 g, yield: 40.14%) as a yellow solid.

### Preparation of Intermediate 183

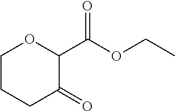

To a solution of intermediate 182 (3.6 g, 17.98 mmol) in toluene (360 mL) was added Rh₂(OAc)₄ (0.08 g, 0.18 mmol). The reaction mixture was stirred at 80° C. for 1 h under nitrogen atmosphere. The reaction mixture was concentrated under reduced pressure to give a residue, which was purified by column chromatography on silica gel eluted with PE/EtOAc (3/1) to afford intermediate 183 (1.23 g, yield: 39.72%) as a yellow oil.

### Preparation of Intermediate 184

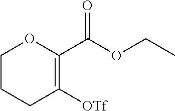 [SMILES: CCOC(=O)C1=C(OS(=O)(=O)C(F)(F)F)CCCO1]

To a solution of intermediate 183 (1.23 g, 7.14 mmol) in DCM (35 mL) was added 2,6-di-tert-butyl-4-methylpyridine (1.5 mL, 7.14 mmol) in DCM (35 mL) under nitrogen atmosphere at 0° C. To the mixture was added Tf₂O (2.014 g, 7.14 mmol), and the reaction mixture was stirred at room temperature for 16 h. The reaction mixture was filtered, and the filtrate was concentrated under reduced pressure to give a residue, which was purified by column chromatography on silica gel eluted with PE/EtOAc (5/1) to afford intermediate 184 (900 mg, yield: 41.41%) as a yellow oil.

The following intermediate was synthesized by an analogous method as described above for intermediate 184.

| Intermediate | | |
|---|---|---|
| No | Structure | Starting Materials |
| 189 | 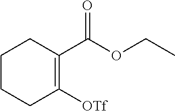 [SMILES: CCOC(=O)C1=C(OS(=O)(=O)C(F)(F)F)CCCC1] | ethyl 2-oxocyclohexane- 1-carboxylate |

### Preparation of Intermediate 185

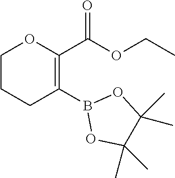 [SMILES: CCOC(=O)C1=C(B2OC(C)(C)C(C)(C)O2)CCCO1]

To a solution of intermediate 184 (900 mg, 2.96 mmol) and 4,4,5,5-tetramethyl-2-(tetramethyl-1,3,2-dioxaborolan-2-yl)-1,3,2-dioxaborolane (901 mg, 3.55 mmol) in dioxane (15 mL) was added KOAc (580 mg, 5.92 mmol) and Pd(dppf)Cl₂ (216 mg, 0.30 mmol). The reaction mixture was stirred at 80° C. for 3 h under nitrogen atmosphere. The reaction mixture was concentrated under reduced pressure to give a residue, which was purified by column chromatography on silica gel eluted with PE/EtOAc (5/1) to afford intermediate 185 (800 mg, yield: 95.85%) as a colorless oil.

The following intermediate was synthesized by an analogous method as described above for intermediate 185.

| Intermediate | | |
|---|---|---|
| No | Structure | Starting Materials |
| 190 | 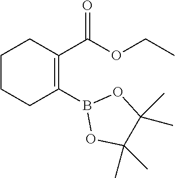 [SMILES: CCOC(=O)C1=C(B2OC(C)(C)C(C)(C)O2)CCCC1] | Intermediate 189 |

### Preparation of Intermediate 186

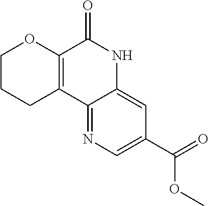 [SMILES: COC(=O)c1cnc2c3c(c(=O)[nH]c2c1)OCCC3]

To a solution of intermediate 185 (400 mg, 1.42 mmol) and methyl 5-amino-6-chloropyridine-3-carboxylate (241 mg, 1.29 mmol) in dioxane/water (5/1, 6 mL) were added Pd(dppf)Cl₂ (94 mg, 0.13 mmol) and K₂CO₃ (356 mg, 2.58 mmol). The reaction mixture was stirred at 100° C. for 3 h under nitrogen atmosphere. The reaction mixture was diluted with water (30 mL). The reaction mixture was extracted with DCM and the combined organic layers were washed with brine, dried over anhydrous sodium sulphate. The mixture was filtered, and the filtrate was evaporated under reduced pressure. The residue was purified by flash column chromatography (MeOH in DCM=0-10%) to afford intermediate 186 (75 mg, yield: 22.36%) as a white solid.

The following intermediate was synthesized by an analogous method as described above for intermediate 186.

| Intermediate | | Starting |
|---|---|---|
| No | Structure | Materials |
| 191 | 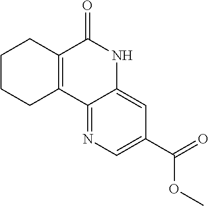 [SMILES: COC(=O)c1cnc2c3c(c(=O)[nH]c2c1)CCCC3] | Intermediate 190 |

### Preparation of Intermediate 194

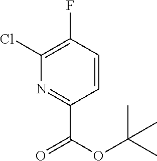

To a solution of 6-chloro-5-fluoropyridine-2-carboxylic acid (800 mg, 4.56 mmol) in t-BuOH (5 mL) were added (Boc)₂O (1.988 g, 9.11 mmol) and DMAP (612 mg, 5.01 mmol). The reaction mixture was stirred at 50° C. for 16 h under N₂ atmosphere. The reaction mixture was poured into water, extracted with EtOAc. The combined organic layers were dried over Na₂SO₄ and filtered. The filtrate was concentrated under reduced pressure. The residue was purified by column chromatography on silica gel eluted with PE/EtOAc (10/1) to provide intermediate 194 (300 mg, yield: 28.42%) as a white solid.

### Preparation of Intermediate 195

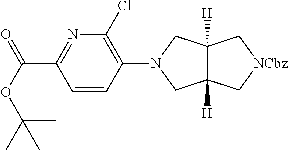 [SMILES: CC(C)(C)OC(=O)c1ccc(N2C[C@H]3CN(C(=O)OCc4ccccc4)C[C@@H]3C2)c(Cl)n1]

To a solution of benzyl (3aS,6aS)-3a,6a-dihydrogenio-octahydropyrrolo[3,4-c]pyrrole-2-carboxylate (212 mg, 0.86 mmol) in DMF (5 mL) were added intermediate 194 (200 mg, 0.86 mmol) and DIEA (558 mg, 4.32 mmol). The reaction mixture was stirred at 100° C. for 16 h under N₂ atmosphere. The reaction mixture was poured into water, the reaction mixture was extracted with EtOAc. The combined organic layers were washed with brine, dried over Na₂SO₄ and filtered. The filtrate was concentrated under reduced pressure. The residue was purified by column chromatography on silica gel eluted with PE/EtOAc (1/1) to provide intermediate 195 (350 mg, yield: 88.52%) as a white solid.

### Preparation of Intermediate 196

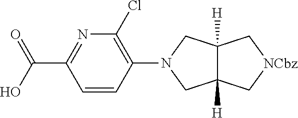 [SMILES: O=C(O)c1ccc(N2C[C@H]3CN(C(=O)OCc4ccccc4)C[C@@H]3C2)c(Cl)n1]

To a solution of intermediate 195 (350 mg, 0.76 mmol) in DCM (2 mL) was added TFA (2 mL, 26.93 mmol) at rt. The reaction mixture was stirred at 40° C. for 4 h. TFA was removed under vacuo, the residue was diluted with water, and the mixture was adjusted to pH 2 with hydrochloride aqueous (1 M). The resulting mixture was extracted with DCM. The organic layer was washed with brine, dried over anhydrous sodium sulfate, filtered, and concentrated under reduced pressure to afford intermediate 196 (300 mg, yield: 97.68%) as a yellow solid.

### Preparation of Intermediate 197

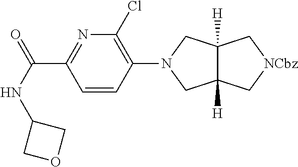 [SMILES: O=C(NC1COC1)c1ccc(N2C[C@H]3CN(C(=O)OCc4ccccc4)C[C@@H]3C2)c(Cl)n1]

To a solution of intermediate 196 (300 mg, 0.75 mmol) in DMF (5 mL) were added oxetan-3-amine (545 mg, 7.46 mmol), HATU (852 mg, 2.24 mmol) and DIEA (482 mg, 3.73 mmol). The reaction mixture was stirred at rt for 2 h. The reaction mixture was diluted with water. The mixture was extracted with DCM. The combined organic layers were washed with brine, dried over Na₂SO₄, and filtered. The filtrate was concentrated under reduced pressure, the residue was purified by column chromatography on silica gel eluted with PE/EA (1/2) to provide intermediate 197 (300 mg, yield: 87.95%) as a yellow solid.

### Preparation of Intermediate 198

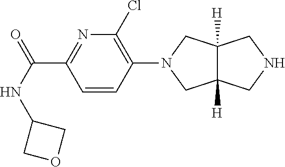 [SMILES: O=C(NC1COC1)c1ccc(N2C[C@H]3CNC[C@@H]3C2)c(Cl)n1]

To a solution of intermediate 197 (300 mg, 0.66 mmol) in EtOH (5 mL) was added PdCl₂ (11 mg, 0.066 mmol). The reaction mixture was stirred at r.t. for 16 hr under H₂ atmosphere. The reaction mixture was filtered, and the filtrate was concentrated under reduced pressure to afford intermediate 198 (110 mg, yield: 51.90%) as a yellow solid.

### Preparation of Intermediate 199

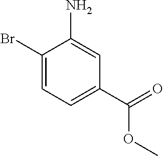

To a solution of methyl 4-bromo-3-nitrobenzoate (2.0 g, 7.69 mmol) in MeOH (20 mL) and H₂O (7 mL) were added Fe (2.2 g, 38 mmol) and NH₄Cl (4.1 g, 77 mmol). The reaction mixture was stirred at 80° C. for 3 h. The reaction mixture was filtered. The filtrate was concentrated under reduced pressure to afford intermediate 199 (1.7 g, yield: 96.1%) as a yellow oil.

### Preparation of Intermediate 200

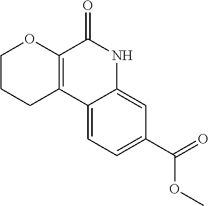 [SMILES: COC(=O)c1ccc2c3c(c(=O)[nH]c2c1)OCCC3]

To a solution of intermediate 199 (270 mg, 1.17 mmol) and intermediate 185 (397 mg, 1.41 mmol) in dioxane/water (6 mL, v/v=10:1) were added K₂CO₃ (324 mg, 2.35 mmol) and Pd(dppf)Cl₂ (86 mg, 0.12 mmol). The reaction mixture was stirred at 80° C. for 3 h under N₂ atmosphere. The reaction mixture was concentrated under reduced pressure, the residue was purified by column chromatography on silica gel eluted with DCM/MeOH (25:1) to afford intermediate 200 (131 mg, yield: 43.0%) as a yellow solid.

### Preparation of Intermediate 203

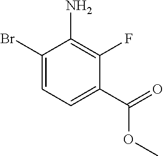

To a solution of methyl 3-amino-2-fluorobenzoate (6000 mg, 35.47 mmol) in HOAc (30 mL) was added dropwise Br₂ (1.8 mL, 35.47 mmol) at 0° C. The reaction mixture was stirred at room temperature for 3 h. The reaction mixture was diluted with water (30 mL). The reaction mixture was extracted with ethyl acetate and the combined organic layers were washed with NaOH solution (1 M), dried over anhydrous sodium sulphate. The mixture was filtered, and the filtrate was evaporated under reduced pressure. The residue was purified by flash column chromatography (EtOAc in PE=0-10% to ACN/H₂O=5-95%) to afford intermediate 203 (550 mg, yield: 6.25%) as a yellow solid.

### Preparation of Intermediate 204

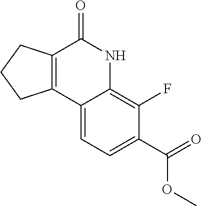 [SMILES: COC(=O)c1ccc2c3c(c(=O)[nH]c2c1F)CCC3]

To a solution of intermediate 203 (630 mg, 2.54 mmol) and intermediate 113 (676 mg, 2.54 mmol) in dioxane-water (5/1, 20 mL) was added Pd(dppf)Cl₂ (186 mg, 0.25 mmol) and K₂CO₃ (1053 mg, 7.62 mmol). The reaction mixture was stirred at 80° C. for 16 h under nitrogen atmosphere. The reaction mixture was diluted with water (30 mL). The reaction mixture was extracted with DCM (30 mL×3) and the combined organic layers were washed with brine, dried over anhydrous sodium sulphate. The mixture was filtered, and the filtrate was evaporated under reduced pressure. The residue was purified by flash column chromatography (EtOAc in PE=0-50%) to afford intermediate 204 (300 mg, yield: 45.21%) as a yellow solid.

### Example 3: Preparation of Compounds

### Preparation of Compound 2

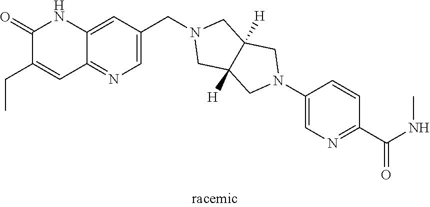 [SMILES: CCc1cc2ncc(CN3C[C@H]4CN(c5ccc(C(=O)NC)nc5)C[C@@H]4C3)cc2[nH]c1=O]

To a mixture of compound intermediate 56 (50 mg, 0.24 mmol) and intermediate 99 (60 mg, 0.24 mmol) in DCE (10 mL) were added sodium triacetoxyborohydride (102 mg, 0.48 mmol) and AcOH (0.01 mL). The reaction mixture was stirred at r.t. overnight. The mixture was concentrated and purified by Prep-HPLC (Column: Xbridge C18 (5 μm 19*150 mm), Mobile Phase A: Water (0.1% ammonium bicarbonate), Mobile Phase B: acetonitrile, UV: 214 nm, Flow rate: 15 mL/min, GT: 10 mins, Gradient: 5-50% (% B)) to give Compound 2 (28 mg, yield: 27.01%) as a white solid.

The following Compound was synthesized by an analogous method as described above for Compound 2.

| Compound No | Structure | Starting Materials |
|---|---|---|
| 1 | 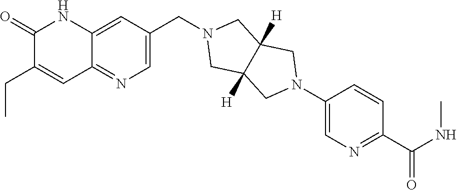 [SMILES: CCc1cc2ncc(CN3C[C@@H]4CN(c5ccc(C(=O)NC)nc5)C[C@@H]4C3)cc2[nH]c1=O] | Intermediate 98 & intermediate 99 |

### Preparation of Compound 3

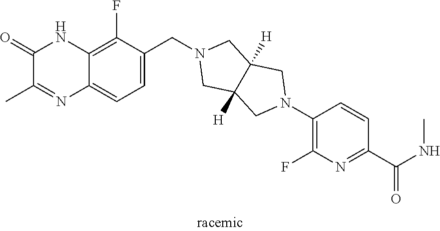 [SMILES: CNC(=O)c1ccc(N2C[C@@H]3CN(Cc4ccc5nc(C)c(=O)[nH]c5c4F)C[C@H]3C2)c(F)n1]

To a solution of intermediate 73 (90 mg, 0.299 mmol) in acetonitrile (10 mL) were added intermediate 12 (81.12 mg, 0.299 mmol) and DIEA (0.049 mL, 0.299 mmol). The mixture was stirred at 80° C. for 2 h under nitrogen atmosphere. The mixture was concentrated, and the residue was dissolved in DMF (1 ml), which was purified by prep-HPLC to give Compound 3 (20 mg, yield: 14%) as a white solid.

The following Compounds were synthesized by an analogous method as described above for Compound 3.

| Compound | | |
|---|---|---|
| No | Structure | Starting Materials |
| 4 | 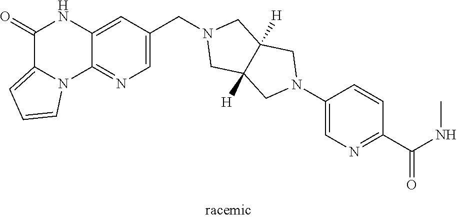 [SMILES: CNC(=O)c1ccc(N2C[C@@H]3CN(Cc4cnc5c(c4)[nH]c(=O)c4cccn45)C[C@H]3C2)cn1] | Intermediate 16 & intermediate 56 |
| 5 | 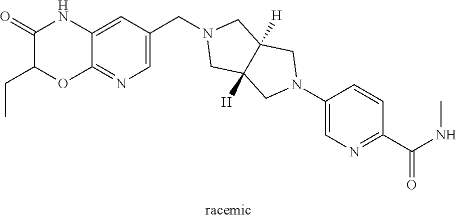 [SMILES: CCC1Oc2ncc(CN3C[C@H]4CN(c5ccc(C(=O)NC)nc5)C[C@@H]4C3)cc2NC1=O] | Intermediate 20 & intermediate 56 |
| 6 | 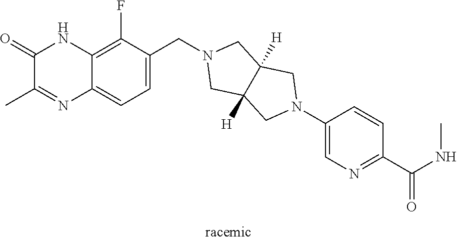 [SMILES: CNC(=O)c1ccc(N2C[C@@H]3CN(Cc4ccc5nc(C)c(=O)[nH]c5c4F)C[C@H]3C2)cn1] | Intermediate 12 & intermediate 56 |
| 7 | 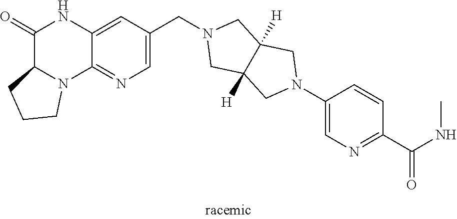 [SMILES: CNC(=O)c1ccc(N2C[C@@H]3CN(Cc4cnc5c(c4)NC(=O)[C@@H]4CCCN54)C[C@H]3C2)cn1] | Intermediate 24 & intermediate 56 |
| 8 | 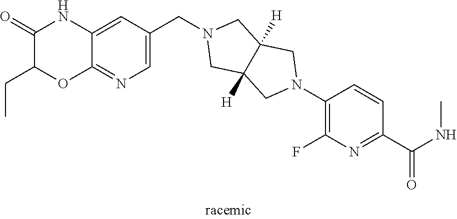 [SMILES: CCC1Oc2ncc(CN3C[C@H]4CN(c5ccc(C(=O)NC)nc5F)C[C@@H]4C3)cc2NC1=O] | Intermediate 20 & intermediate 73 |
| 12 | 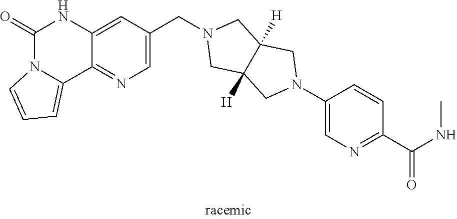 [SMILES: CNC(=O)c1ccc(N2C[C@@H]3CN(Cc4cnc5c(c4)[nH]c(=O)n4cccc54)C[C@H]3C2)cn1] | Intermediate 28 & intermediate 56 |
| 13 | 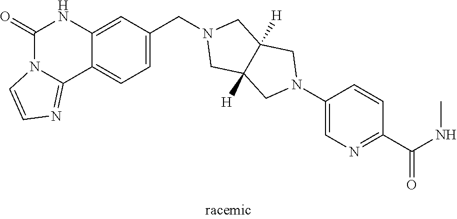 [SMILES: CNC(=O)c1ccc(N2C[C@@H]3CN(Cc4ccc5c(c4)[nH]c(=O)n4ccnc54)C[C@H]3C2)cn1] | Intermediate 31 & intermediate 56 |
| 14 | 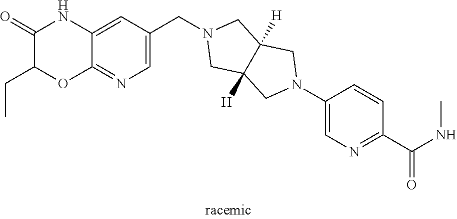 [SMILES: CCC1Oc2ncc(CN3C[C@H]4CN(c5ccc(C(=O)NC)nc5)C[C@@H]4C3)cc2NC1=O] | Intermediate 20 & intermediate 56 |
| 16 | 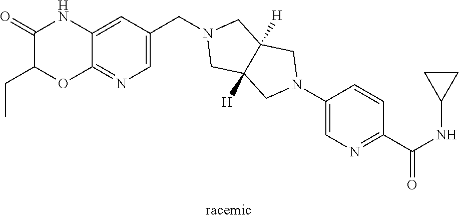 [SMILES: CCC1Oc2ncc(CN3C[C@H]4CN(c5ccc(C(=O)NC6CC6)nc5)C[C@@H]4C3)cc2NC1=O] | Intermediate 20 & intermediate 78 |
| 17 | 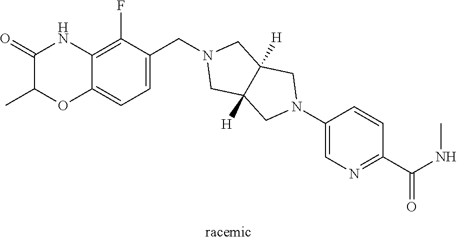 [SMILES: CNC(=O)c1ccc(N2C[C@@H]3CN(Cc4ccc5c(c4F)NC(=O)C(C)O5)C[C@H]3C2)cn1] | Intermediate 36 & intermediate 56 |
| 18 | 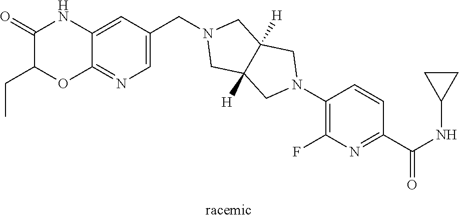 [SMILES: CCC1Oc2ncc(CN3C[C@H]4CN(c5ccc(C(=O)NC6CC6)nc5F)C[C@@H]4C3)cc2NC1=O] | Intermediate 20 & intermediate 81 |
| 19 | 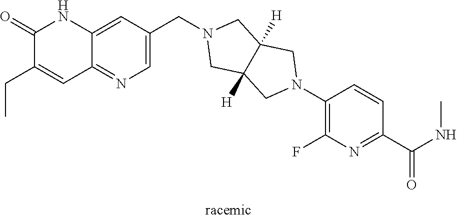 [SMILES: CCc1cc2ncc(CN3C[C@H]4CN(c5ccc(C(=O)NC)nc5F)C[C@@H]4C3)cc2[nH]c1=O] | Intermediate 6 & intermediate 73 |
| 20 | 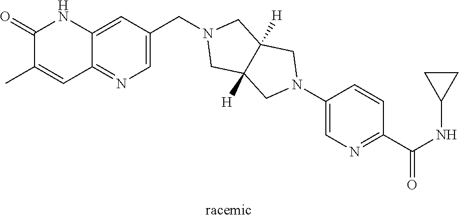 [SMILES: Cc1cc2ncc(CN3C[C@H]4CN(c5ccc(C(=O)NC6CC6)nc5)C[C@@H]4C3)cc2[nH]c1=O] | Intermediate 41 & intermediate 78 |
| 21 | 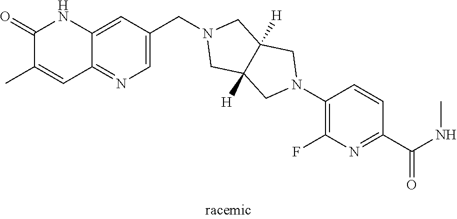 [SMILES: CNC(=O)c1ccc(N2C[C@@H]3CN(Cc4cnc5cc(C)c(=O)[nH]c5c4)C[C@H]3C2)c(F)n1] | Intermediate 41 & intermediate 73 |
| 26 | 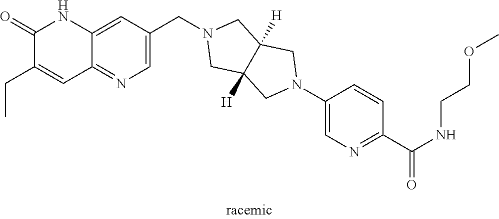 [SMILES: CCc1cc2ncc(CN3C[C@H]4CN(c5ccc(C(=O)NCCOC)nc5)C[C@@H]4C3)cc2[nH]c1=O] | Intermediate 6 & intermediate 89 |

### Preparation of Compound 9

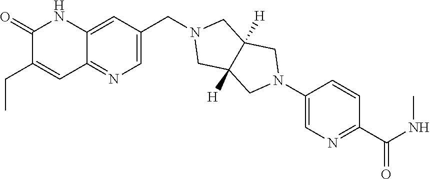 [SMILES: CCc1cc2ncc(CN3C[C@H]4CN(c5ccc(C(=O)NC)nc5)C[C@@H]4C3)cc2[nH]c1=O]

To a solution of intermediate 76 (109 mg, 0.39 mmol) in MeCN (10 mL) was added intermediate 6 (100 mg, 0.39 mmol), DIEA (1 mL, 6.05 mmol) and potassium iodide (13 mg, 0.077 mmol). The reaction mixture was stirred at 80° C. for 2 h. After cooling down to room temperature, the reaction mixture was concentrated under reduced pressure to give a residue, which was purified by prep-HPLC (Xbridge C18 (5 μm 19*150 mm), Mobile Phase A: Water (0.1% NH₄HCO₃), Mobile Phase B: acetonitrile, UV: 214 nm, Flowrate: 15 mL/min, Temperature: r.t., Gradient: 10-40% (% B)) to give Compound 9 (28.8 mg, yield: 17.3%) as a yellow solid. ¹H NMR (400 MHz, DMSO-d₆) 11.82 (s, 1H), 8.41 (d, J=1.6 Hz, 1H), 8.29-8.28 (m, 1H), 7.85 (d, J=2.4 Hz, 1H), 7.79 (d, J=8.8 Hz, 1H), 7.74 (s, 1H), 7.62 (s, 1H), 6.93 (dd, J=8.8, 2.8 Hz, 1H), 4.03-3.94 (m, 2H), 3.53-3.49 (m, 2H), 3.08 (t, J=9.2 Hz, 2H), 2.92-2.89 (m, 2H), 2.77 (d, J=4.8 Hz, 3H), 2.66 (t, J=8.8 Hz, 2H), 2.57-2.51 (m, 2H), 2.40-2.39 (m, 2H), 1.18 (t, J=7.2 Hz, 3H).

The following Compounds were synthesized by an analogous method as described above for Compound 9.

| Compound No | Structure | Starting Materials |
|---|---|---|
| 11 | 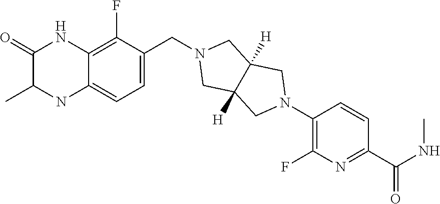 [SMILES: CNC(=O)c1ccc(N2C[C@@H]3CN(Cc4ccc5c(c4F)NC(=O)C(C)N5)C[C@H]3C2)c(F)n1] | Intermediate 12 & intermediate 72 |
| 23 | 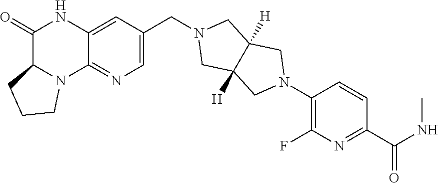 [SMILES: CNC(=O)c1ccc(N2C[C@@H]3CN(Cc4cnc5c(c4)NC(=O)[C@@H]4CCCN54)C[C@H]3C2)c(F)n1] | Intermediate 24 & intermediate 72 |
| 24 | 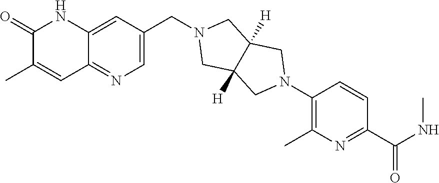 [SMILES: CNC(=O)c1ccc(N2C[C@@H]3CN(Cc4cnc5cc(C)c(=O)[nH]c5c4)C[C@H]3C2)c(C)n1] | Intermediate 41 & intermediate 87 |

### Preparation of Compound 15

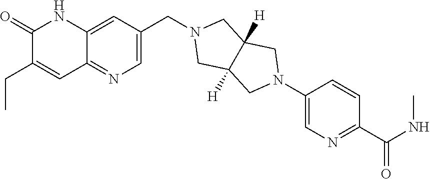 [SMILES: CCc1cc2ncc(CN3C[C@@H]4CN(c5ccc(C(=O)NC)nc5)C[C@H]4C3)cc2[nH]c1=O]

To a solution of intermediate 64 (150 mg, 0.61 mmol), DIEA (393 mg, 3.04 mmol) and potassium iodide (10 mg, 0.061 mmol) in acetonitrile (20 mL) was added intermediate 6 (51 mg, 0.19 mmol). The resulting reaction mixture was stirring at 80° C. for 2 h. The mixture was concentrated to give crude compound, which was purified by Pre-HPLC (Column: RP-PREP-5 Xbrige C18 (5 μm 19*150 mm), Mobile Phase A: Water (0.1% NH₄HCO₃), Mobile Phase B: acetonitrile, UV: 214 nm, Flow rate: 15 mL/min, GT: 10 mins, Temperature: r.t., Gradient: 10-55% (% B)) to afford Compound 15 (130 mg, yield: 49%) as a white solid.

¹H NMR (400 MHz, DMSO-d₆) δ 11.83 (s, 1H), 8.40 (d, J=1.6 Hz, 1H), 8.30-8.26 (m, 1H), 7.85 (d, J=2.8 Hz, 1H), 7.79 (d, J=8.8 Hz, 1H), 7.74 (s, 1H), 7.62 (s, 1H), 6.92 (dd, J=8.8, 2.8 Hz, 1H), 4.03-3.92 (m, 2H), 3.53-3.49 (m, 2H), 3.08 (t, J=9.6 Hz, 2H), 2.92-2.89 (m, 2H), 2.76 (d, J=4.8 Hz, 2H), 2.66 (t, J=10.0 Hz, 2H), 2.59-2.53 (m, 2H), 2.42-2.36 (m, 2H), 1.18 (t, J=7.2 Hz, 3H).

The following Compounds were synthesized by an analogous method as described above for Compound 15.

| Compound No | Structure | Starting Materials |
|---|---|---|
| 10 | 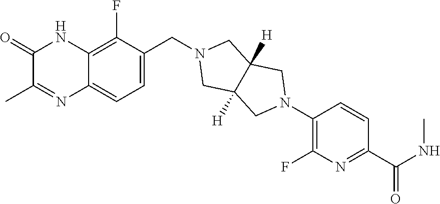 [SMILES: CNC(=O)c1ccc(N2C[C@H]3CN(Cc4ccc5nc(C)c(=O)[nH]c5c4F)C[C@@H]3C2)c(F)n1] | Intermediate 12 & intermediate 67 |
| 25 | 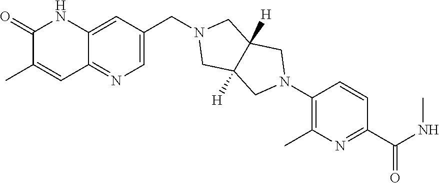 [SMILES: CNC(=O)c1ccc(N2C[C@H]3CN(Cc4cnc5cc(C)c(=O)[nH]c5c4)C[C@@H]3C2)c(C)n1] | Intermediate 41 & intermediate 86 |
| 31 | 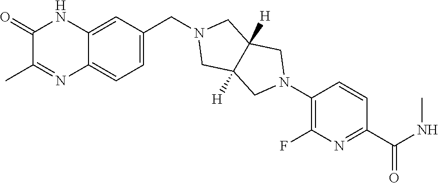 [SMILES: CNC(=O)c1ccc(N2C[C@H]3CN(Cc4ccc5nc(C)c(=O)[nH]c5c4)C[C@@H]3C2)c(F)n1] | Intermediate 52 & intermediate 67 |
| 32 | 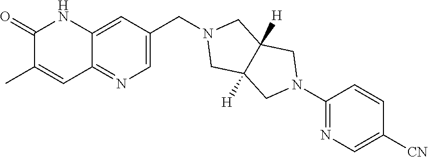 [SMILES: Cc1cc2ncc(CN3C[C@@H]4CN(c5ccc(C#N)cn5)C[C@H]4C3)cc2[nH]c1=O] | Intermediate 41 & intermediate 93 |

### Preparation of Compound 27

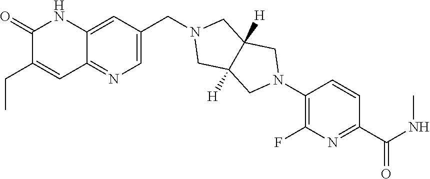 [SMILES: CCc1cc2ncc(CN3C[C@@H]4CN(c5ccc(C(=O)NC)nc5F)C[C@H]4C3)cc2[nH]c1=O]

To a solution intermediate 67 (33 mg, 0.13 mmol) in acetonitrile (6 mL) was added intermediate 6 (33 mg, 0.15 mmol), DIEA (0.062 mL, 0.38 mmol) and potassium iodide (2 mg, 0.013 mmol). The mixture was stirred at 80° C. for 3 h. The reaction was concentrated under reduced pressure to give a residue, which was purified by prep-HPLC (Xbridge C18 (5 μm 19*150 mm), Mobile Phase A: Water (0.1% NH₄HCO₃), Mobile Phase B: acetonitrile, UV: 214 nm, Flowrate: 15 mL/min, Temperature: r.t., Gradient: 45-80% (% B)) to Compound 27 (22.6 mg, yield: 38.80%) as a white solid. ¹H NMR (400 MHz, DMSO-d₆) 11.82 (s, 1H), 8.40 (d, J=1.6 Hz, 1H), 8.18 (d, J=4.8 Hz, 1H), 7.74-7.72 (m, 2H), 7.62 (s, 1H), 7.13-7.08 (m, 1H), 4.02-3.93 (m, 2H), 3.55 (d, J=2.8 Hz, 2H), 3.25 (d, J=8.8 Hz, 2H), 2.90-2.87 (m, 2H), 2.75 (d, J=4.8 Hz, 3H), 2.66-2.62 (m, 2H), 2.57-2.53 (m, 2H), 2.35 (t, J=4.8 Hz, 2H), 1.18 (t, J=7.2 Hz, 3H).

The following Compounds were synthesized by an analogous method as described above for Compound 27.

| Compound No | Structure | Starting Materials |
|---|---|---|
| 22 | 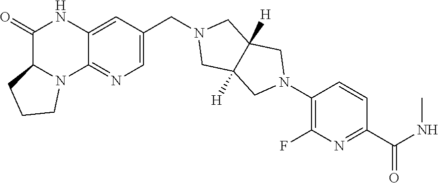 [SMILES: CNC(=O)c1ccc(N2C[C@H]3CN(Cc4cnc5c(c4)NC(=O)[C@@H]4CCCN54)C[C@@H]3C2)c(F)n1] | Intermediate 24 & intermediate 67 |
| 29 | 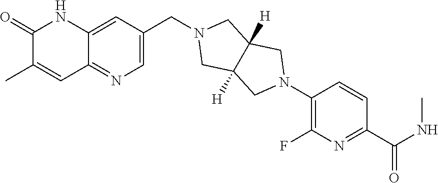 [SMILES: CNC(=O)c1ccc(N2C[C@H]3CN(Cc4cnc5cc(C)c(=O)[nH]c5c4)C[C@@H]3C2)c(F)n1] | Intermediate 41 & intermediate 67 |
| 30 | 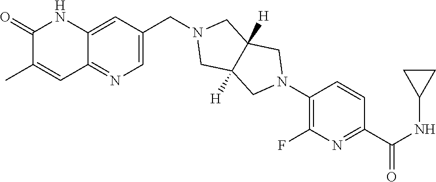 [SMILES: Cc1cc2ncc(CN3C[C@@H]4CN(c5ccc(C(=O)NC6CC6)nc5F)C[C@H]4C3)cc2[nH]c1=O] | Intermediate 41 & intermediate 174 |
| 33 | 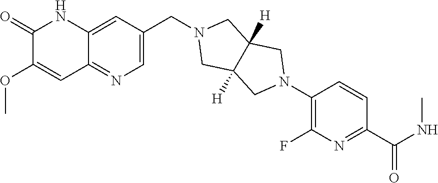 [SMILES: CNC(=O)c1ccc(N2C[C@H]3CN(Cc4cnc5cc(OC)c(=O)[nH]c5c4)C[C@@H]3C2)c(F)n1] | Intermediate 47 & intermediate 67 |
| 34 | 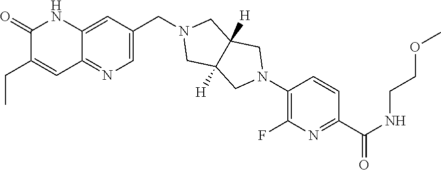 [SMILES: CCc1cc2ncc(CN3C[C@@H]4CN(c5ccc(C(=O)NCCOC)nc5F)C[C@H]4C3)cc2[nH]c1=O] | Intermediate 6 & intermediate 162 |
| 35 | 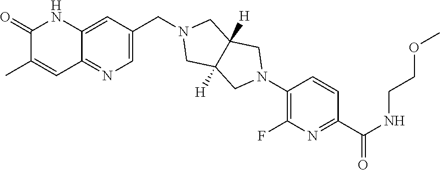 [SMILES: COCCNC(=O)c1ccc(N2C[C@H]3CN(Cc4cnc5cc(C)c(=O)[nH]c5c4)C[C@@H]3C2)c(F)n1] | Intermediate 41 & intermediate 162 |

### Preparation of Compound 28

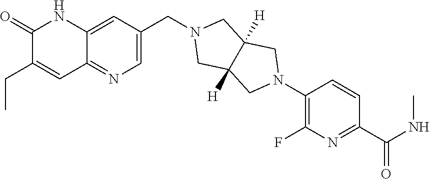 [SMILES: CCc1cc2ncc(CN3C[C@H]4CN(c5ccc(C(=O)NC)nc5F)C[C@@H]4C3)cc2[nH]c1=O]

To a solution of intermediate 72 (80 mg, 0.27 mmol) in MeCN (10 mL) was added intermediate 6 (70 mg, 0.31 mmol), DIEA (0.6 mL, 3.87 mmol) and potassium iodide (10 mg, 0.060 mmol). The mixture was stirred at 80° C. for 2 h. After cooling to rt, the reaction mixture was concentrated under reduced pressure to give a residue, which was purified by prep-HPLC (Sunfire C18 (5 μm 19*150 mm), Mobile Phase A: Water (0.2% HCOOH), Mobile Phase B: acetonitrile, UV: 214 nm, Flowrate: 15 mL/min, Temperature: rt, Gradient: 5-25% (% B)) to give Compound 28 (10 mg, yield: 8.3%) as a white solid. ¹H NMR (400 MHz, DMSO-d₆) 11.83 (s, 1H), 8.41 (d, J=0.8 Hz, 1H), 8.26 (s, 0.5H), 8.18 (d, J=4.8 Hz, 1H), 7.74-7.73 (m, 2H), 7.62 (s, 1H), 7.13-7.08 (m, 1H), 4.02-3.93 (m, 2H), 3.55-3.54 (m, 2H), 3.26 (t, J=8.8 Hz, 2H), 2.91-2.87 (m, 2H), 2.75 (d, J=4.4 Hz, 3H), 2.64 (t, J=9.2 Hz, 2H), 2.57-2.51 (m, 2H), 2.36-2.35 (m, 2H), 1.18 (t, J=7.6 Hz, 3H).

### Preparation of Compound 37

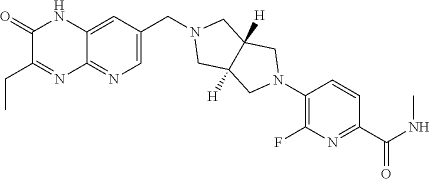 [SMILES: CCc1nc2ncc(CN3C[C@@H]4CN(c5ccc(C(=O)NC)nc5F)C[C@H]4C3)cc2[nH]c1=O]

To a solution of intermediate 105 (43 mg, 0.095 mmol) in DCM (16 mL) and MeOH (1.6 mL) was added MnO₂ (164.5 mg, 1.89 mmol). The reaction mixture was stirred at r.t. for 16 hr under N₂ atmosphere. After filtration by a pad of Celite, filtrate was concentrated under reduced pressure, the residue was purified by column chromatography on silica gel eluted with DCM/MeOH (10/1) to give Compound 37 (11.5 mg, yield: 23.34%) as a yellow solid.

### Preparation of Compound 40

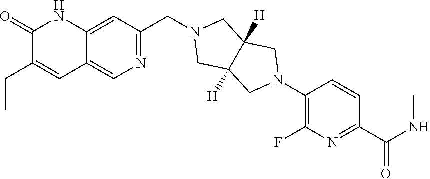 [SMILES: CCc1cc2cnc(CN3C[C@@H]4CN(c5ccc(C(=O)NC)nc5F)C[C@H]4C3)cc2[nH]c1=O]

To a solution of intermediate 108 (80 mg, 0.396 mmol) in DCM (8 mL) and MeOH (2 mL) was added intermediate 67 (104.6 mg, 0.40 mmol), NaBH₃CN (93.2 mg, 1.48 mmol) and 1 drop of AcOH. The resulting reaction mixture was stirred at r.t. for 1 h. The reaction mixture was concentrated under reduced pressure to give a residue, which was purified by silica gel column chromatography eluted with DCM/MeOH (10/1) to give a crude product. The crude product was further purified by prep. HPLC (Column: Xbridge C18 (5 μm 19*150 mm), Mobile Phase A: Water (0.2% HCOOH), Mobile Phase B: acetonitrile, UV: 254 nm, Flow rate: 15 mL/min, gradient condition from 5% B to 25% B.) to give Compound 40 (40 mg, yield: 22.44%) as a white solid. ¹H NMR (400 MHz, DMSO-d₆) 8.71 (s, 1H), 8.18 (d, J=4.4 Hz, 1H), 7.79-7.72 (m, 2H), 7.29 (s, 1H), 710 (dd, J=8.4 Hz, 10.8 Hz, 1H), 4.08-3.98 (m, 2H), 3.76-3.70 (m, 2H), 3.29-3.25 (m, 2H), 2.97-2.94 (m, 2H), 2.78-2.68 (m, 5H), 2.53-2.50 (m, 2H), 2.39-2.36 (m, 2H), 1.17 (t, J=7.2 Hz, 3H).

The following Compounds were synthesized by an analogous method as described above for Compound 40.

| Compound No | Structure | Starting Materials |
|---|---|---|
| 36 | 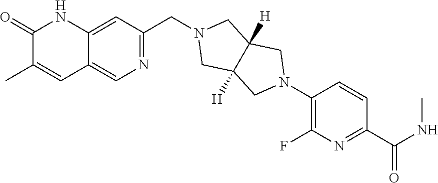 [SMILES: CNC(=O)c1ccc(N2C[C@H]3CN(Cc4cc5[nH]c(=O)c(C)cc5cn4)C[C@@H]3C2)c(F)n1] | Intermediate 111 & intermediate 67 |
| 39 | 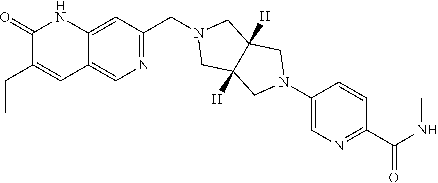 [SMILES: CCc1cc2cnc(CN3C[C@@H]4CN(c5ccc(C(=O)NC)nc5)C[C@@H]4C3)cc2[nH]c1=O] | Intermediate 108 & intermediate 64 |

### Preparation of Compound 46

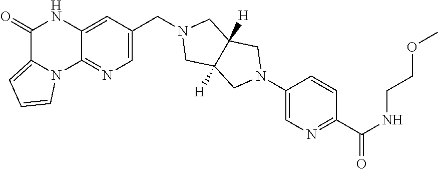 [SMILES: COCCNC(=O)c1ccc(N2C[C@H]3CN(Cc4cnc5c(c4)[nH]c(=O)c4cccn45)C[C@@H]3C2)cn1]

To a solution of intermediate 161 (31.1 mg, 0.11 mmol) and intermediate 16 (25 mg, 0.11 mmol) in CH₃CN (4 mL) were added DIEA (0.053 mL, 0.321 mmol) and KI (17.0 mg, 0.11 mmol). The mixture was stirred at 80° C. for 2 hr. The reaction mixture was concentrated under reduced pressure to give a residue, which was purified by column chromatography on silica gel eluted with DCM/MeOH (10/1) to give a crude compound. The crude product was further purified by prep. HPLC (Column: Xbridge C18 (5 μm 19*150 mm), Mobile Phase A: Water (0.1% ammonium bicarbonate), Mobile Phase B: acetonitrile, UV: 254 nm, Flow rate: 15 mL/min, gradient condition from 10% B to 60% B.) to give Compound 46 (11.6 mg, yield: 22.01%) as a white solid. ¹H NMR (400 MHz, DMSO-d₆) 11.34 (s, 1H), 8.23 (d, J=5.2 Hz, 1H), 8.16 (d, J=2.4 Hz, 1H), 8.12-8.11 (m, 1H), 7.87 (d, J=2.8 Hz, 1H), 7.79 (d, J=8.8 Hz, 1H), 7.65 (d, J=1.6 Hz, 1H), 7.10-7.09 (m, 1H), 6.95-6.93 (m, 1H), 6.73-6.71 (m, 1H), 4.00-3.92 (m, 2H), 3.54-3.50 (m, 2H), 3.46-3.40 (m, 4H), 3.26 (s, 3H), 3.08 (t, J=9.6 Hz, 2H), 2.94-2.90 (m, 2H), 2.67 (t, J=9.6 Hz, 2H), 2.43-2.36 (m, 2H).

The following compound was synthesized by an analogous method as described above for Compound 46.

| Compound No | Structure | Starting Materials |
|---|---|---|
| 64 | 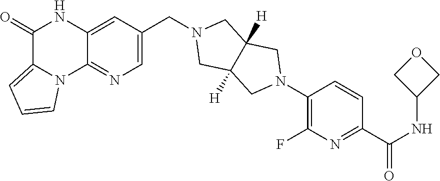 [SMILES: O=C(NC1COC1)c1ccc(N2C[C@H]3CN(Cc4cnc5c(c4)[nH]c(=O)c4cccn45)C[C@@H]3C2)c(F)n1] | Intermediate 16 & intermediate 168 |

### Preparation of Compound 48

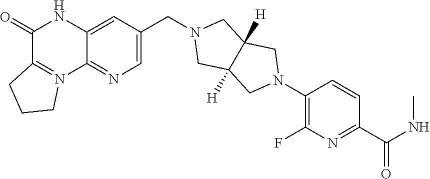

To a solution of intermediate 67 (50 mg, 0.19 mmol) in CH₃CN (8 mL) were added intermediate 117 (44.40 mg, 0.19 mmol), DIEA (0.031 mL, 0.19 mmol) and KI (9.0 mg, 0.02 mmol). The reaction mixture was stirred at 80° C. for 3 h. After cooled down to room temperature, the reaction mixture was concentrated under reduced pressure to give a residue, which was purified by column chromatography on silica gel eluted with DCM/MeOH (10/1) to give a crude product, which was further purified by prep-HPLC (Xbridge C18 5 μm 19*150 mm, 10-50% B); mobile phase: B (ACN), mobile phase: A (H₂O (0.1% NH₄HCO₃)) to give Compound 48 (12.3 mg, 0.026 mmol, 13.62%) as a white solid. 1H NMR (400 MHz, DMSO-d₆) 11.66 (s, 1H), 8.41 (d, J=2.0 Hz, 1H), 8.20-8.17 (m, 1H), 7.74-7.72 (m, 1H), 7.65 (d, J=1.6 Hz, 1H), 7.13-7.08 (m, 1H), 4.02-3.93 (m, 2H), 3.55 (t, J=5.6 Hz, 2H), 3.26 (t, J=7.2 Hz, 2H), 3.16 (t, J=7.6 Hz, 2H), 2.90-2.86 (m, 2H), 2.80 (t, J=7.2 Hz, 2H), 2.74 (d, J=4.8 Hz, 3H), 2.67-2.61 (m, 2H), 2.50-2.32 (m, 2H), 2.15-2.07 (m, 2H).

The following compounds were synthesized by an analogous method as described above for Compound 48.

| Compound No | Structure | Starting Materials |
|---|---|---|
| 47 | 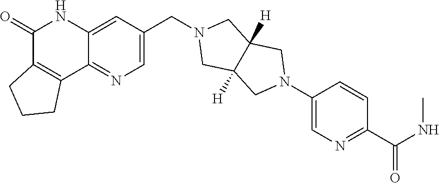 [SMILES: CNC(=O)c1ccc(N2C[C@H]3CN(Cc4cnc5c6c(c(=O)[nH]c5c4)CCC6)C[C@@H]3C2)cn1] | Intermediate 117 & intermediate 64 |
| 49 | 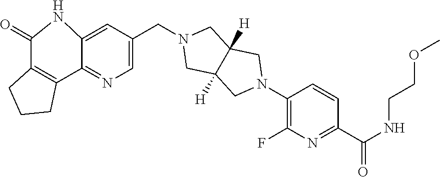 [SMILES: COCCNC(=O)c1ccc(N2C[C@H]3CN(Cc4cnc5c6c(c(=O)[nH]c5c4)CCC6)C[C@@H]3C2)c(F)n1] | Intermediate 117 & intermediate 162 |
| 50 | 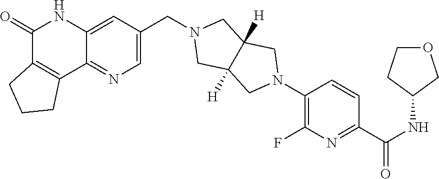 [SMILES: O=C(N[C@@H]1CCOC1)c1ccc(N2C[C@H]3CN(Cc4cnc5c6c(c(=O)[nH]c5c4)CCC6)C[C@@H]3C2)c(F)n1] | Intermediate 117 & intermediate 164 |
| 51 | 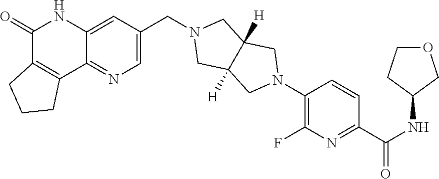 [SMILES: O=C(N[C@H]1CCOC1)c1ccc(N2C[C@H]3CN(Cc4cnc5c6c(c(=O)[nH]c5c4)CCC6)C[C@@H]3C2)c(F)n1] | Intermediate 117 & intermediate 166 |

### Preparation of Compound 52

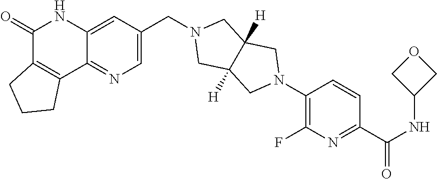 [SMILES: O=C(NC1COC1)c1ccc(N2C[C@H]3CN(Cc4cnc5c6c(c(=O)[nH]c5c4)CCC6)C[C@@H]3C2)c(F)n1]

To a solution of intermediate 168 (60 mg, 0.20 mmol) in MeCN (5 mL) were added intermediate 117 (43.2 mg, 0.20 mmol), DIEA (0.162 mL, 0.979 mmol) and KI (3.3 mg, 0.020 mmol). The resulting reaction mixture was stirred at 80° C. for 2 hr. The reaction mixture was evaporated under vacuum. The residue was diluted with water and extracted with ethyl acetate. The combined organic layers were dried over sodium sulphate, filtered and concentrated to give crude product, which was purified by prep-HPLC (XBridge C18 (5 μm 19*150 mm), Mobile Phase A: Water (0.1% NH₄HCO₃), Mobile Phase B: acetonitrile, UV: 254 nm, Flow rate: 15 mL/min, Gradient: 45-80% (% B)) to give Compound 52 (30 mg, yield: 30.36%) as a white solid. ¹H NMR (400 MHz, DMSO-d₆) 11.65 (s, 1H), 8.86 (d, J=6.8 Hz, 1H), 8.41 (d, J=1.6 Hz, 1H), 7.73 (dd, J=8.4 Hz, 1.6 Hz, 1H), 7.65 (d, J=1.6 Hz, 1H), 7.11 (dd, J=10.8 Hz, 8.4 Hz, 1H), 5.02-4.93 (m, 1H), 4.70-4.63 (m, 4H), 3.43-3.93 (m, 2H), 3.57-3.56 (m, 2H), 3.31-3.25 (m, 2H), 3.18-3.14 (m, 2H), 2.90-2.87 (m, 2H), 2.82-2.79 (m, 2H), 2.67-2.62 (m, 2H), 2.40-2.31 (m, 2H), 2.15-2.07 (m, 2H).

The following compound was synthesized by an analogous method as described above for Compound 48.

| Compound No | Structure | Starting Materials |
|---|---|---|
| 53 | 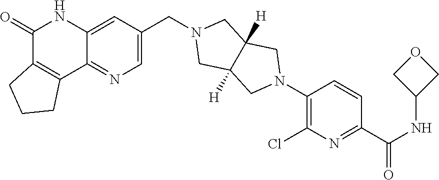 [SMILES: O=C(NC1COC1)c1ccc(N2C[C@H]3CN(Cc4cnc5c6c(c(=O)[nH]c5c4)CCC6)C[C@@H]3C2)c(Cl)n1] | Intermediate 117 & intermediate 198 |

### Preparation of Compound 54

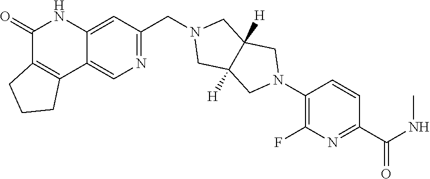 [SMILES: CNC(=O)c1ccc(N2C[C@H]3CN(Cc4cc5[nH]c(=O)c6c(c5cn4)CCC6)C[C@@H]3C2)c(F)n1]

To a solution of intermediate 67 (185 mg, 0.70 mmol) in MeOH (5 mL) was added intermediate 120 (150 mg, 0.70 mmol). After the reaction mixture was stirred at r.t. for 1 hr, NaBH₃CN (132.00 mg, 0.21 mmol) and 1 drop of HOAc were added, and the resulting reaction mixture was stirred at r.t. overnight. The reaction mixture was concentrated to give a residue, it was purified by prep. HPLC, (XBridge C18 (5 μm 19*150 mm), Mobile Phase A: Water (0.2% TFA), Mobile Phase B: acetonitrile, UV: 254 nm, Flow rate: 15 mL/min, Gradient: 45-80% (% B)) to give Compound 54 (30 mg, yield: 9.29%) as a white solid. ¹H NMR (400 MHz, DMSO-d₆) 11.81 (s, 1H), 8.68 (s, 1H), 8.18 (d, J=5.2 Hz, 1H), 7.74 (dd, J=8.0 Hz, 1.6 Hz, 1H), 7.34 (s, 1H), 7.11 (dd, J=10.4 Hz, 8.4 Hz, 1H), 4.17-4.08 (m, 2H), 3.58-3.56 (m, 2H), 3.31-3.27 (m, 2H), 3.17-3.13 (m, 2H), 3.06-3.02 (m, 2H), 2.82-2.74 (m, 7H), 2.40-2.41 (m, 2H), 2.16-2.08 (m, 2H).

The following compounds were synthesized by an analogous method as described above for Compound 54.

| Compound No | Structure | Starting Materials |
|---|---|---|
| 55 | 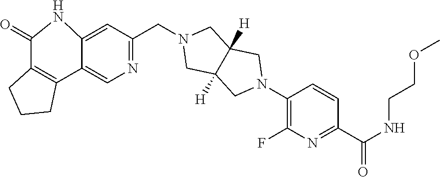 [SMILES: COCCNC(=O)c1ccc(N2C[C@H]3CN(Cc4cc5[nH]c(=O)c6c(c5cn4)CCC6)C[C@@H]3C2)c(F)n1] | Intermediate 120 & intermediate 162 |
| 56 | 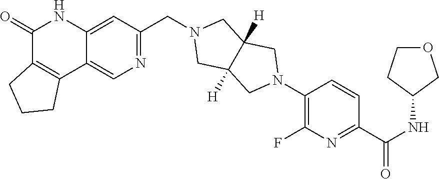 [SMILES: O=C(N[C@@H]1CCOC1)c1ccc(N2C[C@H]3CN(Cc4cc5[nH]c(=O)c6c(c5cn4)CCC6)C[C@@H]3C2)c(F)n1] | Intermediate 120 & intermediate 164 |
| 57 | 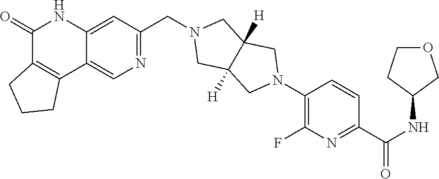 [SMILES: O=C(N[C@H]1CCOC1)c1ccc(N2C[C@H]3CN(Cc4cc5[nH]c(=O)c6c(c5cn4)CCC6)C[C@@H]3C2)c(F)n1] | Intermediate 120 & intermediate 166 |
| 58 | 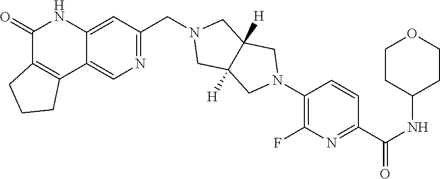 [SMILES: O=C(NC1CCOCC1)c1ccc(N2C[C@H]3CN(Cc4cc5[nH]c(=O)c6c(c5cn4)CCC6)C[C@@H]3C2)c(F)n1] | Intermediate 120 & intermediate 170 |
| 59 | 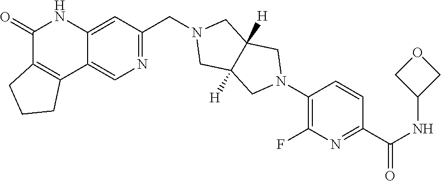 [SMILES: O=C(NC1COC1)c1ccc(N2C[C@H]3CN(Cc4cc5[nH]c(=O)c6c(c5cn4)CCC6)C[C@@H]3C2)c(F)n1] | Intermediate 120 & intermediate 168 |
| 63 | 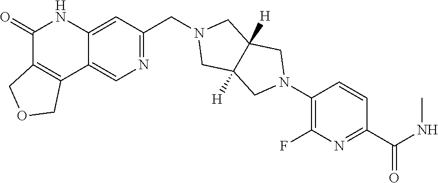 [SMILES: CNC(=O)c1ccc(N2C[C@H]3CN(Cc4cc5[nH]c(=O)c6c(c5cn4)COC6)C[C@@H]3C2)c(F)n1] | Intermediate 150 & intermediate 67 |
| 80 | 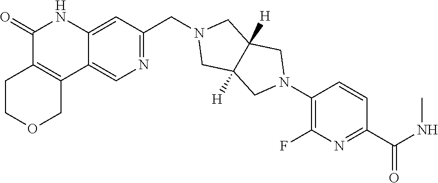 [SMILES: CNC(=O)c1ccc(N2C[C@H]3CN(Cc4cc5[nH]c(=O)c6c(c5cn4)COCC6)C[C@@H]3C2)c(F)n1] | Intermediate 158 & intermediate 67 |

### Preparation of Compound 60

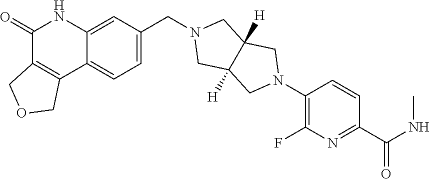 [SMILES: CNC(=O)c1ccc(N2C[C@H]3CN(Cc4ccc5c6c(c(=O)[nH]c5c4)COC6)C[C@@H]3C2)c(F)n1]

To a solution of intermediate 125 in CH₃CN (10 mL) were added intermediate 67 (112 mg, 0.43 mmol), DIEA (166 mg, 1.29 mmol) and potassium iodide (7 mg, 0.04 mmol). The reaction mixture was stirred at 80° C. for 2 h. After cooling down to room temperature, the reaction mixture was concentrated under reduced pressure to give a residue, which was purified by prep. HPLC (C18 (5 μm 19*150 mm), Mobile Phase A: Water (0.2% HCOOH), Mobile Phase B: acetonitrile, UV: 214 nm, Flowrate: 15 mL/min, Temperature: rt, Gradient: 10-25% (% B)) to give Compound 60 (16 mg, yield: 8.14%) as a white solid. ¹H NMR (400 MHz, DMSO-d₆) 11.78 (s, 1H), 8.18-8.14 (m, 1.5H), 7.72 (dd, J=8.4 Hz, J=2.0 Hz, 1H), 7.44-7.41 (m, 2H), 7.21-7.18 (m, 1H), 7.13-7.08 (m, 1H), 5.29 (t, J=3.6 Hz, 2H), 4.96 (t, J=3.6 Hz, 2H), 4.05-3.92 (m, 2H), 3.56-3.53 (m, 2H), 3.28-3.23 (m, 2H), 2.91-2.88 (m, 2H), 2.74 (d, J=4.8 Hz, 3H), 2.68-2.64 (m, 2H), 2.38-2.33 (m, 2H).

The following compounds were synthesized by an analogous method as described above for Compound 60.

| Compound No | Structure | Starting Materials |
|---|---|---|
| 61 | 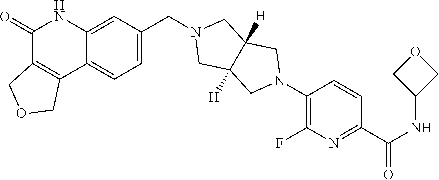 [SMILES: O=C(NC1COC1)c1ccc(N2C[C@H]3CN(Cc4ccc5c6c(c(=O)[nH]c5c4)COC6)C[C@@H]3C2)c(F)n1] | Intermediate 125 & intermediate 168 |
| 62 | 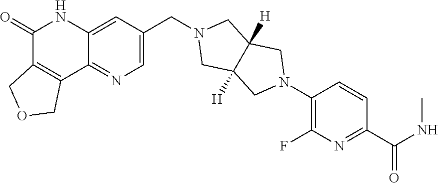 [SMILES: CNC(=O)c1ccc(N2C[C@H]3CN(Cc4cnc5c6c(c(=O)[nH]c5c4)COC6)C[C@@H]3C2)c(F)n1] | Intermediate 147 & intermediate 67 |

### Preparation of Compound 66

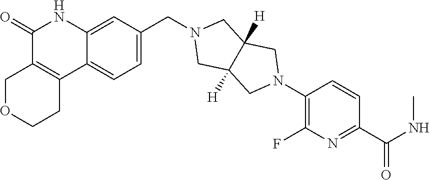 [SMILES: CNC(=O)c1ccc(N2C[C@H]3CN(Cc4ccc5c6c(c(=O)[nH]c5c4)COCC6)C[C@@H]3C2)c(F)n1]

To a solution of intermediate 67 (42.34 mg, 0.16 mmol) in MeCN (5 mL) were added DIEA (0.13 mL, 0.80 mmol), potassium iodide (2.6 mg, 0.016 mmol) and intermediate 129 (40 mg, 0.16 mmol). The resulting solution was stirred at 80° C. for 2 hours. The reaction mixture was evaporated under vacuum. Crude material was diluted with water and extracted with ethyl acetate. The combined organic layers were dried over sodium sulphate, filtered and concentrated to give crude product, which was purified by prep-HPLC (XBridge C18 (5 μm 19*150 mm), Mobile Phase A: Water (0.2% FA), Mobile Phase B: acetonitrile, UV: 254 nm, Flow rate: 15 mL/min, Gradient: 45-80% (% B)) to Compound 66 (30 mg, yield: 37.50%) as a white solid. ¹H NMR (400 MHz, DMSO-d₆) 11.70 (s, 1H), 8.17 (d, J=5.2 Hz, 1H), 8.14 (s, 0.85H), 7.73 (dd, J=8.0 Hz, 2.0 Hz, 1H), 7.62 (d, J=8.0 Hz, 1H), 7.32 (s, 1H), 7.20 (dd, J=8.4 Hz, 0.8 Hz, 1H), 7.10 (dd, J=10.8 Hz, 8.4 Hz, 1H), 4.45 (s, 2H), 3.97-3.91 (m, 4H), 3.57-3.53 (m, 2H), 3.29-3.24 (m, 2H), 2.91-2.86 (m, 4H), 2.74 (d, J=4.8 Hz, 3H), 2.68-2.63 (m, 2H), 2.39-2.30 (m, 2H).

The following compounds were synthesized by an analogous method as described above for Compound 66.

| Compound No | Structure | Starting Materials |
|---|---|---|
| 65 | 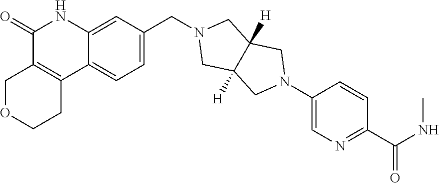 [SMILES: CNC(=O)c1ccc(N2C[C@H]3CN(Cc4ccc5c6c(c(=O)[nH]c5c4)COCC6)C[C@@H]3C2)cn1] | Intermediate 129 & intermediate 64 |
| 67 | 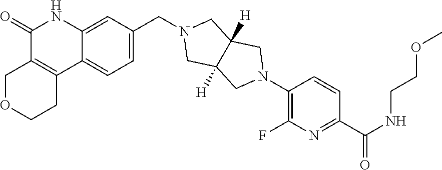 [SMILES: COCCNC(=O)c1ccc(N2C[C@H]3CN(Cc4ccc5c6c(c(=O)[nH]c5c4)COCC6)C[C@@H]3C2)c(F)n1] | Intermediate 129 & intermediate 162 |
| 68 | 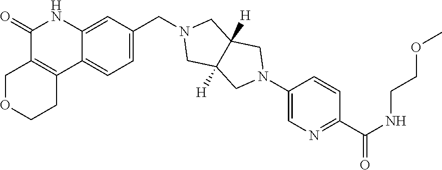 [SMILES: COCCNC(=O)c1ccc(N2C[C@H]3CN(Cc4ccc5c6c(c(=O)[nH]c5c4)COCC6)C[C@@H]3C2)cn1] | Intermediate 129 & intermediate 161 |
| 70 | 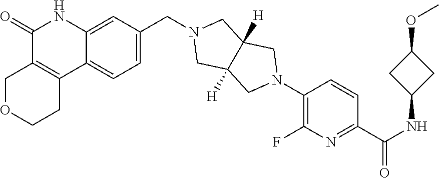 [SMILES: CO[C@H]1C[C@@H](NC(=O)c2ccc(N3C[C@H]4CN(Cc5ccc6c7c(c(=O)[nH]c6c5)COCC7)C[C@@H]4C3)c(F)n2)C1] | Intermediate 129 & intermediate 172 |
| 84 | 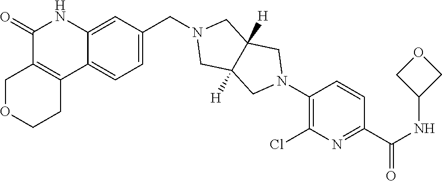 [SMILES: O=C(NC1COC1)c1ccc(N2C[C@H]3CN(Cc4ccc5c6c(c(=O)[nH]c5c4)COCC6)C[C@@H]3C2)c(Cl)n1] | Intermediate 129 & intermediate 198 |
| 85 | 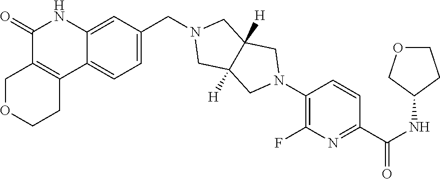 [SMILES: O=C(N[C@H]1CCOC1)c1ccc(N2C[C@H]3CN(Cc4ccc5c6c(c(=O)[nH]c5c4)COCC6)C[C@@H]3C2)c(F)n1] | Intermediate 129 & intermediate 164 |
| 86 | 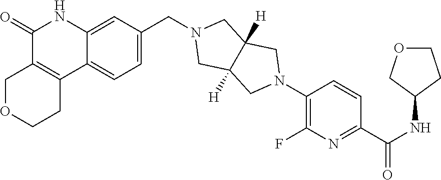 [SMILES: O=C(N[C@@H]1CCOC1)c1ccc(N2C[C@H]3CN(Cc4ccc5c6c(c(=O)[nH]c5c4)COCC6)C[C@@H]3C2)c(F)n1] | Intermediate 129 & intermediate 166 |

### Preparation of Compound 69

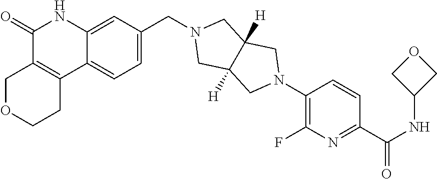 [SMILES: O=C(NC1COC1)c1ccc(N2C[C@H]3CN(Cc4ccc5c6c(c(=O)[nH]c5c4)COCC6)C[C@@H]3C2)c(F)n1]

To a solution of intermediate 129 in MeCN (5 mL) were added intermediate 168 (60 mg, 0.20 mmol), DIEA (0.08 mL, 0.60 mmol) and potassium iodide (3.2 mg, 0.02 mmol). The resulting solution was stirred at 80° C. for 2 hours. The reaction mixture was concentrated under reduced pressure to give a residue, which was purified by prep. HPLC (XBridge C18 (5 μm 19*150 mm), Mobile Phase A: Water (0.2% FA), Mobile Phase B: acetonitrile, UV: 254 nm, Flow rate: 15 mL/min, Gradient: 5-30% (% B)) to give Compound 69 (10 mg, yield: 9.9%) as a white solid. ¹H NMR (400 MHz, DMSO-d₆) 12.70 (s, 1H), 8.86 (d, J=6.8 Hz, 1H), 8.15 (s, 0.71H), 7.74-7.72 (m, 1H), 7.62 (d, J=8.4 Hz, 1H), 7.32 (s, 1H), 7.20 (d, J=8.0 Hz, 1H), 7.13-7.08 (m, 1H), 5.00-4.95 (m, 1H), 4.70-4.64 (m, 4H), 4.45 (s, 2H), 4.01-3.91 (m, 4H), 3.57-3.48 (m, 2H), 3.27 (t, J=8.8 Hz, 2H), 2.91-2.86 (m, 4H), 2.63 (t, J=8.8 Hz, 2H), 2.39-2.33 (m, 2H).

### Preparation of Compound 71

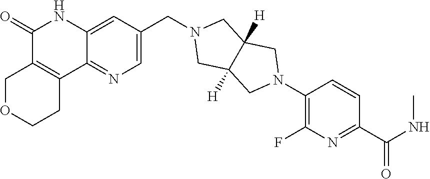 [SMILES: CNC(=O)c1ccc(N2C[C@H]3CN(Cc4cnc5c6c(c(=O)[nH]c5c4)COCC6)C[C@@H]3C2)c(F)n1]

To a solution of intermediate 134 (50 mg, 0.189 mmol) in CH₃CN (8 mL) were added intermediate 67 (47.43 mg, 0.189 mmol), DIEA (73.28 mg, 0.57 mmol) and KI (8.98 mg, 0.02 mmol). The resulting reaction mixture was stirred at 80° C. for 2 hr. The reaction mixture was concentrated under reduced pressure to give a residue, which was purified by silica gel column chromatography eluted with DCM/MeOH (10/1) to give a crude product, which was further purified by prep. HPLC (Xbridge C18 5 μm 19*150 mm5-25% B); mobile phase: B (ACN), mobile phase: A (H₂O (0.2% HCOOH)) to give Compound 71 (13.9 mg, 15.3% yield) as a white solid. ¹H NMR (400 MHz, DMSO-d₆) 11.83 (s, 1H), 8.45 (d, J=1.6 Hz, 1H), 8.20-8.15 (m, 1.7H), 7.72 (dd, J=8.0 Hz, J=1.6 Hz, 1H), 7.65 (d, J=1.2 Hz, 1H), 7.13-7.08 (m, 1H), 4.47 (s, 2H), 4.05-3.95 (m, 2H), 3.92 (t, J=5.6 Hz, 2H), 3.59-3.52 (m, 2H), 3.26 (t, J=8.8 Hz, 2H), 3.00-2.94 (m, 2H), 2.91-2.87 (m, 2H), 2.74 (d, J=4.8 Hz, 3H), 2.65 (t, J=10.4 Hz, 2H), 2.38-2.32 (m, 2H).

The following compounds were synthesized by an analogous method as described above for Compound 71.

| Compound No | Structure | Starting Materials |
|---|---|---|
| 72 | 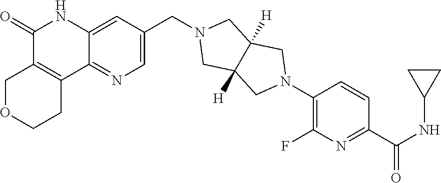 [SMILES: O=C(NC1CC1)c1ccc(N2C[C@@H]3CN(Cc4cnc5c6c(c(=O)[nH]c5c4)COCC6)C[C@H]3C2)c(F)n1] | Intermediate 134 & intermediate 174 |
| 74 | 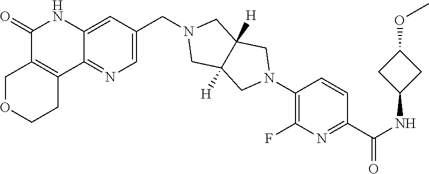 [SMILES: CO[C@H]1C[C@H](NC(=O)c2ccc(N3C[C@H]4CN(Cc5cnc6c7c(c(=O)[nH]c6c5)COCC7)C[C@@H]4C3)c(F)n2)C1] | Intermediate 134 & intermediate 176 |
| 76 | 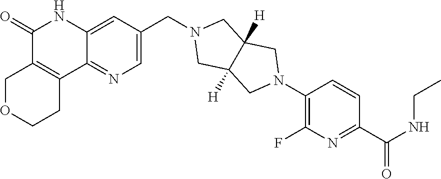 [SMILES: CCNC(=O)c1ccc(N2C[C@H]3CN(Cc4cnc5c6c(c(=O)[nH]c5c4)COCC6)C[C@@H]3C2)c(F)n1] | Intermediate 134 & intermediate 178 |
| 78 | 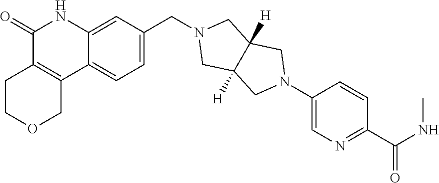 [SMILES: CNC(=O)c1ccc(N2C[C@H]3CN(Cc4ccc5c6c(c(=O)[nH]c5c4)CCOC6)C[C@@H]3C2)cn1] | Intermediate 141 & intermediate 64 |
| 79 | 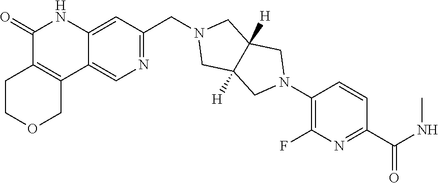 [SMILES: CNC(=O)c1ccc(N2C[C@H]3CN(Cc4cc5[nH]c(=O)c6c(c5cn4)COCC6)C[C@@H]3C2)c(F)n1] | Intermediate 155 & intermediate 67 |

### Preparation of Compound 73

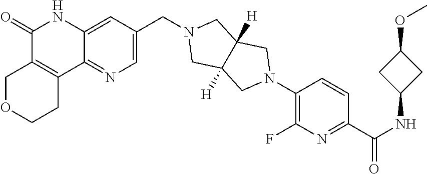 [SMILES: CO[C@H]1C[C@@H](NC(=O)c2ccc(N3C[C@H]4CN(Cc5cnc6c7c(c(=O)[nH]c6c5)COCC7)C[C@@H]4C3)c(F)n2)C1]

To a solution of intermediate 134 (100 mg, 0.40 mmol) and intermediate 172 (133.4 mg, 0.40 mmol) in CH₃CN (8 mL) was added DIEA (154.7 mg, 1.20 mmol) and KI (6.4 mg, 0.04 mmol). The resulting reaction mixture was stirred at 80° C. for 2 hr. The reaction mixture was concentrated under reduced pressure to give a residue, which was purified by column chromatography on silica gel eluted with DCM/MeOH (10/1) to give a crude product. The crude product was further purified by prep-HPLC (Column: Sun Fire C18 5 μm 19*150 mm; mobile phase: B (ACN), mobile phase: A (H₂O (0.2% HCOOH), UV: 254 nm, Flow rate: 15 mL/min, Gradient: 10-25% B) to give Compound 73 (18.5 mg, yield: 8.40%) as a white solid. ¹H NMR (400 MHz, DMSO-d₆) 11.84 (s, 1H), 8.45 (d, J=1.6 Hz, 1H), 8.33 (d, J=8.4 Hz, 1H), 8.18 (s, 0.47H), 7.73-7.70 (m, 1H), 7.65 (d, J=1.2 Hz, 1H), 7.12-7.08 (m, 1H), 4.47 (s, 2H), 4.04-3.91 (m, 5H), 3.59-3.54 (m, 3H), 3.27 (t, J=8.8 Hz, 4H), 3.13 (s, 3H), 2.99-2.96 (m, 2H), 2.97-2.87 (m, 2H), 2.67-2.62 (m, 2H), 2.39-2.33 (m, 2H), 2.05-1.98 (m, 2H).

### Preparation of Compound 77

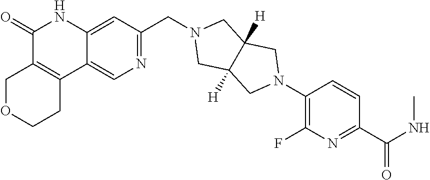 [SMILES: CNC(=O)c1ccc(N2C[C@H]3CN(Cc4cc5[nH]c(=O)c6c(c5cn4)CCOC6)C[C@@H]3C2)c(F)n1]

To a solution of intermediate 67 (50 mg, 0.19 mmol) in DCM (8 mL) and MeOH (2 mL) was added intermediate 137 (52.26 mg, 0.23 mmol), HOAc (35.3 mg, 0.19 mmol) and NaBH(OAc)₃ (198.64 mg, 0.95 mmol). The resulting reaction mixture was stirred at rt for 1 hr. The reaction mixture was concentrated under reduced pressure to give a residue, which was purified by silica gel column chromatography eluted with DCM/MeOH (10/1) to give a crude product. The crude product was further purified by prep-HPLC (Xbridge C18 5 μm 19*150 mm 5-25% B); mobile phase: B (ACN), mobile phase: A (H₂O (0.2% HCOOH)) to give Compound 77 (25.5 mg, yield: 27.96%) as a white solid. ¹H NMR (400 MHz, DMSO-d₆) 11.93 (s, 1H), 8.79 (s, 1H), 8.19-8.15 (m, 1.88H), 7.75-7.72 (m, 1H), 7.32 (s, 1H), 7.13-7.09 (m, 1H), 4.45 (s, 2H), 4.10-4.00 (m, 2H), 3.93 (t, J=5.6 Hz, 2H), 3.61-3.52 (m, 2H), 3.28 (t, J=8.8 Hz, 2H), 3.01-2.91 (m, 4H), 2.75-2.67 (m, 5H), 2.44-2.33 (m, 2H).

### Preparation of Compound 81

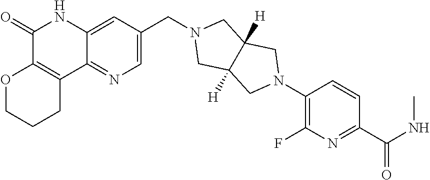 [SMILES: CNC(=O)c1ccc(N2C[C@H]3CN(Cc4cnc5c6c(c(=O)[nH]c5c4)OCCC6)C[C@@H]3C2)c(F)n1]

To a solution of intermediate 188 (55 mg, 0.22 mmol) in MeCN (10 mL) were added intermediate 67 (58 mg, 0.22 mmol), KI (36 mg, 0.22 mmol) and DIEA (28 mg, 0.22 mmol). The reaction mixture was heated to 80° C. and stirred for 2 h. The reaction mixture was evaporated under reduced pressure. The residue was purified by pre-HPLC (SunFire C18 5 μm; 19*150 mm; 10-25% B); mobile phase: B (ACN), mobile phase: A (H₂O (0.2% HCOOH)) to afford Compound 81 (3.9 mg, yield: 3.60%) as a white solid. ¹H NMR (400 MHz, DMSO-d₆) 11.83 (s, 1H), 8.38 (d, J=1.6 Hz, 1H), 8.34 (s, 1.43H), 8.19-8.16 (m, 1H), 7.74-7.71 (m, 1H), 7.57 (d, J=1.2 Hz, 1H), 7.13-7.08 (m, 1H), 4.22 (t, J=4.0 Hz, 2H), 3.99-3.90 (m, 2H), 3.60-3.51 (m, 2H), 3.26 (t, J=8.4 Hz, 2H), 2.91-2.86 (m, 4H), 2.74 (d, J=4.8 Hz, 3H), 2.67-2.60 (m, 2H), 2.38-2.31 (m, 2H), 2.01-1.98 (m, 2H).

### Preparation of Compound 82

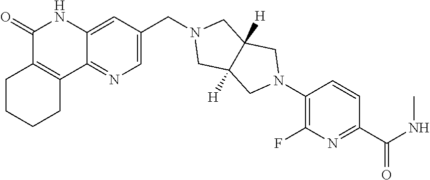 [SMILES: CNC(=O)c1ccc(N2C[C@H]3CN(Cc4cnc5c6c(c(=O)[nH]c5c4)CCCC6)C[C@@H]3C2)c(F)n1]

To a solution of intermediate 193 (105 mg, 0.42 mmol) and intermediate 67 (93 mg, 0.35 mmol) in MeCN (10 mL) were added DIEA (137 mg, 1.06 mol) and KI (6 mg, 0.04 mmol). The reaction mixture was stirred at 80° C. for 2 h. The reaction mixture was concentrated under reduced pressure to give a residue, which was purified by column chromatography on silica gel eluted with DCM/MeOH (10/1) to afford a crude product, which was further purified by prep-HPLC (Column: SunFire C18 5 um 19*150 mm; mobile phase: B (ACN), mobile phase: A (H₂O (0.2% HCOOH), UV: 254 nm, Flow rate: 15 mL/min, Gradient: 5-35% B) to afford Compound 82 (9 mg, yield: 5.32%) as a white solid. ¹H NMR (400 MHz, DMSO-d₆) 11.66 (s, 1H), 8.41 (d, J=1.6 Hz, 1H), 8.17 (d, J=4.8 Hz, 1H), 7.74-7.72 (m, 1H), 7.60 (s, 1H), 7.11-7.10 (m, 1H), 3.98-3.96 (m, 2H), 3.55-3.54 (m, 2H), 3.26-3.21 (m, 4H), 2.95 (s, 2H), 2.90-2.87 (m, 2H), 2.74 (d, J=4.8 Hz, 3H), 2.67-2.61 (m, 2H), 2.37-2.33 (m, 2H), 1.76 (s, 4H).

The following compound was synthesized by an analogous method as described above for Compound 82.

| Compound No | Structure | Starting Materials |
|---|---|---|
| 83 | 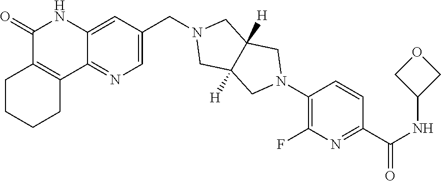 [SMILES: O=C(NC1COC1)c1ccc(N2C[C@H]3CN(Cc4cnc5c6c(c(=O)[nH]c5c4)CCCC6)C[C@@H]3C2)c(F)n1] | Intermediate 193 & intermediate 168 |

### Preparation of Compound 87

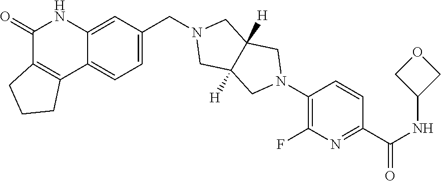 [SMILES: O=C(NC1COC1)c1ccc(N2C[C@H]3CN(Cc4ccc5c6c(c(=O)[nH]c5c4)CCC6)C[C@@H]3C2)c(F)n1]

To a solution of intermediate 168 (65.3 mg, 0.21 mmol) in MeCN (5 mL) were added DIEA (0.18 mL, 1.07 mmol), KI (3.55 mg, 0.02 mmol) and intermediate 181 (50 mg, 0.21 mmol). The resulting solution was stirred at 80° C. for 2 hours. The reaction mixture was evaporated under vacuum. Crude material was diluted with water and extracted with ethyl acetate. The combined organic layers were dried over sodium sulphate, filtered and concentrated to give crude product, which was purified by prep. HPLC (XBridge C18 (5 μm 19*150 mm), Mobile Phase A: Water (0.1% NH₄HCO₃), Mobile Phase B: acetonitrile, UV: 254 nm, Flow rate: 15 mL/min, Gradient: 45-80% (% B)) to give Compound 87 (50 mg, yield: 46.43%) as a white solid. ¹H NMR (400 MHz, DMSO-d₆) 11.50 (s, 1H), 8.86 (d, J=6.8 Hz, 1H), 7.73 (dd, J=8.0 Hz, 1.6 Hz, 1H), 7.47 (d, J=8.0 Hz, 1H), 7.33 (s, 1H), 7.17-7.08 (m, 2H), 5.00-4.93 (m, 1H), 4.70-4.63 (m, 4H), 3.93 (dd, J=26.8 Hz, 14.0 Hz, 2H), 3.57-3.55 (m, 2H), 3.31-3.25 (m, 2H), 3.09-3.06 (m, 2H), 2.89-2.86 (m, 2H) 2.77-2.74 (m, 2H), 2.66-2.61 (m, 2H), 2.36-2.33 (m, 2H), 2.13-2.06 (m, 2H).

The following compound was synthesized by an analogous method as described above for Compound 87.

| Compound No | Structure | Starting Materials |
|---|---|---|
| 91 | 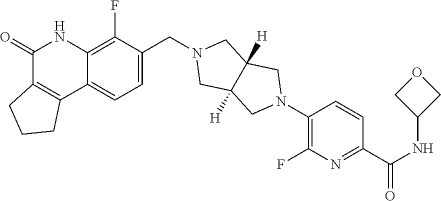 [SMILES: O=C(NC1COC1)c1ccc(N2C[C@H]3CN(Cc4ccc5c6c(c(=O)[nH]c5c4F)CCC6)C[C@@H]3C2)c(F)n1] | Intermediate 206 & intermediate 168 |

### Preparation of Compound 88

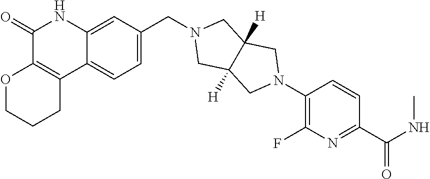 [SMILES: CNC(=O)c1ccc(N2C[C@H]3CN(Cc4ccc5c6c(c(=O)[nH]c5c4)OCCC6)C[C@@H]3C2)c(F)n1]

To a solution of intermediate 67 (50 mg, 0.19 mmol) and intermediate 202 (40 mg, 0.16 mmol) in MeCN (10 mL) were added DIEA (72 mg, 0.56 mmol) and KI (3.1 mg, 0.019 mmol). The reaction mixture was stirred at 80° C. for 2 h. The reaction mixture was concentrated under reduced pressure, the residue was purified by column chromatography on silica gel eluted with DCM/MeOH (10/1) to give a crude product, which was further purified by prep-HPLC (Column: SunFire C18 5 μm 19*150 mm; mobile phase: B (ACN), mobile phase: A (H₂O (0.2% HCOOH), UV: 254 nm, Flow rate: 15 mL/min, Gradient: 5-35% B) to afford Compound 88 (38.5 mg, yield: 42.6%) as a white solid. ¹H NMR (400 MHz, DMSO-d₆) 11.66 (s, 1H), 8.23 (s, 1H), 7.74-7.71 (m, 1H), 7.48 (d, J=8.4 Hz, 1H), 7.25 (s, 1H), 7.16-7.08 (m, 2H), 4.15 (t, J=4.8 Hz, 2H), 3.95-3.85 (m, 2H), 3.55-3.54 (m, 3H), 3.25 (t, J=8.8 Hz, 2H), 2.88-2.85 (m, 2H), 2.80-2.74 (m, 4H), 2.62 (t, J=10 Hz, 2H), 2.37-2.33 (m, 2H), 2.04-2.00 (m, 2H).

The following compounds were synthesized by an analogous method as described above for Compound 82.

| Compound No | Structure | Starting Materials |
|---|---|---|
| 89 | 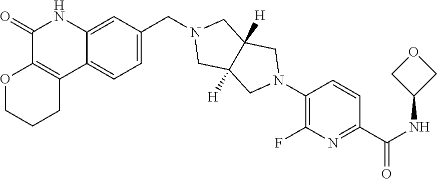 | Intermediate 202 & intermediate 168 |
| 90 | 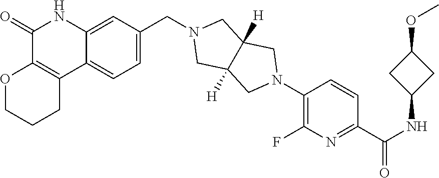 [SMILES: CO[C@H]1C[C@@H](NC(=O)c2ccc(N3C[C@H]4CN(Cc5ccc6c7c(c(=O)[nH]c6c5)OCCC7)C[C@@H]4C3)c(F)n2)C1] | Intermediate 202 & intermediate 172 |

### Example 4: General Protocols for LCMS (Liquid Chromatography/Mass Spectrometry)

The High-Performance Liquid Chromatography (HPLC) measurement was performed using a LC pump, a diode-array (DAD) or a UV detector and a column as specified in the respective methods. Flow from the column was brought to the Mass Spectrometer (MS) which was configured with an atmospheric pressure ion source. It is within the knowledge of the skilled person to set the tune parameters (e.g., scanning range, dwell time, etc.) in order to obtain ions allowing the identification of the compound's nominal monoisotopic molecular weight (MW). Data acquisition was performed with appropriate software.

Compounds are described by their experimental retention times (Rt) and ions. If not specified differently in the table of data, the reported molecular ion corresponds to the [M+H]⁺ (protonated molecule) and/or [M−H]⁻ (deprotonated molecule). All results were obtained with experimental uncertainties that are commonly associated with the method used.

Method 1: Instrument: Agilent Technologies 1200 Series, G1329A; Column: Xbridge C18.5 μm 4.6*50 mm; Mobile phase: A: 0.05% TFA; B: CH₃CN; Gradient: 95% A for 0.01 min, to 40% A in 4.49 min, then to 5% A in 0.30 min, held for 1.00 min, back to 95% A in 0.20 min, held for 0.50 min; Flow rate & column temperature: 1.5 mL/min, 40° C.; Run time: 6.5 min.

Method 2: Instrument: Agilent Technologies 1200 Series, G1329A; Column: Xbridge C18.5 μm 4.6*50 mm; Mobile phase: A: 0.05% TFA; B: CH₃CN; Gradient: 95% A for 0.01 min, to 5% A in 4.49 min, held for 1.30 min, back to 95% A in 0.20 min, held for 0.50 min; Flow rate & column temperature: 1.5 mL/min, 40° C.; Run time: 6.5 min.

Method 3: Instrument: SHIMA DZU SIL-20A; Column: Xbridge C18.5 μm 4.6*50 mm; Mobile phase: A: 0.02% NH₄OAc; B: CH₃CN; Gradient: 95% A for 0.01 min, to 40% A in 4.49 min, then to 5% A in 0.30 min, held for 1.00 min, back to 95% A in 0.20 min, held for 0.50 min; Flow rate & column temperature: 1.5 mL/min, 40° C.; Run time: 6.5 min.

Method 4: Instrument: SHIMA DZU SIL-20A; Column: Proshell-EC-C18, 5 μm 4.6*50 mm; Mobile phase: A: 0.05% TFA; B: CH₃CN; Gradient: 95% A for 0.01 min, to 60% A in 4.49 min, then to 5% A in 0.30 min, held for 1.00 min, back to 95% A in 0.20 min, held for 0.50 min; Flow rate & column temperature: 1.0 mL/min, 40° C.; Run time: 6.5 min.

Method 5: Instrument: Agilent Technologies 1200 Series, G6130A; Column: Xbridge C18, 3.5 μm 4.6*50 mm; Mobile phase: A: 0.05% TFA; B: CH₃CN; Gradient: 95% A for 0.50 min, to 5% A in 3.50 min, held for 1.50 min, back to 95% A in 0.10 min, held for 0.40 min; Flow rate & column temperature: 1.5 mL/min, 40° C.; Run time: 6.0 min.

### Example 5: Analytical Data

The analytical information in the Compounds above or in the Tables below.

| Compound No. | Rt (min) | [M + H]⁺ |
|---|---|---|
| 1 | 2.33 | 433.1 |
| 2 | 2.78 | 433.1 |
| 3 | 2.31 | 455.1 |
| 4 | 2.81 | 444.0 |
| 5 | 1.79 | 437.1 |
| 6 | 3.77 | 437.1 |
| 7 | 3.11 | 448.1 |
| 8 | 2.05 | 455.1 |
| 9 | 2.37 | 433.1 |
| 10 | 2.43 | 455.0 |
| 11 | 2.43 | 455.1 |
| 12 | 2.77 | 444.0 |
| 13 | 2.27 | 444.0 |
| 14 | 2.63 | 437.1 |
| 15 | 2.50 | 433.1 |
| 16 | 2.62 | 463.1 |
| 17 | 2.31 | 440.0 |
| 18 | 2.32 | 481.1 |
| 19 | 2.10 | 451.1 |
| 20 | 1.87 | 445.1 |
| 21 | 1.95 | 437.1 |
| 22 | 2.14 | 466.1 |
| 23 | 1.85 | 466.1 |
| 24 | 1.74 | 433.1 |
| 25 | 2.50 | 433.1 |
| 26 | 2.78 | 477.3 |
| 27 | 2.10 | 451.1 |
| 28 | 3.20 | 451.2 |
| 29 | 1.96 | 437.1 |
| 30 | 2.14 | 463.1 |
| 31 | 2.34 | 437.2 |
| 32 | 2.32 | 387.1 |
| 33 | 2.80 | 453.1 |
| 34 | 3.048 | 495.1 |
| 35 | 3.133 | 481.1 |
| 36 | 2.046 | 437.1 |
| 37 | 2.680 | 452.1 |
| 39 | 2.014 | 433.1 |
| 40 | 2.207 | 451.1 |
| 46 | 2.406 | 488.2 |
| 47 | 2.674 | 445.1 |
| 48 | 2.302 | 463.1 |
| 49 | 2.179 | 507.2 |
| 50 | 1.072 | 519.1 |
| 51 | 0.971 | 519.1 |
| 52 | 0.963 | 505.1 |
| 54 | 2.473 | 463.1 |
| 55 | 1.089 | 507.2 |
| 56 | 0.996 | 519.1 |
| 57 | 1.002 | 519.1 |
| 58 | 1.092 | 533.1 |
| 59 | 0.979 | 505.1 |
| 60 | 2.677 | 464.1 |
| 61 | 0.950 | 506.1 |
| 62 | 0.931 | 465.1 |
| 63 | 0.947 | 465.1 |
| 64 | 0.993 | 504.1 |
| 65 | 1.057 | 460.1 |
| 66 | 0.985 | 478.1 |
| 67 | 1.013 | 522.1 |
| 68 | 0.953 | 504.2 |
| 69 | 0.965 | 520.1 |
| 70 | 1.033 | 548.2 |
| 71 | 0.954 | 479.1 |
| 72 | 1.005 | 505.1 |
| 73 | 1.002 | 549.2 |
| 74 | 1.002 | 549.1 |
| 75 | 0.978 | 531.2 |
| 76 | 1.007 | 493.1 |
| 77 | 0.997 | 479.1 |
| 78 | 0.920 | 460.1 |
| 79 | 0.953 | 479.1 |
| 80 | 0.964 | 479.1 |
| 81 | 0.934 | 479.1 |
| 82 | 1.020 | 477.2 |
| 83 | 1.008 | 519.2 |
| 84 | 0.891 | 536.1 |
| 85 | 0.988 | 534.2 |
| 86 | 0.987 | 534.1 |
| 87 | 1.002 | 504.1 |
| 88 | 0.974 | 478.1 |
| 89 | 0.961 | 520.1 |
| 90 | 1.021 | 548.1 |
| 91 | 0.999 | 522.1 |

### Example 6: PARP1-DNA Enzymatic Trapping Assay

Enzymatic trapping assay is to measure the dissociation process of PARP1 from fluorescein-labeled DNA during the enzymatic activity. Assay was conducted at a total 10 μL of reaction volume, including 10 nM PARP1 and 5 nM DNA probe in the binding buffer (50 mM Tris-HCl pH 8.0, 50 mM NaCl, 1 mM MgCl₂, 0.1 mM EDTA, and 0.01% IGEPAL), and was commenced by adding a final concentration of 2 mM NAD+ substrate.

PARP1 Poly-ADP ribosylation (PARylation) inhibitory assay was conducted with compounds dissolved in DMSO at a starting concentration of 5 μM. Dissolved compounds were added to 384-well microplate and premixed with PARPI enzyme and DNA probe for 20 minutes incubation. Enzymatic reactions were started by adding NAD+ to mixture, and each well was read at 30 minutes by microplate reader (VICTOR Nivo®, PerkinElmer) to detect the fluorescence polarization signal at 480 nm excitation/530 nm dual emission.

All measured IC₅₀ values were calculated using four parameters dose-response inhibition model in GraphPad Prism 8.0.2 (La Jolla, California, USA, www.graphpad.com).

### Example 7: PARP2-FL Displacement Assay

A potent PARP inhibitor (PARPi-FL, Cat No. 6461, Tocris Bioscience) conjugated to BDY FL fluorophore was adopted to characterize the activity of PARP2 by measuring the displacement of binding of PARPi-FL to PARP2. Compounds to be tested were prepared at a concentration of 1-10 mM, and made a 3-fold serial dilution by 100% DMSO in a polypropylene plate (Bio-One small volume microplate, black, Cat No. 784076, Greiner). 100 nL of test compounds were dispensed into a 384-well polypropylene microplate and added 10 μL of reaction solution including premixed 20 nM PARP2 and 3 nM PARPi-FL. After 4 hours of incubation at room temperature, the fluorescence polarization signal at 480/30 nm excitation and 530/30 nm dual emission was measured by VICTOR Nivo® multimode plate reader (PerkinElmer). All of the measured IC₅₀ values were calculated using four parameters dose-response inhibition model in GraphPad Prism 8.0.2 (La Jolla, California, USA, www.graphpad.com).

### Example 8: Cell Proliferation Assay

For PARPi sensitivity, exponentially growing MDA-MB-436 and DLD1 BRCA2^{-/-} cells were seed in 96 or 384-well plates at very low density with the goal of not splitting for at least 7 days (typically 0.3k-1.2k cells/well). Cells were plated on Day 1 and treated with DMSO or increasing concentrations of PARP1 inhibitors on Day 0. At the end of the experiment, cell viability was estimated using Cell-Titer Glo (Promega).

The results for Examples 6 to 8 are shown in the table below.

| | PARP1- | PARP2 | MDA- | DLD1 |
|---|---|---|---|---|
| | DNA | FL | MB-436 | BRCA2^{-/-} |
| | enzymatic | dis- | anti- | anti- |
| Compound | trapping assay | placement | proliferation | proliferation |
| No. | (nM) | assay (nM) | assay (nM) | assay (nM) |
| 1 | 42.9 | N.A. | 410.2 | >500 |
| 2 | 10.7 | N.A. | 0.40 | 1.98 |
| 3 | 41.1 | N.A. | 15.7 | 45.3 |
| 4 | 7.93 | 925 | 0.90 | 0.58 |
| 5 | 111.3 | N.A. | 39.9 | 18.6 |
| 6 | 262.2 | N.A. | 76.1 | 49.3 |
| 7 | 24.8 | N.A. | 2.59 | 4.63 |
| 8 | 47.3 | N.A. | 5.13 | 13.9 |
| 9 | 10.8 | N.A. | 1.76 | 1.29 |
| 10 | 20.6 | N.A. | 7.84 | 4.92 |
| 11 | 323.1 | N.A. | >500 | >500 |
| 12 | 17.3 | N.A. | 5.03 | 3.68 |
| 13 | 46.3 | N.A. | 31.6 | 10.6 |
| 14 | 33.1 | N.A. | 9.85 | 5.59 |
| 15 | 6.25 | 1268 | 0.37 | 0.33 |
| 16 | >500 | N.A. | >500 | >500 |
| 17 | >500 | N.A. | >500 | 467.7 |
| 18 | 27.3 | N.A. | 11.4 | 12.7 |
| 19 | 6.13 | N.A. | 0.59 | 0.59 |
| 20 | 16.6 | N.A. | 3.47 | 4.12 |
| 21 | 17.8 | N.A. | 1.91 | 1.76 |
| 22 | 10.9 | N.A. | 0.69 | 1.09 |
| 23 | 31.1 | N.A. | 20.2 | 34.9 |
| 24 | >500 | N.A. | 406.7 | 370.8 |
| 25 | 20.5 | N.A. | 5.06 | 7.49 |
| 26 | 5.99 | N.A. | 0.69 | 1.60 |
| 27 | 7.52 | 1099 | 0.23 | 0.22 |
| 28 | 9.73 | N.A. | 3.93 | 5.02 |
| 29 | 10.9 | N.A. | 0.59 | 0.87 |
| 30 | 8.09 | N.A. | 0.37 | 0.45 |
| 31 | 8.35 | N.A. | 0.98 | 1.49 |
| 33 | 7.81 | N.A. | 2.48 | 4.13 |
| 34 | 10.5 | N.A. | 0.78 | 1.09 |
| 35 | 23.7 | N.A. | 3.00 | 4.14 |
| 36 | 8.44 | N.A. | 2.08 | 1.51 |
| 37 | 13.5 | N.A. | 4.63 | 5.98 |
| 39 | 9.74 | N.A. | 1.39 | 1.22 |
| 40 | 9.28 | 4046 | 0.62 | 0.77 |
| 46 | 8.21 | 2439 | 1.66 | 3.01 |
| 47 | 9.42 | N.A. | 1.10 | 0.77 |
| 48 | 12.0 | 2046 | 0.74 | 1.02 |
| 49 | 9.61 | 2110 | 1.03 | 2.16 |
| 50 | 7.86 | N.A. | 1.05 | 4.04 |
| 51 | 7.36 | N.A. | 0.70 | 2.96 |
| 52 | 7.41 | 3311 | 0.44 | 1.44 |
| 54 | 6.44 | 7097 | 0.45 | 0.54 |
| 55 | 6.96 | N.A. | 1.24 | 4.66 |
| 56 | 5.17 | N.A. | 0.71 | 3.37 |
| 57 | 18.9 | N.A. | 1.24 | 8.41 |
| 58 | 7.68 | N.A. | 2.51 | 12.9 |
| 59 | 5.17 | N.A. | 0.37 | 1.35 |
| 60 | 8.17 | 2474 | 1.04 | 1.60 |
| 61 | 9.14 | N.A. | 1.35 | 7.97 |
| 62 | 7.82 | N.A. | 1.53 | 5.86 |
| 63 | 101 | N.A. | 7.29 | 141 |
| 64 | 8.01 | 2898 | 0.24 | 0.55 |
| 65 | 6.22 | 334 | 0.36 | 0.63 |
| 66 | 4.87 | 219 | 0.12 | 0.52 |
| 67 | 10.4 | N.A. | 0.64 | 2.16 |
| 68 | 14.6 | N.A. | 0.60 | 12.3 |
| 69 | 2.92 | 676 | 0.24 | 0.81 |
| 70 | 4.20 | 614 | 0.39 | 0.70 |
| 71 | 5.05 | 500 | 0.18 | 0.44 |
| 72 | 9.52 | 832 | 0.35 | 2.11 |
| 73 | 8.29 | 1041 | 0.27 | 1.09 |
| 74 | 8.26 | N.A. | 0.57 | 4.98 |
| 75 | 8.60 | N.A. | 0.93 | 10.2 |
| 76 | 5.30 | 574 | 0.50 | 0.70 |
| 77 | 7.73 | 1629 | 0.41 | 2.70 |
| 78 | 26.7 | N.A. | 1.41 | 9.14 |
| 79 | 9.57 | 4848 | 1.08 | 2.51 |
| 80 | 5.05 | 3881 | 0.84 | 4.75 |
| 81 | 7.60 | N.A. | 1.05 | 4.66 |
| 82 | 13.1 | 185 | 1.38 | 0.55 |
| 83 | 7.66 | 147 | 0.54 | 0.54 |
| 84 | 411 | N.A. | N.A. | N.A. |
| 85 | 8.15 | 811 | 1.05 | 1.10 |
| 86 | 11.7 | 2370 | 2.25 | 1.37 |
| 87 | 5.73 | 1195 | 0.66 | 0.62 |
| 88 | 7.90 | N.A. | 1.42 | 4.56 |
| 89 | 4.32 | N.A. | 1.08 | 8.28 |
| 90 | 5.16 | N.A. | 1.74 | 8.30 |

### Example 9: Efficacy Study of Tumor/Plasma Ratio in Mouse

1×10⁷ MDA-MB-436 cells were implanted subcutaneously onto the flank of female NOD-SCID mice (aged 6-8 weeks weighing approximately 18-22 g). When tumors reached approximately 150-180 mm3, the mice were randomly assigned to treatment groups. The mice were continuously dosed with the Compounds for 28 days. On day 29, blood (50 μL) and tumor tissue samples were collected at 24 hours after the last dose. The collected blood samples were then centrifuged at 4600 rpm for 5 minutes at 4° C. to obtain plasma. To determine compound levels in plasma samples, each plasma sample was prepared using an appropriate dilution factor and compared against an 11-point standard calibration curve (1-5,000 ng/mL) prepared in DMSO and spiked into blank plasma. Acetonitrile (200 μL) was added with internal standard into 20 μL plasma samples, followed by centrifugation at 5,500 rpm for 10 minutes. Supernatant (150 μL) was then diluted in water (150 μL) and analyzed via LC-MS/MS. Tumor tissue samples were weighed, and then homogenized with 80% methanol at a ratio of 1:5 or 1:10 (tumor weight in grams to methanol volume in milliliters). Acetonitrile (300 μL) was added with internal standard into 30 μL tumor homogenates sample. The samples were vortexed for 30 s and then centrifuged at 5,500 rpm for 10 min at 4° C. The supernatant (150 μL) was transferred to 96-well plate and diluted with water (150 μL) and analyzed via LC-MS/MS. If a sample concentration in initial assay was not covered in the range of a given standard curve, proper dilution and concentration procedures were used in repeated testing. The results are shown in the Table below. Compared with the reference compound (AZD5305), the compounds described herein demonstrated a significantly higher tumor/plasma ratio, indicative of better tumor penetration and retention.

| Compound | Plasma (ng/ml) | Tumor (ng/g) | Tumor/plasma Ratio |
|---|---|---|---|
| 52 | 4.3 | 121 | 28 |
| 73 | 3.2 | 206 | 64 |
| 87 | 22 | 244 | 11 |
| 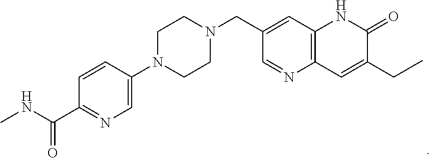 [SMILES: CCc1cc2ncc(CN3CCN(c4ccc(C(=O)NC)nc4)CC3)cc2[nH]c1=O] AZD5305 | 584 | 249 | 0.4 |

The embodiments described above are intended to be merely exemplary, and those skilled in the art will recognize, or will be able to ascertain using no more than routine experimentation, numerous equivalents of specific compounds, materials, and procedures. All such equivalents are considered to be within the scope of the invention and are encompassed by the appended claims.

What is claimed is:

1. A compound of Formula (I): 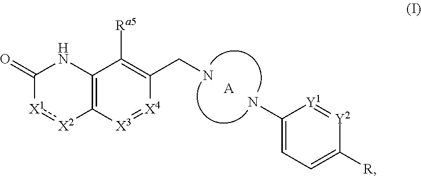
or a stereoisomer, or a mixture of stereoisomers thereof, or a pharmaceutically acceptable salt thereof, wherein: [symbol]
is a single bond or a double bond;
X¹ is CR^{a1}, C(R^{a1})₂, or NR^{a1};
X² is CR^{a2}, C(R^{a2})₂, N, NR^{a2}, or O;
each R^{a1} is independently hydrogen, C₁-C₆ alkyl, C₃-C₈ cycloalkyl, or C₁-C₆ alkoxy;
each R^{a2} is independently hydrogen, C₁-C₆ alkyl, C₃-C₈ cycloalkyl, or C₁-C₆ alkoxy;
or R^{a1} and R^{a2} together with the atoms to which they are attached to form a 3- to 6-membered Ring B;
X³ is CR^{a3} or N; R^{a3} is hydrogen, C₁-C₆ alkyl, or halogen;
X⁴ is CR^{a4} or N; R^{a4} is hydrogen, C₁-C₆ alkyl, or halogen;
R^{a5} is hydrogen, C₁-C₆ alkyl, or halogen;
Ring A is a fused, bridged or spiro heterocyclyl;
Y¹ is CR² or N;
Y² is CR² or N;
each R² is independently hydrogen, halogen, C₁-C₆ alkyl, C₁-C₆ haloalkyl, C₁-C₆ alkoxy, or C₃-C₈ cycloalkyl;
R is a halogen, —CN, C₁-C₆ alkyl, C₁-C₆ alkoxy, —C(═O)—R³, —C(═O)—OR³, —C(═O)—NHR³, —S(O)₂—R³, —NH—C(═O)—R³, or nitro;
R³ is hydrogen, C₁-C₆ alkyl, (C₁-C₆ alkoxy)-(C₁-C₆ alkyl), C₃-C₈ cycloalkyl, or 4- to 10-membered heterocyclyl; and
wherein the alkyl, cycloalkyl, alkoxy, heterocyclyl, Ring A, and Ring B are optionally substituted;
provided that, when 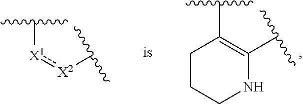
then (i) Ring A is a fused or spiro heterocyclyl; or (ii) R is —C(═O)—NHR³.

2. The compound of claim 1, wherein Y¹ is CR² and Y² is N.

3. The compound of claim 1, wherein Y¹ is N and Y² is CR².

4. The compound of claim 1, wherein Y¹ is CR² and Y² is CR².

5. The compound of any one of claims 1 to 4, wherein R is —C(═O)—NHR³.

6. The compound of any one of claims 1 to 4, wherein R is —CN.

7. The compound of claim 1, which is a compound of Formula (I-A): 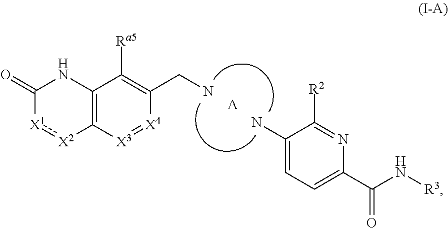
or a stereoisomer, or a mixture of stereoisomers thereof, or a pharmaceutically acceptable salt thereof.

8. The compound of any one of claims 1 to 7, wherein [symbol] is a double bond.

9. The compound of claim 8, wherein X¹ is CR^{a1} and X² is N.

10. The compound of any one of claims 1 to 7, wherein [symbol] is a single bond.

11. The compound of claim 10, wherein X¹ is CH(R^{a1}) and X² is O.

12. The compound of claim 10, wherein X¹ is CH(R^{a1}) and X² is NR^{a2}.

13. The compound of any one of claims 1 to 12, wherein R^{a1} is C₁-C₄ alkyl or C₁-C₄ fluoroalkyl.

14. The compound of claim 13, wherein R^{a1} is methyl, ethyl, isopropyl, cyclopropyl, 1,1-difluoroethyl, 1-fluoroethyl, trifluoromethyl, difluoromethyl, or methoxy.

15. The compound of claim 14, wherein R^{a1} is methyl or ethyl.

16. The compound of any one of claims 1 to 7, wherein R^{a1} and R^{a2} together with the atoms to which they are attached to form a 3- to 6-membered Ring B.

17. The compound of claim 16, which is a compound of Formula (I-B), (I-C), or (I-D): 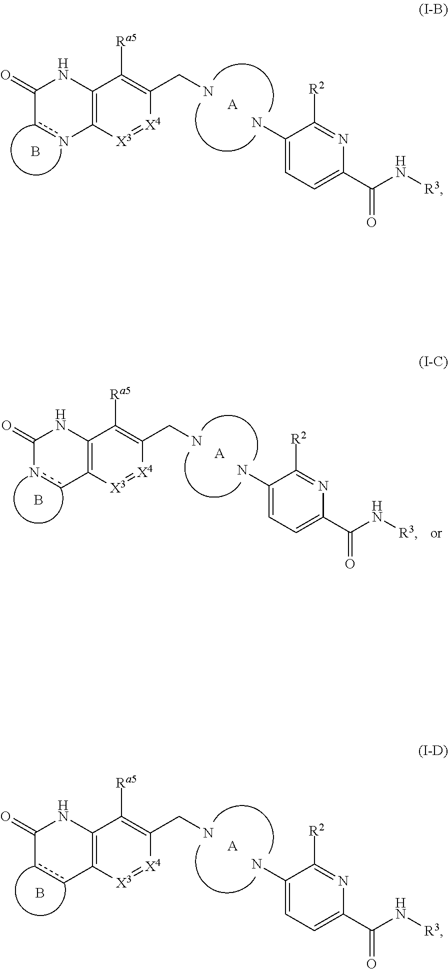
or a stereoisomer, a mixture of stereoisomers, or a pharmaceutically acceptable salt thereof.

18. The compound of claim 16, wherein Ring B is a 4- to 6-membered heterocyclyl.

19. The compound of claim 18, wherein the heterocyclyl is an oxygen-containing heterocyclyl.

20. The compound of claim 16, wherein Ring B is a 5-membered heteroaryl.

21. The compound of claim 16, wherein Ring B is a 5- or 6-membered cycloalkenyl.

22. The compound of any one of claims 1 to 21, wherein Ring B is unsubstituted.

23. The compound of any one of claims 1 to 22, wherein X³ is CR^{a3}.

24. The compound of any one of claims 1 to 22, wherein X³ is N.

25. The compound of any one of claims 1 to 24, wherein X⁴ is CR^{a4}.

26. The compound of any one of claims 1 to 24, wherein X⁴ is N.

27. The compound of claim 1, which is a compound of Formula (II-A), (II-B), (II-C), (II-D), (II-E), (II-F), (II-G), (II-H), (II-I), (II-J), (II-K), (II-L), (II-M), (II-N), (II-O), (II-P), (II-Q), (II-R), (II-S), (II-T), (II-U), (II-V), (II-W), (II-X), (II-Y), (II-Z), (II-AA), (II-AB), (II-AC), (II-AD), (II-AE), (II-AF), (II-AG), (II-AH), (II-AI), or (II-AJ): 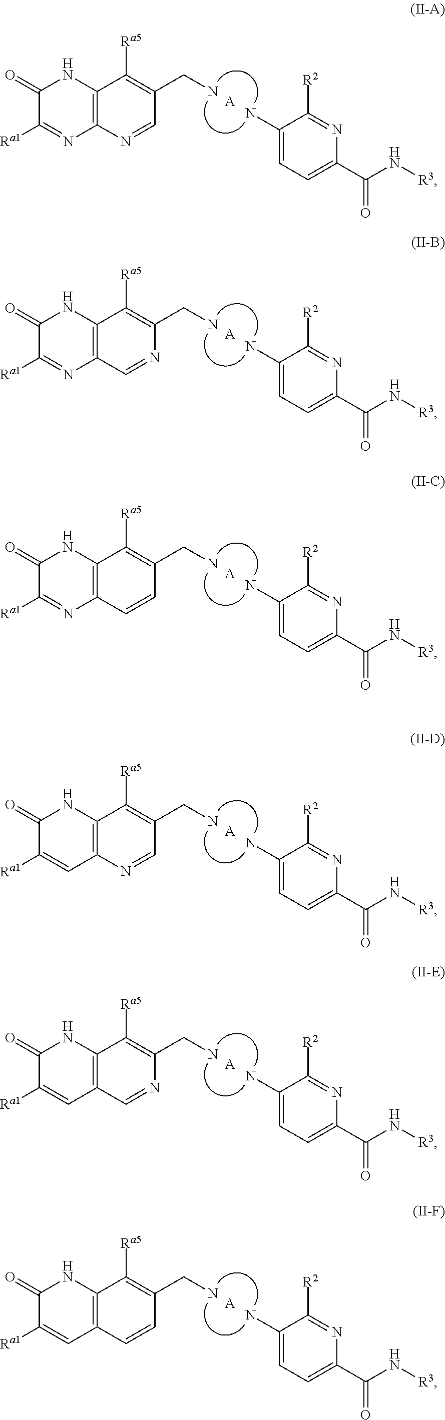 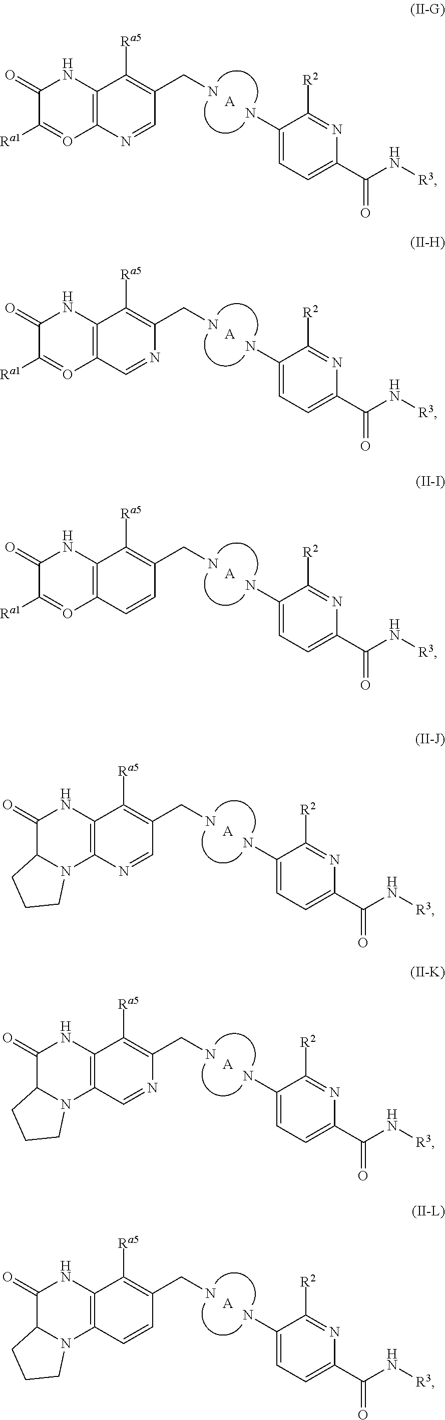 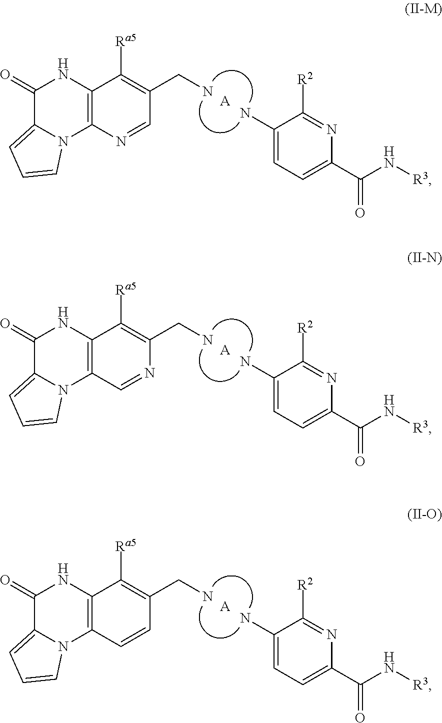 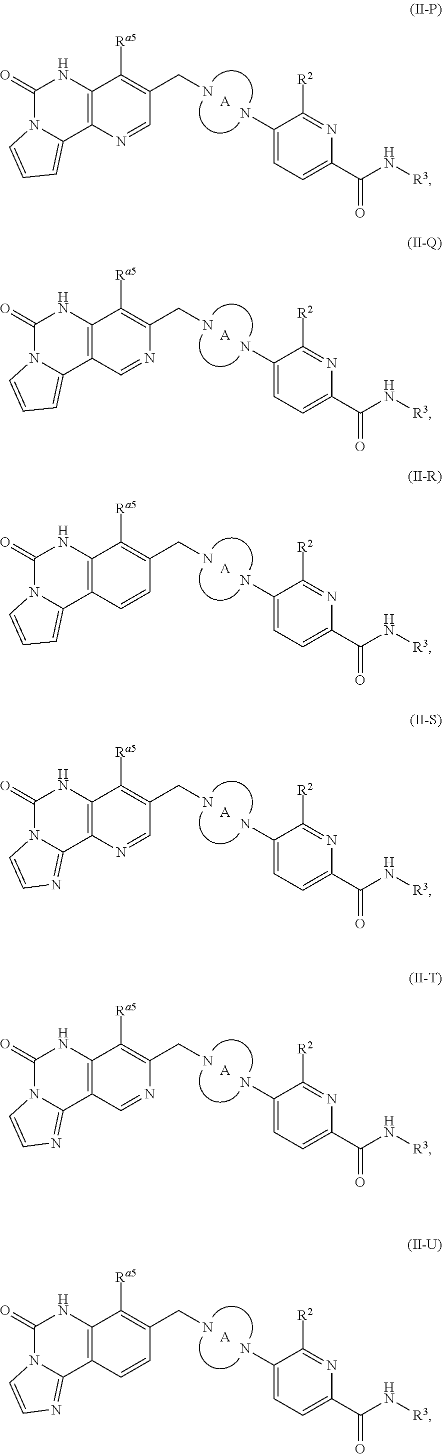 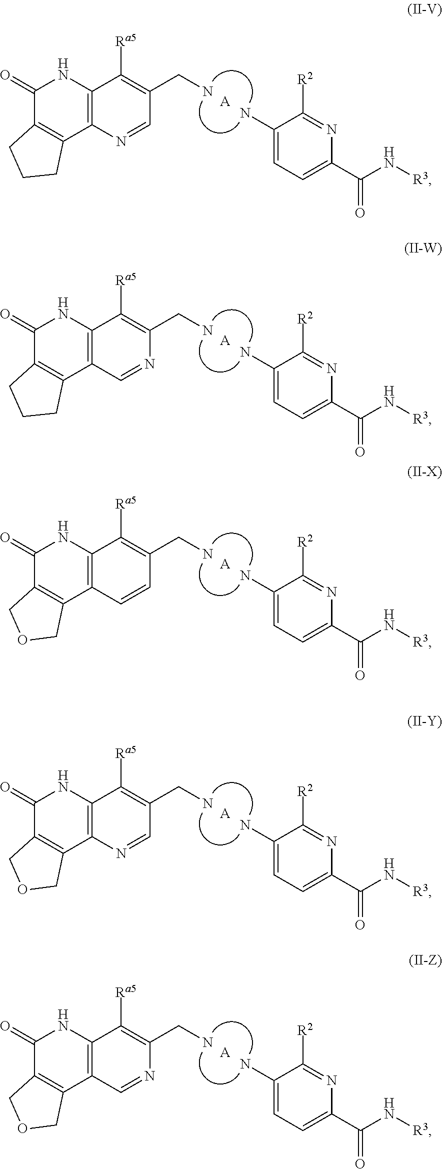 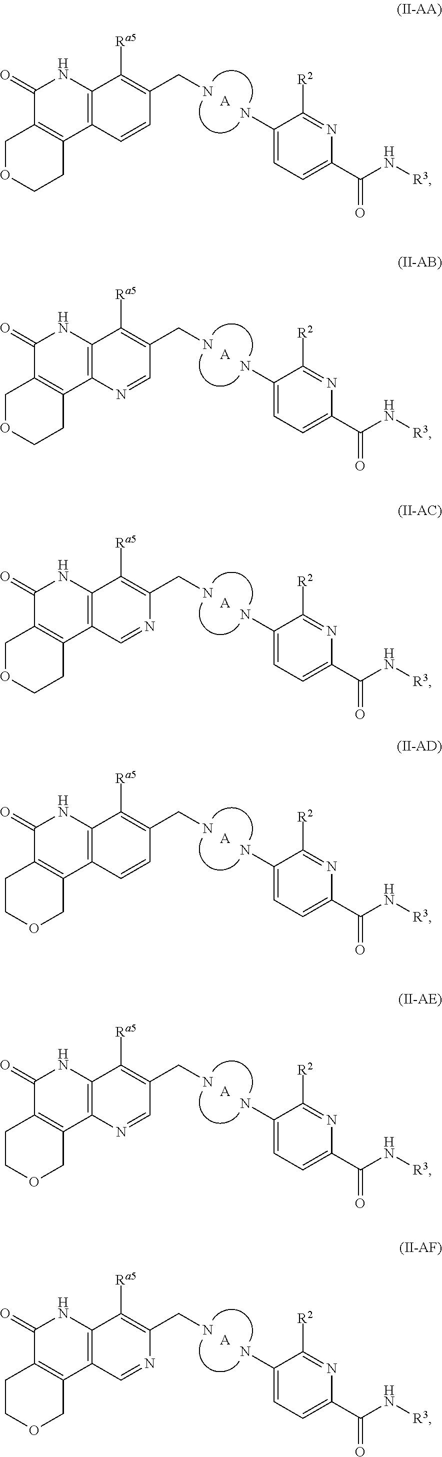 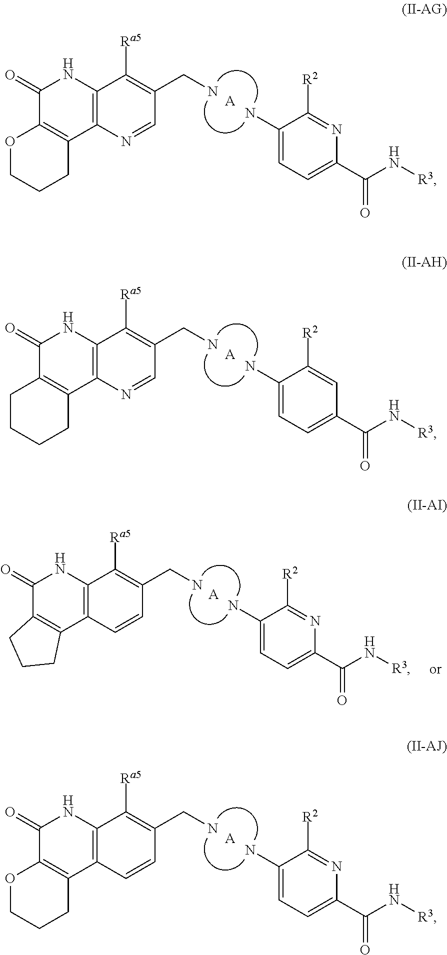
or a stereoisomer, a mixture of stereoisomers, or a pharmaceutically acceptable salt thereof.

28. The compound of any one of claims 1 to 27, wherein R^{a5} is hydrogen.

29. The compound of any one of claims 1 to 27, wherein R^{a5} is fluoro.

30. The compound of any one of claims 1 to 29, wherein Ring A is a fused bicyclic heterocyclyl.

31. The compound of any one of claims 1 to 29, wherein Ring A is a spiro bicyclic heterocyclyl.

32. The compound of any one of claims 1 to 29, wherein Ring A is a bridged bicyclic heterocyclyl.

33. The compound of any one of claims 1 to 32, wherein Ring A contains only two heteroatoms that are both nitrogen.

34. The compound of claim 33, wherein Ring A is 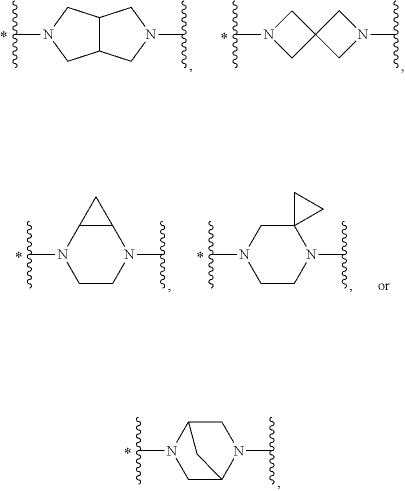 [SMILES: *N1CC2CN(*)CC2C1 ; *N1CC2(C1)CN(*)C2 ; *N1CCN(*)C2CC21 ; *N1CCN(*)C2(CC2)C1 ; *N1CC2CC1CN2*]
wherein * is toward the direction of the methylene group linking Ring A and the ring containing X⁴.

35. The compound of any one of claims 1 to 34, wherein Ring A is unsubstituted.

36. The compound of claim 1, which is a compound of Formula (III-A), (III-B), (III-C), (III-D), (III-E), (III-F), (III-G), (III-H), (III-I), (III-J), (III-K), (III-L), (III-M), (III-N), (III-O), (III-P), (III-Q), (III-R), (III-S), (III-T), (III-U), (III-V), (III-W), (III-X), (III-Y), (III-Z), (III-AA), (III-AB), (III-AC), (III-AD), (III-AE), (III-AF), (III-AG), (III-AH), (III-AI), or (III-AJ): 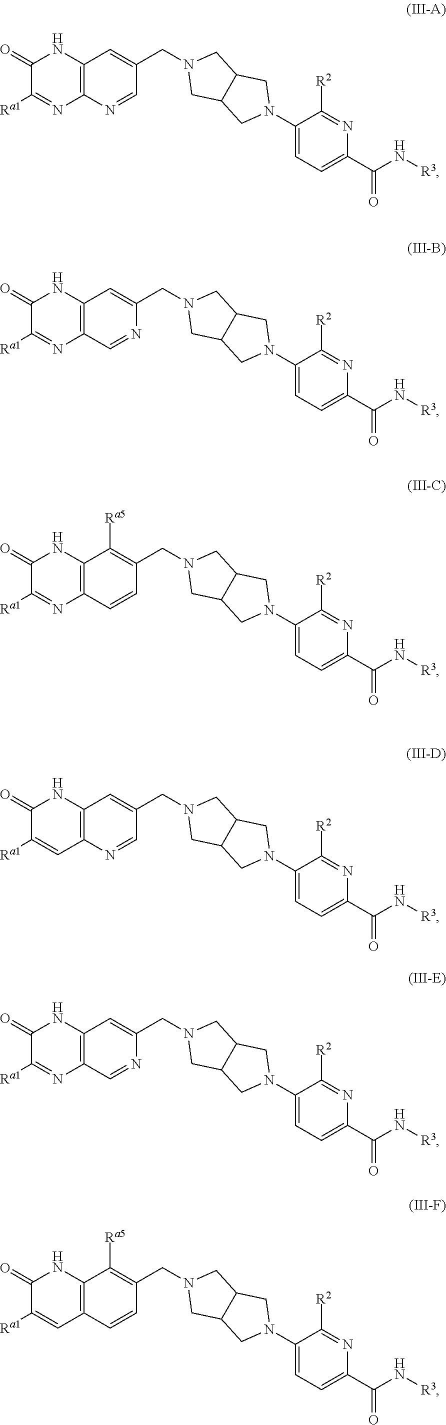 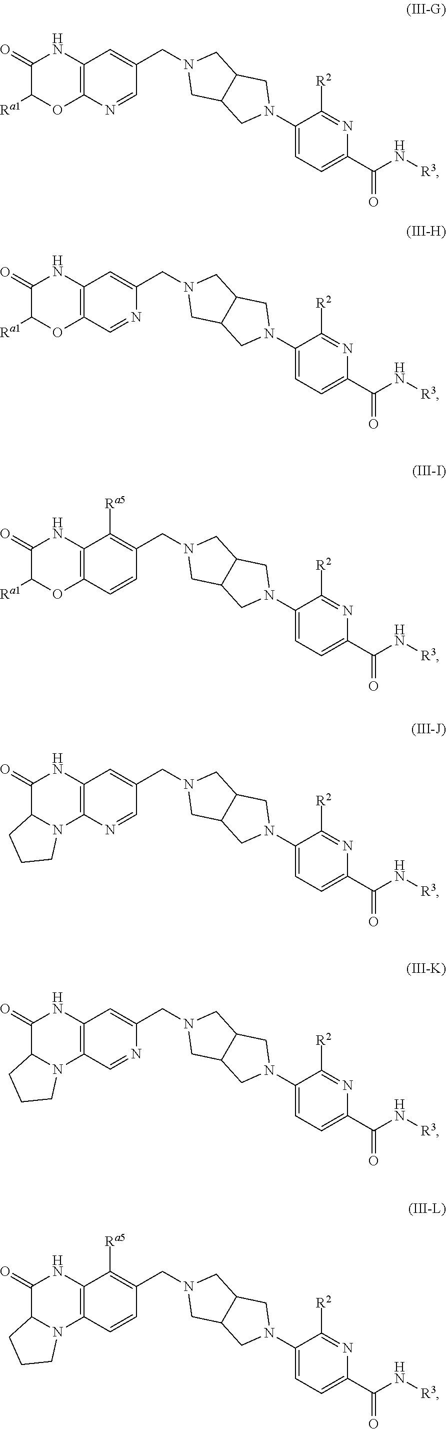 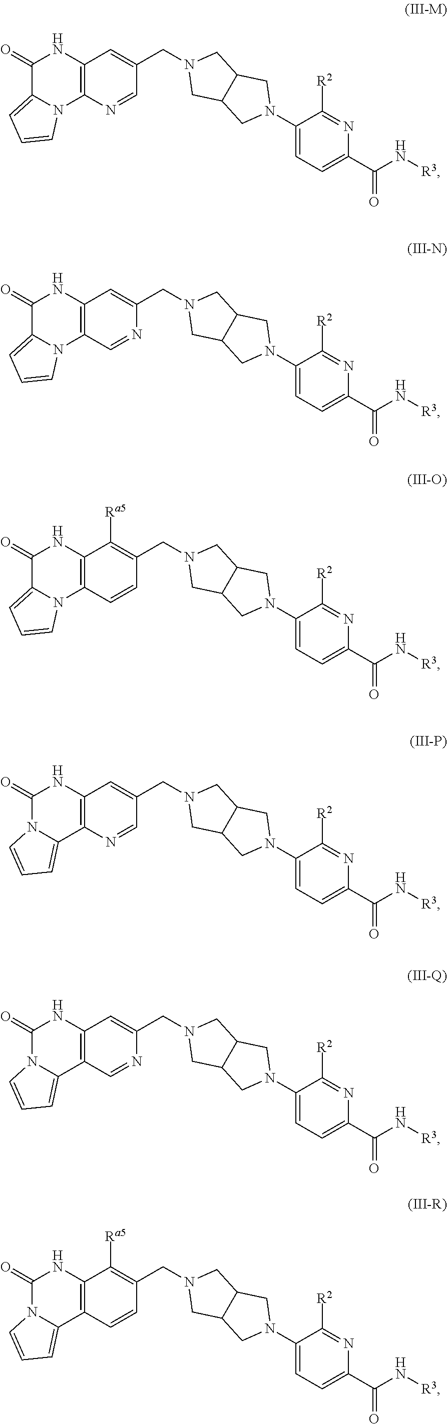 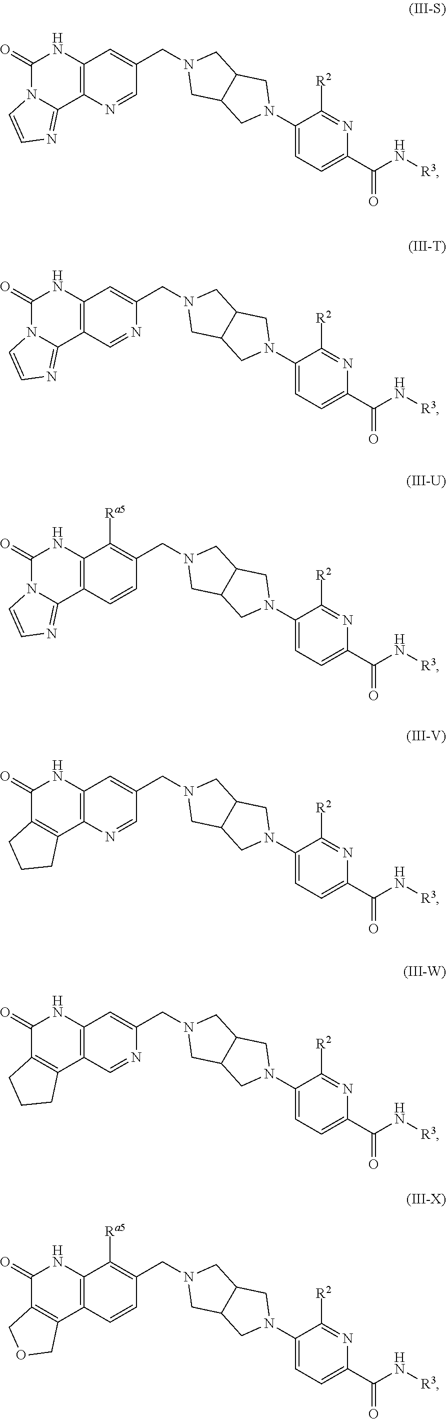 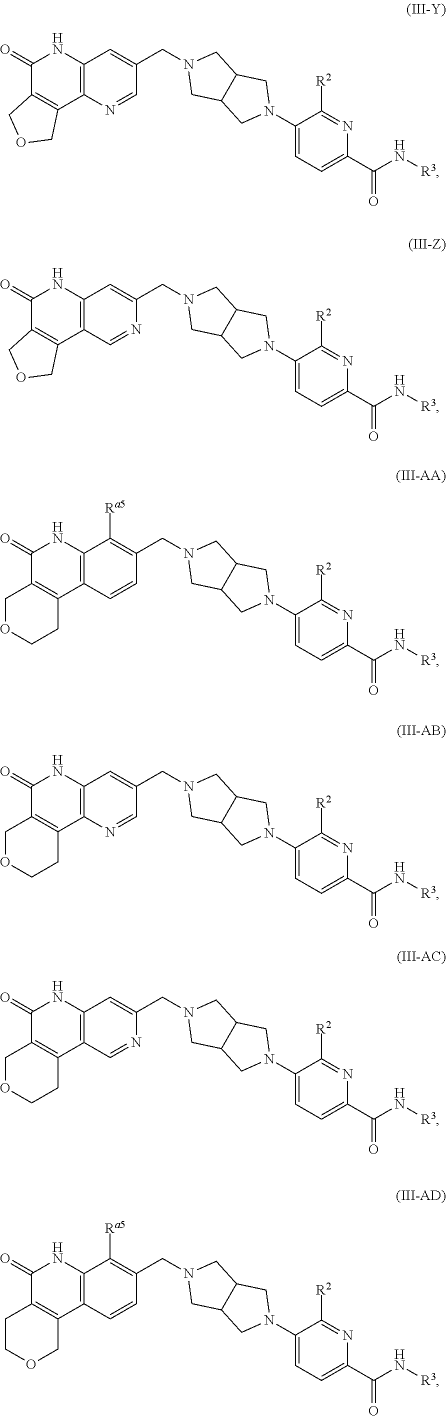 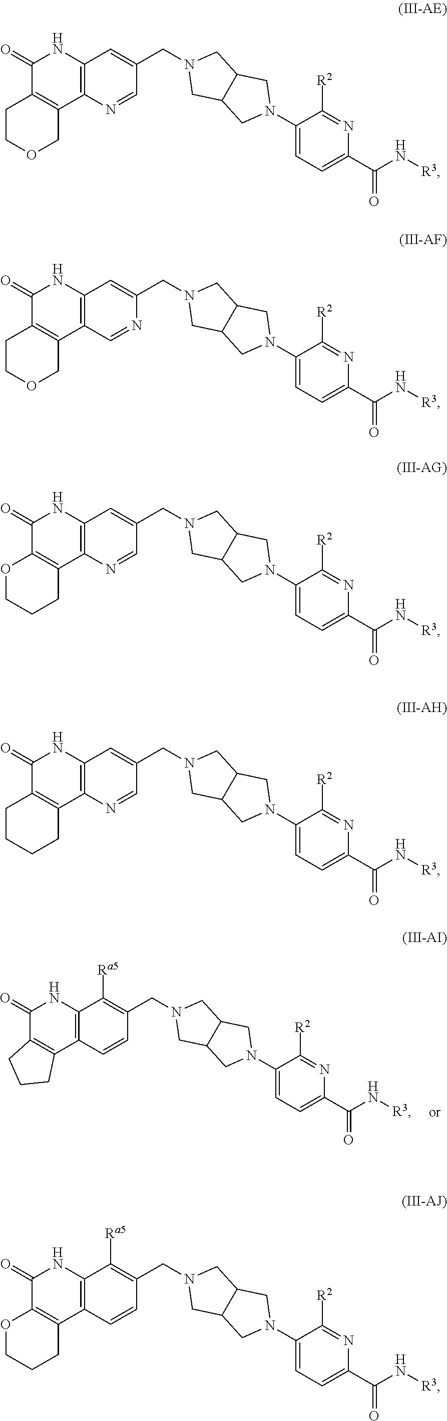
or a stereoisomer, a mixture of stereoisomers, or a pharmaceutically acceptable salt thereof.

37. The compound of any one of claims 1 to 36, wherein Ring A is cis-

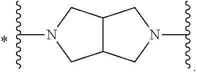 [SMILES: *N1CC2CN(*)CC2C1]

38. The compound of any one of claims 1 to 36, wherein Ring A is trans-

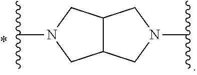 [SMILES: *N1CC2CN(*)CC2C1]

39. The compound of claim 38, wherein Ring A is

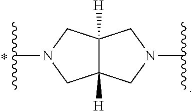 [SMILES: *N1C[C@H]2CN(*)C[C@@H]2C1]

40. The compound of claim 38, wherein Ring A is

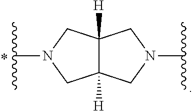 [SMILES: *N1C[C@H]2CN(*)C[C@@H]2C1]

41. The compound of claim 1, which is a compound of Formula (IV-A1), (IV-B1), (IV-C1), (IV-D1), (IV-E1), (IV-F1), (IV-G1), (IV-H1), (IV-11), (IV-J1), (IV-K1), (IV-L1), (IV-M1), (IV-N1), (IV-O1), (IV-P1), (IV-Q1), (IV-R1), (IV-S1), (IV-T1), (IV-U1), (IV-V1), (IV-W1), (IV-X1), (IV-Y1), (IV-Z1), (IV-AA1), (IV-AB1), (IV-AC1), (IV-AD1), (IV-AE1), (IV-AF1), (IV-AG1), (IV-AH1), (IV-AI1), or (IV-AJ1): 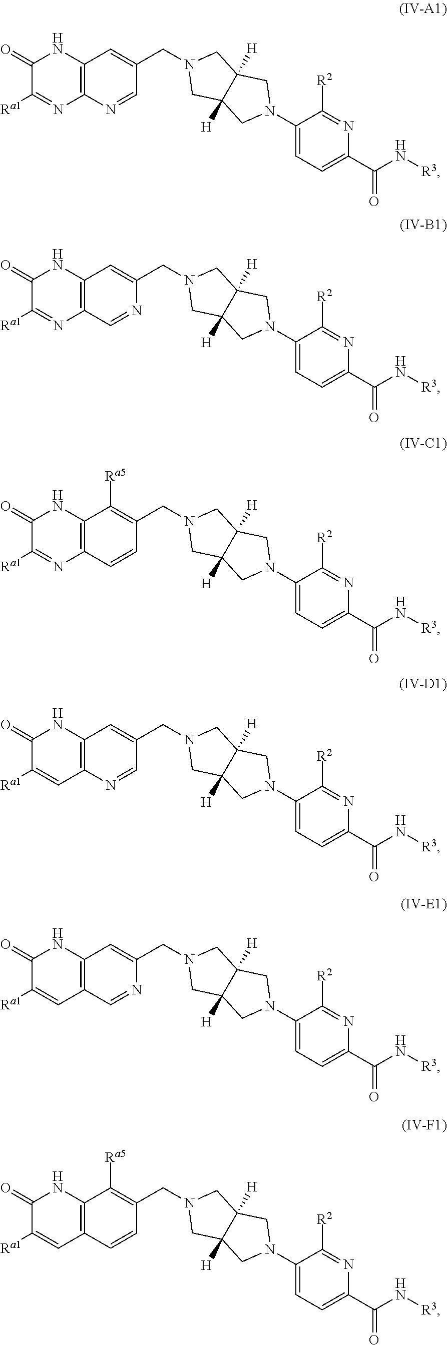 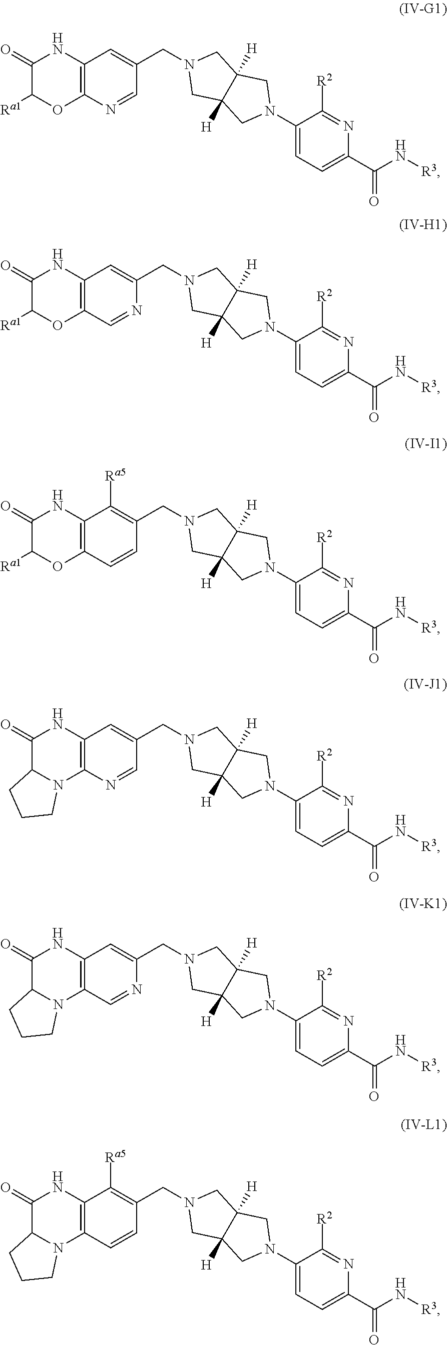 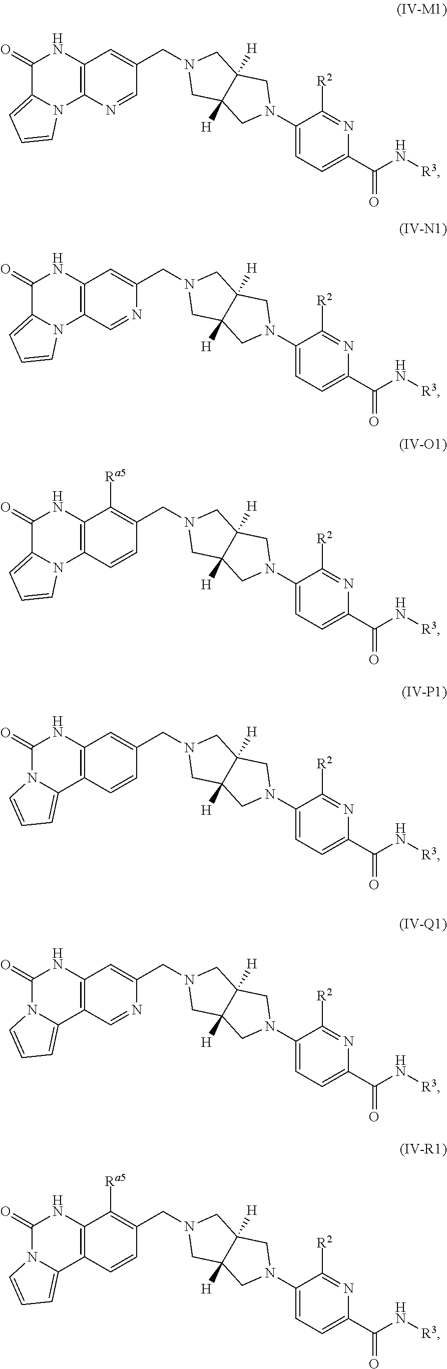 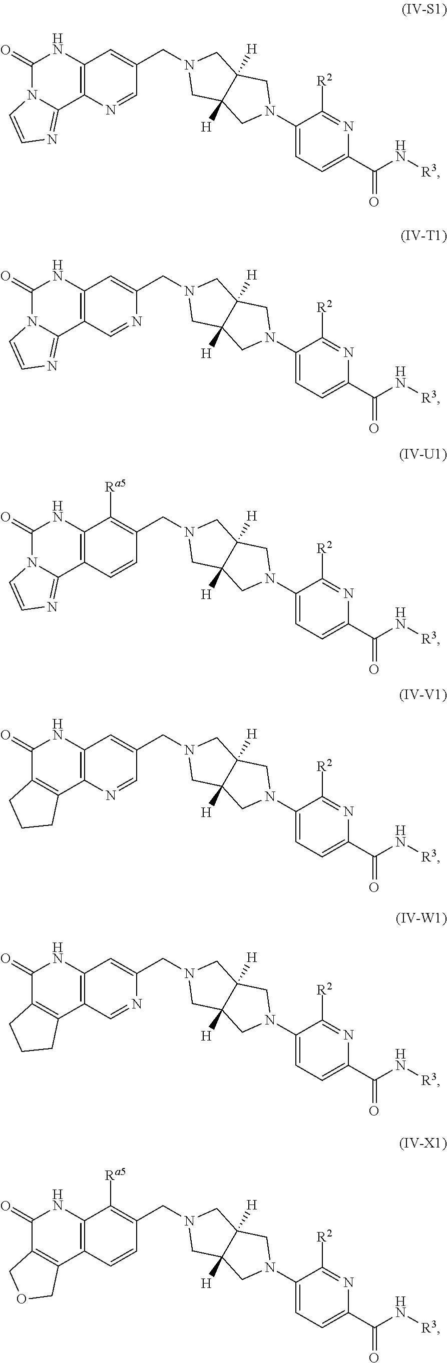 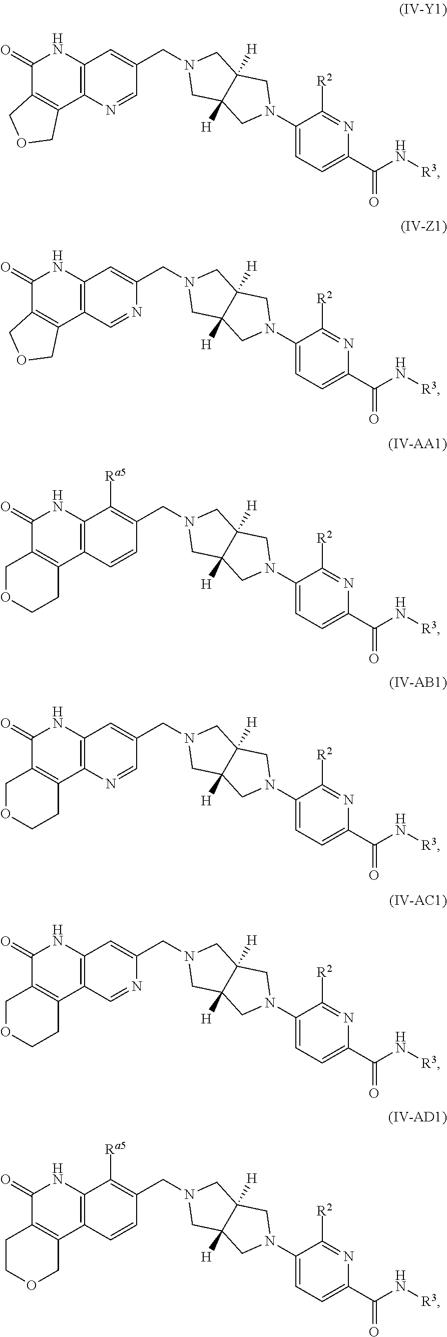 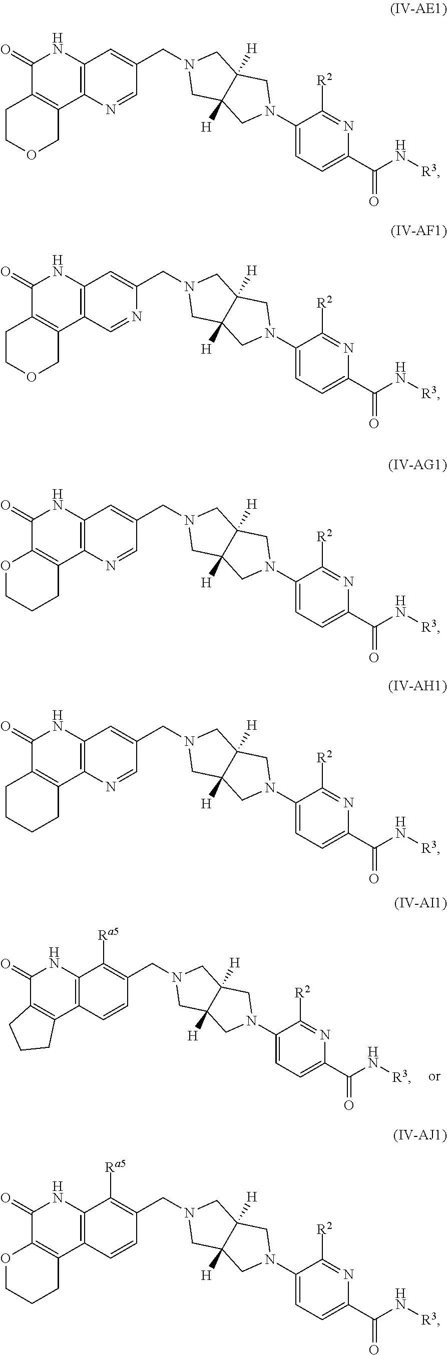
or a stereoisomer, a mixture of stereoisomers, or a pharmaceutically acceptable salt thereof.

42. The compound of claim 1, which is a compound of Formula (IV-A2), (IV-B2), (IV-C2), (IV-D2), (IV-E2), (IV-F2), (IV-G2), (IV-H2), (IV-12), (IV-J2), (IV-K2), (IV-L2), (IV-M2), (IV-N2), (IV-02), (IV-P2), (IV-Q2), (IV-R2), (IV-S2), (IV-T2), (IV-U2), (IV-V2), (IV-W2), (IV-X2), (IV-Y2), (IV-Z2), (IV-AA2), (IV-AB2), (IV-AC2), (IV-AD2), (IV-AE2), (IV-AF2), (IV-AG2), (IV-AH2), (IV-AI2), or (IV-AJ2): 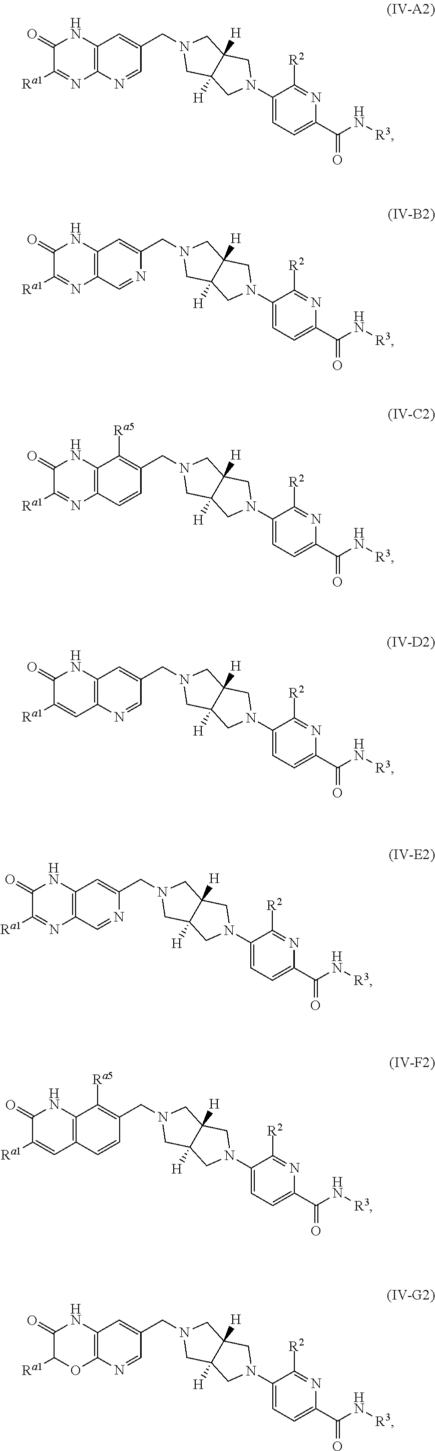 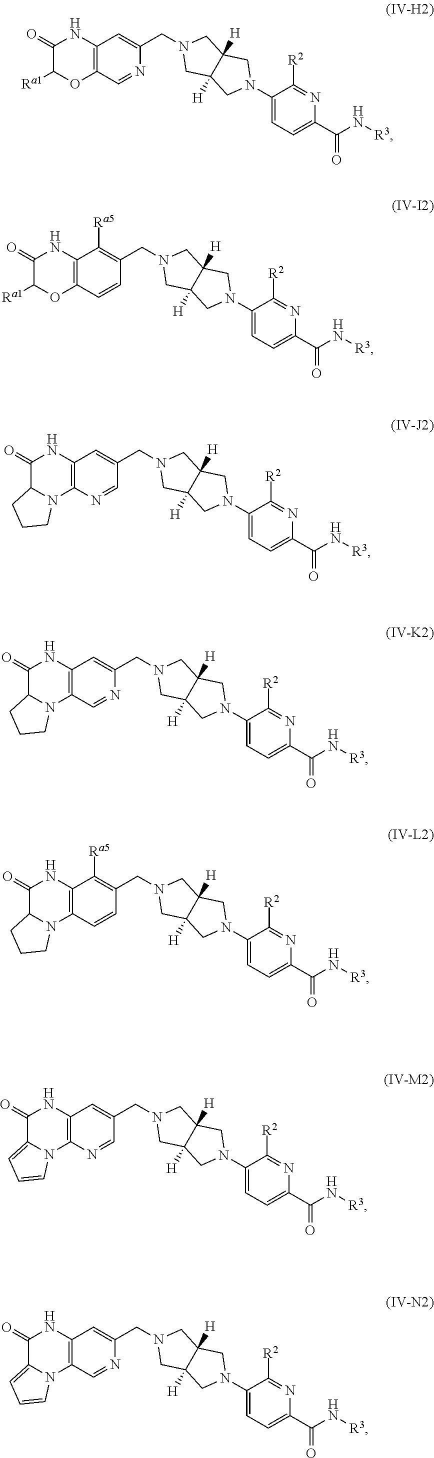 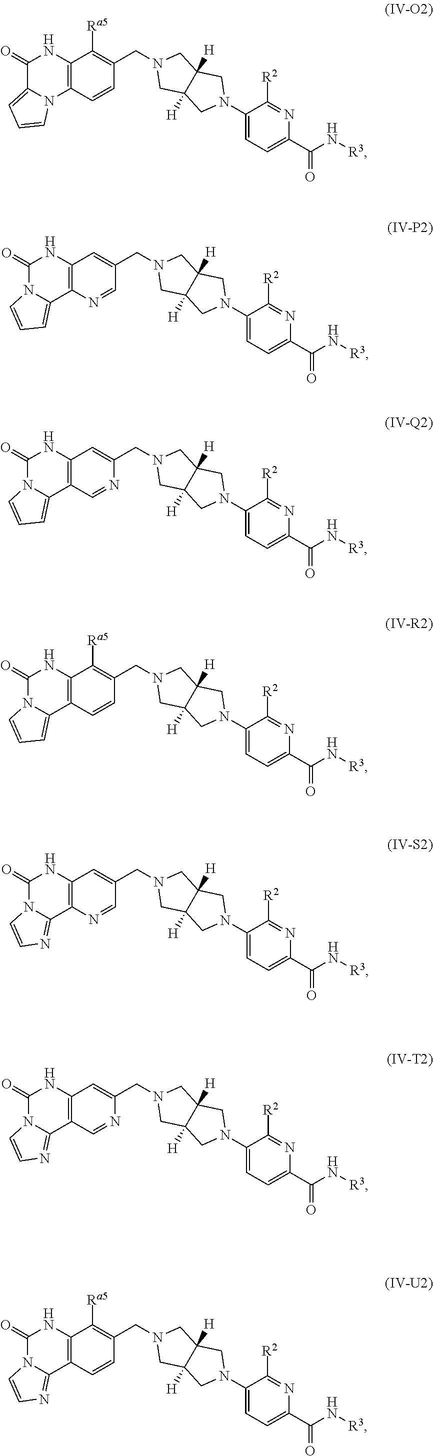 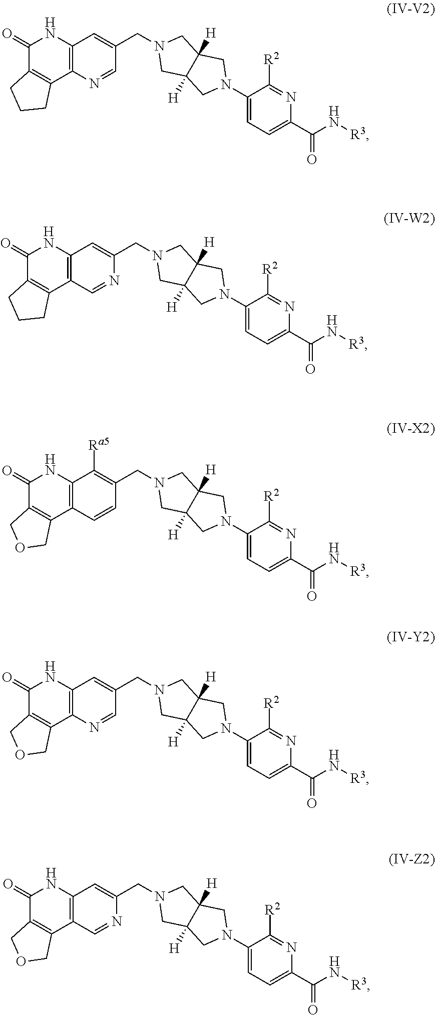 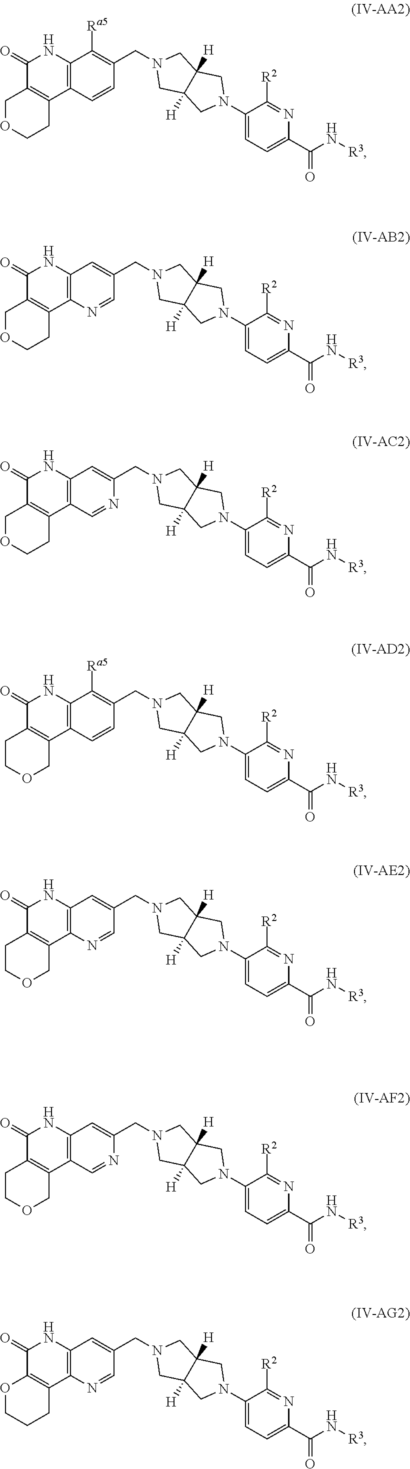 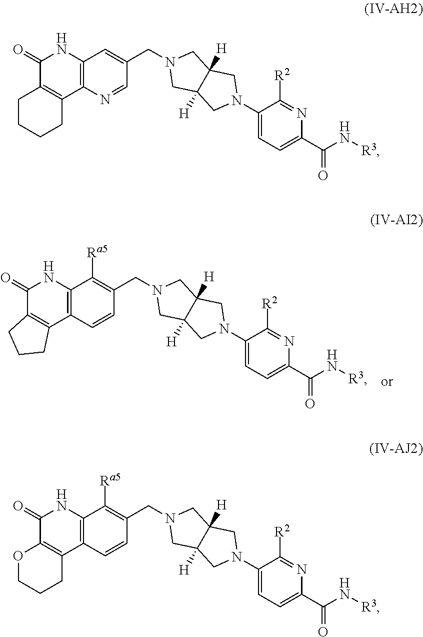
or a stereoisomer, a mixture of stereoisomers, or a pharmaceutically acceptable salt thereof.

43. The compound of claim 42, which is a compound of Formula (IV-V2-1), (IV-AA2-1), (IV-AB2-1), or (IV-AI2-1): 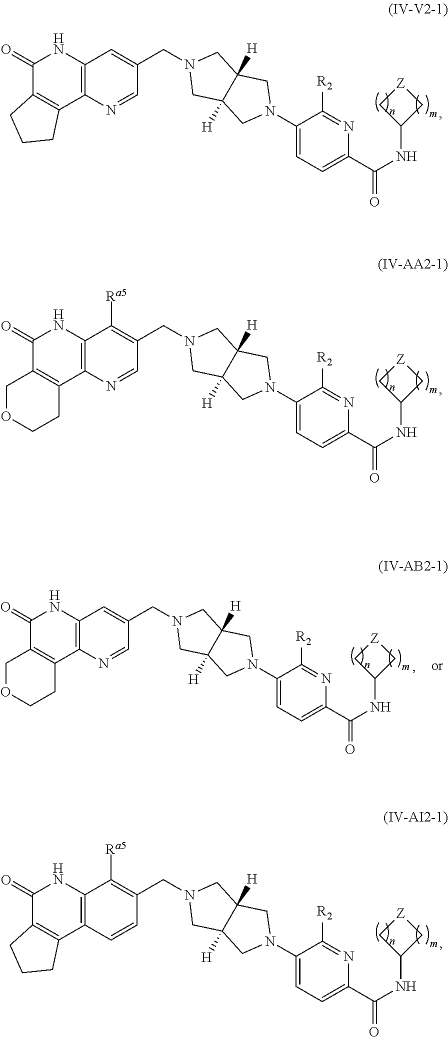
or a stereoisomer, a mixture of stereoisomers, or a pharmaceutically acceptable salt thereof, wherein:
m is 0, 1, 2, 3, or 4;
n is 1, 2, 3, or 4;
Z is O or C(R⁴)₂; and
each R⁴ is independently hydrogen, C₁-C₆ alkyl, or C₁-C₆ alkoxy.

44. The compound of claim 43, wherein Z is O.

45. The compound of claim 43, wherein Z is CHR⁴, and R⁴ is C₁-C₃ alkoxy.

46. The compound of any one of claims 1 to 45, wherein R² is hydrogen, halogen, C₁-C₄ alkyl, C₁-C₄ fluoroalkyl, or C₃-C₆ cycloalkyl.

47. The compound of claim 46, wherein R² is hydrogen, chloro, fluoro, methyl, difluoromethyl, trifluoromethyl, or cyclopropyl.

48. The compound of any one of claims 1 to 47, wherein R³ is hydrogen, C₁-C₄ alkyl, (C₁-C₄ alkoxy)-(C₁-C₄ alkyl), C₃-C₆ cycloalkyl, or 4- to 8-membered heterocyclyl.

49. The compound of claim 48, wherein R³ is a 4- to 6-membered heterocyclyl.

50. The compound of claim 49, wherein the heterocyclyl is an oxygen containing heterocyclyl.

51. The compound of claim 48, wherein R³ is methyl, 2-methoxyethyl, cyclopropyl, cyclobutyl, 3-methoxycyclobutyl, oxetan-3-yl, tetrahydrofuran-3-yl, or tetrahydro-2H-pyran-4-yl.

52. The compound of any one of claims 1 to 51, wherein R³ is substituted with halogen, C₁-C₆ alkyl, or C₁-C₆ alkoxy.

53. The compound of any one of claims 1 to 52, wherein when a carbon at X¹ position is a chiral center, it has the S-configuration.

54. A compound in Table 1, or a pharmaceutically acceptable salt thereof.

55. A pharmaceutical composition, comprising a compound of any one of claims 1 to 54 and a pharmaceutically acceptable excipient.

56. A method of treating a cancer, comprising administering to a subject having the cancer a therapeutically effective amount of a compound of any one of claims 1 to 54 or a pharmaceutical composition of claim 55.

57. The method of claim 56, wherein the cancer is deficient in HR dependent DNA DSB repair pathway.

58. The method of claim 56, wherein the cancer comprises one or more cancer cells having a reduced or abrogated ability to repair DNA DSB by HR relative to normal cells.

59. The method of claim 58, wherein the cancer cells have a BRCA1 or BRCA2 deficient phenotype.

60. The method of claim 59, wherein the cancer cells are deficient in BRCA1 or BRCA2.

61. The method of any one of claims 56 to 60, wherein the subject is heterozygous for a mutation in a gene encoding a component of the HR dependent DNA DSB repair pathway.

62. The method of claim 61, wherein the subject is heterozygous for a mutation in BRCA1 or BRCA2.

63. The method of any one of claims 56 to 62, wherein the cancer is breast cancer, ovarian cancer, pancreas cancer, prostate cancer, hematological cancer, gastrointestinal cancer, lung cancer, or brain cancer.

64. A method of inhibiting PARP1 protein, comprising contacting the PARP1 protein with an effective amount of a compound of any one of claims 1 to 54 or a pharmaceutical composition of claim 55.

65. The method of claim 64, wherein the inhibition occurs in a subject suffering from a PARP1 mediated disease or condition.

66. The method of claim 65, wherein the disease or condition is cancer.

67. The method of claim 66, wherein the cancer is breast cancer, ovarian cancer, pancreas cancer, prostate cancer, hematological cancer, gastrointestinal cancer, lung cancer, or brain cancer.
